(19) United States
(12) Patent Application Publication
(10) Pub. No.: US 2024/0301458 A1
(43) Pub. Date: Sep. 12, 2024

(54) Use of Terminal Transferase Enzyme in Nucleic Acid Synthesis

(71) Applicant: Nuclera Ltd., Cambridge (GB)

(72) Inventors: Michael C. CHEN, Cambridge (GB); Jiahao HUANG, Cambridge (GB); Gordon MCINROY, Cambridge (GB)

(21) Appl. No.: 18/629,077

(22) Filed: Apr. 8, 2024

Related U.S. Application Data
(63) Continuation of application No. 16/616,633, filed on Nov. 25, 2019, now Pat. No. 11,993,800.

(30) Foreign Application Priority Data
May 26, 2017 (GB) 1708503.6
May 30, 2017 (GB) 1708551.5

(51) Int. Cl. C12P 19/34; C12N 9/12

(52) U.S. Cl. CPC C12P 19/34; C12N 9/1264; C12Y 207/07031

(57) ABSTRACT

The invention relates to the use of a modified terminal transferase enzyme in a method of adding one or more nucleotides to the 3′ end of a nucleic acid. The invention also relates to methods of nucleic acid synthesis and sequencing comprising the use of said modified terminal transferase enzyme, to kits comprising said modified terminal transferase enzyme and to the use of said kits in methods of nucleic acid synthesis and sequencing.

### FIELD OF THE INVENTION

The invention relates to the use of a modified terminal transferase enzyme in a method of adding one or more nucleotides to the 3′ end of a nucleic acid. The invention also relates to methods of nucleic acid synthesis and sequencing comprising the use of said modified terminal transferase enzyme, to kits comprising said modified terminal transferase enzyme and to the use of said kits in methods of nucleic acid synthesis and sequencing.

### BACKGROUND OF THE INVENTION

Nucleic acid synthesis is vital to modern biotechnology. The rapid pace of development in the biotechnology arena has been made possible by the scientific community's ability to artificially synthesize DNA, RNA and proteins.

Artificial DNA synthesis—a £1.8 billion and growing market—allows biotechnology and pharmaceutical companies to develop a range of peptide therapeutics, such as insulin for the treatment of diabetes. It allows researchers to characterise cellular proteins to develop new small molecule therapies for the treatment of diseases our aging population faces today, such as heart disease and cancer. It even paves the way forward to creating life, as the Venter Institute demonstrated in 2010 when they placed an artificially synthesised genome into a bacterial cell.

However, current DNA synthesis technology does not meet the demands of the biotechnology industry. While the benefits of DNA synthesis are numerous, an oft-mentioned problem prevents the further growth of the artificial DNA synthesis industry, and thus the biotechnology field. Despite being a mature technology, it is practically impossible to synthesise a DNA strand greater than 200 nucleotides in length, and most DNA synthesis companies only offer up to 120 nucleotides. In comparison, an average protein-coding gene is of the order of 2000-3000 nucleotides, and an average eukaryotic genome numbers in the billions of nucleotides. Thus, all major gene synthesis companies today rely on variations of a ‘synthesise and stitch’ technique, where overlapping 40-60-mer fragments are synthesised and stitched together by PCR (see Young, L. et al. (2004) Nucleic Acid Res. 32, e59). Current methods offered by the gene synthesis industry generally allow up to 3 kb in length for routine production.

The reason DNA cannot be synthesised beyond 120-200 nucleotides at a time is due to the current methodology for generating DNA, which uses synthetic chemistry (i.e., phosphoramidite technology) to couple a nucleotide one at a time to make DNA. As the efficiency of each nucleotide-coupling step is 95.0-99.0% efficient, it is mathematically impossible to synthesise DNA longer than 200 nucleotides in acceptable yields. The Venter Institute illustrated this laborious process by spending 4 years and 20 million USD to synthesise the relatively small genome of a bacterium (see Gibson, D. G. et al. (2010) Science 329, 52-56).

Known methods of DNA sequencing use template-dependent DNA polymerases to add 3′-reversibly terminated nucleotides to a growing double-stranded substrate (see, Bentley, D. R. et al. (2008) Nature 456, 53-59). In the ‘sequencing-by-synthesis’ process, each added nucleotide contains a dye, allowing the user to identify the exact sequence of the template strand. Albeit on double-stranded DNA, this technology is able to produce strands of between 500-1000 bps long. However, this technology is not suitable for de novo nucleic acid synthesis because of the requirement for an existing nucleic acid strand to act as a template.

There is therefore a need to provide improved methods of nucleic acid synthesis and sequencing that is able to overcome the problems associated with currently available methods.

### SUMMARY OF THE INVENTION

According to a first aspect of the invention, the use of a modified terminal transferase enzyme in a method of adding one or more nucleotides to the 3′ end of a nucleic acid, characterised in that said enzyme comprises a mutated BRCA-1 C-terminal (BRCT) domain.

According to a second aspect of the invention, there is provided a method of nucleic acid synthesis, which comprises the steps of:
(a) providing an initial initiator sequence;
(b) adding a reversibly blocked nucleotide triphosphate to said initiator sequence in the presence of a modified terminal transferase enzyme as defined herein;
(c) removal of all reagents from the initiator sequence;
(d) cleaving the blocking group from the reversibly blocked nucleotide added in step (b) to said initiator sequence; and
(e) removal of the cleaving agent.

According to a further aspect of the invention, there is provided a method of nucleic acid synthesis which is performed in a microfluidic device comprising the steps of:
(a) providing an initial initiator sequence bound to a surface within a microfluidic device;
(b) adding a reversibly blocked nucleotide triphosphate to said initiator sequence in the presence of a modified terminal transferase enzyme as defined herein;
(c) removal of all reagents from the initiator sequence;
(d) cleaving the blocking group from the reversibly blocked nucleotide added in step (b) to said initiator sequence; and
(e) removal of the cleaving agent.

According to a further aspect of the invention, there is provided a kit comprising a modified terminal transferase enzyme as defined herein, optionally in combination with one or more components selected from: an initiator sequence, a microfluidic device or chip, one or more reversibly blocked nucleotide triphosphates, inorganic pyrophosphatase, such as purified, recombinant inorganic pyrophosphatase from Saccharomyces cerevisiae, and a cleaving agent; further optionally together with instructions for use of the kit in accordance with the method as defined herein.

According to a further aspect of the invention, there is provided the use of a kit as defined herein in a method of nucleic acid synthesis or nucleic acid sequencing.

### BRIEF DESCRIPTION OF THE FIGURES

FIG. 1: A terminal transferase, TdT, engineered with N-terminal truncations retains full or better catalytic activity, including full truncations of the N-terminal BRCT domain from TdT, corresponding to approximately 21% of the protein molecular weight. In this experiment, a TdT containing a 21% N-terminal truncation (“Truncated TdT” or ΔNTE-TdT) added more modified 2′-deoxynucleotide triphosphates (biotin-16-dUTP) to the 3′-end of a DNA initiator molecule (N control) when compared to “Wild-type TdT.” TdT was incubated with a DNA initiator and biotin-16-dUTP for 5 min at 37° C. The DNA was visualized by a Cy-5 fluorescent dye covalently attached to the 5′-end of the initiator molecule. The reaction products were analyzed by denaturing PAGE. The gel is shown at a 90° angle for space consideration.

FIG. 2: Single-step incorporation of modified nucleotide triphosphates. An engineered, full length TdT can add 3′-O-modified reversibly terminated nucleotide triphosphates in a quantitative fashion. The full length TdT was able to add a 3′-O-azidomethyl 2′-deoxythymidine triphosphate and a 3′-O-azidomethyl 2′-deoxyguanosine triphosphate quantitatively. Reactions were analysed by denaturing PAGE.

FIG. 3: A schematic of a typical POLX family polymerase with terminal transferase activity. NT-POLXc and POLXc are domain annotations that indicate a DNA Polymerase beta-like domain, which conveys terminal transferase and nucleotidyltransferase activity. N-terminal to the NT-POLXc/POLXc domain is the BRCT domain, which contains a dimer interface motif as well as the signature BRCT sequence consisting of the sequence Trp-X-X-X-Cys/Ser, where X is any amino acid.

FIG. 4: An N-terminal truncation of TdT (“Truncated TdT”) readily dissociates from a 5′-Cy5 labeled oligonucleotide, whereas the full-length version of TdT only shows comparable dissociation at >1 M NaCl concentrations. TdT (5 μM) was incubated with a 5′-Cy5 labeled 2′-deoxyoligonucleotide (1 μM) for 30 min at 37° C. Prior to fluorescence intensity measurements, all samples were brought up to the specified ionic strengths and filtered through a 0.22 micron spin filter. Fluorescence intensity was determined using a fluorescence plate reader.

FIG. 5: TdT engineered with N-terminal truncations shows superior performance in a cyclic DNA synthesis process. In this experiment, the cyclic process described in the text was repeated to yield an N+2 product. Lane 1 (N): N control DNA initiator. Lanes 2 and 3: full length engineered TdT variant. Lanes 4-6: N-terminal truncated engineered TdT variant. The DNA was visualized by the Cy-5 fluorescent dye covalently attached to the 5′-end of the initiator molecule. Reactions were analysed by denaturing PAGE.

FIG. 6: TdT engineered with N-terminal truncations is capable of catalysing the addition of 2, 5, 10, and more modified nucleotides carrying a reversible terminator. For each cycle, engineered truncated TdT was introduced with a reversibly terminated nucleotide to a DNA initiator. Following addition, the DNA initiator was washed to remove any remaining modified nucleotides. The nucleotide was then “deprotected” and additional wash steps followed to remove any remaining deprotecting agent. This cyclic process was repeated until the indicated number of reversibly terminated nucleotides were added (i.e., N+X, where X is the number of cycles). Reactions were analysed by denaturing PAGE.

FIG. 7: Eleven orthologs of TdT, with variance up to 41.0% identity of the L. oculatus TdT and also with variance up to 39.8% identity of S. harrisii TdT, are demonstrably more soluble when the BRCT domain is truncated (top). Expression yields within E. coli are a standard measure of the (1) behavior and (2) solubility of a protein. Higher yields generally indicate well-behaved, highly soluble proteins. As a result of the BRCT truncation on a wide range of TdT orthologs covering most ortholog classes within the chordata phylum. TdT solubility increases as evidenced by the consistent >2-fold improvement in solubility (bottom). Truncations were made in the genes of the eleven TdT orthologs by site-directed mutagenesis following standard protocols. Expression was performed in E. coli in 3-ml of Terrific Broth overnight at 20° C. following standard induction by IPTG. Lysis was performed using 1×BugBuster in 20 mM HEPES KOH (pH 7.5), 300 mM KCI, 10% glycerol, and 1 mM PMSF. Following lysis, proteins were purified using a HisPur Ni-NTA 96-well spin plate. Proteins were purified to >80% homogeneity as assessed by standard SDS-PAGE. Concentrations and yields were determined using the NanoOrange Protein Quantitation Kit (Thermo Fisher) on the QuantiFluor fluorimeter (Promega). Each expression was performed in triplicate.

FIG. 8: Surface accessibility tests between wild-type and BRCT-truncated TdT enzymes conclude TdT with BRCT domains increase fouling of DNA-immobilized surfaces preventing terminal transferase activity. In all lanes, TdT orthologs were incubated in a well containing a surface 5′-immobilized single-stranded piece of DNA. TdT was incubated in a suitable buffer for proper terminal transferase activity in the well for 1 h at 37° C. Following the 1 h incubation, commercial B. taurus TdT, pyrophosphatase, and thymidine 5′-triphosphate (free 3′-OH) was added to the well. The reactions were analyzed by 20% denaturing PAGE (19:1) and imaged by virtue of an internal fluorophore on the single-stranded piece of DNA. Lanes 1-8 contain wild-type TdT orthologs (respectively, S. harrisii, S. scrofa, O. garnettii, C. lanigera, B. taurus, P. panicus, and M. musculus). Lanes 9-19 contain BRCT-truncated TdT orthologs (respectively, S. harrisii, S. scrofa, O. garnettii, C. lanigera, B. taurus, D. novemcinctus, M. domestica, P. nyererei, M. brandtii, P. panicus, and M. musculus). Not all wild-type TdT orthologs could be tested due to low expression yield as a result of high insolubility. BRCT truncation surprisingly prevents the surface fouling seen with wild-type TdTs over a wide range of TdT orthologs (up to 39.8% identity). Surface fouling, protein aggregation, and general protein misbehavior prevents efficient multi-cycle addition of nucleotides to surface immobilized DNA. Thus, BRCT truncation is necessary for the de novo, TdT-mediated synthesis of DNA.

### DETAILED DESCRIPTION OF THE INVENTION

According to a first aspect of the invention, the use of a modified terminal transferase enzyme in a method of adding one or more nucleotides to the 3′ end of a nucleic acid, characterised in that said enzyme comprises a mutated BRCA-1 C-terminal (BRCT) domain.

De novo enzymatic-based nucleic acid synthesis is an alternative method to phosphoramidite DNA synthesis. One method to achieve the former is the use of polymerases that act as terminal transferases (e.g., DNA nucleotidylexotransferase (DNTT/TdT), DNA polymerase mu (POLM or Polμ), DNA polymerase lambda (POLL or Polλ), DNA polymerase theta (POLQ or PolΘ), and prokaryotic polymerases) to add nucleotide triphosphates one at a time in a random or sequence-specific fashion. In order to achieve sequence control, modified nucleotides such as those blocked via the nitrogenous base (as described in GB Patent Application No. 1701396.2) or the sugar moiety (as described in WO 2016/128731 and U.S. Pat. No. 6,232,465), must be used in the synthesis process. Terminal transferase-mediated de novo DNA synthesis in a sequence-controlled fashion thus requires a cyclic process of (1) addition of a modified solution or solid phase nucleotide to the 3′-end of a nucleic acid (N species) to form a blocked N+1 species and (2) deprotection of the added nucleotide to regenerate an active N+1 species. Such a cyclic process requires a well-behaved terminal transferase enzyme that (a) readily releases from the N+1 species, (b) does not greatly foul the areas or surfaces in which the nucleotide addition occurs, and (c) is stable (e.g., not prone to aggregation or inactivation) during the nucleotide addition.

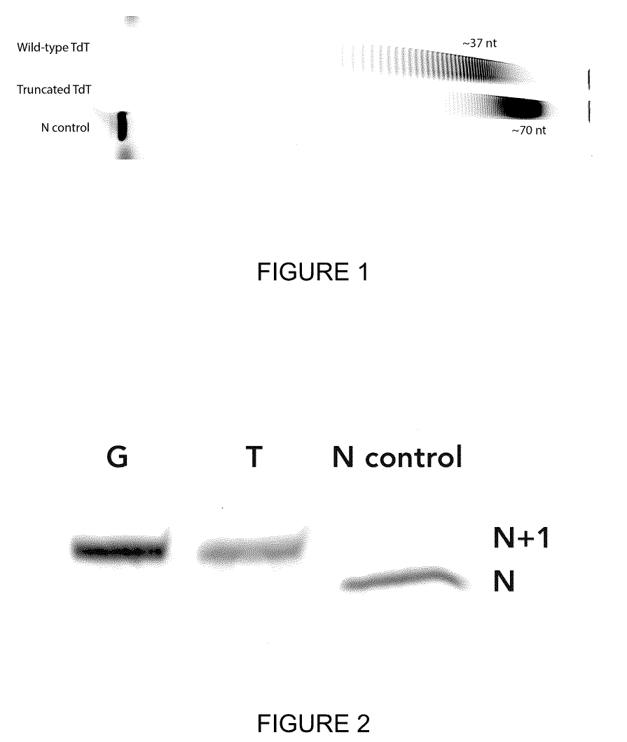

The inventors have surprisingly found that TdT appears to not only retain activity, but furthermore gains activity in solution after removing 21% of the wild-type mass of TdT from the N-terminus (ΔNTE-TdT), which corresponds to the deletion of the BRCT domain (see FIG. 1). Previously, it had only been demonstrated that TdT retains catalytic activity in crystalline form when residues are deleted from the N-terminus, but activity was not shown in solution^{1,2}. Protein activity, macromolecule conformation, and substrate accessibility are often significantly different between a solution phase macromolecule and the same macromolecule locked in a crystal lattice. According to Mozzarelli and Rossi, “ligand binding, catalysis and allosteric regulation occur in the crystalline environment but intermolecular interactions may hinder function-associated transitions and alter activity with respect to solution³. ” In a well-known example, the first atomic model of duplex DNA was not elucidated in the familiar B-DNA conformation+, but rather a Z-DNA conformation, the biological function of which has yet to be conclusively determined.

Indeed, in vivo observations of the effect of N-terminal deletions on TdT reinforce the inventors' surprising finding that TdT gains activity upon the N-terminal deletion of 21% of the wild-type TdT mass (ΔNTE-TdT). In a reconstituted V(D)J recombination assay in HEK₂₉₃T cells, Schatz and colleagues demonstrated that the absence of the BRCT domain on the N-terminus of murine TdT resulted in a consistent reduction of coding joint N-nucleotide addition to levels similar to that of an inactive long isoform of murine TdT (TdTL)⁵. In Mus musculus, TdT is alternatively spliced into two isoforms, known as TdTS and TdTL. TdTS was shown to be active at physiological temperatures, while TdTL was shown to be inactive at physiological temperatures⁶. Given that TdT lacking a BRCT domain displays consistently reduced or non-existent activity (similar to that of TdTL) in vivo, it would be expected that recombinant, engineered TdT with N-terminal BRCT domain truncations would result in reduced or non-existent terminal transferase activity. FIG. 1 clearly shows ΔNTE-TdT surprisingly both retains and gains terminal transferase catalytic activity.

The surprising result shown in FIG. 1 can be extended to the highly homologous cousins of TdT. DNA polymerases mu and lambda. Indeed, it is well known in the literature that DNA polymerases mu and lambda are easily converted to highly active terminal transferases following mutations guided by homology to wild-type TdT sequences. For example, Blanco and colleagues demonstrated that a point mutation mimicking TdT (R₃₈₇K in human DNA polymerase mu) converted DNA polymerase mu from a weak terminal transferase capable of adding tens of nucleotides to a strong terminal transferase capable of adding hundreds of nucleotides⁷. Since we demonstrate novel terminal transferase activity from TdT lacking a BRCT domain, it thus follows that polymerase mu and polymerase lambda carrying TdT-mimicking point mutations are active as terminal transferases even if the BRCT domain is absent.

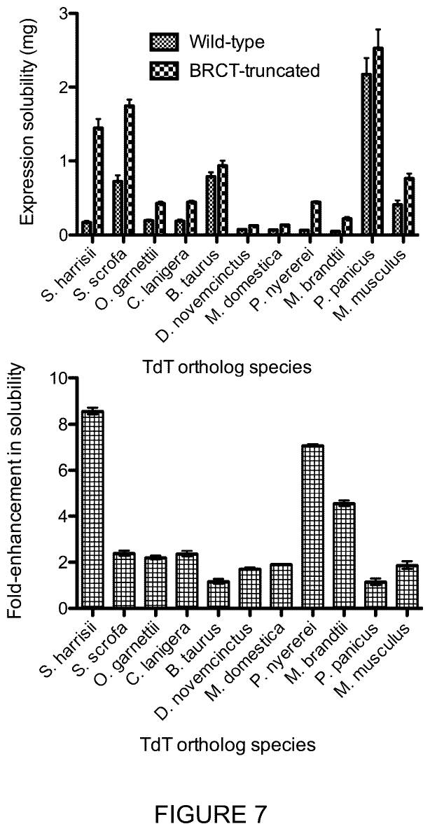

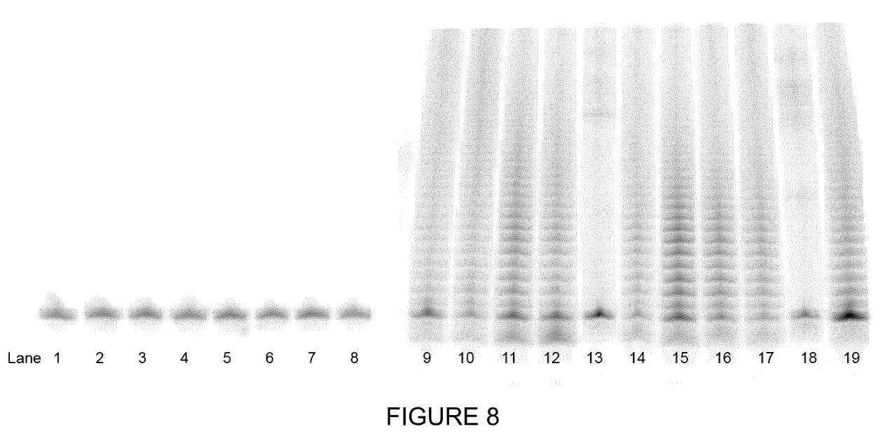

Data is also presented that demonstrates that solubility is increased (see FIG. 7) and surface accessibility to immobilized DNA is increased (see FIG. 8) when the BRCT domain is truncated. These results are observed for TdTs having over 40% sequence identity from each other. Without being bound by theory, it is believed that the data presented herein provides excellent plausibility that all TdTs having BRCT deletions according to the invention find utility in an enhanced method of nucleic acid synthesis.

Thus, in one embodiment the terminal transferase enzyme is from the DNA polymerase X family, such as terminal deoxynucleotidyl transferase (TdT), DNA polymerase λ (Polλ) and DNA polymerase μ (Polμ). In a yet further embodiment, the terminal transferase enzyme is selected from terminal deoxynucleotidyl transferase (TdT).

References herein to “TdT” refer to a terminal deoxynucleotidyl transferase (TdT) enzyme and include references to purified and recombinant forms of said enzyme. TdT is also commonly known as DNTT (DNA nucleotidylexotransferase) and any such terms should be used interchangeably.

References herein to “Polλ” refer to DNA Polymerase λ (also known as POLL or DNA Polymerase Lambda) is a protein found in eukaryotes. In humans, it is encoded by the POLLA gene. Pol λ is a member of the X family of DNA polymerases. It is thought to resynthesize missing nucleotides during non-homologous end joining (NHEJ), a pathway of DNA double-strand break (DSB) repair.

References herein to “Polμ” refer to DNA Polymerase μ (also known as POLM or DNA Polymerase Mu) is a polymerase enzyme found in eukaryotes. In humans, this protein is encoded by the POLM gene. Polμ is a member of the X family of DNA polymerases. It participates in resynthesis of damaged or missing nucleotides during the non-homologous end joining (NHEJ) pathway of DNA repair.

References herein to the term “BRCA-1 C-terminal (BRCT) domain” refer to the C-terminal domain of a breast cancer susceptibility protein). This domain is found predominantly in proteins involved in cell cycle checkpoint functions responsive to DNA damage, for example as found in the breast cancer DNA-repair protein BRCA1. The domain is an approximately 100 amino acid tandem repeat, which appears to act as a phospho-protein binding domain. For example, the BRCT domain is present in TdT from amino acid residues 26-130.

References herein to the term “mutated BRCA-1 C-terminal (BRCT) domain” refer to any inactivated form of the BRCT domain. Examples of a mutated BRCT domain include one or more mutations selected from: a deletion, substitution or an insertion.

In one embodiment, said enzyme comprises a truncated BRCT domain, such as an N-terminal truncated BRCT domain (e.g. an N-terminal truncation to approximately 21% of the TdT wild-type molecular weight). This embodiment of the invention is surprisingly crucial to multi-step de novo enzymatic DNA synthesis. N-terminal truncations endow wild-type and engineered TdTs with much higher cycling efficiencies that greatly enhance the industrial utility of TdT-mediated nucleic acid synthesis.

In an alternative embodiment, said BRCT domain is absent.

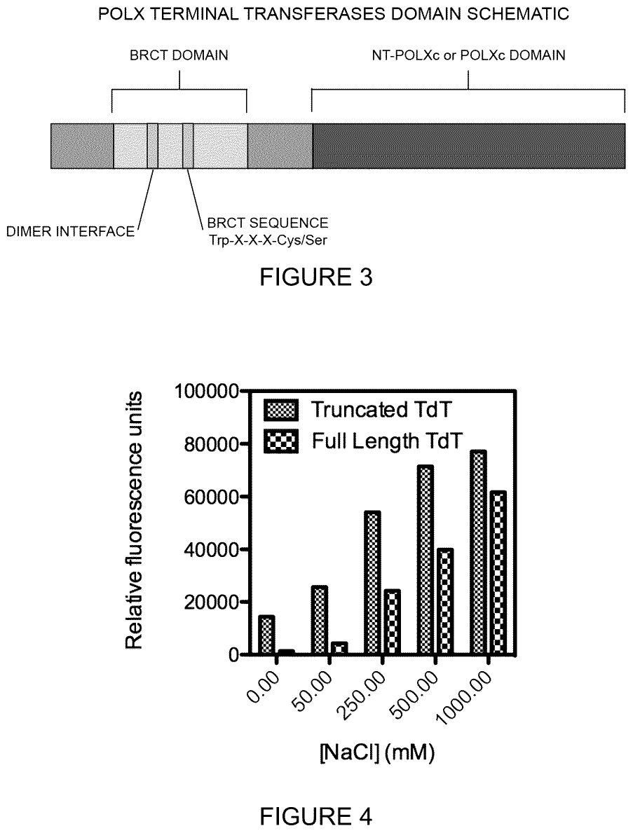

The absence or truncation of a BRCT domain in a terminal transferase enzyme is defined as the following:
any amino acid sequences having:
   (1) terminal transferase activity; and
   (2) containing a domain belonging to the NT-POLXc/POLXc DNA polymerase beta thumb superfamily (cd00141, cl25961, smart00483, pfam14791, COG1796);
and not containing:
   (3) a domain belonging to the BRCT superfamily (c100038, cd00027, smart00292, pfam00533, pfam12738, pfam16589, pfam16759, pfam16770);
      as defined by the Conserved Domain Database (CDD)⁸ maintained by the US National Center for Biotechnology Information (NCBI) identified via Conserved Domain Searches (CD-Search)⁹. A schematic domain is presented in FIG. 3. A protein domain shall be identified as belonging to either NT-POLXc/POLXc (cd00141) or BRCT (cd00027) if their threshold bit score is greater than 199.344 or 29.2119, respectively. The threshold bit score for each annotated domain is set by the NCBI CDD curators as a conservative measure of whether or not a protein sequence belongs to a specific domain. The bit score of a query sequence is calculated to determine if it is above the threshold set by NCBI curators. A bit score is calculated by first performing an alignment using the position-specific scoring matrix (PSSM) specific for each domain annotation to maximize a raw alignment score, where the cost to open a gap is 11 and the cost to extend a gap by one is 1. The domain-specific PSSMs for the domain annotations given above is provided in Tables 1 and 2 for NT-POLXc/POLXc (cd00141) or BRCT (cd00027), respectively. The raw alignment score is then used to calculate a bit score using the following equation:

$${S}^{′} = \frac{λ S - ln ( K )}{ln ( 2 )}$$

where S′ is equal to the bit score, S is the raw alignment score, and λ and K are normalizing factors obtained as described by Gertz (Gertz, E.M. BLAST scoring parameters. 2005.). The process of calculating a bit score is typically performed through a publicly available algorithm called a reverse-position-specific BLAST (RPS-BLAST) against the CDD database, as described by Marchler-Bauer and colleagues⁸⁻⁹.

TABLE 1
Position-specific scoring matrix (PSSM) for NT-POLXc (cd00141) domain
where a threshold bit score of 199.344 is required to qualify as a member of this family.
The PSSM was obtained from the NCBI CDD. In the upper left hand corner table header,
P signifies position, C signifies the consensus sequence, and M signifies the master
sequence of known structure. In the rest of the table, letters indicate standard amino
acid single letter nomenclature.

| P | C | M | A | G | I | L | V | M | F | W | P | C | S | T | Y | N | Q | H | K | R | D | E |
|---|---|---|---|---|---|---|---|---|---|---|---|---|---|---|---|---|---|---|---|---|---|---|
| 1 | Q | 4-G | 2 | −3 | −2 | −4 | −5 | −4 | −5 | 1 | −5 | −6 | 0 | 0 | 0 | −1 | 4 | 4 | 2 | 2 | −4 | 2 |
| 2 | E | 5-G | −1 | −1 | −1 | 0 | −5 | −4 | −1 | 2 | −1 | −1 | −4 | −2 | −5 | −1 | −1 | 2 | 2 | 0 | 3 | 4 |
| 3 | I | 6-I | −5 | −7 | 5 | 2 | 3 | 0 | 4 | −5 | −6 | −5 | −5 | −1 | −3 | −3 | −6 | −6 | −6 | −6 | −7 | −6 |
| 4 | A | 7-T | 4 | −4 | 4 | −4 | 1 | −3 | −5 | −6 | −5 | 0 | −1 | 4 | −5 | −5 | −2 | −5 | −4 | −5 | −5 | −5 |
| 5 | D | 8-D | 1 | −1 | −6 | −4 | −6 | −5 | −6 | 2 | −5 | 2 | −1 | −4 | −5 | 1 | −3 | 2 | 0 | 2 | 4 | 3 |
| 6 | I | 9-M | 3 | −5 | 4 | 0 | 2 | 2 | 1 | −6 | −5 | 0 | −4 | −4 | −4 | −5 | −1 | −5 | 1 | −5 | −5 | −1 |
| 7 | L | 10-L | −5 | −7 | 0 | 5 | −3 | 1 | 6 | −4 | −7 | −5 | −6 | −5 | −2 | −7 | −6 | −6 | −6 | −6 | −7 | −6 |
| 8 | E | 11-T | −4 | −1 | −5 | −5 | −1 | 1 | −5 | −6 | −5 | −6 | 2 | 2 | 1 | 0 | 1 | −4 | 0 | −4 | 1 | 5 |
| 9 | E | 12-E | −3 | −5 | 1 | −5 | 0 | −4 | −6 | −6 | −2 | −6 | −1 | 0 | −5 | −4 | 3 | −4 | 1 | 3 | −3 | 5 |
| 10 | L | 13-L | −5 | −7 | 4 | 5 | 0 | 5 | −3 | −5 | −6 | −5 | −5 | 0 | −5 | −6 | −5 | −6 | −6 | −6 | −7 | −6 |
| 11 | A | 14-A | 6 | 0 | −2 | −1 | −4 | 0 | −5 | −6 | −5 | −4 | −3 | −1 | −5 | −5 | −4 | 0 | −1 | −1 | −5 | −1 |
| 12 | D | 15-N | −3 | −5 | −5 | −2 | −3 | −5 | −6 | −6 | −5 | −6 | −3 | 1 | −1 | 2 | −3 | −4 | 3 | −3 | 5 | 3 |
| 13 | L | 16-F | 1 | −6 | 1 | 4 | −2 | 0 | 0 | −4 | −6 | 1 | −1 | −4 | 5 | −1 | −4 | 2 | −1 | 0 | −6 | −5 |
| 14 | L | 17-E | −3 | −6 | −3 | 3 | −4 | 3 | 0 | −3 | −6 | 1 | −1 | −4 | 6 | −5 | −4 | 0 | −4 | 0 | −2 | 1 |
| 15 | E | 18-K | −3 | −3 | −6 | −6 | −6 | −5 | −7 | −6 | −5 | −6 | −1 | 1 | −5 | −3 | 0 | −4 | 2 | 2 | 0 | 6 |
| 16 | L | 19-N | 0 | −3 | 4 | 3 | 1 | −3 | 4 | −5 | −6 | −5 | −5 | −4 | −4 | 3 | −5 | −1 | −5 | −2 | −2 | −5 |
| 17 | L | 20-V | −2 | −5 | −4 | 1 | 1 | −4 | −5 | −6 | −5 | 0 | −2 | 0 | −5 | 2 | 3 | −4 | 3 | 3 | −4 | 2 |
| 18 | G | 21-S | −1 | 5 | −2 | −2 | −2 | −2 | −2 | −2 | −2 | −2 | 0 | −1 | −2 | 1 | 0 | −2 | −2 | −2 | 0 | 0 |
| 19 | G | 22-Q | −1 | 4 | −7 | −7 | −6 | −6 | −7 | −6 | −5 | −6 | −3 | −4 | −6 | 1 | −1 | 0 | 1 | 1 | 2 | 4 |
| 20 | N | 23-A | 0 | −4 | −6 | −6 | −3 | −6 | −7 | −7 | −5 | −6 | −1 | −4 | −6 | 6 | −1 | −4 | −3 | −4 | 5 | 3 |
| 21 | P | 24-I | −1 | −5 | 2 | −5 | −4 | −1 | −6 | −6 | 4 | −6 | −1 | 0 | −5 | −4 | 1 | 1 | 3 | 2 | 1 | 1 |
| 22 | F | 25-H | −5 | 1 | 0 | −4 | −2 | −4 | 7 | 7 | −7 | −6 | −5 | −5 | 3 | −5 | −5 | 5 | −5 | −2 | −6 | −6 |
| 23 | R | 26-K | −4 | −2 | −6 | −6 | −6 | −5 | −2 | −6 | −2 | −7 | −1 | −4 | −5 | −4 | −2 | −4 | 5 | 7 | −5 | −2 |
| 24 | V | 27-Y | 1 | −5 | 3 | −2 | 3 | −3 | −4 | −5 | −2 | 4 | 1 | 1 | 3 | −1 | −5 | 0 | −5 | −5 | −5 | −5 |
| 25 | R | 28-N | −5 | −3 | −1 | 2 | −2 | −3 | −0 | −6 | −6 | −6 | −2 | −4 | −5 | 3 | 0 | −4 | −1 | 6 | −5 | −4 |
| 26 | A | 29-A | 6 | 1 | −5 | −5 | −2 | −4 | −6 | −6 | −4 | −4 | 2 | −2 | −5 | −4 | −1 | −5 | −4 | −5 | −5 | −2 |
| 27 | Y | 30-Y | −5 | −7 | −5 | −4 | −5 | −4 | 4 | −1 | −7 | −6 | −5 | −5 | 9 | −6 | −5 | −2 | −5 | −5 | −7 | −6 |
| 28 | R | 31-R | −2 | −2 | −5 | 0 | −5 | 2 | −5 | −5 | −5 | −6 | 1 | −4 | 1 | −4 | 0 | 0 | −1 | 7 | −5 | −4 |
| 29 | K | 32-K | −4 | −5 | −6 | −1 | −5 | 1 | −6 | −6 | −5 | −6 | 0 | −4 | −5 | 2 | 0 | −4 | 5 | 5 | −4 | −2 |
| 30 | A | 33-A | 7 | −2 | −4 | −5 | −2 | 1 | −6 | −6 | −4 | −4 | −1 | −3 | −5 | −5 | −4 | −5 | −4 | −5 | −5 | −4 |
| 31 | A | 34-A | 5 | −4 | 4 | −2 | −1 | −4 | −5 | −6 | −5 | −4 | 1 | −3 | −5 | −5 | −4 | −5 | −1 | −1 | −5 | −5 |
| 32 | R | 35-S | 1 | −2 | −6 | −6 | −5 | −5 | −6 | −6 | −2 | −6 | 3 | −3 | −5 | 2 | 2 | −4 | 0 | 4 | −2 | 2 |
| 33 | A | 36-V | 4 | −5 | 1 | −1 | 4 | −3 | −2 | −6 | −5 | −4 | 2 | 1 | −5 | −5 | −5 | −5 | −5 | −5 | −5 | −5 |
| 34 | L | 37-I | −5 | −7 | 6 | 4 | 2 | 3 | −3 | −6 | −6 | −5 | −6 | −4 | −5 | −7 | −6 | −6 | −6 | −6 | −7 | −6 |
| 35 | E | 38-A | 1 | −3 | −2 | −3 | −5 | −5 | −6 | −6 | −5 | −6 | 0 | −4 | −2 | −4 | 0 | −4 | 4 | 2 | −1 | 4 |
| 36 | S | 39-K | 1 | −1 | −6 | −2 | −5 | −5 | −2 | −6 | −5 | −5 | 4 | −3 | −5 | 2 | −1 | 0 | 2 | 1 | 1 | 1 |
| 37 | L | 40-Y | −5 | −6 | −3 | 4 | 0 | 0 | 2 | −4 | −6 | 1 | −3 | −2 | 5 | −5 | 1 | 4 | −5 | −5 | −2 | −5 |
| 38 | P | 41-P | −4 | −2 | −6 | −6 | −6 | −6 | −7 | −7 | 7 | −6 | −1 | 0 | −5 | 1 | 0 | 3 | −2 | −1 | −2 | −2 |
| 39 | E | 42-H | −1 | −5 | −4 | −2 | −1 | −4 | 3 | −5 | −5 | 0 | −1 | 2 | 3 | −4 | −2 | 1 | 2 | 2 | −4 | 3 |
| 40 | P | 43-K | −1 | −5 | −6 | −6 | −6 | −5 | −7 | −7 | 6 | −6 | 1 | −4 | −6 | −1 | −1 | −4 | 3 | 1 | 2 | 1 |
| 41 | I | 44-I | −1 | −7 | 6 | 2 | 4 | −2 | −4 | −6 | −6 | −4 | −5 | −1 | −5 | −6 | −6 | −7 | −6 | −6 | −7 | −6 |
| 42 | E | 45-K | 2 | −5 | −5 | −5 | −5 | −5 | −6 | −6 | 0 | −5 | −1 | 3 | −1 | −4 | 2 | 0 | 1 | 1 | −1 | 3 |
| 43 | S | 46-S | −1 | −4 | −5 | −6 | −3 | −5 | −6 | −6 | −4 | 0 | 5 | 3 | −5 | −1 | −1 | −4 | −1 | −2 | 0 | 3 |
| 44 | L | 47-G | 0 | 2 | 1 | 3 | 0 | 5 | −3 | −5 | −6 | −1 | −2 | −4 | 3 | −5 | −4 | −5 | −1 | −5 | −6 | −1 |
| 45 | E | 48-A | 2 | −5 | −2 | 0 | −1 | −4 | 1 | 3 | −5 | −5 | −1 | −2 | 1 | −4 | 0 | 0 | 2 | −2 | 1 | 3 |
| 46 | E | 49-E | 1 | −3 | −6 | −6 | −6 | −5 | −6 | −6 | −5 | −6 | −1 | −4 | −1 | −3 | 4 | −4 | −1 | 1 | 3 | 5 |
| 47 | A | 50-A | 4 | −5 | 0 | 1 | −1 | 0 | −5 | −6 | −2 | 0 | −3 | −1 | −5 | −2 | −4 | −5 | −1 | 3 | 0 | 1 |
| 48 | K | 51-K | −1 | −2 | −4 | −1 | −2 | 1 | −5 | −5 | −2 | 2 | 1 | 2 | −4 | −4 | 1 | 3 | 3 | 2 | −2 | 1 |
| 49 | K | 52-K | 0 | 2 | −2 | −6 | −5 | −5 | −6 | −6 | −5 | −6 | 1 | −4 | −5 | −1 | −3 | 2 | 4 | 2 | 0 | 2 |
| 50 | L | 53-L | −5 | −7 | 5 | 5 | −1 | 3 | −3 | −5 | −6 | −5 | −6 | −4 | −5 | −7 | −5 | −6 | −6 | −6 | −7 | −2 |
| 51 | P | 54-P | −4 | −5 | −6 | −6 | −6 | −6 | −7 | −7 | 8 | −6 | −2 | −4 | −6 | 1 | −4 | 0 | −2 | 1 | 1 | 4 |
| 52 | G | 55-G | −3 | 6 | −7 | −6 | −6 | −6 | −2 | 1 | −6 | 3 | −4 | −1 | −1 | 1 | −5 | 0 | −1 | −5 | −2 | −5 |
| 53 | I | 56-V | −4 | −7 | 6 | 1 | 4 | 0 | −1 | −6 | −6 | 3 | −5 | −4 | −5 | −7 | −6 | −7 | −6 | −6 | −7 | −6 |
| 54 | G | 57-G | −3 | 8 | −7 | −7 | −7 | −6 | −7 | −6 | −6 | −6 | −1 | −5 | −7 | −4 | −5 | −5 | −5 | −6 | −5 | −5 |
| 55 | K | 58-T | −1 | −3 | −6 | −3 | −3 | −5 | −6 | −7 | 1 | −6 | −2 | −3 | −6 | −3 | −1 | −4 | 5 | 0 | 3 | 3 |
| 56 | K | 59-K | 2 | 3 | −6 | −6 | −5 | −5 | −6 | −6 | −5 | −6 | 0 | −1 | −5 | −2 | 0 | 3 | 4 | 2 | 0 | −3 |
| 57 | I | 60-I | −4 | −7 | 6 | 2 | 0 | 5 | −4 | −6 | −6 | −5 | −3 | 2 | −5 | −6 | −5 | −6 | −5 | −6 | −6 | −6 |
| 58 | A | 61-A | 6 | −1 | −2 | −2 | −1 | −4 | −5 | −6 | −5 | −4 | −1 | −2 | −1 | −2 | −4 | −5 | 1 | 0 | −5 | −2 |
| 59 | E | 62-E | 0 | 1 | −6 | −2 | −6 | −5 | −6 | −6 | −5 | −6 | 1 | −4 | −5 | −1 | 2 | 2 | 3 | 0 | 3 | 3 |
| 60 | K | 63-K | −4 | −5 | −1 | −1 | 1 | −4 | −6 | −6 | −5 | −6 | −4 | −4 | −5 | −1 | −2 | 0 | 7 | 1 | −4 | 1 |
| 61 | I | 64-I | −5 | −7 | 8 | 1 | −1 | 2 | −3 | 2 | −6 | −5 | −5 | −1 | 0 | −6 | −6 | −6 | −6 | −6 | −7 | −6 |
| 62 | E | 65-D | 0 | −3 | 0 | −3 | −1 | −1 | −6 | 1 | −1 | −6 | 0 | −2 | −5 | −1 | 4 | −4 | 2 | −1 | 2 | 3 |
| 63 | E | 66-E | −4 | −3 | −5 | −3 | −1 | −5 | −1 | −6 | −5 | −7 | −4 | −4 | −5 | −4 | 0 | −4 | −3 | −4 | 1 | 7 |
| 64 | I | 67-F | −5 | −7 | 6 | 2 | −1 | 0 | 5 | −4 | −7 | −5 | −6 | −5 | 3 | −7 | −6 | −5 | −6 | −6 | −7 | −6 |
| 65 | L | 68-L | 1 | −6 | 4 | 4 | 2 | 2 | −1 | −5 | −6 | −5 | −5 | −4 | −4 | −6 | −5 | −6 | −5 | −2 | −7 | −6 |
| 66 | E | 69-A | 0 | −5 | −6 | −4 | −5 | −5 | −6 | −6 | −5 | −6 | 1 | 2 | −5 | −1 | 2 | −4 | 1 | 3 | 1 | 4 |
| 67 | T | 70-T | −3 | −1 | −5 | −2 | −4 | −4 | −1 | −6 | −5 | −5 | 1 | 6 | −5 | −1 | −1 | 0 | −4 | −2 | −2 | 1 |
| 68 | G | 71-G | −3 | 7 | −7 | −7 | −7 | −6 | −7 | −6 | −6 | −6 | −4 | −5 | −6 | 0 | −5 | 0 | −5 | −2 | −5 | −5 |
| 69 | K | 72-K | −4 | −3 | −5 | −6 | −2 | −5 | −6 | −6 | −5 | −6 | 1 | 2 | −4 | −3 | −3 | 5 | 4 | 3 | −2 | 2 |
| 70 | L | 73-L | −4 | −6 | 1 | 5 | 0 | 2 | 0 | −5 | −2 | 4 | 2 | −2 | −4 | −6 | −5 | −6 | −5 | −5 | −6 | −6 |
| 71 | R | 74-R | 1 | −2 | −6 | −5 | −3 | −5 | 0 | −6 | 1 | −6 | 1 | −2 | −1 | −4 | 2 | −4 | 2 | 4 | −2 | 2 |
| 72 | K | 75-K | −2 | 0 | −2 | −1 | −5 | −4 | −5 | −5 | 0 | −6 | −1 | −2 | 2 | −4 | 0 | −4 | 5 | 1 | −2 | 2 |
| 73 | L | 76-L | 1 | −6 | −1 | 5 | 1 | −2 | −3 | 1 | −6 | −5 | −5 | −4 | 3 | −6 | −5 | 4 | −5 | −5 | −6 | −1 |
| 74 | E | 77-E | −1 | −3 | −6 | −2 | −6 | −5 | −6 | −6 | −5 | −6 | −3 | −4 | −6 | 1 | 1 | −4 | 1 | −3 | 3 | 6 |
| 75 | E | 78-K | −1 | −5 | −6 | −3 | −3 | −5 | −2 | −6 | −5 | −6 | −2 | −2 | 0 | 1 | −1 | 3 | 3 | 1 | 2 | 4 |
| 76 | L | 79-I | −1 | −6 | 4 | 4 | 1 | 1 | 1 | −5 | −6 | −5 | −2 | −2 | −4 | −6 | −5 | −6 | −5 | −5 | −2 | 0 |
| 77 | R | 80-R | 0 | −3 | −5 | 1 | −1 | 0 | −5 | −6 | −5 | −6 | 1 | −4 | −5 | −1 | 2 | 2 | 1 | 5 | 0 | 1 |
| 78 | E | 81-Q | −1 | 1 | −5 | −3 | −5 | −5 | −6 | −6 | −2 | 2 | 0 | 2 | −5 | 2 | 1 | −1 | 2 | 1 | −1 | 3 |
| 79 | D | 82-D | −2 | 1 | −4 | −4 | −4 | 0 | −4 | −4 | 0 | −4 | 2 | −3 | −4 | −2 | 1 | −3 | 1 | −1 | 5 | 2 |
| 80 | V | 84-T | −1 | −3 | 1 | 1 | 2 | −3 | −2 | −5 | −5 | −5 | 1 | 0 | 2 | −4 | −4 | −4 | 1 | 3 | 0 | −1 |
| 81 | P | 85-S | −2 | −5 | −4 | −3 | 1 | −5 | −5 | −5 | 6 | −5 | 1 | 0 | 3 | −2 | −4 | −4 | −4 | −2 | −2 | 1 |
| 82 | P | 86-S | 0 | −5 | −2 | −2 | 0 | −4 | −6 | −6 | 4 | −6 | −1 | −2 | −5 | −4 | 2 | −4 | 2 | 2 | −1 | 2 |
| 83 | G | 87-S | 2 | 4 | 1 | −5 | 2 | −4 | −6 | −6 | −5 | 0 | 2 | 1 | −5 | −4 | −4 | −1 | −1 | −5 | 0 | −1 |
| 84 | L | 88-I | −3 | −7 | 3 | 4 | 3 | 4 | 2 | −5 | −6 | −5 | −5 | 0 | −4 | −6 | −5 | −6 | −6 | −6 | −7 | −6 |
| 85 | L | 89-N | −1 | −5 | 0 | 2 | 0 | −3 | −5 | −6 | −5 | −5 | 0 | −2 | −5 | 2 | −1 | −4 | 3 | 2 | −1 | 0 |
| 86 | L | 90-F | −1 | −3 | −2 | 3 | −4 | −1 | 1 | −5 | −2 | −5 | −1 | −2 | 3 | −5 | 0 | −4 | −4 | −5 | 0 | 4 |
| 87 | L | 91-L | −5 | −7 | 0 | 4 | −2 | 3 | 7 | −4 | −7 | −5 | −6 | −5 | −2 | −7 | −6 | −5 | −6 | −6 | −7 | −6 |
| 88 | L | 92-T | −2 | −3 | −3 | 3 | −3 | 2 | −4 | −5 | −5 | −5 | 0 | 4 | 0 | −4 | 0 | 1 | 1 | −4 | −5 | −2 |
| 89 | R | 93-R | −1 | 2 | −6 | −6 | −6 | −5 | −6 | −6 | −5 | −6 | 1 | −4 | −5 | 3 | 2 | −4 | 3 | 4 | 0 | 2 |
| 90 | V | 94-V | −2 | −7 | 5 | 1 | 5 | −2 | −4 | −6 | −6 | 4 | −5 | −4 | −5 | −7 | −6 | −7 | −6 | −6 | −7 | −6 |
| 91 | P | 95-S | −5 | −6 | −5 | −5 | −5 | −5 | 3 | 7 | 4 | −6 | −1 | 0 | 3 | −5 | 0 | 3 | −2 | −1 | −5 | 1 |
| 92 | G | 96-G | −3 | 8 | −7 | −7 | −7 | −6 | −7 | −6 | −6 | −6 | −4 | −5 | −7 | −4 | −5 | −5 | −5 | −6 | −1 | −5 |
| 93 | V | 97-I | 0 | −6 | 5 | 2 | 5 | 1 | −4 | −6 | −6 | −4 | −5 | −4 | −5 | −6 | −6 | −6 | −6 | −6 | −7 | −6 |
| 94 | G | 98-G | −1 | 8 | −7 | −7 | −7 | −6 | −7 | −6 | −6 | −6 | −4 | −5 | −7 | −4 | −5 | −5 | −5 | −6 | −5 | −6 |
| 95 | P | 99-P | 1 | −2 | −2 | −2 | 0 | −5 | −6 | −7 | 7 | −6 | −2 | −3 | −6 | −5 | −4 | −5 | −2 | −1 | −5 | −2 |
| 96 | K | 100-S | 1 | −2 | −5 | −5 | −3 | −5 | −1 | −6 | −5 | −6 | 0 | 1 | −5 | 0 | −1 | −4 | 6 | 1 | −4 | −1 |
| 97 | T | 101-A | 1 | −5 | −1 | −5 | −4 | 0 | −5 | −6 | −5 | −5 | −3 | 6 | −1 | −4 | −1 | 1 | 3 | 2 | −5 | −4 |
| 98 | A | 102-A | 6 | −4 | 2 | −2 | 2 | −4 | −5 | −6 | −5 | −4 | 0 | −3 | −5 | −5 | −5 | −5 | −4 | −5 | −5 | −5 |
| 99 | R | 103-R | 2 | −1 | −6 | −3 | −5 | −5 | −6 | −6 | −5 | −6 | −1 | −4 | 0 | 0 | 2 | −4 | 3 | 4 | −2 | 1 |
| 100 | K | 104-K | −2 | −5 | −1 | 0 | −2 | 2 | −5 | −6 | −5 | −6 | 0 | 1 | −5 | −1 | 1 | 0 | 4 | 3 | −4 | 1 |
| 101 | L | 105-F | −3 | −7 | 0 | 4 | −2 | −2 | 3 | 10 | −7 | −5 | −6 | −5 | 1 | −7 | −6 | −6 | −6 | −6 | −7 | −6 |
| 102 | Y | 106-V | −3 | −6 | −2 | −3 | 1 | −4 | 3 | 6 | −6 | −6 | −5 | −5 | 8 | 0 | −5 | 3 | −5 | 0 | −6 | −5 |
| 103 | E | 107-D | 1 | −3 | −6 | −1 | −5 | −4 | −2 | −6 | −5 | −6 | −1 | −4 | −5 | 2 | 3 | −4 | 0 | 3 | 1 | 4 |
| 104 | L | 108-E | −1 | −6 | −3 | 4 | −3 | 4 | −1 | −5 | −5 | −5 | −4 | −4 | −5 | −5 | 1 | −5 | 2 | −1 | −1 | −1 |
| 105 | G | 109-G | −3 | 7 | −7 | −7 | −7 | −6 | −7 | −6 | −6 | −6 | −4 | −5 | −6 | 0 | −2 | −5 | −5 | −5 | −2 | −3 |
| 106 | I | 110-I | −2 | −7 | 5 | 0 | 3 | −3 | 4 | 4 | −6 | 0 | −5 | −4 | 2 | −6 | −6 | 0 | −6 | −6 | −7 | −6 |
| 107 | R | 111-K | −4 | −3 | −6 | −5 | −3 | −1 | −6 | −6 | −5 | −6 | 2 | 2 | −5 | −3 | −1 | 0 | 2 | 5 | 0 | 2 |
| 108 | T | 112-T | −3 | −4 | −5 | −5 | −4 | −5 | −6 | −6 | −5 | 1 | 4 | 6 | −5 | 2 | −4 | −5 | −4 | −4 | 2 | −4 |
| 109 | L | 113-L | −2 | −7 | 2 | 5 | 2 | −2 | 1 | −5 | −6 | −5 | −5 | −4 | −4 | −7 | −6 | −6 | −3 | −6 | −7 | −6 |
| 110 | E | 114-E | 2 | −2 | −6 | −6 | −3 | −5 | −6 | −7 | −5 | −6 | −2 | −2 | −6 | −3 | 0 | −4 | −3 | −4 | 4 | 6 |
| 111 | D | 115-D | 0 | −2 | −6 | −6 | −6 | −6 | −7 | −7 | −5 | −7 | −2 | −4 | −6 | −3 | 2 | −4 | −1 | 0 | 6 | 4 |
| 112 | L | 116-L | −5 | −7 | 4 | 5 | 2 | −2 | −1 | −5 | −6 | −5 | −6 | −4 | −4 | −7 | −6 | −6 | −2 | −6 | −7 | −6 |
| 113 | R | 117-R | −1 | −5 | 0 | −1 | −2 | −4 | −6 | −6 | −5 | −6 | −4 | −4 | −1 | −4 | −1 | −4 | 4 | 5 | −4 | 4 |
| 114 | K | 118-K | 1 | −3 | −6 | −6 | −5 | −5 | −6 | −6 | −1 | −6 | 0 | 2 | −1 | 2 | 1 | 0 | 3 | 2 | 0 | 2 |
| 115 | A | 119-N | 4 | −1 | −3 | −2 | −2 | −4 | −5 | −5 | −1 | −5 | −3 | −4 | 0 | 1 | 1 | 3 | 1 | −4 | 0 | 0 |
| 116 | A | 120-E | 3 | 0 | 0 | −1 | −1 | −3 | −4 | −4 | 0 | 4 | −1 | −1 | −4 | −3 | −3 | −3 | 2 | 0 | −3 | 1 |
| 117 | G | — | −1 | 5 | −3 | −3 | −2 | −2 | −3 | 3 | −2 | −2 | 1 | −1 | −2 | 0 | 0 | −2 | −2 | −2 | −2 | 0 |
| 118 | A | 121-D | 2 | −1 | −2 | 0 | −2 | −4 | 1 | 2 | 1 | −5 | −3 | −4 | −4 | −4 | −1 | 2 | 0 | −4 | 3 | 2 |
| 119 | K | 122-K | −2 | −3 | −5 | −3 | 0 | −4 | −6 | −6 | −2 | −6 | 1 | 0 | 0 | 0 | −1 | −4 | 6 | 2 | −4 | −3 |
| 120 | L | 123-L | −4 | −6 | −3 | 4 | −1 | 3 | 1 | −5 | −5 | −5 | 2 | 2 | −4 | −5 | −4 | 0 | 0 | 0 | −6 | −5 |
| 121 | E | 124-N | 2 | −4 | −1 | −5 | −4 | −5 | −6 | −6 | −1 | −5 | 0 | 4 | −5 | 4 | −1 | −4 | −3 | −4 | −1 | 4 |
| 122 | Q | 125-H | 1 | −5 | −6 | −2 | −5 | −4 | −5 | 6 | 0 | −6 | −3 | 0 | −4 | −1 | 3 | 5 | 2 | 2 | −4 | 1 |
| 123 | N | 126-H | 0 | −1 | −5 | −5 | −1 | 4 | −6 | −6 | −5 | −5 | −2 | 2 | −5 | 4 | 3 | 4 | 2 | −2 | −1 | −3 |
| 124 | I | 127-Q | −4 | −6 | 6 | 0 | −2 | 1 | −4 | −6 | −5 | −5 | −4 | −4 | −5 | −5 | 7 | −4 | −4 | −4 | −5 | −3 |
| 125 | L | 128-R | −2 | −6 | −3 | 5 | −2 | −2 | −4 | −6 | −5 | −5 | −5 | −4 | −5 | −5 | 2 | −5 | 3 | 2 | −5 | −1 |
| 126 | I | 129-I | 1 | −5 | 4 | −2 | 1 | −3 | −1 | −6 | −5 | 0 | −4 | −1 | 0 | −1 | 1 | 0 | 2 | 0 | −2 | 1 |
| 127 | G | 130-G | 1 | 7 | −7 | −7 | −6 | −6 | −6 | −6 | −5 | −6 | −1 | −4 | −6 | 2 | −5 | −5 | −5 | −5 | −4 | −5 |
| 128 | L | 131-L | −3 | −7 | 5 | 4 | 1 | 2 | 1 | 2 | −6 | −5 | −6 | −2 | −4 | −7 | −6 | −6 | −6 | −6 | −7 | −6 |
| 129 | E | 132-K | 1 | −1 | −5 | 0 | −1 | −1 | −6 | −6 | −2 | −6 | −1 | −4 | −5 | −4 | 1 | −4 | 3 | 2 | −2 | 4 |
| 130 | Y | 133-Y | −1 | −6 | −2 | 1 | −2 | 2 | 4 | −3 | −6 | −5 | −3 | −2 | 6 | −5 | −4 | 5 | −5 | 0 | −6 | −2 |
| 131 | Y | 134-F | 2 | −6 | −2 | −2 | 0 | 0 | 3 | 2 | −6 | −5 | −4 | −5 | 8 | −5 | −5 | 3 | −3 | −5 | −6 | −5 |
| 132 | E | 135-G | −4 | −1 | −6 | −6 | −6 | −5 | 0 | −6 | −5 | −7 | −1 | −2 | −5 | −3 | 0 | 0 | 2 | 3 | 4 | 4 |
| 133 | D | 136-D | 0 | −5 | −6 | −6 | −6 | −6 | −7 | −7 | −5 | −6 | −1 | −2 | −6 | −1 | −1 | −4 | 1 | 2 | 6 | 2 |
| 134 | F | 137-F | 0 | −6 | 2 | 1 | −2 | −3 | 5 | 6 | −3 | −5 | −1 | −4 | 0 | −5 | 1 | −5 | −2 | 2 | −6 | −3 |
| 135 | Q | 138-E | −1 | −2 | −2 | 0 | −1 | −4 | −5 | −6 | −1 | 1 | 2 | −2 | −5 | 0 | 3 | −4 | 1 | 3 | −2 | 2 |
| 136 | Q | 139-K | −2 | 2 | −6 | −1 | −5 | −4 | −6 | −6 | −5 | 1 | 1 | 1 | −5 | −4 | 4 | −4 | 2 | 1 | −2 | 3 |
| 137 | R | 140-R | −2 | −5 | −6 | −6 | −6 | −5 | −6 | −6 | −2 | 2 | −2 | −1 | −1 | −4 | −2 | 0 | 2 | 7 | −5 | −3 |
| 138 | I | 141-I | −4 | 6 | 6 | −2 | 2 | 6 | 3 | −5 | −6 | −5 | −3 | 0 | −4 | −6 | 0 | 0 | −5 | −1 | −6 | −5 |
| 139 | P | 142-P | −1 | −5 | −5 | 0 | −5 | −4 | −6 | −6 | 7 | −5 | 2 | −1 | −1 | −2 | −4 | −5 | −2 | 0 | −5 | −4 |
| 140 | R | 143-R | −5 | −6 | 2 | 3 | −4 | −3 | −4 | 1 | −6 | −6 | −5 | −5 | 0 | −5 | −3 | −4 | 0 | 6 | −6 | −4 |
| 141 | E | 144-E | 2 | 1 | −1 | −6 | −5 | −5 | −2 | 1 | −2 | −5 | 2 | −4 | −5 | 0 | −1 | 0 | −2 | −1 | 2 | 4 |
| 142 | E | 145-E | 0 | −5 | −5 | −5 | 0 | −1 | −6 | −6 | −5 | −6 | −3 | −2 | 0 | −4 | 0 | 0 | 0 | −1 | 0 | 6 |
| 143 | A | 146-M | 6 | −1 | −2 | −4 | 3 | 3 | −5 | −6 | −5 | 3 | −1 | −1 | −5 | −5 | −4 | −5 | −5 | −5 | −6 | −5 |
| 144 | L | 147-L | −2 | −1 | −2 | 2 | −1 | 1 | 0 | −5 | −5 | −6 | −4 | 2 | 2 | −2 | 0 | −4 | 1 | 0 | 0 | 3 |
| 145 | A | 148-Q | 2 | −5 | −6 | −3 | −5 | −5 | −6 | −6 | 3 | −6 | −1 | 0 | −5 | −4 | 3 | −4 | 1 | 2 | −2 | 3 |
| 146 | I | 149-M | −3 | −6 | 5 | 2 | 2 | 4 | −1 | −5 | −6 | −5 | −5 | 0 | 1 | −5 | −5 | 5 | −5 | −5 | −6 | −1 |
| 147 | A | 150-Q | 4 | 1 | −5 | −2 | −1 | −4 | 0 | −5 | −5 | −5 | −1 | −2 | 2 | −4 | 1 | −4 | −4 | −2 | −4 | 3 |
| 148 | E | 151-D | 0 | −3 | −6 | −3 | −5 | 1 | −6 | −6 | −5 | 0 | −1 | −2 | −5 | 0 | 3 | −4 | 1 | 2 | 1 | 5 |
| 149 | I | 152-I | −2 | −3 | 4 | 1 | 0 | −3 | −5 | −6 | −2 | −5 | −3 | 1 | −5 | −5 | −3 | −1 | 1 | 2 | −4 | 3 |
| 150 | I | 153-V | −4 | −7 | 6 | 3 | 4 | 1 | −3 | −6 | −6 | −5 | −6 | −4 | −5 | −7 | −6 | −7 | −6 | −6 | −7 | −6 |
| 151 | K | 154-L | −1 | −3 | 1 | 2 | 0 | 0 | −5 | −6 | −5 | −6 | −4 | −4 | −5 | −4 | 2 | −4 | 4 | 2 | −4 | 2 |
| 152 | E | 155-N | 2 | 0 | −5 | −2 | −3 | −5 | −6 | −6 | −2 | −6 | 1 | −4 | −5 | 1 | 0 | −4 | 3 | 1 | 0 | 3 |
| 153 | A | 156-E | 4 | −3 | −1 | −5 | −2 | −4 | 1 | −5 | −5 | 1 | −3 | −1 | 3 | −4 | −1 | 6 | −3 | 1 | −5 | 1 |
| 154 | L | 157-V | 2 | −3 | 1 | 4 | 3 | 4 | 1 | −5 | −6 | −4 | −4 | −2 | −4 | −6 | −5 | −6 | −5 | −6 | −6 | −6 |
| 155 | R | 158-K | −2 | −5 | −5 | −2 | −3 | −5 | −6 | 1 | 1 | 0 | −2 | −1 | −5 | 1 | 1 | 3 | 2 | 4 | 0 | 2 |
| 156 | E | 159-K | 1 | 0 | −2 | −3 | −3 | −5 | −6 | 1 | −1 | −5 | 1 | 2 | −5 | 0 | 1 | −4 | 1 | −2 | 2 | 2 |
| 157 | V | 160-V | −1 | −2 | 3 | 2 | 3 | 0 | 3 | −4 | −5 | 2 | −4 | 1 | −3 | −2 | −2 | 0 | −4 | −4 | −5 | −2 |
| 158 | D | 161-D | −1 | 0 | −4 | 0 | −2 | −4 | −4 | −5 | 4 | −4 | −1 | −1 | −4 | 1 | −3 | 1 | 0 | −3 | 4 | −1 |
| 159 | P | 162-S | 0 | 3 | −1 | −3 | −2 | −4 | −5 | −5 | 6 | −5 | −1 | −2 | −1 | −4 | −3 | 0 | 0 | −4 | −4 | 1 |
| 160 | V | 163-E | 1 | 2 | 0 | −1 | 2 | −4 | −5 | −5 | −5 | −5 | −2 | −1 | −1 | 1 | 0 | −4 | 1 | −4 | 2 | 1 |
| 161 | L | 164-Y | 1 | −6 | 2 | 2 | 0 | 2 | 1 | −5 | −5 | 1 | −3 | −4 | 2 | −5 | −2 | −5 | −1 | 1 | −5 | 2 |
| 162 | Q | 165-I | −2 | −3 | 2 | 0 | −1 | −4 | −2 | 2 | −5 | 1 | 0 | −1 | −5 | 1 | 4 | 0 | 1 | 1 | −2 | 1 |
| 163 | V | 166-A | 3 | −4 | 3 | 0 | 4 | 3 | −1 | −6 | −1 | 3 | −1 | −4 | −5 | −6 | −5 | −6 | −5 | −6 | −6 | −5 |
| 164 | E | 167-T | 0 | −5 | 0 | −4 | 2 | −1 | −5 | −6 | −5 | 1 | −1 | 4 | −5 | −4 | −1 | 0 | −4 | −4 | −4 | 4 |
| 165 | I | 168-V | 0 | −6 | 5 | 2 | 3 | 0 | −4 | 1 | 1 | 0 | −5 | −1 | −1 | −6 | −5 | −6 | −5 | −2 | −6 | −6 |
| 166 | A | 169-C | 5 | −2 | −4 | −4 | 0 | 3 | −5 | −6 | −5 | 7 | −3 | 2 | −5 | −5 | −1 | −5 | −5 | −5 | −5 | −5 |
| 167 | G | 170-G | −3 | 8 | −7 | −7 | −7 | −6 | −7 | −6 | −6 | −6 | −4 | −5 | −7 | −4 | −5 | −6 | −5 | −6 | −5 | −6 |
| 168 | S | 171-S | −2 | 3 | −6 | −6 | −5 | −5 | −6 | −6 | −4 | −5 | 7 | −2 | −5 | −3 | −4 | −4 | −4 | −4 | 0 | −4 |
| 169 | Y | 172-F | −3 | −7 | 0 | 2 | 0 | −3 | 5 | −2 | −6 | −5 | −5 | −1 | 7 | −6 | −5 | −3 | −6 | −6 | −7 | −6 |
| 170 | R | 173-R | −5 | −6 | −7 | −6 | −6 | −5 | −6 | −6 | −6 | −7 | −2 | −4 | −5 | −1 | −2 | −4 | −1 | 8 | −5 | −3 |
| 171 | R | 174-R | −5 | −6 | −7 | −6 | −6 | −5 | −6 | −6 | −6 | −7 | −4 | −5 | −5 | −4 | −2 | −4 | −1 | 9 | −5 | −3 |
| 172 | G | 175-G | −2 | 6 | −6 | −6 | −6 | 3 | −2 | 3 | −6 | −6 | −4 | −5 | −2 | −4 | −2 | −5 | −2 | 1 | −5 | −3 |
| 173 | K | 176-A | 1 | −5 | −6 | −2 | −5 | −4 | −6 | −6 | −5 | −6 | 0 | −2 | 0 | −4 | 0 | −4 | 6 | 4 | −4 | −3 |
| 174 | E | 177-E | 2 | −5 | −5 | −2 | −5 | −1 | −6 | −6 | 3 | −6 | 0 | 0 | −5 | −4 | −1 | −4 | 0 | −4 | 0 | 5 |
| 175 | T | 178-S | −1 | −5 | −1 | −5 | −1 | −4 | −5 | −6 | −5 | −5 | 1 | 7 | −1 | −3 | 0 | −5 | 0 | −1 | −2 | −4 |
| 176 | V | 179-S | −3 | 2 | 1 | −2 | 5 | −4 | −5 | −6 | −6 | 7 | 3 | −3 | −5 | −5 | −5 | −6 | −5 | −6 | −6 | −5 |
| 177 | G | 180-G | −1 | 7 | −7 | −7 | −6 | −6 | −6 | −6 | −5 | −6 | −4 | −5 | −5 | −4 | −4 | 4 | −1 | −1 | −2 | −5 |
| 178 | D | 181-D | −2 | −5 | −7 | −7 | −7 | −6 | −7 | −8 | −5 | −7 | −4 | −4 | −7 | −2 | −4 | −5 | −4 | −5 | 8 | −2 |
| 179 | I | 182-M | −1 | −7 | 5 | 3 | 4 | 3 | −4 | −6 | −6 | −4 | −5 | −4 | −5 | −7 | −6 | −6 | −6 | −6 | −7 | −6 |
| 180 | D | 183-D | −5 | −5 | −7 | −7 | −7 | −7 | −7 | −8 | −5 | −7 | −4 | −4 | −7 | −2 | −3 | −4 | −4 | −5 | 8 | 1 |
| 181 | I | 184-V | −3 | −7 | 6 | 2 | 4 | 2 | 3 | −5 | −6 | −5 | −5 | −4 | 0 | −7 | −6 | −6 | −6 | −6 | −7 | −6 |
| 182 | L | 185-L | −4 | −7 | 1 | 6 | 3 | 1 | −3 | −5 | −6 | −5 | −5 | −1 | −5 | −7 | −6 | −6 | −6 | −6 | −7 | −6 |
| 183 | V | 186-L | 2 | −3 | 4 | 2 | 4 | −2 | 1 | −6 | −6 | −4 | −5 | −4 | −4 | −6 | −6 | −6 | −6 | −6 | −6 | −6 |
| 184 | T | 187-T | 2 | −2 | −4 | −5 | −2 | −4 | −1 | −5 | −5 | 0 | 2 | 6 | 0 | −4 | −4 | 1 | −4 | −5 | −5 | −4 |
| 185 | H | 188-H | 2 | 0 | −5 | −5 | −5 | 2 | −5 | −6 | −1 | 0 | 3 | 0 | −4 | −3 | −3 | 7 | 1 | 2 | −5 | −4 |
| 186 | P | 189-P | −1 | −5 | −6 | −6 | −5 | −5 | −7 | −7 | 6 | 0 | 0 | 0 | −6 | −2 | −2 | −5 | 1 | −1 | 2 | 1 |
| 187 | D | 190-S | −4 | 1 | −5 | −5 | −5 | −5 | −1 | −5 | −4 | 0 | 0 | −1 | −1 | 0 | −3 | 3 | −2 | −1 | 6 | 2 |
| 188 | A | 191-F | 1 | 2 | −1 | −2 | −2 | −2 | 1 | −2 | −2 | 3 | −2 | 0 | 2 | −2 | 0 | −2 | 1 | −2 | −1 | 0 |
| 189 | T | 192-T | 0 | 0 | −2 | −2 | −2 | −2 | −2 | −2 | −1 | −2 | −1 | 3 | −2 | 2 | 1 | −2 | 1 | −1 | 2 | 0 |
| 190 | S | 193-S | 0 | 0 | −1 | −1 | −2 | −2 | −2 | −2 | −1 | −2 | 3 | −1 | −2 | 1 | 0 | −2 | 0 | −1 | 2 | 0 |
| 191 | R | 199-P | 0 | −2 | −2 | −2 | −1 | −2 | −2 | −2 | 1 | −2 | 0 | 1 | −2 | 0 | 2 | 1 | 1 | 2 | −1 | 1 |
| 192 | G | 200-K | −2 | 3 | −1 | −1 | −2 | −2 | −2 | −2 | 0 | −2 | −1 | 0 | 0 | 0 | 1 | 2 | 0 | 1 | 0 | −1 |
| 193 | L | 201-L | −1 | −1 | −1 | 2 | 1 | 1 | 2 | −2 | −2 | −2 | 0 | −2 | −2 | 0 | 0 | −2 | −2 | −2 | 1 | 1 |
| 194 | L | 202-L | 0 | 1 | −1 | 3 | −1 | 1 | 1 | 1 | 4 | −5 | −4 | −1 | −4 | −5 | −4 | −5 | −1 | 1 | −5 | −5 |
| 195 | E | 203-H | 2 | −1 | −5 | −2 | −3 | −5 | −6 | −6 | 1 | −5 | 1 | 0 | −5 | −1 | −1 | 3 | 3 | −2 | −1 | 3 |
| 196 | K | 204-Q | 3 | −5 | −6 | −3 | −5 | −5 | −6 | −6 | 2 | −6 | 0 | −4 | −5 | −2 | 1 | 2 | 4 | 2 | −1 | 1 |
| 197 | V | 205-V | −2 | −6 | 2 | 4 | 5 | −2 | −1 | −6 | −6 | −5 | −3 | −4 | −4 | −2 | −5 | −6 | −5 | −6 | −1 | −3 |
| 198 | V | 206-V | −4 | −7 | 4 | 2 | 4 | 5 | −4 | −6 | −6 | −5 | −5 | 0 | −5 | −6 | −5 | −1 | −5 | −2 | −3 | −6 |
| 199 | D | 207-E | −2 | −5 | −1 | −1 | −2 | −5 | −6 | −6 | −2 | 0 | −2 | −2 | −5 | −3 | 1 | 3 | 0 | 1 | 5 | 3 |
| 200 | A | 208-Q | 3 | −1 | −5 | −4 | −2 | −4 | −2 | −6 | −5 | 1 | 0 | −1 | −1 | −4 | 1 | 5 | 2 | 3 | −4 | 0 |
| 201 | L | 209-L | −5 | −7 | −1 | 5 | −3 | 0 | 6 | 1 | −7 | −5 | −6 | −5 | −2 | −2 | −6 | −5 | −6 | −1 | −7 | −6 |
| 202 | V | 210-Q | 0 | −6 | −1 | 1 | 3 | −3 | −5 | −6 | −5 | 3 | −4 | 3 | −5 | −4 | 1 | 2 | 2 | −2 | −5 | 0 |
| 203 | E | 211-K | 0 | −1 | −2 | −5 | −5 | 1 | −6 | −6 | 0 | −6 | 1 | 1 | −5 | 1 | 2 | 1 | 1 | 1 | 1 | 2 |
| 204 | L | 212-V | −2 | −6 | 1 | 1 | −1 | 4 | 1 | 1 | −2 | 2 | 1 | −1 | 2 | −5 | 3 | 0 | 1 | −4 | −5 | 0 |
| 205 | G | 213-H | −4 | 4 | −5 | 0 | −3 | −4 | −5 | −6 | 5 | −6 | −4 | −4 | −5 | 1 | −4 | 0 | −1 | −5 | 0 | −1 |
| 206 | F | 214-F | −2 | 2 | −1 | 1 | −1 | −1 | 5 | −4 | −5 | −5 | −4 | −1 | 4 | −1 | 0 | −4 | −4 | −2 | 0 | 1 |
| 207 | V | 215-I | −2 | −6 | 4 | 3 | 5 | −2 | −4 | −6 | −5 | −4 | −5 | −2 | −4 | −6 | −5 | −6 | 0 | −5 | −6 | −2 |
| 208 | T | 216-T | −1 | −4 | −1 | 1 | −2 | −3 | −4 | −5 | −4 | −4 | −1 | 4 | −4 | −1 | −1 | −4 | 1 | 1 | 1 | 3 |
| 209 | E | 217-D | −2 | −2 | −5 | −5 | −4 | −4 | 0 | −4 | −1 | 4 | 1 | −2 | 3 | −3 | −1 | 2 | −3 | 0 | 4 | 3 |
| 210 | V | 218-T | −2 | 1 | 2 | −2 | 4 | −3 | −4 | −5 | −4 | −4 | −1 | 2 | 0 | −4 | −3 | −4 | −4 | −4 | 1 | 2 |
| 211 | L | 219-L | 1 | −1 | 3 | 3 | 0 | −2 | −3 | 2 | −4 | −4 | 0 | 0 | −1 | −4 | −1 | 1 | −4 | −4 | 0 | −1 |
| 212 | S | 220-S | 2 | −1 | −2 | −2 | 0 | 3 | −1 | −5 | −4 | −4 | 3 | −1 | −4 | 2 | −3 | −4 | −1 | 0 | 0 | −1 |
| 213 | K | 221-K | 0 | 2 | −5 | −3 | −5 | −5 | −6 | −6 | −5 | 1 | 1 | −2 | −5 | −3 | 2 | 2 | 3 | 0 | 3 | 0 |
| 214 | G | 222-G | −2 | 6 | −1 | −1 | −2 | −4 | −5 | −6 | −5 | 0 | −3 | 1 | −5 | 0 | −2 | 3 | −4 | −5 | 1 | −3 |
| 215 | D | 223-E | −4 | 0 | −1 | −2 | −2 | −4 | 3 | −5 | 1 | 0 | 0 | 0 | −4 | −3 | 1 | −4 | −2 | −4 | 4 | 3 |
| 216 | T | 224-T | −2 | 0 | −5 | −5 | −2 | −5 | −6 | −6 | −1 | −5 | −2 | 5 | −5 | 0 | 2 | −4 | 3 | −2 | 0 | 1 |
| 217 | K | 225-K | −4 | −3 | −5 | −5 | −1 | −5 | −6 | −6 | −5 | −6 | −1 | −1 | −5 | −4 | 0 | −4 | 7 | 3 | −4 | −3 |
| 218 | A | 226-F | 2 | −1 | −4 | −2 | 1 | 0 | 3 | 5 | −6 | 5 | 1 | −2 | 5 | −5 | −5 | −4 | −5 | −5 | −6 | −5 |
| 219 | S | 227-M | −3 | −5 | −4 | 0 | −4 | 6 | 4 | −4 | −5 | −5 | 4 | 1 | 2 | −4 | −4 | 0 | 1 | −2 | −5 | −4 |
| 220 | G | 228-G | −1 | 5 | 1 | −1 | 4 | 3 | −1 | −6 | −6 | 0 | −4 | −2 | −5 | −5 | −5 | −6 | −5 | −6 | −2 | −6 |
| 221 | I | 229-V | −2 | −2 | 5 | 0 | 4 | 1 | −2 | −6 | −6 | 0 | −3 | −4 | −5 | −5 | −5 | −6 | −2 | 3 | 1 | −5 |
| 222 | L | 230-C | −4 | −2 | 1 | 5 | 0 | −2 | −3 | 2 | −6 | 7 | −1 | −4 | 0 | −6 | −6 | −6 | −6 | −6 | −7 | −6 |
| 223 | K | 231-Q | −1 | 0 | −3 | −6 | −5 | −5 | −6 | −6 | −5 | −6 | 1 | −1 | −5 | 0 | 2 | 0 | 4 | 4 | −2 | 1 |
| 224 | L | 232-L | −2 | −1 | −1 | 4 | 1 | −2 | −1 | −5 | −5 | 0 | −1 | 0 | −1 | 1 | −4 | −4 | 0 | −4 | −1 | 1 |
| 225 | P | 233-P | −2 | −1 | 0 | −2 | −2 | −2 | −2 | −2 | 5 | −2 | 0 | −2 | 0 | −2 | 1 | −2 | 1 | −2 | 0 | 1 |
| 226 | G | 234-S | −2 | 1 | −2 | −1 | −2 | −2 | −2 | −2 | 0 | 0 | −1 | −2 | −2 | 1 | 2 | 3 | 1 | −1 | 2 | 1 |
| 227 | G | 240-E | 0 | 2 | −1 | −1 | −2 | −2 | −2 | −2 | 0 | −2 | 1 | 1 | −2 | 1 | 1 | −2 | −1 | −1 | 1 | 0 |
| 228 | W | 241-Y | 0 | −2 | 1 | −2 | −2 | −2 | −2 | 7 | 0 | −2 | 0 | −1 | 1 | 0 | 0 | −2 | 0 | 2 | −2 | 0 |
| 229 | K | 242-P | −2 | −2 | 1 | 0 | −1 | 1 | −2 | 2 | 3 | −2 | −2 | 0 | 3 | −2 | 0 | −2 | 2 | 1 | −2 | −2 |
| 230 | G | 243-H | 1 | 4 | −6 | −6 | −5 | −5 | −2 | 3 | −5 | 4 | −4 | −1 | 0 | −1 | −1 | 6 | −4 | −1 | −2 | −2 |
| 231 | R | 244-R | −4 | −6 | 2 | 0 | 3 | 0 | −2 | −6 | −6 | −5 | −4 | 0 | −5 | −5 | −1 | −5 | −3 | 6 | −6 | −4 |
| 232 | R | 245-R | −2 | −5 | −6 | −6 | −6 | −5 | −6 | −6 | 0 | −7 | −2 | −2 | −5 | −1 | 4 | −4 | −2 | 7 | −2 | −1 |
| 233 | V | 246-I | 1 | −6 | 4 | 2 | 5 | −2 | −4 | −6 | −6 | 2 | −5 | −4 | −5 | −6 | −6 | −6 | −6 | −6 | −7 | −6 |
| 234 | D | 247-D | −5 | −5 | −7 | −7 | −7 | −7 | 7 | −8 | −5 | −7 | −4 | −4 | −7 | −2 | −3 | −4 | −4 | −5 | 8 | 0 |
| 235 | L | 248-I | −5 | −7 | 5 | 5 | −1 | 1 | 4 | −5 | −6 | −5 | −6 | −5 | −3 | −7 | −6 | −6 | −6 | −6 | −7 | −7 |
| 236 | R | 249-R | −2 | −6 | 3 | 1 | 1 | −3 | −1 | −6 | −6 | −6 | −5 | −4 | −5 | −5 | −1 | −5 | 0 | 6 | −6 | −4 |
| 237 | V | 250-L | −1 | −6 | 3 | 3 | 5 | 2 | −1 | −6 | −6 | 5 | −5 | 0 | −5 | −6 | −6 | −6 | −6 | −6 | −7 | −6 |
| 238 | V | 251-I | 0 | −6 | 2 | 0 | 6 | −3 | −2 | −5 | −2 | 4 | −2 | 1 | 1 | −6 | −5 | −6 | −5 | −6 | −6 | −6 |
| 239 | P | 252-P | −2 | −5 | −6 | −6 | −6 | −6 | −7 | −7 | 7 | −6 | 0 | −2 | −6 | −2 | −4 | −5 | 1 | −4 | 0 | 1 |
| 240 | P | 253-K | 0 | −5 | −5 | −5 | −3 | −5 | 0 | 6 | 4 | −6 | −2 | −4 | 5 | −5 | −4 | 2 | 1 | −2 | 0 | 1 |
| 241 | E | 254-D | 1 | −2 | −3 | −6 | −5 | −5 | −6 | −7 | −5 | −6 | 1 | −4 | −5 | −1 | 0 | 0 | −1 | −2 | 4 | 5 |
| 242 | E | 255-Q | 0 | −2 | −2 | −4 | −3 | −4 | −6 | −6 | −5 | 0 | 3 | −1 | 0 | −4 | 4 | −4 | −3 | 0 | −3 | 4 |
| 243 | F | 256-Y | −2 | −6 | 0 | −1 | −4 | −4 | 6 | 5 | −6 | −6 | −5 | −5 | 6 | −6 | −5 | −3 | −2 | 2 | −7 | −6 |
| 244 | G | 257-Y | 1 | 6 | −6 | −6 | −3 | −5 | −2 | −5 | 2 | −6 | −2 | −5 | 2 | −4 | −5 | −1 | −5 | −5 | −5 | −5 |
| 245 | A | 258-C | 5 | −4 | −5 | −5 | −4 | −4 | 2 | −5 | −5 | 7 | 1 | 0 | 2 | −5 | −5 | −5 | −5 | −5 | −5 | −5 |
| 246 | A | 259-G | 6 | 3 | −5 | 0 | −4 | −4 | −5 | −6 | −5 | 1 | −1 | 1 | −5 | −5 | −4 | −5 | −4 | −5 | −5 | −5 |
| 247 | L | 260-V | −5 | −7 | 1 | 5 | 2 | 2 | −1 | −5 | −6 | −5 | −5 | −2 | −4 | −1 | −5 | −6 | −5 | −1 | −1 | −6 |
| 248 | L | 261-L | −5 | −6 | 3 | 4 | 0 | 0 | −4 | −5 | −6 | −5 | −5 | −4 | −4 | −5 | 4 | 6 | −4 | −4 | −6 | −4 |
| 249 | Y | 262-Y | −3 | 2 | −5 | −2 | −5 | −4 | −1 | −2 | −6 | −6 | −5 | −5 | 8 | −5 | −4 | 5 | −5 | −2 | −6 | −5 |
| 250 | F | 263-F | −6 | −6 | −4 | −2 | −5 | −4 | 8 | 8 | −7 | −6 | −3 | −5 | 2 | −6 | −6 | −5 | −6 | −1 | −7 | −6 |
| 251 | T | 264-T | −3 | −5 | −1 | −5 | −3 | −4 | −6 | −6 | −4 | −4 | 1 | 8 | −5 | −3 | −4 | −5 | −4 | −5 | −4 | −4 |
| 252 | G | 265-G | −3 | 8 | −7 | −7 | −7 | −6 | −7 | −6 | −6 | −6 | −1 | −5 | −7 | −4 | −5 | −5 | −5 | −6 | −5 | −5 |
| 253 | S | 266-S | −2 | 0 | −6 | −6 | −5 | −5 | −6 | −6 | −4 | −5 | 6 | −2 | −5 | 3 | −3 | −4 | −1 | −4 | −1 | −1 |
| 254 | K | 267-D | −1 | −5 | −2 | −6 | −3 | −5 | −6 | 1 | −5 | −6 | −3 | −1 | −5 | 1 | 2 | 2 | 5 | 2 | 4 | −3 |
| 255 | Q | 268-I | 1 | −4 | 1 | −1 | −1 | 1 | 1 | −5 | −2 | −5 | −2 | 0 | −4 | 1 | 3 | 4 | −3 | −4 | 1 | 2 |
| 256 | F | 269-F | −5 | −6 | −4 | −2 | −5 | −4 | 8 | −3 | −6 | −6 | −5 | −5 | 2 | −4 | −5 | 8 | −5 | −5 | −6 | −5 |
| 257 | N | 270-N | −2 | −1 | −6 | −3 | −6 | −5 | −6 | −6 | −5 | −6 | −2 | −2 | −1 | 8 | 0 | 0 | −4 | −4 | −2 | 0 |
| 258 | R | 271-K | −2 | −2 | 3 | −4 | 2 | −4 | −5 | −6 | −5 | −6 | −4 | −2 | −5 | −4 | −3 | −5 | 3 | 6 | −5 | −4 |
| 259 | A | 272-N | 3 | −4 | −6 | −6 | −5 | −5 | −6 | 2 | −5 | −5 | 2 | −2 | −5 | 0 | 2 | 1 | 2 | 1 | 3 | 0 |
| 260 | L | 273-M | −4 | −7 | 2 | 5 | 2 | 7 | −1 | −5 | −6 | 1 | −5 | −4 | −4 | −6 | −5 | −6 | −6 | −6 | −7 | −6 |
| 261 | R | 274-R | −5 | −6 | −7 | −6 | −6 | −5 | −6 | −6 | −5 | −7 | −4 | −4 | −5 | −4 | 1 | −4 | 0 | 8 | −5 | −3 |
| 262 | R | 275-A | 2 | −3 | 0 | 2 | −4 | −3 | −5 | −6 | −5 | −5 | −3 | 2 | −1 | −2 | 0 | 1 | 0 | 4 | −1 | 0 |
| 263 | L | 276-H | −2 | −6 | 3 | 3 | −3 | 1 | 1 | 1 | −6 | −5 | −5 | −5 | 4 | −5 | −4 | 3 | −1 | 4 | −6 | −3 |
| 264 | A | 277-A | 7 | −3 | −5 | −5 | −4 | 0 | −6 | −6 | −4 | 1 | −1 | −3 | −5 | −5 | −4 | −5 | −4 | −5 | −5 | −4 |
| 265 | K | 278-L | −1 | −5 | 0 | 2 | −2 | −3 | −5 | −6 | −5 | −5 | −1 | 0 | −5 | 2 | 3 | −4 | 4 | 1 | −4 | 0 |
| 266 | E | 279-E | 1 | −3 | −6 | −6 | −3 | −5 | −6 | −6 | −5 | −6 | 0 | 0 | −5 | −3 | 3 | 1 | 3 | −1 | 1 | 4 |
| 267 | K | 280-K | −3 | −5 | −5 | −1 | −5 | 4 | −6 | −6 | −5 | −6 | −2 | −4 | −5 | 0 | 1 | 2 | 5 | 4 | −4 | −2 |
| 268 | G | 281-G | −3 | 7 | −7 | −7 | −6 | −6 | −7 | −6 | −5 | −6 | −1 | −5 | −6 | −2 | 0 | −5 | −1 | −1 | −2 | −5 |
| 269 | L | 282-F | −5 | −7 | −1 | −3 | −3 | 7 | 4 | −3 | −6 | −5 | −3 | −5 | 5 | −6 | −5 | −4 | −5 | −5 | −6 | −3 |
| 270 | K | 283-T | −4 | −5 | −5 | −3 | −2 | 2 | −6 | 1 | −5 | −5 | 2 | 3 | −5 | −1 | −3 | −4 | 5 | 2 | −2 | −3 |
| 271 | L | 284-I | −5 | −7 | 3 | 6 | 1 | 1 | 1 | −5 | −6 | −5 | −6 | −4 | 0 | −7 | −6 | −6 | −6 | −6 | −7 | −6 |
| 272 | N | 285-N | −4 | −4 | −6 | −6 | −6 | −5 | −6 | −7 | −5 | −5 | 4 | 1 | −6 | 6 | −3 | −4 | −4 | −4 | 3 | −3 |
| 273 | E | 286-E | −1 | −5 | −7 | −6 | −6 | −5 | −7 | −6 | −5 | −6 | 1 | −4 | −5 | 3 | 3 | 1 | −3 | −3 | 1 | 6 |
| 274 | Y | 287-Y | −5 | −2 | −6 | −5 | −5 | −5 | 0 | −3 | −6 | −6 | −2 | −2 | 7 | −4 | −4 | 8 | −2 | 1 | −1 | −4 |
| 275 | G | 288-T | 1 | 7 | −6 | −6 | −6 | −6 | −6 | −6 | −5 | −5 | 0 | 1 | −6 | −4 | −5 | −5 | −5 | −5 | −4 | −2 |
| 276 | L | 289-I | −5 | −7 | 4 | 5 | 3 | −2 | −1 | −5 | −6 | −5 | −6 | −4 | −4 | −7 | −6 | −6 | −6 | −6 | −7 | −6 |
| 277 | F | 290-R | −1 | −6 | −1 | −4 | −4 | −4 | 6 | −3 | −6 | −6 | 0 | −2 | 5 | −1 | −4 | −3 | −1 | −3 | −5 | −2 |
| 278 | D | 291-P | 2 | 1 | 6 | 6 | 6 | 2 | 6 | 7 | 3 | 1 | 1 | 1 | 5 | 1 | 3 | 0 | 3 | 4 | 4 | 2 |
| 279 | G | 292-L | 1 | 3 | −2 | 1 | 0 | 0 | −5 | −6 | −2 | −5 | −4 | −1 | −5 | 2 | −1 | −5 | 1 | 2 | 0 | −4 |
| 280 | V | 293-G | 0 | 2 | −2 | −2 | 3 | −2 | −3 | −3 | −2 | −2 | 0 | 2 | −2 | −2 | 0 | −2 | 0 | −1 | 2 | 0 |
| 281 | D | 298-A | 1 | −1 | −3 | −3 | −2 | −3 | −4 | −4 | 1 | −3 | 0 | 1 | −3 | 1 | 1 | −3 | 2 | −1 | 2 | 1 |
| 282 | G | 299-G | −4 | 6 | −7 | −7 | −6 | −6 | −7 | −6 | −5 | −6 | −3 | −1 | −6 | 1 | −4 | −5 | 1 | −1 | 2 | 2 |
| 283 | E | 300-E | −1 | −5 | −2 | −3 | −1 | −5 | −5 | 2 | −5 | −1 | 0 | −1 | −1 | 1 | 1 | 3 | 0 | 2 | 1 | 4 |
| 284 | R | 301-P | −1 | −6 | 1 | 1 | 1 | −3 | 2 | −5 | 3 | 3 | −2 | −2 | −1 | −5 | −4 | −5 | 0 | 4 | −6 | −3 |
| 285 | L | 302-L | −2 | −7 | 3 | 5 | 3 | −2 | −1 | −6 | −6 | −5 | −5 | −4 | −4 | −6 | −5 | −6 | −2 | −5 | −2 | −3 |
| 286 | P | 303-P | 3 | −1 | −2 | −2 | −5 | −5 | −2 | −6 | 5 | −5 | −2 | 0 | −5 | −4 | −3 | 1 | 0 | −4 | −4 | 3 |
| 287 | G | 304-V | 1 | 3 | 0 | −2 | 2 | −4 | 0 | −6 | −5 | 3 | 2 | 1 | −5 | −4 | −5 | −5 | −2 | −5 | 0 | −5 |
| 288 | E | 305-D | 1 | −1 | −5 | −5 | −5 | 2 | 0 | −6 | 2 | −6 | −1 | 0 | −4 | 0 | −3 | 3 | −1 | 2 | 0 | 3 |
| 289 | T | 306-S | −1 | −5 | −5 | −3 | −4 | −4 | −6 | −6 | −2 | 1 | 3 | 5 | −5 | 1 | −1 | −1 | −4 | −2 | 1 | 0 |
| 290 | E | 307-E | −4 | −6 | −7 | −6 | −6 | −5 | −7 | −6 | −5 | −7 | −3 | −4 | −5 | −4 | −1 | −3 | −2 | −3 | −2 | 8 |
| 291 | E | 308-K | 1 | −5 | −2 | −5 | −5 | 0 | −5 | −5 | −5 | −6 | −2 | −4 | 2 | −4 | −1 | 0 | 3 | 3 | −1 | 5 |
| 292 | E | 309-D | −1 | 0 | −6 | −6 | −2 | −5 | −7 | −7 | −5 | −6 | −2 | −4 | −6 | −1 | −1 | −4 | 0 | 2 | 5 | 5 |
| 293 | I | 310-I | −4 | −7 | 6 | −1 | 5 | 0 | 0 | −6 | −6 | −4 | −5 | −4 | −4 | −7 | −6 | −7 | −6 | −6 | −7 | −6 |
| 294 | F | 311-F | −5 | −7 | −4 | −1 | −4 | 1 | 8 | −2 | −7 | −6 | −6 | −5 | 7 | −6 | −6 | −3 | −6 | −6 | −7 | −6 |
| 295 | E | 312-D | 2 | −5 | −4 | −6 | −5 | −5 | −6 | −6 | −5 | −6 | −2 | −2 | −5 | −3 | 2 | 2 | 1 | 3 | 3 | 4 |
| 296 | A | 313-Y | 2 | −1 | 2 | 1 | −2 | 1 | −1 | −5 | −5 | 0 | −2 | −2 | 3 | −5 | 0 | 5 | −1 | −1 | −5 | −2 |
| 297 | L | 314-I | −3 | −7 | 3 | 6 | 1 | −1 | 1 | −5 | −6 | −5 | −6 | −4 | −4 | −7 | −6 | −6 | −6 | −6 | −7 | −6 |
| 298 | G | 315-Q | −3 | 7 | −7 | −7 | −7 | −6 | −7 | −6 | −1 | −6 | −1 | −5 | −6 | −1 | 0 | −5 | −5 | −5 | −1 | −5 |
| 299 | L | 316-W | −2 | −7 | −1 | 5 | 1 | 5 | 2 | 4 | −6 | −5 | −5 | −4 | −4 | −7 | −5 | −6 | −6 | −6 | −7 | −6 |
| 300 | P | 317-K | 0 | −5 | −6 | −4 | −6 | −5 | −7 | −7 | 5 | −6 | −1 | −4 | −6 | −4 | 1 | −4 | 2 | 0 | 3 | 3 |
| 301 | Y | 318-Y | −5 | −6 | −5 | −3 | −5 | −4 | 2 | 9 | −7 | 0 | −5 | −5 | 8 | −6 | −5 | −3 | −2 | −1 | −7 | −6 |
| 302 | I | 319-R | −2 | −6 | 5 | 1 | 1 | −3 | −4 | 1 | −2 | −5 | −5 | −4 | −5 | −5 | −2 | −5 | 1 | 4 | −6 | −5 |
| 303 | E | 320-E | −1 | −5 | −6 | −6 | −6 | −6 | −7 | −7 | 6 | −6 | −2 | −4 | −6 | −4 | −3 | 0 | −1 | −4 | −3 | 6 |
| 304 | P | 321-P | −4 | −6 | −6 | −6 | −6 | −6 | −7 | −7 | 9 | −6 | −4 | −4 | −6 | −5 | −5 | −6 | −4 | −6 | −5 | −5 |
| 305 | E | 322-K | −3 | −5 | −6 | −6 | −6 | −5 | −6 | 6 | −2 | −6 | −1 | 0 | −4 | −4 | 0 | 4 | 2 | −3 | −3 | 6 |
| 306 | L | 323-D | −5 | −6 | 1 | 3 | −4 | 0 | 0 | −6 | −5 | −6 | −4 | −4 | −4 | −4 | 2 | 4 | −4 | −4 | 2 | 4 |
| 307 | R | 324-R | −5 | −6 | −7 | −6 | −6 | −5 | −6 | −6 | −6 | −7 | −4 | −5 | −5 | −4 | −2 | −4 | −1 | 8 | −1 | −1 |

TABLE 2
Position-specific scoring matrix (PSSM) for BRCT (cd00027) (SEQ ID NO: 88) domain where a threshold bit score of
29.2119 is required to qualify as a member of this family. The PSSM was obtained from the NCBI CDD. In the upper
left hand corner table header, P signifies position, C signifies the consensus sequence, and M signifies the master sequence
of known structure. In the rest of the table, letters indicate standard amino acid single letter nomenclature.

| P | C | M | A | G | I | L | V | M | F | W | P | C | S | T | Y | N | Q |
|---|---|---|---|---|---|---|---|---|---|---|---|---|---|---|---|---|---|
| 1 | G | 8-G | −3 | 5 | −5 | −7 | −1 | −10 | −3 | −11 | −5 | −10 | −5 | −4 | −3 | 3 | −1 |
| 2 | L | 9-K | −9 | −11 | 1 | 3 | 2 | 1 | 2 | −10 | −1 | 6 | −2 | −4 | −3 | −5 | −2 |
| 3 | T | 10-H | −1 | −5 | 0 | −2 | 2 | −1 | −9 | 2 | −5 | −10 | 2 | 3 | 0 | −2 | −1 |
| 4 | F | 11-F | −4 | −11 | 5 | −1 | 3 | 3 | 7 | −8 | −11 | −3 | −10 | −9 | 1 | −6 | −10 |
| 5 | V | 12-F | 0 | −5 | −1 | 0 | 3 | −1 | 3 | −8 | −10 | 6 | 0 | −5 | 5 | −10 | −9 |
| 6 | I | 13-L | −4 | −11 | 5 | 3 | 3 | −7 | 4 | −9 | −6 | 3 | −4 | −4 | −1 | −11 | −5 |
| 7 | T | 14-Y | −2 | 0 | −4 | −1 | −1 | 1 | −1 | 0 | −5 | 1 | 3 | 5 | 3 | 0 | −9 |
| 8 | G | 15-G | −2 | 5 | −1 | −6 | −2 | −1 | −3 | −11 | −1 | −2 | 2 | −8 | −10 | 0 | −4 |
| 9 | D | 16-E | −1 | −1 | 0 | 0 | −2 | 0 | 2 | −1 | −1 | 2 | 1 | 0 | 1 | −2 | −2 |
| 10 | L | 17-F | −1 | −4 | −1 | 2 | 0 | −3 | 3 | −8 | −1 | 1 | −1 | 1 | −2 | 0 | −1 |
| 11 | P | 18-P | −1 | −1 | −1 | −2 | −1 | 2 | −1 | −3 | 2 | −1 | −1 | 1 | −1 | 1 | 0 |
| 12 | S | 19-G | −2 | 1 | −1 | −2 | −3 | 0 | −5 | −5 | 2 | 0 | 2 | 0 | −2 | 1 | 2 |
| 13 | E | 20-D | −2 | −4 | 0 | −1 | −2 | 3 | 2 | −10 | 2 | 1 | 1 | 0 | 1 | 2 | 1 |
| 14 | E | 21-E | −2 | −3 | −4 | −1 | −2 | −9 | −2 | −10 | 1 | −2 | 1 | 1 | −2 | 2 | 1 |
| 15 | R | 22-R | −2 | −10 | 0 | −1 | −3 | 2 | −3 | −10 | −10 | −10 | −3 | −3 | 0 | −2 | −2 |
| 16 | D | 23-R | 0 | −2 | −1 | −2 | −5 | −2 | −2 | −1 | −5 | −1 | 2 | 1 | −2 | 0 | 0 |
| 17 | E | 24-K | −2 | −4 | 1 | −2 | −4 | −4 | 0 | −1 | −2 | −10 | −1 | −2 | 0 | 0 | 2 |
| 18 | L | 25-L | 1 | −7 | 3 | 5 | 1 | 3 | −2 | 3 | −10 | −9 | −3 | −2 | 0 | −4 | −9 |
| 19 | K | 26-I | −2 | −5 | 1 | −4 | −2 | 1 | −9 | −10 | −9 | −2 | 0 | 2 | 1 | −5 | 3 |
| 20 | E | 27-R | 0 | −4 | −6 | −5 | −10 | −9 | 0 | −2 | −9 | −11 | −2 | 0 | −4 | 2 | 0 |
| 21 | L | 28-Y | 0 | −10 | 2 | 4 | −2 | 3 | 2 | 1 | −10 | 0 | −2 | −2 | −1 | −5 | 1 |
| 22 | I | 29-V | 2 | −11 | 6 | 2 | 3 | −2 | 2 | −10 | −10 | −3 | −6 | −3 | −3 | −11 | −10 |
| 23 | E | 30-T | −3 | −6 | −1 | −1 | 1 | 1 | −10 | −10 | −4 | −10 | −1 | −1 | −3 | −1 | 2 |
| 24 | K | 31-A | 2 | −4 | −2 | 0 | −6 | 0 | −4 | −1 | −5 | 1 | 0 | −1 | −2 | 1 | 1 |
| 25 | L | 32-F | −1 | −1 | −2 | 4 | −3 | 3 | 3 | −9 | −10 | 2 | −4 | −4 | 1 | 2 | −3 |
| 26 | G | 33-N | −4 | 7 | −11 | −4 | −11 | −10 | 0 | −10 | −10 | −3 | −8 | −9 | −2 | −3 | −9 |
| 27 | G | 34-G | 3 | 6 | −4 | −5 | −4 | −1 | 0 | −2 | −4 | −2 | −8 | −9 | −10 | −5 | −3 |
| 28 | K | 35-E | −1 | −9 | −3 | −4 | −1 | −9 | −10 | −11 | −9 | −3 | 1 | 3 | −2 | 0 | 0 |
| 29 | V | 36-L | −6 | −11 | 4 | 0 | 6 | 0 | 2 | −9 | −4 | 0 | −4 | −4 | 3 | −11 | −10 |
| 30 | T | 37-E | −1 | −3 | −1 | 1 | 2 | 1 | −5 | −10 | −3 | −1 | 1 | 4 | −2 | −3 | −2 |
| 31 | S | 38-D | −2 | 0 | −4 | −6 | −3 | −9 | −1 | 2 | 1 | −2 | 2 | 2 | −2 | 1 | 0 |
| 32 | S | 39-Y | −2 | −5 | −10 | −4 | −3 | −4 | 0 | −10 | −3 | −1 | 3 | 0 | 1 | 0 | 1 |
| 33 | V | 40-M | −1 | −2 | 2 | 2 | 3 | 1 | 2 | 1 | 0 | −1 | −2 | −3 | 1 | −3 | −2 |
| 34 | S | 41-S | −2 | −4 | −3 | −4 | −1 | −2 | −1 | −10 | 1 | −1 | 4 | 2 | −4 | 1 | 1 |
| 35 | K | 42-D | −2 | 0 | −1 | −2 | −2 | −5 | −1 | 1 | 1 | −5 | 1 | 0 | 0 | 1 | −2 |
| 36 | K | 43-R | −3 | −2 | −5 | −4 | −2 | −9 | −10 | −10 | −2 | −2 | 2 | 2 | 0 | 1 | 2 |
| 37 | T | 44-V | 1 | −10 | 1 | −3 | 3 | −2 | −1 | −10 | −10 | 4 | −2 | 4 | −4 | 0 | −2 |
| 38 | T | 45-Q | −4 | −9 | −5 | −6 | −4 | −9 | −2 | −11 | −2 | −2 | 1 | 7 | −9 | −3 | −1 |
| 39 | H | 46-F | −6 | −10 | 1 | −1 | −2 | −8 | 4 | −9 | −11 | 0 | −9 | −9 | 5 | −1 | −8 |
| 40 | V | 47-V | −2 | −11 | 3 | 4 | 5 | −3 | 2 | −10 | −5 | 3 | −4 | −5 | −1 | −11 | −5 |
| 41 | I | 48-I | −4 | −11 | 7 | 0 | 6 | −7 | −2 | −10 | −11 | −9 | −10 | −8 | −9 | −11 | −10 |
| 42 | V | 49-T | 2 | −3 | 1 | 0 | 4 | 1 | 1 | −10 | −10 | 3 | 1 | 1 | 0 | −10 | −9 |
| 43 | G | 50-A | −1 | 3 | −3 | −5 | −2 | −9 | −10 | −2 | 2 | 1 | 1 | −2 | 0 | 1 | 0 |
| 44 | S | 51-Q | −4 | 0 | −4 | −7 | −4 | −9 | −5 | −11 | −1 | −1 | 2 | 3 | −2 | 0 | −1 |
| 45 | D | 52-E | 0 | −3 | −1 | −1 | −3 | −1 | −1 | −10 | 2 | −1 | 0 | 0 | 1 | 3 | −4 |
| 46 | A | 53-W | 2 | 0 | −1 | −2 | −2 | −2 | −1 | −1 | 2 | 0 | 1 | −1 | −1 | 1 | −1 |
| 47 | G | 54-D | −1 | 3 | −5 | −2 | −2 | −3 | −3 | 0 | −1 | 0 | −2 | 1 | −2 | 2 | 0 |
| 48 | P | 55-P | 1 | 1 | −2 | −3 | −5 | −2 | −2 | −7 | 2 | −2 | 1 | 1 | −3 | −3 | 1 |
| 49 | K | 56-S | −3 | −3 | 0 | −1 | 1 | −6 | −1 | −7 | −4 | −2 | 1 | 0 | −2 | −1 | −4 |
| 50 | K | 57-F | −1 | −2 | −2 | 1 | 1 | 0 | 1 | −3 | −1 | 4 | −1 | −1 | 0 | 0 | −2 |
| 51 | L | 59-E | −2 | −6 | −1 | 3 | 1 | 1 | 3 | −5 | −2 | 2 | −3 | −5 | 5 | −6 | −3 |
| 52 | L | 60-A | 1 | −4 | 0 | 2 | −3 | 2 | 1 | 0 | −3 | −2 | −2 | −1 | −4 | 0 | 2 |
| 53 | K | 61-L | 1 | −5 | 0 | 1 | −1 | 0 | 0 | −8 | −3 | 1 | 0 | −2 | −2 | −1 | 0 |
| 54 | A | 62-M | 5 | 0 | 1 | −2 | −1 | −3 | −4 | 4 | −5 | 0 | −1 | −3 | 1 | −5 | −1 |
| 55 | I | 63-D | −2 | −6 | 4 | 0 | 0 | −4 | 0 | −1 | 0 | −3 | 0 | −4 | −9 | −1 | 2 |
| 56 | K | 64-N | 1 | −3 | −1 | −1 | −2 | −2 | 1 | −10 | −2 | 1 | 1 | −2 | −2 | 1 | 1 |
| 57 | L | 65-P | −2 | −3 | −5 | 2 | −2 | −2 | −1 | 3 | 1 | 1 | 1 | −2 | 1 | 2 | −3 |
| 58 | G | 66-S | −3 | 5 | −11 | −6 | −3 | −10 | −4 | −11 | −2 | −10 | −1 | −9 | −4 | 4 | −1 |
| 59 | I | 67-L | −1 | −4 | 5 | 0 | 3 | −8 | −2 | −1 | 0 | 1 | −2 | 1 | −2 | 10 | −5 |
| 60 | P | 68-A | 0 | −6 | 2 | −4 | −1 | −3 | −3 | 5 | 4 | −2 | −4 | −3 | −4 | −3 | 0 |
| 61 | I | 69-F | −3 | −11 | 6 | 1 | 4 | 0 | 2 | −10 | −11 | 4 | −9 | −1 | 0 | −11 | −4 |
| 62 | V | 70-V | −2 | −4 | 3 | 2 | 6 | 4 | −8 | 1 | −10 | −9 | −10 | −8 | −4 | −3 | −10 |
| 63 | T | 71-R | −8 | −3 | −10 | −4 | −10 | 0 | −10 | −11 | −2 | −1 | 2 | 3 | −3 | 3 | −2 |
| 64 | P | 72-P | −1 | −4 | 1 | −7 | 0 | −1 | 0 | −2 | 4 | 0 | −1 | −2 | −3 | −1 | −1 |
| 65 | E | 73-R | −3 | 0 | −1 | −10 | −5 | −3 | −1 | −2 | −9 | −10 | 3 | 0 | −9 | −1 | 3 |
| 66 | W | 74-W | −5 | −5 | −9 | −2 | −3 | −9 | 3 | 12 | −11 | −10 | −10 | −10 | 3 | −11 | −10 |
| 67 | L | 75-I | −5 | −11 | 5 | 4 | 4 | 4 | 2 | −1 | −11 | −9 | −4 | −9 | −8 | −11 | −10 |
| 68 | L | 76-Y | −3 | −4 | 0 | 2 | 0 | −1 | 0 | 4 | −6 | 0 | −4 | 2 | 2 | −1 | 0 |
| 69 | D | 77-S | 2 | −6 | −6 | −1 | −4 | −9 | −1 | −10 | −9 | −10 | 0 | −3 | −1 | −1 | 0 |
| 70 | C | 78-C | −3 | −6 | 1 | 0 | −2 | −1 | −5 | −10 | −10 | 9 | 4 | −3 | −2 | −9 | −9 |
| 71 | L | 79-N | −2 | −5 | 4 | 3 | 1 | 2 | 2 | 2 | −6 | 1 | −9 | −3 | 1 | −3 | −5 |
| 72 | K | 80-E | 0 | −2 | −5 | −5 | −3 | 0 | −10 | −11 | −4 | 1 | 0 | −1 | −2 | −1 | 2 |

| P | C | M | H | K | R | D | E |
|---|---|---|---|---|---|---|---|
| 1 | G | 8-G | −1 | 1 | −2 | 2 | −1 |
| 2 | L | 9-K | 2 | 3 | −4 | −10 | −6 |
| 3 | T | 10-H | 0 | 2 | 2 | −9 | 0 |
| 4 | F | 11-F | −10 | −11 | −11 | −11 | −11 |
| 5 | V | 12-F | 3 | −4 | −3 | −5 | −6 |
| 6 | I | 13-L | −10 | −10 | −10 | −11 | −11 |
| 7 | T | 14-Y | 1 | −5 | −5 | −1 | −9 |
| 8 | G | 15-G | −2 | −3 | 1 | −1 | −1 |
| 9 | D | 16-E | −9 | −1 | −1 | 2 | 0 |
| 10 | L | 17-F | −1 | 1 | −1 | 1 | −2 |
| 11 | P | 18-P | −1 | 1 | 0 | 1 | 1 |
| 12 | S | 19-G | −1 | 1 | −1 | 0 | 0 |
| 13 | E | 20-D | 0 | −1 | −3 | −5 | 2 |
| 14 | E | 21-E | −2 | 0 | 1 | 2 | 3 |
| 15 | R | 22-R | −1 | 3 | 6 | 0 | −3 |
| 16 | D | 23-R | 3 | 2 | −1 | 3 | 2 |
| 17 | E | 24-K | 0 | 1 | 0 | 3 | 3 |
| 18 | L | 25-L | −10 | −3 | −5 | −11 | −6 |
| 19 | K | 26-I | 0 | 4 | 1 | −4 | 2 |
| 20 | E | 27-R | −1 | 3 | 3 | 2 | 4 |
| 21 | L | 28-Y | −3 | 2 | 0 | −5 | −4 |
| 22 | I | 29-V | −2 | −10 | −10 | −11 | −6 |
| 23 | E | 30-T | 0 | 3 | 3 | −8 | 4 |
| 24 | K | 31-A | 0 | 3 | 1 | −1 | 2 |
| 25 | L | 32-F | 4 | 0 | −1 | −5 | −9 |
| 26 | G | 33-N | 0 | −2 | −3 | −1 | −10 |
| 27 | G | 34-G | −10 | 0 | −9 | −9 | −9 |
| 28 | K | 35-E | −1 | 5 | 2 | 0 | 1 |
| 29 | V | 36-L | −2 | −4 | −4 | −11 | −10 |
| 30 | T | 37-E | 2 | −2 | −1 | −5 | 1 |
| 31 | S | 38-D | −4 | 1 | −1 | 1 | 1 |
| 32 | S | 39-Y | −3 | 0 | 0 | 4 | 1 |
| 33 | V | 40-M | −1 | −1 | −6 | −10 | −1 |
| 34 | S | 41-S | −3 | −3 | −2 | 2 | −2 |
| 35 | K | 42-D | 0 | 2 | 2 | 1 | 1 |
| 36 | K | 43-R | −9 | 3 | 0 | 3 | 1 |
| 37 | T | 44-V | 0 | −2 | −2 | −2 | −4 |
| 38 | T | 45-Q | −2 | −6 | −9 | 3 | −4 |
| 39 | H | 46-F | 10 | −9 | −5 | −10 | −9 |
| 40 | V | 47-V | −1 | −10 | −10 | −11 | −10 |
| 41 | I | 48-I | −11 | −10 | −11 | −11 | −11 |
| 42 | V | 49-T | −10 | −9 | −5 | −10 | −10 |
| 43 | G | 50-A | 1 | 2 | −9 | 2 | 0 |
| 44 | S | 51-Q | 1 | 1 | 2 | 2 | 2 |
| 45 | D | 52-E | 1 | −2 | −1 | 3 | 1 |
| 46 | A | 53-W | 0 | −4 | −1 | 0 | 2 |
| 47 | G | 54-D | −8 | −1 | 1 | 2 | −2 |
| 48 | P | 55-P | 0 | 1 | 0 | 0 | 1 |
| 49 | K | 56-S | −1 | 4 | 1 | 0 | 2 |
| 50 | K | 57-F | −3 | 3 | −1 | −1 | −2 |
| 51 | L | 59-E | 1 | 2 | −4 | −6 | −1 |
| 52 | L | 60-A | −6 | 2 | 0 | 0 | 1 |
| 53 | K | 61-L | 1 | 3 | 0 | −1 | −1 |
| 54 | A | 62-M | −8 | 0 | −1 | −8 | −2 |
| 55 | I | 63-D | −1 | 3 | 0 | 0 | 1 |
| 56 | K | 64-N | −1 | 2 | 1 | 0 | 1 |
| 57 | L | 65-P | 3 | −1 | 1 | −3 | −1 |
| 58 | G | 66-S | 2 | 2 | 0 | −1 | −1 |
| 59 | I | 67-L | −3 | 1 | 0 | −3 | −5 |
| 60 | P | 68-A | 3 | 2 | 1 | −1 | −1 |
| 61 | I | 69-F | −3 | −10 | −10 | −11 | −11 |
| 62 | V | 70-V | −11 | −10 | −10 | −11 | −10 |
| 63 | T | 71-R | 3 | 3 | 1 | 2 | −6 |
| 64 | P | 72-P | 3 | −2 | −3 | −1 | 3 |
| 65 | E | 73-R | 0 | −1 | −2 | 3 | 3 |
| 66 | W | 74-W | −2 | −10 | −6 | −5 | −4 |
| 67 | L | 75-I | −3 | −6 | −6 | −11 | −11 |
| 68 | L | 76-Y | 0 | 2 | 0 | −1 | 1 |
| 69 | D | 77-S | −2 | −1 | −1 | 5 | 3 |
| 70 | C | 78-C | −2 | −4 | −6 | −10 | −10 |
| 71 | L | 79-N | −4 | −2 | −2 | −4 | −2 |
| 72 | K | 80-E | 0 | 4 | 2 | 2 | 2 |

Alternatively, the absence or truncation of a BRCT domain in a terminal transferase enzyme is defined as the following:
any modifications, mutations, deletions, or insertions of the two conserved motifs of the wild-type, natural BRCT domain consisting of the “dimer interface” and/or the “BRCT sequence” (with a characteristic sequence of Trp-X-X-X-Cys/Ser) as to make the terminal transferase better behaved and/or dissociate from nucleic acids more rapidly. The dimer interface shall be defined as positions 16, 20, 23, 26, and 28 in the cd00027 PSSM with a consensus sequence of Asp/Glu (16), Glu/Lys (20), Glu/Arg/Lys (23), Gly (26), and Lys/Thr (28). The BRCT sequence shall be defined as positions 66 and 70 in the cd00027 PSSM with a consensus sequence of Trp (66) and Cys/Ser (70). 10 Better behaved can mean less prone to aggregation, greater enzymatic turnover rates, better multi-step cycling efficiencies, and/or maintains longer activity under storage or reaction conditions. Conserved motifs are as annotated by the Conserved Domain Database (CDD).

Alternatively, the absence or truncation of a BRCT domain in a terminal transferase enzyme is defined as the following:
any amino acid sequences that have a 90% or more sequence identity to the terminal transferase sequence list provided in Appendix 1 or a fragment thereof that do not contain a BRCT superfamily annotation (cl00038, cd00027, smart00292, pfam00533, pfam12738, pfam16589, pfam16759, pfam16770) as defined by the Conserved Domain Database (CDD)⁸ maintained by the US National Center for Biotechnology Information (NCBI) identified via a Conserved Domain Search (CD-Search)⁹.

Alternatively, the absence or truncation of a BRCT domain in a terminal transferase enzyme is defined as the following:
any mutation, modification or truncation of the N-terminal portion (defined as first 200 amino acids). Such a truncation is shown herein to result in greater multi-step cycling efficiency.

In one embodiment, the modified terminal transferase enzyme is immobilised on a solid support. In an alternative embodiment, the modified terminal transferase enzyme is in solution phase. Detailed methodology of providing a terminal transferase enzyme in the solution phase or immobilised on a solid support are provided in GB Patent Application No. 1701396.2, the contents of which are herein incorporated by reference.

### Nucleic Acid Synthesis Method

In one embodiment of the invention, there is provided a use according to the first aspect of the invention in a method of nucleic acid synthesis.

According to a second aspect of the invention, there is provided a method of nucleic acid synthesis, which comprises the steps of:
(a) providing an initial initiator sequence;
(b) adding a reversibly blocked nucleotide triphosphate to said initiator sequence in the presence of a modified terminal transferase enzyme as defined herein;
(c) removal of all reagents from the initiator sequence;
(d) cleaving the blocking group from the reversibly blocked nucleotide added in step (b) to said initiator sequence; and
(e) removal of the cleaving agent.

References herein to a ‘method of nucleic acid synthesis’ include methods of synthesising lengths of DNA (deoxyribonucleic acid) or RNA (ribonucleic acid) wherein a strand of nucleic acid (n) is extended by adding a further nucleotide (n+1). In one embodiment, the nucleic acid is DNA. In an alternative embodiment, the nucleic acid is RNA.

References herein to ‘method of DNA synthesis’ refer to a method of DNA strand synthesis wherein a DNA strand (n) is extended by adding a further nucleotide (n+1). The method described herein provides a novel use of the terminal deoxynucleotidyl transferases of the invention and nucleotide triphosphate having a 3′-O-azidomethyl substituent to sequentially add nucleotides in de novo DNA strand synthesis which has several advantages over the DNA synthesis methods currently known in the art.

In a further embodiment greater than 1 nucleotide is added by repeating steps (b) to (e).

It will be understood that steps (b) to (e) of the method may be repeated multiple times to produce a DNA or RNA strand of a desired length. Therefore, in one embodiment, greater than 1 nucleotide is added to the initiator sequence, such as greater than 5, 10, 20, 30, 40, 50, 60, 70, 80, 90, 100, 110 or 120 nucleotides are added to the initiator sequence by repeating steps (b) to (e). In a further embodiment, greater than 200 nucleotides are added, such as greater than 300, 400, 500, 600, 700, 800, 900, 1000, 1250, 1500, 1750, 2000, 2250, 2500, 2750, 3000, 4000, 5000, 6000, 7000, 8000, 9000 or 10000 nucleotides.

References herein to ‘nucleotide triphosphates’ refer to a molecule containing a nucleoside (i.e. a base attached to a deoxyribose or ribose sugar molecule) bound to three phosphate groups. Examples of nucleotide triphosphates that contain deoxyribose are: deoxyadenosine triphosphate (dATP), deoxyguanosine triphosphate (dGTP), deoxycytidine triphosphate (dCTP) or deoxythymidine triphosphate (dTTP). Examples of nucleotide triphosphates that contain ribose are: adenosine triphosphate (ATP), guanosine triphosphate (GTP), cytidine triphosphate (CTP) or uridine triphosphate (UTP). Other types of nucleosides may be bound to three phosphates to form nucleotide triphosphates, such as naturally occurring modified nucleosides and artificial nucleosides.

References herein to ‘reversibly blocked’ nucleotides include nucleotides containing reversibly terminating moieties at the sugar moiety (3′-blocked nucleotide triphosphates) and the nitrogenous base moiety (base-blocked nucleotide triphosphates). A reversible terminator is a chemical moiety that can be added to the 3′-end of a nucleic acid initiator by a polymerase or terminal transferase and prevents further addition of nucleotides. If and only if the reversible terminator is cleaved by a cleaving agent can the polymerase or terminal transferase add additional nucleotides.

### 3′-Blocked Nucleotide Triphosphates

Therefore, references herein to ‘3’-blocked nucleotide triphosphates' refer to nucleotide triphosphates (e.g., dATP, dGTP, dCTP or dTTP) which have an additional group on the 3′ end which prevents further addition of nucleotides, i.e., by replacing the 3′-OH group with a protecting group.

It will be understood that references herein to ‘3’-blocked', ‘3’-blocking group' or ‘3’-protecting group’ refer to the group attached to the 3′ end of the nucleotide triphosphate which prevents further nucleotide addition. The present method uses reversible 3′-blocking groups which can be removed by cleavage to allow the addition of further nucleotides. By contrast, irreversible 3′-blocking groups refer to dNTPs where the 3′-OH group can neither be exposed nor uncovered by cleavage.

There exist several documented reversible protecting groups, such as 2-cyanoethyl, azidomethyl, aminoxy, and allyl, which can be applied to the method described herein. Examples of suitable protecting groups are described in Greene's Protective Groups in Organic Synthesis, (Wuts, P.G.M. & Greene, T.W. (2012) 4th Ed., John Wiley & Sons).

In one embodiment, the 3′-blocked nucleotide triphosphate is blocked by a reversible protecting group. In an alternative embodiment, the 3′-blocked nucleotide triphosphate is blocked by an irreversible protecting group.

Therefore, in one embodiment, the 3′-blocked nucleotide triphosphate is blocked by either a 3′-O-methyl, 3′-azido, 3′-O-azidomethyl, 3′-aminoxy, 3′-O-(2-cyanoethyl), 3′-O-(2-cyanoethoxy), or 3′-O-allyl group. In a further embodiment, the 3′-blocked nucleotide triphosphate is blocked by either a 3′-O-azidomethyl, 3′-aminoxy, 3′-O-(2-cyanoethyl), 3′-O-(2-cyanoethoxy), or 3′-O-allyl group.

### Base-Blocked Nucleotide Triphosphates

Therefore, references herein to base-blocked nucleotide triphosphates’ refer to nucleotide triphosphates (e.g., dATP, dGTP, dCTP or dTTP) which have an additional group on the nitrogenous base which prevents further addition of nucleotides. Reversibly terminating moieties located on the nitrogenous base may be any molecular moiety that if and only if cleaved with a cleaving agent allows the addition of subsequent nucleotides. The reversible terminator may be located on guanine at the 7 or 8 position; adenine at the 7, 8, or N6 positions; and the pyrimidines at the 5 position.

There exist several documented reversible protecting groups that can be attached to the nitrogenous base, including photocleavable substituted nitrobenzyl groups, peptides, and other chemical/support moieties mentioned in the alternating-phase section of this patent.

### Cleaving Agent

References herein to ‘cleaving agent’ refer to a substance which is able to cleave the 3′-blocking group from the reversibly blocked nucleotide triphosphate.

The reversible blocking groups described herein may all be quantitatively removed in aqueous solution with documented compounds which may be used as cleaving agents (for example, see: Wuts, P.G.M. & Greene, T.W. (2012) 4th Ed., John Wiley & Sons; Hutter, D. et al. (2010) Nucleosides Nucleotides Nucleic Acids 29, 879-895; EP 1560838 and U.S. Pat. No. 7,795,424).

In one embodiment, the cleaving agent is a chemical cleaving agent. In an alternative embodiment, the cleaving agent is an enzymatic cleaving agent. In a further embodiment, the cleaving agent is electromagnetic radiation, for instance ultraviolet or visible light.

It will be understood by the person skilled in the art that the selection of cleaving agent is dependent on the type of reversibly blocked nucleotide used. For instance, tris(2-carboxyethyl)phosphine (TCEP) can be used to cleave a 3′-O-azidomethyl group, palladium complexes can be used to cleave a 3′-O-allyl group, ammonium hydroxide can be used to cleave a 3′-O-(2-cyanoethyl)/3′-O-2-(cyanoethoxy)methyl group, or sodium nitrite can be used to cleave a 3′-aminoxy group. Therefore, in one embodiment, the cleaving agent is selected from: tris(2-carboxyethyl)phosphine (TCEP), a palladium complex or sodium nitrite.

In one embodiment, the cleaving agent is added in the presence of a cleavage solution comprising a denaturant, such as urea, guanidinium chloride, formamide or betaine. The addition of a denaturant has the advantage of being able to disrupt any undesirable secondary structures in the DNA. In a further embodiment, the cleavage solution comprises one or more buffers. It will be understood by the person skilled in the art that the choice of buffer is dependent on the exact cleavage chemistry and cleaving agent required.

### Initiator Sequences

References herein to an ‘initial initiator sequence’ refer to a short oligonucleotide with a free 3′-end which the reversibly blocked nucleotide triphosphate can be attached to for the first addition of a reversibly blocked nucleotide triphosphate by a terminal transferase enzyme. In one embodiment, the initial initiator sequence is a DNA initiator sequence. In an alternative embodiment, the initial initiator sequence is an RNA initiator sequence.

References herein to an ‘initiator sequence’ refer to an oligonucleotide with a free 3′-end which the reversibly blocked nucleotide triphosphate can be attached to. In one embodiment, the initiator sequence is a DNA initiator sequence. In an alternative embodiment, the initiator sequence is an RNA initiator sequence.

References herein to a ‘DNA initiator sequence’ refer to a sequence of DNA which the reversibly blocked nucleotide triphosphate can be attached to, i.e. DNA will be synthesised from the end of the DNA initiator sequence.

In one embodiment, the initial initiator sequence is between 5 and 100 nucleotides long, such as between 10 and 90 nucleotides long, in particular between 5 and 20 nucleotides long.

In one embodiment, the initiator sequence is single-stranded. In an alternative embodiment, the initiator sequence is double-stranded. It will be understood by persons skilled in the art that a 3′-overhang (i.e., a free 3′-end) allows for efficient addition.

In one embodiment, the initiator sequence is immobilised on a solid support. This allows the modified terminal transferase enzyme and the cleaving agent to be removed (in steps (c) and (e), respectively) without washing away the synthesised nucleic acid. The initiator sequence may be attached to a solid support stable under aqueous conditions so that the method can be easily performed via a flow setup.

In one embodiment, the initiator sequence is immobilised on a solid support via a reversible interacting moiety, such as a chemically-cleavable linker, an antibody/immunogenic epitope, a biotin/biotin binding protein (such as avidin or streptavidin), or glutathione-GST tag. Therefore, in a further embodiment, the method additionally comprises extracting the resultant nucleic acid by removing the reversible interacting moiety in the initiator sequence, such as by incubating with proteinase K.

In a further embodiment, the initiator sequence is immobilised on a solid support via a chemically-cleavable linker, such as a disulfide, allyl, or azide-masked hemiaminal ether linker. Therefore, in one embodiment, the method additionally comprises extracting the resultant nucleic acid by cleaving the chemical linker through the addition of tris(2-carboxyethyl)phosphine (TCEP) or dithiothreitol (DTT) for a disulfide linker; palladium complexes for an allyl linker; or TCEP for an azide-masked hemiaminal ether linker.

In one embodiment, the resultant nucleic acid is extracted and amplified by polymerase chain reaction using the nucleic acid bound to the solid support as a template. The initiator sequence could therefore contain an appropriate forward primer sequence and an appropriate reverse primer could be synthesised.

In an alternative embodiment, the immobilised initiator sequence contains at least one restriction site. Therefore, in a further embodiment, the method additionally comprises extracting the resultant nucleic acid by using one or more restriction enzymes.

The use of restriction enzymes and restriction sites to cut nucleic acids in a specific location is well known in the art. The choice of restriction site and enzyme can depend on the desired properties, for example whether ‘blunt’ or ‘sticky’ ends are required. Examples of restriction enzymes include: AluI, BamHI, EcoRI, EcoRII, EcoRV, HaeII, HgaI, HindIII, HinfI, NotI, PstI, PvuII, SalI, Sau3A, ScaI, SmaI, TaqI and XbaI.

In an alternative embodiment, the initiator sequence contains at least one uridine. Treatment with uracil-DNA glycosylase (UDG) generates an abasic site. Treatment on an appropriate substrate with an apurinic/apyrimidinic (AP) site endonuclease will extract the nucleic acid strand.

### Buffers

In one embodiment, the modified terminal transferase enzyme of the invention is added in the presence of an extension solution comprising one or more buffers (e.g., Tris or cacodylate), one or more salts (e.g., Na⁺, K⁺, Mg²⁺, Mn²⁺, Cu²⁺, Zn²⁺, Co²⁺, etc., all with appropriate counterions, such as Cl⁻) and inorganic pyrophosphatase (e.g., the Saccharomyces cerevisiae homolog). It will be understood that the choice of buffers and salts depends on the optimal enzyme activity and stability.

The use of an inorganic pyrophosphatase helps to reduce the build-up of pyrophosphate due to nucleotide triphosphate hydrolysis by terminal transferase. Therefore, the use of an inorganic pyrophosphatase has the advantage of reducing the rate of (1) backwards reaction and (2) terminal transferase strand dismutation. In one embodiment, the inorganic pyrophosphatase comprises purified, recombinant inorganic pyrophosphatase from Saccharomyces cerevisiae.

In one embodiment, step (b) is performed at a pH range between 5 and 10. Therefore, it will be understood that any buffer with a buffering range of pH 5-10 could be used, for example cacodylate, Tris, HEPES or Tricine, in particular cacodylate or Tris.

In one embodiment, step (d) is performed at a temperature less than 99 C, such as less than 95° C., 90° C., 85° C., 80° C., 75° C., 70° C., 65° C., 60° C., 55° C., 50° C., 45° C., 40° C., 35° C., or 30° C. It will be understood that the optimal temperature will depend on the cleavage agent utilised. The temperature used helps to assist cleavage and disrupt any secondary structures formed during nucleotide addition.

In one embodiment, steps (c) and (e) are performed by applying a wash solution. In one embodiment, the wash solution comprises the same buffers and salts as used in the extension solution described herein. This has the advantage of allowing the wash solution to be collected after step (c) and recycled as extension solution in step (b) when the method steps are repeated.

### Devices

In one embodiment, the method is performed within a flow instrument, such as a microfluidic or column-based flow instrument. The method described herein can easily be performed in a flow setup which makes the method simple to use. It will be understood that examples of commercially available DNA synthesisers (e.g., MerMade 192E from BioAutomation or H-8 SE from K&A) may be optimised for the required reaction conditions and used to perform the method described herein.

In one embodiment, the method is performed on a plate or microarray setup. For example, nucleotides may be individually addressed through a series of microdispensing nozzles using any applicable jetting technology, including piezo and thermal jets. This highly parallel process may be used to generate hybridization microarrays and is also amenable to DNA fragment assembly through standard molecular biology techniques.

In one embodiment, there is provided a method which is performed in a microfluidic device. Thus, according to a further aspect of the invention, there is provided a method of nucleic acid synthesis which is performed in a microfluidic device comprising the steps of:
(a) providing an initial initiator sequence bound to a surface within a microfluidic device;
(b) adding a reversibly blocked nucleotide triphosphate to said initiator sequence in the presence of a modified terminal transferase enzyme as defined herein;
(c) removal of all reagents from the initiator sequence;
(d) cleaving the blocking group from the reversibly blocked nucleotide added in step (b) to said initiator sequence; and
(e) removal of the cleaving agent.

References herein to microfluidic device include continuous-flow microfluidic devices, droplet-based microfluidic devices, programmable digital microfluidic device, digital microfluidic devices, microarray devices (such as DNA chips), optofluidic devices and acoustic droplet ejection (ADE) devices.

In a further embodiment, greater than 1 nucleotide is added by repeating steps (b) to (e).

In a further embodiment, the surface within the microfluidic device in step (a) may be patterned to yield initiators bound at defined locations. Therefore in a further embodiment the microfluidic device may have a reaction chamber or a plurality of reaction chambers, such as greater than 100, 1000 or 10000 reaction chambers.

In one embodiment, the method additionally comprises amplifying the resultant nucleic acid. Methods of DNA/RNA amplification are well known in the art. For example, in a further embodiment, the amplification is performed by polymerase chain reaction (PCR). This step has the advantage of being able to amplify and extract the resultant nucleic acid all in one step.

The template independent nucleic acid synthesis method described herein has the capability to add a nucleic acid sequence of defined composition and length to an initiator sequence. Therefore, it will be understood by persons skilled in the art, that the method described herein may be used as a novel way to introduce adapter sequences to a nucleic acid library.

If the initiator sequence is not one defined sequence, but instead a library of nucleic acid fragments (for example generated by sonication of genomic DNA, or for example messenger RNA) then this method is capable of de novo synthesis of ‘adapter sequences’ on every fragment. The installation of adapter sequences is an integral part of library preparation for next-generation library nucleic acid sequencing methods, as they contain sequence information allowing hybridisation to a flow cell/solid support and hybridisation of a sequencing primer.

Currently used methods include single-stranded ligation; however, this technique is limited because ligation efficiency decreases strongly with increasing fragment length. Consequently, current methods are unable to attach sequences longer than 100 nucleotides in length. Therefore, the method described herein allows for library preparation in an improved fashion to that which is currently possible.

Therefore, in one embodiment, an adapter sequence is added to the initiator sequence. In a further embodiment, the initiator sequence may be a nucleic acid from a library of nucleic acid fragments.

### Alternating-Phase Processes

### General Alternating-Phase Process

According to a fifth aspect of the invention, there is provided an alternating-phase polymer synthesis method which comprises the steps of:
(a) providing a monomer immobilised to a support moiety via a cleavable linker;
(b) providing a polymer of length (N);
(c) providing a modified terminal transferase enzyme as defined herein to couple the polymer to the immobilised monomer to create an immobilised, coupled polymer of length (N+1);
(d) removing any uncoupled polymers; and
(e) cleaving the immobilised, coupled polymer of length (N+1) from the support moiety.

Full details of the fifth aspect of the invention are provided in GB Patent Application No. 1701396.2, the description and figures of which are herein incorporated by reference.

It will be appreciated that greater than one monomer may be added by providing the product of step (e) to an additional monomer immobilised to a support moiety and then repeating steps (b) and (e) until a polymer of desired length is synthesised.

In one embodiment, the removing in step (d) comprises a washing step. Such a washing step serves the purpose of providing an error correction step by removing all unbound polymers.

In one embodiment, the cleaving in step (e) comprises light, pH, temperature, voltage and the like.

In one embodiment, an isolation or capture step is conducted following step (e).

It will be appreciated that the polymer may either be in solution phase or is itself immobilised to a support moiety via a cleavable linker.

### Alternating-Phase Nucleic Acid Synthesis Process

One embodiment of the general alternating-phase process described herein as the fifth aspect of the invention is referred to herein as “Alternating-Phase Nucleic Acid Synthesis Process”.

In one embodiment of the fifth aspect of the invention, the monomer is a deoxynucleotide triphosphate (dNTP) or nucleotide triphosphate (NTP) immobilised to a support moiety via a cleavable linker.

In one embodiment of the fifth aspect of the invention, the polymer is an initiator nucleic acid sequence of length (N).

Thus, according to a sixth aspect of the invention, there is provided a nucleic acid synthesis method which comprises the steps of:
(a) providing a deoxynucleotide triphosphate (dNTP) or nucleotide triphosphate (NTP) immobilised to a support moiety via a cleavable linker;
(b) providing an initiator nucleic acid sequence of length (N);
(c) adding a modified terminal transferase enzyme as defined herein to couple the initiator nucleic acid sequence to the immobilised dNTP/NTP to create an immobilised, coupled sequence of length (N+1);
(d) removing any uncoupled initiator nucleic acid sequences; and
(e) cleaving the immobilised, coupled sequence of length (N+1) from the support moiety.

Full details of the sixth aspect of the invention are provided in GB Patent Application No. 1701396.2, the description and figures of which are herein incorporated by reference.

It will be appreciated that greater than one dNTP/NTP may be added by providing the product of step (e) to an additional deoxynucleotide triphosphate (dNTP) or nucleotide triphosphate (NTP) immobilised to a support moiety and then repeating steps (b) and (e) until a nucleic acid of desired length is synthesised.

In one embodiment, the removing in step (d) comprises a washing step. Such a washing step serves the purpose of providing an error correction step by removing all unbound initiator nucleic acid sequences.

In one embodiment, the cleaving in step (e) comprises light, pH, temperature, voltage and the like.

In one embodiment, an isolation or capture step is conducted following step (e).

### Process Variant 1

One embodiment of the alternating-phase nucleic acid synthesis process described herein as the sixth aspect of the invention is referred to herein as “process variant 1”. In general, this variant to the process relates to the inclusion of a trap strand which is immobilised to the same support moiety as the dNTP/NTP.

Therefore, in one embodiment of the sixth aspect of the invention, the method additionally comprises providing a nucleic acid trap strand sequence which is complimentary to, and capable of hybridising to, the initiator nucleic acid sequence, wherein said trap strand is immobilised at the 3′-end to the same support moiety as the dNTP/NTP in step (a).

Thus, according to a seventh aspect of the invention, there is provided a nucleic acid synthesis method which comprises the steps of:
(a) providing a deoxynucleotide triphosphate (dNTP) or nucleotide triphosphate (NTP) immobilised to a support moiety via a cleavable linker;
(b) providing an initiator nucleic acid sequence of length (N);
(c) providing a nucleic acid trap strand sequence which is complimentary to, and capable of hybridising to, the initiator nucleic acid sequence, wherein said trap strand is immobilised at the 3′-end to the same support moiety as the dNTP/NTP in step (a);
(d) adding a modified terminal transferase enzyme as defined herein to couple the initiator nucleic acid sequence to the immobilised dNTP/NTP to create an immobilised, coupled sequence of length (N+1);
(e) providing a reaction temperature greater than the melting temperature of any trap strand/initiator sequence duplexes;
(f) removing any uncoupled initiator nucleic acid sequences;
(g) providing a reaction temperature lower than the melting temperature of any trap strand/initiator sequence duplexes;
(h) cleaving the immobilised, coupled sequence of length (N+1) from the support moiety; and
(i) providing a reaction temperature greater than the melting temperature of any trap strand/initiator sequence duplexes to separate the trap strand/initiator sequence duplexes.

Full details of the seventh aspect of the invention are provided in GB Patent Application No. 1701396.2, the description and figures of which are herein incorporated by reference.

It will be appreciated that greater than one dNTP/NTP may be added by providing the product of step (i) to an additional support moiety having immobilised thereon a required deoxynucleotide triphosphate (dNTP) or nucleotide triphosphate (NTP) and an immobilised trap strand and then repeating steps (d) and (i) until a nucleic acid of desired length is synthesised.

It will also be appreciated that the 5′ end of the initiator nucleic acid sequence may hybridize to the trap strand to form a duplex. In one embodiment, the duplex is at least 10, 20 or 30 base pairs in length.

Additionally or alternatively, the modified terminal transferase enzyme in step (d) adds the immobilised dNTP/NTP to the 3′ end of the initiator nucleic acid sequence.

In one embodiment, the temperature provided in steps (e) and (i) is selected to prevent the formation of duplexes, such a temperature will suitably be approximately 95° C.

In one embodiment, the removing in step (f) comprises a washing step. Such a washing step serves the purpose of providing an error correction step by removing all unbound initiator nucleic acid sequences. In a further embodiment, step (f) is conducted at the same temperature as step (e).

In one embodiment, the temperature provided in step (g) is selected to allow the formation of duplexes via hybridisation.

In one embodiment, the cleaving in step (h) comprises light, pH, temperature, voltage and the like. In a further embodiment, the cleaving in step (h) comprises a cleavage agent selected from a reducing agent (i.e. TCEP) or a specific pH buffer. Such a cleavage agent cleaves the cleavable linker connecting the 3′ end of the coupled sequence of length (N+1) from the support moiety. In one embodiment, the temperature provided in step (h) is any temperature below the melting temperature of any trap strand/initiator sequence duplexes in order to facilitate cleavage.

In one embodiment, following cleavage in step (h), a washing step may be performed in order to remove any leftover cleavage agent.

### Process Variant 2

A further embodiment to the alternating-phase nucleic acid synthesis process described herein as the sixth aspect of the invention is referred to herein as “process variant 2”. In general, this variant to the process relates to the fact that the dNTP/NTP is immobilized to a mobile phase support moiety and the initiator nucleic acid sequence is immobilised to a solid phase support moiety.

Therefore, in one embodiment of the sixth aspect of the invention, the method additionally comprises providing the dNTP/NTP immobilized to a mobile phase support moiety via a cleavable linker and an initiator nucleic acid sequence of length (N) immobilised to a solid phase support moiety via a cleavable linker.

Thus, according to an eighth aspect of the invention, there is provided a nucleic acid synthesis method which comprises the steps of:
(a) providing a deoxynucleotide triphosphate (dNTP) or nucleotide triphosphate (NTP) immobilised to a mobile phase support moiety via a cleavable linker or a dNTP/NTP containing a reversible terminator or blocking moiety via the nitrogenous base;
(b) providing an initiator nucleic acid sequence of length (N) immobilised to a solid phase support moiety via a cleavable linker;
(c) adding a modified terminal transferase enzyme as defined herein to couple the mobile phase immobilised dNTP/NTP to the solid phase immobilised initiator nucleic acid sequence to create an immobilised, coupled sequence of length (N+1);
(d) removing any uncoupled initiator nucleic acid sequences; and
(e) cleaving the mobile phase support moiety from the immobilised, coupled sequence of length (N+1).

Full details of the eighth aspect of the invention are provided in GB Patent Application No. 1701396.2, the description and figures of which are herein incorporated by reference.

In one embodiment, the solid phase support moiety comprises the base of a reaction well and the mobile phase support moiety comprises a bead in solution phase within said reaction well.

In one embodiment, the 5′ end of the initiator nucleic acid sequence is immobilised to the solid phase support moiety and the 3′ end is free from the surface.

In one embodiment, following addition of the modified terminal transferase enzyme in step (c), an exonuclease, such as a 3′-5′ exonuclease (e.g. exonuclease I from E. coli) may be added. This step provides the advantage of degrading any N species which remain as an error correction step to prevent deletions or mutations.

In one embodiment, the cleaving in step (e) comprises light, pH, temperature, voltage and the like. In a further embodiment, the cleaving in step (b) comprises a cleavage agent selected from a reducing agent (i.e. TCEP), light, heat or a specific pH buffer.

In one embodiment, following the cleaving step (e) a washing step is performed. Such a washing step serves the purpose of removing all solutions used in the previous steps.

dNTP/NTPs

References herein to ‘deoxynucleotide triphosphate (dNTP)’ refer to a molecule containing a nucleoside (i.e. a base attached to a deoxyribose or ribose sugar molecule) bound to three phosphate groups. Examples of nucleotide triphosphates that contain deoxyribose are: deoxyadenosine triphosphate (dATP), deoxyguanosine triphosphate (dGTP), deoxycytidine triphosphate (dCTP) or deoxythymidine triphosphate (dTTP). Examples of “nucleotide triphosphates (NTPs) that contain ribose are: adenosine triphosphate (ATP), guanosine triphosphate (GTP), cytidine triphosphate (CTP) or uridine triphosphate (UTP). Other types of nucleosides may be bound to three phosphates to form nucleotide triphosphates, such as naturally occurring modified nucleosides and artificial nucleosides.

### Support Moieties

It will be appreciated that the support moiety will either comprise a solid phase support moiety or a mobile phase support moiety. It will also be appreciated that the solid phase support moiety or mobile phase support moiety for the dNTP/NTP and/or initiator nucleic acid sequence to be immobilised will be selected from any suitable substrate capable of allowing a dNTP/NTP and/or initiator nucleic acid sequence to be immobilised. Solid phase support moieties typically comprise a surface, material, or particle that remains stationary during the entirety of the synthesis process. Mobile phase support moieties (e.g., particles, beads, nanomaterials, etc.) typically comprise a surface, material, or bead greater than or equal to 1 nm in size, such as 1-1000 nm, in particular 1-100 nm, especially ≥2 nm, ≥3 nm, ≥5 nm or ≥10 nm, that may be mobile or stationary during different portions of the synthesis process.

Examples of suitable solid/mobile phase support moieties may be selected from: a solid surface, (e.g., glass, silicon, gold, plastic etc.), such as a flat surface in particular a 96/384-well plate or a hydrophobic substrate (such as Teflon); a particle, bead, nanoparticle, and/or nanobead including quantum dots (e.g., CdSeS/ZnS, InP/ZnS, and/or CuInS2ZnS), magnetic particles (e.g., iron oxide), metal/metalloid/metal alloy particles (e.g., gold, silver, and/or selenium), metal oxide particles (e.g., oxides of Al, Mg, Zr, Ce, Ti, Zn, Fe, Sn), silica particles, agarose particles, polystyrene particles, carbon-based, i.e. organic, particles (e.g., graphene and/or graphene oxide, nucleic acids, proteins and carbohydrates); and any aforementioned surface, particle, bead, nanoparticle, and/or nanobead that is functionalized or passivated (e.g., with polyethylene glycol, gold, etc.), each of which may be ≥1 nm, such as 1-1000 nm, in particular 1-100 nm, especially ≥1 nm, ≥2 nm, ≥3 nm, ≥5 nm or ≥10 nm in any dimension.

In one embodiment, the solid phase support moiety is selected from a solid surface (e.g., glass, silicon, gold, plastic etc.), such as a flat surface in particular a 96/384-well plate or a hydrophobic substrate (such as Teflon); a solid phase particle, a polymer, and a membrane.

In one embodiment, the mobile phase support moiety is selected from: a mobile phase particle, nanoparticle, ultrafine particle, nanomaterial, or any other material greater than or equal to 1 nm in size, such as 1-1000 nm, in particular 1-100 nm, especially ≥1 nm, ≥2 nm, ≥3 nm, ≥5 nm or ≥10 nm. When using the terms nanoparticle, ultrafine particle, or nanomaterial, they apply to both soluble and insoluble particles.

Examples of suitable polymers may be selected from: polyethylene glycols and polyethylene oxides of any molecular weight; natural polymers and biopolymers of any molecular weight (e.g., dextran, cellulose, collagen, lignins, polyamino acids, chitosan/chitin, nucleic acids, and/or any other carbohydrate or starches); biodegradable polymers (e.g., polylactide, polyglycolide, polyphosphoesters, caprolactone, etc.); Pi-conjugated polymers (e.g., cyano-polyphenylene vinylene, polyaniline, polyfluorenes, poly(fluorine vinylenes), polypyridines, etc.); hydrophilic polymers (e.g., poly(vinyl alcohol), poly(acrylic acid), polyvinylpyrrolidone, poly(2-oxazoline), etc.); polysiloxane polymers; hydrophobic polymers (e.g., styrenes, olefins, esters, ethers, carbonates, etc.); and any aforementioned polymer that is functionalized with a chemical or biochemical moiety allowing for covalent or noncovalent attachment of molecules.

Examples of suitable membranes may be selected from: a lipid bilayer; a lipid monolayer; a vesicle or micelle; a membrane formed by polymers (e.g., cellulose-based, polyvinylidene fluoride, etc.); and any aforementioned membrane that is functionalized with a chemical or biochemical moiety allowing for covalent or noncovalent attachment of molecules.

In one embodiment, the support moiety (i.e. the mobile phase support moiety) comprises a spherical or globular particle which is ≥1 nm in diameter. In a further embodiment, the support moiety comprises a spherical or globular particle which is 1-1000 nm, such as 1-100 nm, in particular ≥1 nm, ≥2 nm, ≥3 nm, ≥5 nm or ≥10 nm in diameter.

In an alternative embodiment, the support moiety (i.e. the mobile phase support moiety) comprises a rod or rod-like particle which is ≥1 nm in any dimension. In a further embodiment, the support moiety comprises a rod or rod-like particle which is 1-1000 nm, such as 1-100 nm, in particular ≥1 nm, ≥2 nm, ≥3 nm, ≥5 nm or ≥10 nm in any dimension.

In an alternative embodiment, the support moiety (i.e. the mobile phase support moiety) comprises a flat structure such as a surface which is ≥1 nm in any dimension. In a further embodiment, the support moiety comprises a flat structure such as a surface which is 1-1000 nm, such as 1-100 nm, in particular ≥1 nm, ≥2 nm, ≥3 nm, ≥5 nm or ≥10 nm in any dimension.

In one embodiment, the support moiety (i.e. the mobile phase support moiety) has a molecular weight >1,000 Da, such as >5,000, in particular, >10,000 Da, especially >25,000 Da.

In one embodiment, the dNTP/NTP is immobilized on a solid phase particle or immobilized by depositing directly onto the surface where nucleic acid synthesis will occur. If the dNTP/NTP is immobilized on a solid phase particle, the solid phase particle will be immobilized onto the surface where nucleic acid synthesis will occur. Alternatively, the solid phase particle may be first immobilized on the surface where nucleic acid synthesis will occur. The dNTP/NTP is then immobilized onto the solid phase particle. In an alternative embodiment, the dNTP/NTP is immobilized on a mobile phase particle. The dNTP/NTP immobilized on a mobile phase particle is subsequently immobilized to a solid phase support moiety following addition to an initiator strand immobilized to a solid phase support moiety as per the process described in process variant 2.

In one embodiment the method of immobilization of solid phase particles is magnetic.

In one embodiment, the dNTP/NTP is immobilized to the solid phase support via the nitrogenous base (i.e. purine or pyrimidine moiety) or the triphosphate moiety or the sugar moiety.

In a further embodiment, the immobilisation comprises an azide-alkyne 1,3-dipolar cycloaddition, a tetrazine/alkene-based cycloaddition, a gold-sulfur bond, a nucleophilic addition of an amine to an epoxide group, a biotin-streptavidin/avidin interaction, the Michael addition of a sulfhydryl group to a Michael acceptor (e.g., maleimide), the oxidation of two sulfhydryl groups to form a disulfide bond, an antibody-antigen interaction (e.g., digoxigenin-anti-digoxigenin), etc.

It will be understood that the immobilization linker contains a cleavable linker. Thus, in one embodiment, immobilization is reversible and/or cleavable.

In one embodiment, the cleavable linker is capable of being cleaved by electromagnetic radiation (e.g., 350 nm light) or a reducing agent or an oxidizing agent or heat or electrochemical or a combination thereof.

In one embodiment, the 5′ immobilized strand (i.e., the trap strand) contains a functional group, such as an azido group, capable of linking the strand on the 5′-end to the solid support surface.

### Cleavable Linkers

It will be appreciated that a cleavable linker is a broadly stable moiety that connects two or more units. However, upon exposure to the cleavage condition the linker is disrupted, and thus separation of the two units connected by the linker occurs. To offer utility, the cleavage condition must be compatible with the system of interest. There are many chemically cleavable linkers available in the art. Some suitable non-limiting examples include:

A linker comprising an azide masked hemiaminal ether sites (—OCHN₃—), which may be cleaved by an azide to amine reduction, triggering a spontaneous breakdown of the revealed hemiaminal ether. Suitable reducing agents include phosphines (e.g., TCEP) , thiols (e.g., DTT, EDT) and metal-ligand complexes, including organometallic Ru—, Ir—, Cr—, Rh— and Co— complexes. An example of a suitable metal-ligand complexes is organometallic (Ru(bpy)3 2+) and salts thereof, including Ru(bpy)₃Cl₂.

Other compositions for protected hemiaminal ethers include allyl or allyl carbamate moieties, which may be cleaved using transition metals complexed with water soluble ligands, e.g., Pd with water soluble phosphine ligands); sulfmoc, which may be cleaved with a mild base, e.g. 1% Na₂CO₃; m-chloro-p-acyloxybenzyl carbamate, which may be cleaved with mild base, e.g.: 0.1 M NaOH; and 4-azidobenzyl carbamate, which may be cleaved with reducing agents, e.g.: TCEP, DTT).

A linker comprising a phosphine moiety, which may be cleaved through incubation with azide reagents, for example alkyl or aryl azides. The aza-ylid generated may react with a suitably positioned ester moiety to facilitate cleavage.

A linker comprising a silicon containing site, which may be cleaved in the presence of fluoride ions, such as KF and tetra-n-butylammonium fluoride (TBAF).

A linker comprising a disulfide site, which may be cleaved by reduction with phosphine or thiol reagents.

A linker comprising a cyanoethyl site, which may be cleaved under basic conditions, such as solutions of NH₃ or 10% K₂CO₃.

A linker comprising a photocleavable site, which may be cleaved by UV light, ideally of a wavelength orthogonal to the system of interest. Suitable photocleavable sites are well known in the art. For example, an orthonitrobenzyl group may be cleaved by UV at 365 nm.

Other suitable cleavage sites are well known in the art.

Immobilised dNTP/NTPs

In one embodiment, the nucleotide is blocked with a compound of formula (I):

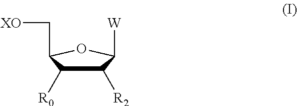

wherein R₀ represents a hydroxyl protecting group;
R₂ represents hydrogen, hydroxyl, —N₃, alkoxy, alkyl, alkenyl, alkynyl, —O-2-(cyanoethoxy)methyl, —O-(2-cyanoethyl), —O-azidomethyl, -aminoxy, or —O-allyl;
X represents hydrogen or one or more phosphate groups; and
W represents a base.

In an alternative embodiment, the nucleotide is blocked with a compound of formula (II):

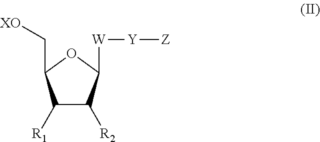

wherein R₁ and R₂ independently represent H or OH or a protected derivative thereof;
X represents hydrogen or one or more phosphate groups;
W represents a base;
Y represents a cleavable linker; and
Z represents a blocking group or support moiety.

In one embodiment, X represents a monophosphate, diphosphate, triphosphate or tetraphosphate group.

In one embodiment, W is selected from a nitrogenous base. In a further embodiment, W is selected from a purine or pyrimidine moiety. In a yet further embodiment, the base is selected from adenine, guanine, uracil, thymine or cytosine.

In one embodiment, the support moiety defined as Z is as defined herein.

In a further embodiment, the support moiety additionally comprises a nucleic acid trap strand sequence as defined herein.

### Kits

According to a further aspect of the invention, there is provided a kit comprising a modified terminal transferase enzyme as defined herein, optionally in combination with one or more components selected from: an initiator sequence, a microfluidic device or chip, one or more reversibly blocked nucleotide triphosphates, inorganic pyrophosphatase, such as purified, recombinant inorganic pyrophosphatase from Saccharomyces cerevisiae, and a cleaving agent; further optionally together with instructions for use of the kit in accordance with the method as defined herein.

Suitably a kit according to the invention may also contain one or more components selected from the group: an extension solution, a wash solution and/or a cleaving solution as defined herein; optionally together with instructions for use of the kit in accordance with any of the methods defined herein.

According to a further aspect of the invention, there is provided the use of a kit as defined herein in a method of nucleic acid synthesis.

The following studies and protocols illustrate embodiments of the methods described herein:

Full length TdT can be engineered to accept 3′-reversibly blocked nucleotide triphosphates (see WO 2016/128731), which provide a means of sequence control in an enzymatic-based nucleic acid synthesis platform, as evidenced by FIG. 2. However, full length TdT possesses several traits that make it ill suited as an enzyme for use in cyclical processes, which are not observed in single addition assays. Full length TdT is prone to aggregation in solution, rapidly fouls a large variety of surfaces, and strongly associates with DNA as a monomeric species, multimeric species, or an aggregate, as evidenced by FIG. 4. In FIG. 4, the inventors have demonstrated that full-length TdT does not readily dissociate from a DNA initiator molecule whereas truncated TdT does readily dissociate. It follows that the incorporation efficiency of subsequent modified nucleotides may suffer due to the misbehavior of full length TdT. Possible causes include restriction of access to the added 3′ reversible terminator, preventing either deprotection by chemical or enzymatic means, or persistent binding of an incompetent enzyme form.

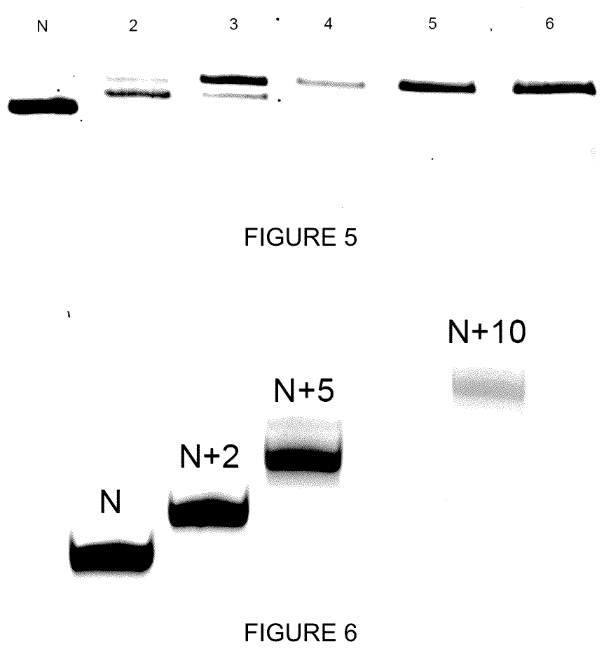

Indeed, full length engineered TdTs are generally incapable of adding modified nucleotide triphosphates quantitatively in series, as evidenced by FIGS. 5 and 6. The multi-step cycling assay was performed as follows: the (1) reversibly terminated nucleotide triphosphate is added onto the 3′-end of an immobilized DNA initiator molecule via TdT catalysis, (2) unreacted nucleotide triphosphate and enzyme are washed off, (3) the added reversibly terminated nucleotide is deprotected, (4) the deprotection agent is washed off, and (5) the process repeats from step (1). Due to the cyclic nature of TdT-mediated nucleic acid synthesis, multi-step incorporation efficiency rather than single-step incorporation efficiency must be used to judge the quality of TdT enzyme variants. Thus, the tendency to associate strongly with DNA is the reason for poor multi-step modified nucleotide triphosphate incorporation efficiencies (FIGS. 3-4), despite the quantitative single-step conversion of the same modified nucleotides triphosphates shown in FIG. 2.

As mentioned above, the inventors made a series of truncations to wild-type and engineered variants of TdT as part of our search for a better TdT variant. Surprisingly, the inventors found that an engineered TdT lacking the BRCT domain or a fragment thereof is capable of adding modified nucleotide triphosphates quantitatively in series, whereas the full length engineered TdT enzyme is unable to do such sequential additions. As shown in FIG. 4, truncated TdT does not associate as strongly with DNA, thus resulting in better multi-step incorporation efficiencies. N-terminal mutations and truncations of TdT also reduce the Stokes radius of the enzyme, resulting in less steric issues if the DNA initiator molecule is immobilized to a surface, as well as better penetration of the enzyme into a porous matrix. FIG. 5 shows full-length TdT exhibits poor conversion of an initiator to an N+2 product. In stark contrast, N-terminal mutations of TdT result in drastic increases in cycle incorporation efficiencies, as evidenced by the ability to add more than ten reversibly blocked nucleotides in series to the 3′-end of a DNA initiator molecule, as shown in FIG. 6.

### REFERENCES

1. Sukumar, N., Boulé, J. B. & Expert-Bezançon, N. Crystallization of the catalytic domain of murine terminal deoxynucleotidyl transferase. . . . Section D: Biological . . . (2000). doi: 10.1107/S090744490001297X
2. Delarue, M. et al. Crystal structures of a template-independent DNA polymerase: murine terminal deoxynucleotidyltransferase. 21, 427-439 (2002).
3. Mozzarelli, A. & Rossi, G. L. Protein function in the crystal. Annual review of biophysics and . . . (1996).
4. Wang, A. H. et al. Molecular structure of a left-handed double helical DNA fragment at atomic resolution. Nature 282, 680-686 (1979).
5. Repasky, J. A. E., Corbett, E., Boboila, C. & Schatz, D. G. Mutational analysis of terminal deoxynucleotidyltransferase-mediated N-nucleotide addition in V(D)J recombination. J. Immunol. 172, 5478-5488 (2004).
6. Thai, T. H., Purugganan, M. M., Roth, D. B. & Kearney, J. F. Distinct and opposite diversifying activities of terminal transferase splice variants. Nature immunology (2002).
7. Andrade, P., Martín, M. J., Juárez, R., López de Saro, F. & Blanco, L. Limited terminal transferase in human DNA polymerase mu defines the required balance between accuracy and efficiency in NHEJ. Proc. Natl. Acad. Sci. U.S.A. 106, 16203-16208 (2009).
8. Marchler-Bauer, A. et al. CDD/SPARCLE: functional classification of proteins via subfamily domain architectures. Nucl. Acids Res. 45, D200-D203 (2017).
9. Marchler-Bauer, A. & Bryant, S. H. CD-Search: protein domain annotations on the fly. Nucleic Acids Res. 32, W327-31 (2004).

APPENDIX 1
Terminal transferase enzyme sequences

>tr|W5MK82|W5MK82_LEPOC Uncharacterized protein OS=Lepisosteus oculatus GN=DNTT PE=4
SV=1;
>tr|A0A1S3N3Q5|A0A1S3N3Q5_SALSA DNA nucleotidylexotransferase-like OS=Salmo salar
GN=LOC106576788 PE=4 SV=1;
>tr|A0A1S3RVH9|A0A1S3RVH9_SALSA DNA nucleotidylexotransferase isoform X1 OS=Salmo salar
GN=LOC106605322 PE=4 SV=1;
>tr|A0A1S3RVI4|A0A1S3RVI4_SALSA DNA nucleotidylexotransferase isoform X2 OS=Salmo salar
GN=LOC106605322 PE=4 SV=1;
>tr|W5U8U3|W5U8U3_ICTPU DNA nucleotidylexotransferase OS=Ictalurus punctatus GN=dntt PE=2
SV=1;
>sp|Q92089|TDT_ONCMY DNA nucleotidylexotransferase OS=Oncorhynchus mykiss GN=dntt PE=2
SV=1;
>tr|Q6T422|Q6T422_GINCI Terminal deoxynucleotidyl transferase OS=Ginglymostoma cirratum
GN=TdT PE=2 SV=1;
>tr|W5L524|W5L524_ASTMX Uncharacterized protein OS=Astyanax mexicanus GN=DNTT PE=4
SV=1;
>tr|A0A1S3FAV4|A0A1S3FAV4_DIPOR DNA nucleotidylexotransferase isoform X1 OS=Dipodomys
ordii GN=Dntt PE=4 SV=1;
>tr|Q5EB91|Q5EB91_RAT Deoxynucleotidyltransferase, terminal OS=Rattus norvegicus GN=Dntt PE=2
SV=1;
>tr|E9PT58|E9PT58_RAT DNA nucleotidylexotransferase OS=Rattus norvegicus GN=Dntt PE=4 SV=1;
>tr|H3DI66|H3DI66_TETNG Uncharacterized protein OS=Tetraodon nigroviridis GN=DNTT PE=4
SV=1;
>tr|A0A1S3FA64|A0A1S3FA64_DIPOR DNA nucleotidylexotransferase isoform X2 OS=Dipodomys
ordii GN=Dntt PE=4 SV=1;
>tr|G1PDC9|G1PDC9_MYOLU Uncharacterized protein OS=Myotis lucifugus GN=DNTT PE=4 SV=1;
>tr|G3QFE9|G3QFE9_GORGO Uncharacterized protein OS=Gorilla gorilla gorilla GN=DNTT PE=4
SV=1;
>tr|G7PDN0|G7PDN0_MACFA Putative uncharacterized protein OS=Macaca fascicularis
GN=EGM_18244 PE=4 SV=1;
>tr|F7A3Y1|F7A3Y1_MACMU DNA nucleotidylexotransferase isoform 1 OS=Macaca mulatta
GN=DNTT PE=2 SV=1;
>tr|I3KC46|I3KC46_ORENI Uncharacterized protein OS=Oreochromis niloticus GN=DNTT PE=4 SV=1;
>tr|A0A096P5U2|A0A096P5U2_PAPAN Uncharacterized protein OS=Papio anubis GN=DNTT PE=4
SV=1;
>tr|G1RSJ0|G1RSJ0_NOMLE Uncharacterized protein OS=Nomascus leucogenys GN=DNTT PE=4
SV=1;
>sp|P06526|TDT_BOVIN DNA nucleotidylexotransferase OS=Bos taurus GN=DNTT PE=1 SV=2;
>tr|A0A140T8D0|A0A140T8D0_BOVIN DNA nucleotidylexotransferase OS=Bos taurus GN=DNTT
PE=4 SV=1;
>tr|Q3UZ80|Q3UZ80_MOUSE Putative uncharacterized protein OS=Mus musculus GN=Dntt PE=2
SV=1;
>tr|F1SBG2|F1SBG2_PIG Uncharacterized protein OS=Sus scrofa GN=DNTT PE=4 SV=1;
>sp|P09838-2|TDT_MOUSE Isoform TDT-S of DNA nucleotidylexotransferase OS=Mus musculus
GN=Dntt;
>tr|F6V4S9|F6V4S9_HORSE Uncharacterized protein OS=Equus caballus GN=DNTT PE=4 SV=1;
>tr|H2Q2B9|H2Q2B9_PANTR Uncharacterized protein OS=Pan troglodytes GN=DNTT PE=4 SV=1;
>tr|L8IDA9|L8IDA9_9CETA DNA nucleotidylexotransferase (Fragment) OS=Bos mutus
GN=M91_12325 PE=4 SV=1;
>tr|H2NB52|H2NB52_PONAB Uncharacterized protein OS=Pongo abelii GN=DNTT PE=4 SV=1;
>tr|D2H5M3|D2H5M3_AILME Putative uncharacterized protein (Fragment) OS=Ailuropoda melanoleuca
GN=PANDA_005205 PE=4 SV=1;
>sp|P04053|TDT_HUMAN DNA nucleotidylexotransferase OS=Homo sapiens GN=DNTT PE=1 SV=3;
>tr|F6RGZ5|F6RGZ5_CALJA Uncharacterized protein OS=Callithrix jacchus GN=DNTT PE=4 SV=1;
>tr|A0A1S3AC31|A0A1S3AC31_ERIEU DNA nucleotidylexotransferase isoform X1 OS=Erinaceus
europaeus GN=DNTT PE=4 SV=1;
>tr|G3SSH5|G3SSH5_LOXAF Uncharacterized protein OS=Loxodonta africana GN=DNTT PE=4 SV=1;
>tr|M3Z065|M3Z065_MUSPF Uncharacterized protein OS=Mustela putorius furo GN=DNTT PE=4
SV=1;
>tr|A4PCE6|A4PCE6_OTOGA Deoxynucleotidyltransferase, terminal OS=Otolemur garnettii GN=DNTT
PE=4 SV=1;
>tr|G1L0B5|G1L0B5_AILME Uncharacterized protein (Fragment) OS=Ailuropoda melanoleuca
GN=DNTT PE=4 SV=1;
>tr|W5PCG3|W5PCG3_SHEEP Uncharacterized protein OS=Ovis aries GN=DNTT PE=4 SV=1;
>tr|F1P657|F1P657_CANLF Uncharacterized protein OS=Canis lupus familiaris GN=DNTT PE=4 SV=2;
>tr|G3VQ55|G3VQ55_SARHA Uncharacterized protein OS=Sarcophilus harrisii GN=DNTT PE=4 SV=1;
>tr|H0UYE5|H0UYE5_CAVPO Uncharacterized protein OS=Cavia porcellus GN=DNTT PE=4 SV=1;
>tr|U3JZX7|U3JZX7_FICAL Uncharacterized protein OS=Ficedula albicollis GN=DNTT PE=4 SV=1;
>tr|M3W767|M3W767_FELCA Uncharacterized protein OS=Felis catus GN=DNTT PE=4 SV=1;
>sp|P04053-2|TDT_HUMAN Isoform 2 of DNA nucleotidylexotransferase OS=Homo sapiens
GN=DNTT;
>tr|K7FHL8|K7FHL8_PELSI Uncharacterized protein OS=Pelodiscus sinensis GN=DNTT PE=4 SV=1;
>tr|A0A1S3ACC1|A0A1S3ACC1_ERIEU DNA nucleotidylexotransferase isoform X2 OS=Erinaceus
europaeus GN=DNTT PE=4 SV=1;
>tr|A0A091ECZ8|A0A091ECZ8_CORBR DNA nucleotidylexotransferase OS=Corvus brachyrhynchos
GN=N302_13526 PE=4 SV=1;
>tr|G3VQ54|G3VQ54_SARHA Uncharacterized protein OS=Sarcophilus harrisii GN=DNTT PE=4 SV=1;
>tr|Q6T421|Q6T421_RAJEG Terminal deoxynucleotidyl transferase OS=Raja eglanteria GN=TdT PE=2
SV=1;
>sp|P09838|TDT_MOUSE DNA nucleotidylexotransferase OS=Mus musculus GN=Dntt PE=1 SV=3;
>tr|H2ZX52|H2ZX52_LATCH Uncharacterized protein OS=Latimeria chalumnae GN=DNTT PE=4
SV=1;
>tr|G1SII3|G1SII3_RABIT Uncharacterized protein OS=Oryctolagus cuniculus GN=DNTT PE=4 SV=2;
>tr|A0A087V8F5|A0A087V8F5_BALRE DNA nucleotidylexotransferase OS=Balearica regulorum
gibbericeps GN=N312_00864 PE=4 SV=1;
>tr|A0A1L8FJ83|A0A1L8FJ83_XENLA Uncharacterized protein OS=Xenopus laevis
GN=XELAEV_18034610mg PE=4 SV=1;
>sp|P42118|TDT_XENLA DNA nucleotidylexotransferase OS=Xenopus laevis GN=dntt PE=2 SV=1;
>tr|Q75U67|Q75U67_TAKRU Terminal deoxynucleotidyl transferase OS=Takifugu rubripes GN=TdT
PE=2 SV=1;
>tr|A0A093EPJ1|A0A093EPJ1_GAVST DNA nucleotidylexotransferase OS=Gavia stellata
GN=N328_03245 PE=4 SV=1;
>tr|G5BEU5|G5BEU5_HETGA DNA nucleotidylexotransferase OS=Heterocephalus glaber
GN=GW7_11927 PE=4 SV=1;
>tr|A2VDB4|A2VDB4_XENLA Dntt-A protein (Fragment) OS=Xenopus laevis GN=dntt-A PE=2 SV=1;
>tr|A0A087X2V1|A0A087X2V1_POEFO Uncharacterized protein OS=Poecilia formosa GN=DNTT
PE=4 SV=2;
>sp|O02789|TDT_MONDO DNA nucleotidylexotransferase OS=Monodelphis domestica GN=DNTT
PE=2 SV=1;
>tr|G3UED4|G3UED4_LOXAF Uncharacterized protein OS=Loxodonta africana GN=DNTT PE=4
SV=1;
>tr|H0ZFJ8|H0ZFJ8_TAEGU Uncharacterized protein OS=Taeniopygia guttata GN=DNTT PE=4 SV=1;
>tr|M3ZZI8|M3ZZI8_XIPMA Uncharacterized protein OS=Xiphophorus maculatus GN=DNTT PE=4
SV=1;
>tr|F6UMV3|F6UMV3_MONDO DNA nucleotidylexotransferase OS=Monodelphis domestica
GN=DNTT PE=4 SV=1;
>tr|A0A091H8B9|A0A091H8B9_BUCRH DNA nucleotidylexotransferase OS=Buceros rhinoceros
silvestris GN=N320_10189 PE=4 SV=1;
>tr|A0A091ULI4|A0A091ULI4_NIPNI DNA nucleotidylexotransferase OS=Nipponia nippon
GN=Y956_01479 PE=4 SV=1;
>tr|F1P317|F1P317_CHICK DNA nucleotidylexotransferase OS=Gallus gallus GN=DNTT PE=4 SV=1;
>tr|A0A0D9R168|A0A0D9R168_CHLSB Uncharacterized protein OS=Chlorocebus sabaeus GN=DNTT
PE=4 SV=1;
>sp|P36195|TDT_CHICK DNA nucleotidylexotransferase OS=Gallus gallus GN=DNTT PE=2 SV=1;
>tr|A0A0A0AT06|A0A0A0AT06_CHAVO DNA nucleotidylexotransferase OS=Charadrius vociferus
GN=N301_10607 PE=4 SV=1;
>tr|H2TS88|H2TS88_TAKRU Uncharacterized protein OS=Takifugu rubripes GN=dntt PE=4 SV=1;
>tr|A0A091VHH3|A0A091VHH3_OPIHO DNA nucleotidylexotransferase OS=Opisthocomus hoazin
GN=N306_09804 PE=4 SV=1;
>tr|A4PCD8|A4PCD8_LEMCA Deoxynucleotidyltransferase, terminal OS=Lemur catta GN=DNTT PE=4
SV=1;
>tr|A0A091KGW0|A0A091KGW0_9GRUI DNA nucleotidylexotransferase OS=Chlamydotis macqueenii
GN=N324_12983 PE=4 SV=1;
>tr|A0A087QZ53|A0A087QZ53_APTFO DNA nucleotidylexotransferase OS=Aptenodytes forsteri
GN=AS27_02643 PE=4 SV=1;
>tr|A0A093HUU2|A0A093HUU2_STRCA DNA nucleotidylexotransferase OS=Struthio camelus australis
GN=N308_09005 PE=4 SV=1;
>sp|A4PCD4|TDT_EULMA DNA nucleotidylexotransferase OS=Eulemur macaco GN=DNTT PE=3
SV=1;
>tr|A0A093NX62|A0A093NX62_PYGAD DNA nucleotidylexotransferase OS=Pygoscelis adeliae
GN=AS28_07241 PE=4 SV=1;
>tr|A0A091TYT5|A0A091TYT5_PHORB DNA nucleotidylexotransferase OS=Phoenicopterus ruber
ruber GN=N337_08342 PE=4 SV=1;
>tr|A0A091QDL2|A0A091QDL2_MERNU DNA nucleotidylexotransferase OS=Merops nubicus
GN=N331_12273 PE=4 SV=1;
>tr|A0A091QU30|A0A091QU30_LEPDC DNA nucleotidylexotransferase OS=Leptosomus discolor
GN=N330_14539 PE=4 SV=1;
>tr|A4PCE2|A4PCE2_MICMU Deoxynucleotidyltransferase, terminal OS=Microcebus murinus
GN=DNTT PE=4 SV=1;
>tr|H9GAY7|H9GAY7_ANOCA Uncharacterized protein OS=Anolis carolinensis GN=DNTT PE=4
SV=2;
>tr|A0A091II02|A0A091II02_CALAN DNA nucleotidylexotransferase OS=Calypte anna
GN=N300_07464 PE=4 SV=1;
>tr|A0A091FKF8|A0A091FKF8_9AVES DNA nucleotidylexotransferase OS=Cuculus canorus
GN=N303_09150 PE=4 SV=1;
>tr|A0A093IEV1|A0A093IEV1_PICPB DNA nucleotidylexotransferase OS=Picoides pubescens
GN=N307_06181 PE=4 SV=1;
>tr|F6VL88|F6VL88_ORNAN Uncharacterized protein OS=Ornithorhynchus anatinus GN=DNTT PE=4
SV=1;
>tr|A0A091JFK2|A0A091JFK2_9AVES DNA nucleotidylexotransferase OS=Egretta garzetta
GN=Z169_12857 PE=4 SV=1;
>tr|A0A0Q3QZM8|A0A0Q3QZM8_AMAAE DNA nucleotidylexotransferase OS=Amazona aestiva
GN=AAES_113066 PE=4 SV=1;
>tr|Q5J2Q9|Q5J2Q9_DANRE Terminal deoxynucleotidyl transferase OS=Danio rerio GN=dntt PE=2
SV=1;
>sp|O57486|TDT_AMBME DNA nucleotidylexotransferase OS=Ambystoma mexicanum GN=DNTT
PE=2 SV=2;
>tr|G1NAM2|G1NAM2_MELGA Uncharacterized protein OS=Meleagris gallopavo GN=DNTT PE=4
SV=2;
>tr|G3NEP6|G3NEP6_GASAC Uncharacterized protein OS=Gasterosteus aculeatus GN=DNTT PE=4
SV=1;
>tr|H3DI67|H3DI67_TETNG Uncharacterized protein OS=Tetraodon nigroviridis GN=DNTT PE=4
SV=1;
>tr|G3NEP3|G3NEP3_GASAC Uncharacterized protein OS=Gasterosteus aculeatus GN=DNTT PE=4
SV=1;
>tr|B3DKA1|B3DKA1_DANRE Dntt protein OS=Danio rerio GN=dntt PE=2 SV=1;
>tr|G3NEP2|G3NEP2_GASAC Uncharacterized protein OS=Gasterosteus aculeatus GN=DNTT PE=4
SV=1;
>tr|L9JG14|L9JG14_TUPCH DNA nucleotidylexotransferase OS=Tupaia chinensis
GN=TREES_T100012289 PE=4 SV=1;
>tr|A0A093PQ85|A0A093PQ85_9PASS DNA nucleotidylexotransferase OS=Manacus vitellinus
GN=N305_05476 PE=4 SV=1;
>tr|A0A091DV02|A0A091DV02_FUKDA DNA nucleotidylexotransferase OS=Fukomys damarensis
GN=H920_02650 PE=4 SV=1;
>tr|G3UPN2|G3UPN2_MELGA Uncharacterized protein OS=Meleagris gallopavo GN=DNTT PE=4
SV=1;
>tr|A0A091MNY8|A0A091MNY8_9PASS DNA nucleotidylexotransferase (Fragment) OS=Acanthisitta
chloris GN=N310_00875 PE=4 SV=1;
>tr|H2TS93|H2TS93_TAKRU Uncharacterized protein OS=Takifugu rubripes GN=dntt PE=4 SV=1;
>tr|H2TS92|H2TS92_TAKRU Uncharacterized protein OS=Takifugu rubripes GN=dntt PE=4 SV=1;
>tr|H2TS91|H2TS91_TAKRU Uncharacterized protein OS=Takifugu rubripes GN=dntt PE=4 SV=1;
>tr|H2TS90|H2TS90_TAKRU Uncharacterized protein OS=Takifugu rubripes GN=dntt PE=4 SV=1;
>tr|H2TS87|H2TS87_TAKRU Uncharacterized protein OS=Takifugu rubripes GN=dntt PE=4 SV=1;
>tr|L5MBP7|L5MBP7_MYODS DNA nucleotidylexotransferase OS=Myotis davidii
GN=MDA_GLEAN10023458 PE=4 SV=1;
>tr|H2TS86|H2TS86_TAKRU Uncharacterized protein OS=Takifugu rubripes GN=dntt PE=4 SV=1;
>tr|A0A099YST9|A0A099YST9_TINGU DNA nucleotidylexotransferase (Fragment) OS=Tinamus
guttatus GN=N309_05590 PE=4 SV=1;
>tr|G3GZU6|G3GZU6_CRIGR DNA nucleotidylexotransferase OS=Cricetulus griseus GN=I79_003393
PE=4 SV=1;
>tr|S7NPM4|S7NPM4_MYOBR DNA nucleotidylexotransferase OS=Myotis brandtii
GN=D623_10025731 PE=4 SV=1;
>tr|A0A061IE05|A0A061IE05_CRIGR DNA nucleotidylexotransferase OS=Cricetulus griseus
GN=H671_3g9305 PE=4 SV=1;
>tr|A0A151P063|A0A151P063_ALLMI DNA nucleotidylexotransferase OS=Alligator mississippiensis
GN=DNTT PE=4 SV=1;
>tr|H2TS89|H2TS89_TAKRU Uncharacterized protein OS=Takifugu rubripes GN=dntt PE=4 SV=1;
>tr|M5B5N0|M5B5N0_PLEWA DNA polymerase mu OS=Pleurodeles waltl GN=polymerase mu PE=2
SV=1;
>tr|A0A1A6H3C9|A0A1A6H3C9_NEOLE Uncharacterized protein OS=Neotoma lepida
GN=A6R68_13117 PE=4 SV=1;
>tr|A0A147ABX1|A0A147ABX1_FUNHE DNA nucleotidylexotransferase (Fragment) OS=Fundulus
heteroclitus PE=4 SV=1;
>tr|A0A1A7ZQP3|A0A1A7ZQP3_NOTFU Deoxynucleotidyltransferase, terminal OS=Nothobranchius
furzeri GN=DNTT PE=4 SV=1;
>tr|L5JPN7|L5JPN7_PTEAL DNA nucleotidylexotransferase OS=Pteropus alecto
GN=PAL_GLEAN10018329 PE=4 SV=1;
>tr|I3JZZ4|I3JZZ4_ORENI Uncharacterized protein OS=Oreochromis niloticus PE=4 SV=1;
>tr|V9KWD7|V9KWD7_CALMI Terminal deoxynucleotidyl transferase (Fragment) OS=Callorhinchus
milii PE=2 SV=1;
>tr|H2VEE3|H2VEE3_TAKRU Uncharacterized protein OS=Takifugu rubripes GN=polm PE=4 SV=1;
>sp|O57486-2|TDT_AMBME Isoform 2 of DNA nucleotidylexotransferase OS=Ambystoma mexicanum
GN=DNTT;
>tr|H2VEE1|H2VEE1_TAKRU Uncharacterized protein OS=Takifugu rubripes GN=polm PE=4 SV=1;
>tr|H3D7S0|H3D7S0_TETNG Uncharacterized protein OS=Tetraodon nigroviridis PE=4 SV=1;
>tr|A0A1A8MSX8|A0A1A8MSX8_9TELE Polymerase (DNA directed), mu OS=Nothobranchius pienaari
GN=POLM PE=4 SV=1;
>tr|A0A1A6HWD2|A0A1A6HWD2_NEOLE Uncharacterized protein OS=Neotoma lepida
GN=A6R68_23458 PE=4 SV=1;
>tr|Q66HH0|Q66HH0_RAT DNA polymerase mu OS=Rattus norvegicus GN=Polm PE=2 SV=1;
>tr|A0A1S3GI13|A0A1S3GI13_DIPOR DNA-directed DNA/RNA polymerase mu OS=Dipodomys ordii
GN=Polm PE=4 SV=1;
>tr|A0A1A8ERH9|A0A1A8ERH9_9TELE Polymerase (DNA directed), mu OS=Nothobranchius
korthausae GN=POLM PE=4 SV=1;
>tr|M3ZG06|M3ZG06_XIPMA Uncharacterized protein OS=Xiphophorus maculatus PE=4 SV=1;
>tr|I3MJQ3|I3MJQ3_ICTTR Uncharacterized protein OS=Ictidomys tridecemlineatus GN=DNTT PE=4
SV=1;
>tr|G3NAV1|G3NAV1_GASAC Uncharacterized protein OS=Gasterosteus aculeatus PE=4 SV=1;
>tr|W5UBD8|W5UBD8_ICTPU DNA-directed DNA/RNA polymerase mu OS=Ictalurus punctatus
GN=Polm PE=2 SV=1;
>tr|G3HMA0|G3HMA0_CRIGR DNA polymerase mu OS=Cricetulus griseus GN=I79_011851 PE=4
SV=1;
>tr|A0A146NRB1|A0A146NRB1_FUNHE DNA-directed DNA/RNA polymerase mu OS=Fundulus
heteroclitus PE=4 SV=1;
>tr|W5LJC9|W5LJC9_ASTMX Uncharacterized protein OS=Astyanax mexicanus PE=4 SV=1;
>tr|Q7ZUU0|Q7ZUU0_DANRE Polm protein OS=Danio rerio GN=polm PE=2 SV=1;
>tr|A0A060W4U6|A0A060W4U6_ONCMY Uncharacterized protein OS=Oncorhynchus mykiss
GN=GSONMT00066594001 PE=4 SV=1;
>tr|Q5IBN3|Q5IBN3_DANRE DNA polymerase mu OS=Danio rerio GN=polm PE=1 SV=1;
>tr|A0A1A8D2K8|A0A1A8D2K8_9TELE Polymerase (DNA directed), mu OS=Nothobranchius kadleci
GN=POLM PE=4 SV=1;
>tr|F6SV89|F6SV89_MONDO Uncharacterized protein OS=Monodelphis domestica GN=POLM PE=4
SV=1;
>tr|I3M0V3|I3M0V3_ICTTR Uncharacterized protein OS=Ictidomys tridecemlineatus GN=POLM PE=4
SV=1;
>tr|A0A091T942|A0A091T942_NESNO DNA nucleotidylexotransferase (Fragment) OS=Nestor notabilis
GN=N333_11068 PE=4 SV=1;
>sp|Q9JIW4|DPOLM_MOUSE DNA-directed DNA/RNA polymerase mu OS=Mus musculus GN=Polm
PE=1 SV=2;
>tr|A0A1A8UBQ3|A0A1A8UBQ3_NOTFU Polymerase (DNA directed), mu OS=Nothobranchius furzeri
GN=POLM PE=4 SV=1;
>tr|A0A091LWE4|A0A091LWE4_CARIC DNA nucleotidylexotransferase (Fragment) OS=Cariama
cristata GN=N322_09261 PE=4 SV=1;
>tr|K7GPU0|K7GPU0_PIG Uncharacterized protein OS=Sus scrofa GN=DNTT PE=4 SV=1;
>tr|A0A096NAR8|A0A096NAR8_PAPAN Uncharacterized protein OS=Papio anubis GN=POLM PE=4
SV=1;
>tr|Q924W4|Q924W4_MOUSE DNA polymerase mu OS=Mus musculus GN=DNAPOLmu PE=4 SV=1;
>sp|Q9NP87|DPOLM_HUMAN DNA-directed DNA/RNA polymerase mu OS=Homo sapiens
GN=POLM PE=1 SV=1;
>tr|A0A1A8J9K9|A0A1A8J9K9_NOTKU Polymerase (DNA directed), mu OS=Nothobranchius kuhntae
GN=POLM PE=4 SV=1;
>tr|U3DX09|U3DX09_CALJA DNA-directed DNA/RNA polymerase mu OS=Callithrix jacchus
GN=POLM PE=2 SV=1;
>tr|Q7TN90|Q7TN90_MOUSE Polymerase (DNA directed), mu OS=Mus musculus GN=Polm PE=2
SV=1;
>tr|G3SE66|G3SE66_GORGO Uncharacterized protein OS=Gorilla gorilla gorilla GN=POLM PE=4
SV=1;
>tr|U3FAC2|U3FAC2_CALJA DNA-directed DNA/RNA polymerase mu OS=Callithrix jacchus
GN=POLM PE=2 SV=1;
>tr|K7BGH5|K7BGH5_PANTR Polymerase (DNA directed), mu OS=Pan troglodytes GN=POLM PE=2
SV=1;
>tr|H2QUI0|H2QUI0_PANTR Uncharacterized protein OS=Pan troglodytes GN=POLM PE=4 SV=1;
>tr|A0A096MCQ1|A0A096MCQ1_POEFO Uncharacterized protein OS=Poecilia formosa PE=4 SV=1;
>tr|F7BJ05|F7BJ05_HORSE Uncharacterized protein OS=Equus caballus GN=POLM PE=4 SV=1;
>tr|G1PJG7|G1PJG7_MYOLU Uncharacterized protein OS=Myotis lucifugus GN=POLM PE=4 SV=1;
>tr|A0A0D9RSI4|A0A0D9RSI4_CHLSB Uncharacterized protein OS=Chlorocebus sabaeus GN=POLM
PE=4 SV=1;
>tr|H2L4T8|H2L4T8_ORYLA Uncharacterized protein OS=Oryzias latipes GN=DNTT PE=4 SV=1;
>tr|A0A1A7ZHN0|A0A1A7ZHN0_NOTFU Polymerase (DNA directed), mu OS=Nothobranchius furzeri
GN=POLM PE=4 SV=1;
>tr|G1TSU6|G1TSU6_RABIT Uncharacterized protein OS=Oryctolagus cuniculus GN=POLM PE=4
SV=1;
>tr|F1SSF6|F1SSF6_PIG Uncharacterized protein OS=Sus scrofa GN=POLM PE=4 SV=1;
>tr|A0A093PRU9|A0A093PRU9_PHACA DNA nucleotidylexotransferase (Fragment) OS=Phalacrocorax
carbo GN=N336_06051 PE=4 SV=1;
>tr|H0WUS0|H0WUS0_OTOGA Uncharacterized protein OS=Otolemur garnettii GN=POLM PE=4
SV=1;
>tr|A0A094KEU6|A0A094KEU6_ANTCR DNA nucleotidylexotransferase (Fragment) OS=Antrostomus
carolinensis GN=N321_01336 PE=4 SV=1;
>tr|H0VRU3|H0VRU3_CAVPO Uncharacterized protein OS=Cavia porcellus GN=POLM PE=4 SV=1;
>tr|A0A091ECM1|A0A091ECM1_FUKDA DNA-directed DNA/RNA polymerase mu OS=Fukomys
damarensis GN=H920_05682 PE=4 SV=1;
>tr|G5ASQ9|G5ASQ9_HETGA DNA polymerase mu OS=Heterocephalus glaber GN=GW7_14800 PE=4
SV=1;
>tr|G3VIL2|G3VIL2_SARHA Uncharacterized protein (Fragment) OS=Sarcophilus harrisii GN=POLM
PE=4 SV=1;
>tr|Q0VFA6|Q0VFA6_XENTR Uncharacterized protein (Fragment) OS=Xenopus tropicalis PE=2 SV=1;
>tr|A0A1S2ZTW0|A0A1S2ZTW0_ERIEU DNA-directed DNA/RNA polymerase mu OS=Erinaceus
europaeus GN=POLM PE=4 SV=1;
>tr|A0A061IPE9|A0A061IPE9_CRIGR DNA-directed DNA/RNA polymerase mu OS=Cricetulus griseus
GN=H671_1g1703 PE=4 SV=1;
>tr|F1MPJ5|F1MPJ5_BOVIN Uncharacterized protein OS=Bos taurus GN=POLM PE=4 SV=2;
>tr|L5KMY1|L5KMY1_PTEAL DNA polymerase mu OS=Pteropus alecto GN=PAL_GLEAN10002258
PE=4 SV=1;
>tr|G3T5I6|G3T5I6_LOXAF Uncharacterized protein (Fragment) OS=Loxodonta africana GN=POLM
PE=4 SV=1;
>tr|A0A0R4IL28|A0A0R4IL28_DANRE Polymerase (DNA directed), mu OS=Danio rerio GN=polm
PE=1 SV=1;
>tr|W5Q5W9|W5Q5W9_SHEEP Uncharacterized protein OS=Ovis aries GN=POLM PE=4 SV=1;
>tr|F6YZ98|F6YZ98_XENTR Uncharacterized protein (Fragment) OS=Xenopus tropicalis GN=polm
PE=4 SV=1;
>tr|A0A1D5QIN2|A0A1D5QIN2_MACMU Uncharacterized protein OS=Macaca mulatta GN=POLM
PE=4 SV=1;
>tr|A0A1A7XD24|A0A1A7XD24_9TELE Polymerase (DNA directed), mu (Fragment) OS=Aphyosemion
striatum GN=POLM PE=4 SV=1;
>tr|A0A093F2E3|A0A093F2E3_TYTAL DNA nucleotidylexotransferase (Fragment) OS=Tyto alba
GN=N341_11661 PE=4 SV=1;
>tr|V8NA58|V8NA58_OPHHA DNA-directed DNA/RNA polymerase mu (Fragment) OS=Ophiophagus
hannah GN=POLM PE=4 SV=1;
>tr|A0A1A8NJB1|A0A1A8NJB1_9TELE Polymerase (DNA directed), mu OS=Nothobranchius rachovii
GN=POLM PE=4 SV=1;
>tr|M3X1D8|M3X1D8_FELCA Uncharacterized protein OS=Felis catus GN=POLM PE=4 SV=1;
>tr|A0A1A8CS80|A0A1A8CS80_9TELE Polymerase (DNA directed), mu OS=Nothobranchius kadleci
GN=POLM PE=4 SV=1;
>tr|A0A1A8JAN0|A0A1A8JAN0_NOTKU Polymerase (DNA directed), mu OS=Nothobranchius kuhntae
GN=POLM PE=4 SV=1;
>tr|F6SUN4|F6SUN4_XENTR Uncharacterized protein (Fragment) OS=Xenopus tropicalis GN=polm
PE=4 SV=2;>tr|A0A151NPI6|A0A151NPI6_ALLMI DNA-directed DNA/RNA polymerase mu
OS=Alligator mississippiensis GN=POLM PE=4 SV=1;
>tr|H2VEE2|H2VEE2_TAKRU Uncharacterized protein OS=Takifugu rubripes GN=polm PE=4 SV=1;
>tr|A0A1L8GS64|A0A1L8GS64_XENLA Uncharacterized protein OS=Xenopus laevis
GN=XELAEV_18020298mg PE=4 SV=1;
>tr|S7NKX1|S7NKX1_MYOBR DNA-directed DNA/RNA polymerase mu OS=Myotis brandtii
GN=D623_10014907 PE=4 SV=1;
>tr|A0A091PH27|A0A091PH27_HALAL DNA nucleotidylexotransferase OS=Haliaeetus albicilla
GN=N329_12936 PE=4 SV=1;
>tr|Q5NCI3|Q5NCI3_MOUSE DNA-directed DNA/RNA polymerase mu OS=Mus musculus GN=Polm
PE=1 SV=1;
>tr|A0A091L3R3|A0A091L3R3_CATAU DNA nucleotidylexotransferase OS=Cathartes aura
GN=N323_12768 PE=4 SV=1;
>tr|V9L4E7|V9L4E7_CALMI DNA-directed DNA/RNA polymerase mu (Fragment) OS=Callorhinchus
milii PE=2 SV=1;
>tr|A0A093JK89|A0A093JK89_EURHL DNA nucleotidylexotransferase OS=Eurypyga helias
GN=N326_11527 PE=4 SV=1;
>tr|U3KMM5|U3KMM5_RABIT Uncharacterized protein OS=Oryctolagus cuniculus GN=POLM PE=4
SV=1;
>tr|A0A1A8P9J9|A0A1A8P9J9_9TELE Deoxynucleotidyltransferase, terminal (Fragment)
OS=Nothobranchius rachovii GN=DNTT PE=4 SV=1;
>tr|A0A1A8KA73|A0A1A8KA73_NOTKU Polymerase (DNA directed), mu (Fragment)
OS=Nothobranchius kuhntae GN=POLM PE=4 SV=1;
>tr|S9XEA8|S9XEA8_CAMFR DNA nucleotidylexotransferase isoform 1-like protein OS=Camelus ferus
GN=CB1_000155020 PE=4 SV=1;
>tr|L5LFP9|L5LFP9_MYODS DNA-directed DNA/RNA polymerase mu OS=Myotis davidii
GN=MDA_GLEAN10006449 PE=4 SV=1;
>tr|M3Z3D2|M3Z3D2_MUSPF Uncharacterized protein OS=Mustela putorius furo GN=POLM PE=4
SV=1;
>tr|A0A146X6G9|A0A146X6G9_FUNHE DNA nucleotidylexotransferase (Fragment) OS=Fundulus
heteroclitus PE=4 SV=1;
>tr|S9YVX3|S9YVX3_CAMFR DNA-directed DNA/RNA polymerase mu OS=Camelus ferus
GN=CB1_000193022 PE=4 SV=1;
>tr|W4Y2P6|W4Y2P6_STRPU Uncharacterized protein OS=Strongylocentrotus purpuratus PE=4 SV=1;
>tr|A0A087YLM2|A0A087YLM2_POEFO Uncharacterized protein OS=Poecilia formosa PE=4 SV=2;
>tr|G1MGP9|G1MGP9_AILME Uncharacterized protein (Fragment) OS=Ailuropoda melanoleuca
GN=POLM PE=4 SV=1;
>tr|A0A091K5R0|A0A091K5R0_COLST DNA nucleotidylexotransferase (Fragment) OS=Colius striatus
GN=N325_07143 PE=4 SV=1;
>tr|B1H1C4|B1H1C4_XENTR Uncharacterized protein OS=Xenopus tropicalis GN=polm PE=2 SV=1;
>tr|A0A091T176|A0A091T176_9AVES DNA nucleotidylexotransferase (Fragment) OS=Pelecanus
crispus GN=N334_13201 PE=4 SV=1;
>tr|R0K6L2|R0K6L2_ANAPL DNA nucleotidylexotransferase (Fragment) OS=Anas platyrhynchos
GN=LOC101804368 PE=4 SV=1;
>tr|W5NDT9|W5NDT9_LEPOC Uncharacterized protein OS=Lepisosteus oculatus PE=4 SV=1;
>tr|A0A091SF87|A0A091SF87_9GRUI DNA nucleotidylexotransferase (Fragment) OS=Mesitornis
unicolor GN=N332_10317 PE=4 SV=1;
>sp|Q9NP87-2|DPOLM_HUMAN Isoform 2 of DNA-directed DNA/RNA polymerase mu OS=Homo
sapiens GN=POLM;
>tr|A0A0P7UAY6|A0A0P7UAY6_9TELE DNA-directed DNA/RNA polymerase mu-like
OS=Scleropages formosus GN=Z043_116844 PE=4 SV=1;
>tr|H2PM88|H2PM88_PONAB Uncharacterized protein OS=Pongo abelii GN=POLM PE=4 SV=1;
>tr|A0A1S3LYF6|A0A1S3LYF6_SALSA DNA-directed DNA/RNA polymerase mu-like OS=Salmo salar
GN=LOC106569276 PE=4 SV=1;
>tr|L7N3V6|L7N3V6_XENTR Uncharacterized protein OS=Xenopus tropicalis GN=polm PE=4 SV=1;
>tr|A0A0F8AHZ5|A0A0F8AHZ5_LARCR DNA nucleotidylexotransferase OS=Larimichthys crocea
GN=EH28_08861 PE=4 SV=1;
>tr|A0A091ND14|A0A091ND14_APAVI DNA nucleotidylexotransferase (Fragment) OS=Apaloderma
vittatum GN=N311_03749 PE=4 SV=1;
>tr|A0A1A8HR87|A0A1A8HR87_NOTKU Deoxynucleotidyltransferase, terminal OS=Nothobranchius
kuhntae GN=DNTT PE=4 SV=1;
>tr|A0A1A8UVJ9|A0A1A8UVJ9_NOTFU Deoxynucleotidyltransferase, terminal OS=Nothobranchius
furzeri GN=DNTT PE=4 SV=1;
>tr|A0A1A8RA57|A0A1A8RA57_9TELE Polymerase (DNA directed), mu (Fragment)
OS=Nothobranchius pienaari GN=POLM PE=4 SV=1;
>tr|S4RJG9|S4RJG9_PETMA Uncharacterized protein OS=Petromyzon marinus PE=4 SV=1;
>tr|A0A1A7ZHB4|A0A1A7ZHB4_NOTFU Polymerase (DNA directed), mu (Fragment)
OS=Nothobranchius furzeri GN=POLM PE=4 SV=1;
>tr|Q5FVA7|Q5FVA7_XENTR Poll.2 protein (Fragment) OS=Xenopus tropicalis GN=poll.2 PE=2 SV=1;
>tr|H9KX16|H9KX16_CALJA Uncharacterized protein OS=Callithrix jacchus PE=4 SV=1;
>tr|C3YIS5|C3YIS5_BRAFL Putative uncharacterized protein OS=Branchiostoma floridae
GN=BRAFLDRAFT_59678 PE=4 SV=1;
>tr|A0A146XGI0|A0A146XGI0_FUNHE DNA-directed DNA/RNA polymerase mu (Fragment)
OS=Fundulus heteroclitus PE=4 SV=1;
>tr|G9KHQ3|G9KHQ3_MUSPF Polymerase, mu (Fragment) OS=Mustela putorius furo PE=2 SV=1;
>tr|V3ZNY9|V3ZNY9_LOTGI Uncharacterized protein (Fragment) OS=Lottia gigantea
GN=LOTGIDRAFT_72364 PE=4 SV=1;
>tr|B3S8X2|B3S8X2_TRIAD Putative uncharacterized protein OS=Trichoplax adhaerens
GN=TRIADDRAFT_60774 PE=4 SV=1;
>tr|H2ZGS8|H2ZGS8_CIOSA Uncharacterized protein OS=Ciona savignyi PE=4 SV=1;
>tr|H9GW56|H9GW56_CANLF Uncharacterized protein OS=Canis lupus familiaris PE=4 SV=2;
>tr|A0A1A8E0N8|A0A1A8E0N8_9TELE Polymerase (DNA directed), mu OS=Nothobranchius kadleci
GN=POLM PE=4 SV=1;
>tr|A0A146RC47|A0A146RC47_FUNHE DNA-directed DNA/RNA polymerase mu OS=Fundulus
heteroclitus PE=4 SV=1;
>tr|A0A1A8AD54|A0A1A8AD54_NOTFU Polymerase (DNA directed), mu (Fragment)
OS=Nothobranchius furzeri GN=POLM PE=4 SV=1;
>tr|A0A146N7G7|A0A146N7G7_FUNHE DNA-directed DNA/RNA polymerase mu OS=Fundulus
heteroclitus PE=4 SV=1;
>tr|A0A0S7LMJ9|A0A0S7LMJ9_9TELE DPOLM (Fragment) OS=Poeciliopsis prolifica GN=DPOLM
PE=4 SV=1;
>tr|L8IE28|L8IE28_9CETA DNA polymerase mu OS=Bos mutus GN=M91_15825 PE=4 SV=1;
>tr|A0A060XXZ4|A0A060XXZ4_ONCMY Uncharacterized protein OS=Oncorhynchus mykiss
GN=GSONMT00003902001 PE=4 SV=1;
>tr|Q58DV2|Q58DV2_BOVIN Polymerase (DNA directed), mu OS=Bos taurus GN=POLM PE=2 SV=1;
>tr|A0A091TVD3|A0A091TVD3_PHALP DNA nucleotidylexotransferase (Fragment) OS=Phaethon
lepturus GN=N335_13210 PE=4 SV=1;
>tr|Q4S1X0|Q4S1X0_TETNG Chromosome undetermined SCAF14764, whole genome shotgun sequence
OS=Tetraodon nigroviridis GN=GSTENG00025355001 PE=4 SV=1;
>tr|A0A146X6L6|A0A146X6L6_FUNHE DNA nucleotidylexotransferase (Fragment) OS=Fundulus
heteroclitus PE=4 SV=1;
>tr|A0A1A8FJT0|A0A1A8FJT0_9TELE Polymerase (DNA directed), mu (Fragment) OS=Nothobranchius
korthausae GN=POLM PE=4 SV=1;
>tr|K7FEN8|K7FEN8_PELSI Uncharacterized protein OS=Pelodiscus sinensis GN=POLM PE=4 SV=1;
>tr|V8NQW9|V8NQW9_OPHHA DNA nucleotidylexotransferase (Fragment) OS=Ophiophagus hannah
GN=DNTT PE=4 SV=1;
>tr|T1JNH2|T1JNH2_STRMM Uncharacterized protein OS=Strigamia maritima PE=4 SV=1;
>tr|K7FEM8|K7FEM8_PELSI Uncharacterized protein OS=Pelodiscus sinensis GN=POLM PE=4 SV=1;
>tr|A0A146XH72|A0A146XH72_FUNHE DNA-directed DNA/RNA polymerase mu OS=Fundulus
heteroclitus PE=4 SV=1;
>tr|Q4RN80|Q4RN80_TETNG Chromosome undetermined SCAF15016, whole genome shotgun sequence
(Fragment) OS=Tetraodon nigroviridis GN=GSTENG00031681001 PE=4 SV=1;
>tr|M7B2F2|M7B2F2_CHEMY DNA-directed DNA/RNA polymerase mu OS=Chelonia mydas
GN=UY3_16640 PE=4 SV=1;
>tr|A0A0P7UYV0|A0A0P7UYV0_9TELE Uncharacterized protein (Fragment) OS=Scleropages formosus
GN=Z043_101012 PE=4 SV=1;
>tr|A0A1A7YQL2|A0A1A7YQL2_9TELE Polymerase (DNA directed), mu (Fragment)
OS=Aphyosemion striatum GN=POLM PE=4 SV=1;
>tr|H9GJR5|H9GJR5_ANOCA Uncharacterized protein OS=Anolis carolinensis PE=4 SV=1;
>tr|K1PM26|K1PM26_CRAGI DNA polymerase mu OS=Crassostrea gigas GN=CGI_10007307 PE=4
SV=1;
>tr|C5H604|C5H604_HORSE Terminal deoxynucleotidyltransferase (Fragment) OS=Equus caballus
GN=DNTT PE=2 SV=1;
>tr|A0A0S7LMI4|A0A0S7LMI4_9TELE DPOLM (Fragment) OS=Poeciliopsis prolifica GN=DPOLM
PE=4 SV=1;
>tr|I1FU11|I1FU11_AMPQE Uncharacterized protein OS=Amphimedon queenslandica PE=4 SV=1;
>tr|H9GS78|H9GS78_ANOCA Uncharacterized protein OS=Anolis carolinensis GN=POLM PE=4 SV=2;
>tr|A0A0P5QDN2|A0A0P5QDN2_9CRUS DNA-directed DNA/RNA polymerase mu OS=Daphnia
magna PE=4 SV=1;
>tr|A0A165AGI8|A0A165AGI8_9CRUS Uncharacterized protein OS=Daphnia magna
GN=APZ42_016503 PE=4 SV=1;
>tr|A0A0P6IJT6|A0A0P6IJT6_9CRUS DNA-directed DNA/RNA polymerase mu OS=Daphnia magna
PE=4 SV=1;
>tr|A0A1S3J3X7|A0A1S3J3X7_LINUN DNA-directed DNA/RNA polymerase mu-like isoform X1
OS=Lingula unguis GN=LOC106169975 PE=4 SV=1;
>tr|A0A1S3J3Y6|A0A1S3J3Y6_LINUN DNA-directed DNA/RNA polymerase mu-like isoform X2
OS=Lingula unguis GN=LOC106169975 PE=4 SV=1;
>tr|F7CVN4|F7CVN4_CALJA Uncharacterized protein OS=Callithrix jacchus PE=4 SV=1;
>tr|E9HD36|E9HD36_DAPPU Putative uncharacterized protein OS=Daphnia pulex
GN=DAPPUDRAFT_300508 PE=4 SV=1;
>tr|A0A0P6AAE9|A0A0P6AAE9_9CRUS DNA-directed DNA/RNA polymerase mu OS=Daphnia magna
PE=4 SV=1;
>tr|G7P2A2|G7P2A2_MACFA Putative uncharacterized protein OS=Macaca fascicularis
GN=EGM_12557 PE=4 SV=1;
>tr|A0A0P5GM38|A0A0P5GM38_9CRUS DNA-directed DNA/RNA polymerase mu OS=Daphnia magna
PE=4 SV=1;
>tr|G7MLE4|G7MLE4_MACMU Uncharacterized protein OS=Macaca mulatta GN=EGK_13738 PE=4
SV=1;
>tr|Q6PIY2|Q6PIY2_HUMAN DNA-directed DNA/RNA polymerase mu OS=Homo sapiens GN=POLM
PE=1 SV=1;
>tr|U9U304|U9U304_RHIID Uncharacterized protein OS=Rhizophagus irregularis (strain DAOM 181602
/ DAOM 197198 / MUCL 43194) GN=GLOINDRAFT_287 PE=4 SV=1;
>tr|A0A0B6ZDL7|A0A0B6ZDL7_9EUPU Uncharacterized protein (Fragment) OS=Arion vulgaris
GN=ORF59157 PE=4 SV=1;
>tr|F1QNC9|F1QNC9_DANRE Deoxynucleotidyltransferase, terminal OS=Danio rerio GN=dntt PE=4
SV=1;
>tr|A0A0P5PJF6|A0A0P5PJF6_9CRUS DNA-directed DNA/RNA polymerase mu (Fragment)
OS=Daphnia magna PE=4 SV=1;
>tr|A0A0P5WYI3|A0A0P5WYI3_9CRUS DNA-directed DNA/RNA polymerase mu OS=Daphnia magna
PE=4 SV=1;
>tr|H9KVL9|H9KVL9_CALJA Uncharacterized protein OS=Callithrix jacchus PE=4 SV=1;
>tr|A0A0N7ZWR4|A0A0N7ZWR4_9CRUS DNA-directed DNA/RNA polymerase mu (Fragment)
OS=Daphnia magna PE=4 SV=1;
>tr|Q9H980|Q9H980_HUMAN DNA-directed DNA/RNA polymerase mu OS=Homo sapiens GN=POLM
PE=1 SV=1;
>tr|H9KVM0|H9KVM0_CALJA Uncharacterized protein OS=Callithrix jacchus PE=4 SV=1;
>tr|H9KW19|H9KW19_CALJA Uncharacterized protein OS=Callithrix jacchus PE=4 SV=1;
>tr|A0A075AZ03|A0A075AZ03_9FUNG DNA polymerase family X lyase domain-containing protein
OS=Rozella allomycis CSF55 GN=O9G_000755 PE=4 SV=1;
>tr|A0A1A8BGS3|A0A1A8BGS3_9TELE Deoxynucleotidyltransferase, terminal OS=Nothobranchius
kadleci GN=DNTT PE=4 SV=1;
>tr|U6CZ52|U6CZ52_NEOVI DNA-directed DNA/RNA polymerase mu (Fragment) OS=Neovison vison
GN=DPOLM PE=2 SV=1;
>tr|A0A0D2UGV9|A0A0D2UGV9_CAPO3 Uncharacterized protein OS=Capsaspora owczarzaki (strain
ATCC 30864) GN=CAOG_004995 PE=4 SV=1;
>tr|F6PMA5|F6PMA5_MACMU Uncharacterized protein OS=Macaca mulatta GN=POLM PE=4 SV=2;
>sp|Q9NP87-3|DPOLM_HUMAN Isoform 3 of DNA-directed DNA/RNA polymerase mu OS=Homo
sapiens GN=POLM;
>tr|A0A168R3T5|A0A168R3T5_ABSGL Uncharacterized protein OS=Absidia glauca
GN=ABSGL_11926.1 scaffold 12357 PE=4 SV=1;
>tr|A0A0C3BGD0|A0A0C3BGD0_9HOMO Uncharacterized protein OS=Serendipita vermifera MAFF
305830 GN=M408DRAFT_327483 PE=4 SV=1;
>tr|A0A0C2SZ00|A0A0C2SZ00_AMAMU Uncharacterized protein OS=Amanita muscaria Koide BX008
GN=M378DRAFT_8228 PE=4 SV=1;
>tr|A0A0W0FVE5|A0A0W0FVE5_9AGAR Uncharacterized protein OS=Moniliophthora roreri
GN=WG66_7104 PE=4 SV=1;
>tr|S8DTS4|S8DTS4_FOMPI Uncharacterized protein OS=Fomitopsis pinicola (strain FP-58527)
GN=FOMPIDRAFT_52938 PE=4 SV=1;
>tr|H2L5F7|H2L5F7_ORYLA Uncharacterized protein OS=Oryzias latipes PE=4 SV=1;
>tr|V2XUA2|V2XUA2_MONRO Dna polymerase mu OS=Moniliophthora roreri (strain MCA 2997)
GN=Moror_17783 PE=4 SV=1;
>tr|G3QPE8|G3QPE8_GORGO Uncharacterized protein OS=Gorilla gorilla gorilla GN=POLM PE=4
SV=1;
>tr|H9KW10|H9KW10_CALJA Uncharacterized protein OS=Callithrix jacchus PE=4 SV=1;
>tr|A0A061ALJ8|A0A061ALJ8_RHOTO RHTO0S01e16908g1_1 OS=Rhodosporidium toruloides
GN=RHTO0S_01e16908g PE=4 SV=1;
>tr|A0A0G2KCT4|A0A0G2KCT4_DANRE Uncharacterized protein OS=Danio rerio PE=4 SV=1;
>tr|A0A0P5PVU2|A0A0P5PVU2_9CRUS DNA-directed DNA/RNA polymerase mu OS=Daphnia magna
PE=4 SV=1;
>tr|A0A0P5MXJ7|A0A0P5MXJ7_9CRUS DNA-directed DNA/RNA polymerase mu OS=Daphnia magna
PE=4 SV=1;
>tr|A0A0P5NMK9|A0A0P5NMK9_9CRUS DNA-directed DNA/RNA polymerase mu OS=Daphnia
magna PE=4 SV=1;
>tr|A0A060VUK5|A0A060VUK5_ONCMY Uncharacterized protein OS=Oncorhynchus mykiss
GN=GSONMT00078040001 PE=4 SV=1;
>tr|V8P7H4|V8P7H4_OPHHA DNA polymerase lambda (Fragment) OS=Ophiophagus hannah
GN=POLL PE=4 SV=1;
>tr|A0A137QJR4|A0A137QJR4_9AGAR DNA-directed DNA/RNA polymerase mu OS=Leucoagaricus
sp. SymC.cos GN=AN958_08787 PE=4 SV=1;
>tr|A0A164P2X5|A0A164P2X5_9HOMO Nucleotidyltransferase OS=Sistotremastrum niveocremeum
HHB9708 GN=SISNIDRAFT_446120 PE=4 SV=1;
>tr|F7HXH0|F7HXH0_CALJA Uncharacterized protein OS=Callithrix jacchus PE=4 SV=1;
>tr|G1QTM9|G1QTM9_NOMLE Uncharacterized protein OS=Nomascus leucogenys GN=POLM PE=4
SV=1;
>tr|Q4RN81|Q4RN81_TETNG Chromosome undetermined SCAF15016, whole genome shotgun sequence
(Fragment) OS=Tetraodon nigroviridis GN=GSTENG00031680001 PE=4 SV=1;
>tr|A0A167PH29|A0A167PH29_9BASI Nucleotidyltransferase OS=Calocera viscosa TUFC12733
GN=CALVIDRAFT_478151 PE=4 SV=1;
>tr|A0A165MRI2|A0A165MRI2_EXIGL Nucleotidyltransferase OS=Exidia glandulosa HHB12029
GN=EXIGLDRAFT_724655 PE=4 SV=1;
>tr|A0A068SFG6|A0A068SFG6_9FUNG Dna polymerase mu OS=Lichtheimia corymbifera
JMRC:FSU:9682 GN=LCOR_11539.1 PE=4 SV=1;
>tr|B0D8M6|B0D8M6_LACBS Predicted protein OS=Laccaria bicolor (strain S238N-H82 / ATCC MYA-
4686) GN=LACBIDRAFT_296372 PE=4 SV=1;
>tr|A0A165JL69|A0A165JL69_9BASI Nucleotidyltransferase OS=Calocera cornea HHB12733
GN=CALCODRAFT_514361 PE=4 SV=1;
>tr|M3WN40|M3WN40_FELCA Uncharacterized protein OS=Felis catus GN=POLL PE=4 SV=1;
>tr|A0A0P4YRE9|A0A0P4YRE9_9CRUS Putative DNA-directed DNA/RNA polymerase mu
OS=Daphnia magna PE=4 SV=1;
>tr|M3WQL3|M3WQL3_FELCA Uncharacterized protein OS=Felis catus GN=POLL PE=4 SV=1;
>tr|A0A1E1X922|A0A1E1X922_9ACAR Putative dna polymerase lambda OS=Amblyomma aureolatum
PE=2 SV=1;
>tr|E5FGJ8|E5FGJ8_PLECU DNA polymerase lambda (Fragment) OS=Plecturocebus cupreus GN=POLL
PE=2 SV=1;
>tr|A0A146IDE1|A0A146IDE1_9AGAR Uncharacterized protein OS=Mycena chlorophos
GN=MCHLO_14360 PE=4 SV=1;
>tr|A0A0B7G3X1|A0A0B7G3X1_THACB DNA-directed DNA/RNA polymerase mu OS=Homo sapiens
GN=POLM PE=1 SV=1 OS=Thanatephorus cucumeris (strain AG1-IB / isolate 7/3/14)
GN=RSOLAG1IB_05578 PE=4 SV=1;
>tr|A0A1B7NDI9|A0A1B7NDI9_9HOMO Nucleotidyltransferase OS=Rhizopogon vinicolor AM-OR11-
026 GN=K503DRAFT_862764 PE=4 SV=1;
>tr|A0A0C3NTP8|A0A0C3NTP8_PHLGI Uncharacterized protein OS=Phlebiopsis gigantea 11061_1
CR5-6 GN=PHLGIDRAFT_29452 PE=4 SV=1;
>tr|A0A165Z2P2|A0A165Z2P2_9HOMO Nucleotidyltransferase OS=Sistotremastrum suecicum
HHB10207 ss-3 GN=SISSUDRAFT_992585 PE=4 SV=1;
>tr|L5JPX4|L5JPX4_PTEAL DNA polymerase lambda OS=Pteropus alecto GN=PAL_GLEAN10018254
PE=4 SV=1;
>tr|A0A077W6Z0|A0A077W6Z0_9FUNG Uncharacterized protein OS=Lichtheimia ramosa
GN=LRAMOSA00609 PE=4 SV=1;
>tr|I3MHE8|I3MHE8_ICTTR Uncharacterized protein OS=Ictidomys tridecemlineatus GN=POLL PE=4
SV=1;
>tr|G1T998|G1T998_RABIT Uncharacterized protein OS=Oryctolagus cuniculus GN=POLL PE=4 SV=2;
>tr|U3AR34|U3AR34_CALJA DNA polymerase lambda isoform a OS=Callithrix jacchus GN=POLL
PE=2 SV=1;
>tr|G4TBJ0|G4TBJ0_SERID Related to DNA polymerase Tdt-N OS=Serendipita indica (strain DSM
11827) GN=PIIN_02548 PE=4 SV=1;
>tr|A0A168NYM3|A0A168NYM3_ABSGL Uncharacterized protein OS=Absidia glauca
GN=ABSGL_07199.1 scaffold 8717 PE=4 SV=1;
>tr|E5FGI5|E5FGI5_HYLAG DNA polymerase lambda (Fragment) OS=Hylobates agilis GN=POLL
PE=2 SV=1;
>tr|Q5K8N6|Q5K8N6_CRYNJ Beta DNA polymerase, putative OS=Cryptococcus neoformans var.
neoformans serotype D (strain JEC21 / ATCC MYA-565) GN=CNL05040 PE=4 SV=2;
>tr|X8JQU6|X8JQU6_9HOMO Finger of DNA polymerase lambda domain protein OS=Rhizoctonia
solani AG-3 Rhs1AP GN=RSOL_474080 PE=4 SV=1;
>tr|A0A074S8B0|A0A074S8B0_9HOMO Finger of DNA polymerase lambda domain protein
OS=Rhizoctonia solani 123E GN=V565_004060 PE=4 SV=1;
>tr|G3W992|G3W992_SARHA Uncharacterized protein OS=Sarcophilus harrisii GN=POLL PE=4 SV=1;
>tr|A0A066W4N7|A0A066W4N7_9HOMO Uncharacterized protein (Fragment) OS=Rhizoctonia solani
AG-8 WAC10335 GN=RSAG8_03653 PE=4 SV=1;
>tr|A0A146SIK4|A0A146SIK4_FUNHE DNA nucleotidylexotransferase-like protein (Fragment)
OS=Fundulus heteroclitus PE=4 SV=1;
>tr|S7NFH1|S7NFH1_MYOBR DNA polymerase lambda OS=Myotis brandtii GN=D623_10025942
PE=4 SV=1;
>tr|A0A0D0ADW7|A0A0D0ADW7_9HOMO Unplaced genomic scaffold CY34scaffold_406, whole
genome shotgun sequence OS=Suillus luteus UH-Slu-Lm8-n1 GN=CY34DRAFT_527157 PE=4 SV=1;
>tr|G1P3K8|G1P3K8_MYOLU Uncharacterized protein OS=Myotis lucifugus GN=POLL PE=4 SV=1;
>tr|L8J317|L8J317_9CETA DNA polymerase lambda OS=Bos mutus GN=M91_06669 PE=4 SV=1;
>tr|A0A0P5Q569|A0A0P5Q569_9CRUS DNA-directed DNA/RNA polymerase mu OS=Daphnia magna
PE=4 SV=1;
>tr|A0A0H2S450|A0A0H2S450_9HOMO Nucleotidyltransferase OS=Schizopora paradoxa
GN=SCHPADRAFT_865782 PE=4 SV=1;
>tr|A0A060XWV6|A0A060XWV6_ONCMY Uncharacterized protein OS=Oncorhynchus mykiss
GN=GSONMT00003903001 PE=4 SV=1;
>tr|E5FGJ5|E5FGJ5_SYMSY DNA polymerase lambda (Fragment) OS=Symphalangus syndactylus
GN=POLL PE=2 SV=1;
>tr|A4II30|A4II30_XENTR Poll protein OS=Xenopus tropicalis GN=poll PE=2 SV=1;
>tr|F6S5I4|F6S5I4_XENTR Uncharacterized protein OS=Xenopus tropicalis GN=poll PE=4 SV=1;
>tr|A0A1L8FJD6|A0A1L8FJD6_XENLA Uncharacterized protein OS=Xenopus laevis
GN=XELAEV_18034660mg PE=4 SV=1;
>tr|F6Q4I4|F6Q4I4_XENTR Uncharacterized protein OS=Xenopus tropicalis GN=poll PE=4 SV=1;
>tr|U6DAU0|U6DAU0_NEOVI Polymerase (DNA directed), lambda (Fragment) OS=Neovison vison
GN=Q5JQP8 PE=2 SV=1;
>tr|M5XMY2|M5XMY2_PRUPE Uncharacterized protein OS=Prunus persica
GN=PRUPE_ppa018614mg PE=4 SV=1;
>tr|Q55M33|Q55M33_CRYNB Uncharacterized protein OS=Cryptococcus neoformans var. neoformans
serotype D (strain B-3501A) GN=CNBI1790 PE=4 SV=1;
>tr|E2RBL7|E2RBL7_CANLF Uncharacterized protein OS=Canis lupus familiaris GN=POLL PE=4
SV=1;
>tr|A0A151MLI3|A0A151MLI3_ALLMI DNA polymerase lambda OS=Alligator mississippiensis
GN=POLL PE=4 SV=1;
>tr|A0A077X0T8|A0A077X0T8_9FUNG Uncharacterized protein OS=Lichtheimia ramosa
GN=LRAMOSA05278 PE=4 SV=1;
>tr|A0A1Q3E079|A0A1Q3E079_LENED Dna polymerase mu OS=Lentinula edodes
GN=LENED_002089 PE=4 SV=1;
>tr|H0V5L3|H0V5L3_CAVPO Uncharacterized protein OS=Cavia porcellus GN=POLL PE=4 SV=1;
>tr|E5FGJ9|E5FGJ9_NOMLE DNA polymerase lambda (Fragment) OS=Nomascus leucogenys
GN=POLL PE=2 SV=1;
>tr|G1RXX0|G1RXX0_NOMLE Uncharacterized protein OS=Nomascus leucogenys GN=POLL PE=4
SV=1;
>tr|E1BHH1|E1BHH1_BOVIN Uncharacterized protein OS=Bos taurus GN=POLL PE=4 SV=1;
>tr|G3I577|G3I577_CRIGR DNA polymerase lambda OS=Cricetulus griseus GN=I79_018621 PE=4
SV=1;
>tr|A0A168HEP9|A0A168HEP9_MUCCL Uncharacterized protein OS=Mucor circinelloides f. lusitanicus
CBS 277.49 GN=MUCCIDRAFT_167147 PE=4 SV=1;
>tr|A0A0D9QZP5|A0A0D9QZP5_CHLSB Uncharacterized protein OS=Chlorocebus sabaeus GN=POLL
PE=4 SV=1;
>tr|A0A194S0Q7|A0A194S0Q7_RHOGW Uncharacterized protein OS=Rhodotorula graminis (strain
WP1) GN=RHOBADRAFT_54045 PE=4 SV=1;
>tr|A0A091EXH7|A0A091EXH7_CORBR DNA polymerase lambda OS=Corvus brachyrhynchos
GN=N302_13582 PE=4 SV=1;
>tr|E5FGJ6|E5FGJ6_SAISC DNA polymerase lambda (Fragment) OS=Saimiri sciureus GN=POLL PE=2
SV=1;
>tr|L5MB70|L5MB70_MYODS DNA polymerase lambda OS=Myotis davidii
GN=MDA_GLEAN10023378 PE=4 SV=1;
>tr|F7DJM0|F7DJM0_CALJA Uncharacterized protein OS=Callithrix jacchus GN=POLL PE=4 SV=1;
>tr|G1NCL9|G1NCL9_MELGA Uncharacterized protein OS=Meleagris gallopavo GN=POLL PE=4
SV=1;
>tr|A0A093H2Y1|A0A093H2Y1_STRCA DNA polymerase lambda OS=Struthio camelus australis
GN=N308_06698 PE=4 SV=1;
>tr|A9TCX5|A9TCX5_PHYPA Predicted protein OS=Physcomitrella patens subsp. patens
GN=PHYPADRAFT_193840 PE=4 SV=1;
>tr|J9VLT9|J9VLT9_CRYNH DNA polymerase mu subunit OS=Cryptococcus neoformans var. grubii
serotype A (strain H99 / ATCC 208821 / CBS 10515 / FGSC 9487) GN=CNAG_05116 PE=4 SV=2;
>tr|A0A1B9I360|A0A1B9I360_9TREE DNA polymerase mu subunit OS=Kwoniella pini CBS 10737
GN=I206_04466 PE=4 SV=1;
>tr|A0A1A6GV71|A0A1A6GV71_NEOLE Uncharacterized protein OS=Neotoma lepida
GN=A6R68_01967 PE=4 SV=1;
>tr|A7SK52|A7SK52_NEMVE Predicted protein OS=Nematostella vectensis GN=v1g190712 PE=4
SV=1;
>tr|E5FGJ7|E5FGJ7_MIOTA DNA polymerase lambda (Fragment) OS=Miopithecus talapoin GN=POLL
PE=2 SV=1;
>tr|E5FGJ2|E5FGJ2_TRAFR DNA polymerase lambda (Fragment) OS=Trachypithecus francoisi
GN=POLL PE=2 SV=1;
>tr|E5FGJ1|E5FGJ1_ALOSA DNA polymerase lambda (Fragment) OS=Alouatta sara GN=POLL PE=2
SV=1;
>tr|E5FGI9|E5FGI9_COLGU DNA polymerase lambda (Fragment) OS=Colobus guereza GN=POLL
PE=2 SV=1;
>tr|E5FGK0|E5FGK0_CERWO DNA polymerase lambda (Fragment) OS=Cercopithecus wolfi
GN=POLL PE=2 SV=1;
>tr|A0A0N8C1F3|A0A0N8C1F3_9CRUS DNA-directed DNA/RNA polymerase mu (Fragment)
OS=Daphnia magna PE=4 SV=1;
>tr|G3T3S9|G3T3S9_LOXAF Uncharacterized protein OS=Loxodonta africana GN=POLL PE=4 SV=1;
>tr|W5Q4Y8|W5Q4Y8_SHEEP Uncharacterized protein OS=Ovis aries GN=POLL PE=4 SV=1;
>tr|A0A093PQD4|A0A093PQD4_9PASS DNA polymerase lambda OS=Manacus vitellinus
GN=N305_05530 PE=4 SV=1;
>tr|A0A091IEX6|A0A091IEX6_CALAN DNA polymerase lambda OS=Calypte anna GN=N300_12499
PE=4 SV=1;
>tr|D2GXU6|D2GXU6_AILME Putative uncharacterized protein (Fragment) OS=Ailuropoda melanoleuca
GN=PANDA_001756 PE=4 SV=1;
>tr|E5FGJ0|E5FGJ0_9PRIM DNA polymerase lambda (Fragment) OS=Gorilla gorilla GN=POLL PE=2
SV=1;
>tr|G1MGY1|G1MGY1_AILME Uncharacterized protein OS=Ailuropoda melanoleuca GN=POLL PE=4
SV=1;
>tr|G3QQQ5|G3QQQ5_GORGO Uncharacterized protein OS=Gorilla gorilla gorilla GN=POLL PE=4
SV=1;
>tr|Q245F6|Q245F6_TETTS Helix hairpin-helix protein OS=Tetrahymena thermophila (strain SB210)
GN=TTHERM_00732550 PE=4 SV=2;
>tr|W5N189|W5N189_LEPOC Uncharacterized protein OS=Lepisosteus oculatus PE=4 SV=1;
>tr|E5FGI7|E5FGI7_PONPY DNA polymerase lambda (Fragment) OS=Pongo pygmaeus GN=POLL
PE=2 SV=1;
>tr|E5FGJ4|E5FGJ4_PONAB DNA polymerase lambda (Fragment) OS=Pongo abelii GN=POLL PE=2
SV=1;
>tr|H2NBD3|H2NBD3_PONAB Uncharacterized protein OS=Pongo abelii GN=POLL PE=4 SV=1;
>tr|H2ZVH2|H2ZVH2_LATCH Uncharacterized protein OS=Latimeria chalumnae GN=POLL PE=4
SV=1;
>tr|A0A0C9MT17|A0A0C9MT17_9FUNG DNA polymerase beta OS=Mucor ambiguus
GN=MAM1_0127d06016 PE=4 SV=1;
>tr|G2HIX0|G2HIX0_PANTR DNA polymerase lambda OS=Pan troglodytes PE=2 SV=1;
>tr|E5FGI8|E5FGI8_MACFA DNA polymerase lambda (Fragment) OS=Macaca fascicularis GN=POLL
PE=2 SV=1;
>tr|F7HD68|F7HD68_MACMU DNA polymerase lambda isoform a OS=Macaca mulatta GN=POLL
PE=2 SV=1;
>sp|Q4R380|DPOLL_MACFA DNA polymerase lambda OS=Macaca fascicularis GN=POLL PE=2
SV=1;
>tr|W5N181|W5N181_LEPOC Uncharacterized protein OS=Lepisosteus oculatus PE=4 SV=1;
>tr|A0A0L8GFS8|A0A0L8GFS8_OCTBM Uncharacterized protein OS=Octopus bimaculoides
GN=OCBIM_22034122mg PE=4 SV=1;
>tr|A0A0D0T025|A0A0D0T025_9TREE Unplaced genomic scaffold supercont1.13, whole genome
shotgun sequence OS=Cryptococcus gattii VGII Ram5 GN=I313_05063 PE=4 SV=1;
>tr|A0A095DD01|A0A095DD01_CRYGR DNA polymerase mu subunit OS=Cryptococcus gattii serotype
B (strain R265) GN=CNBG_4574 PE=4 SV=1;
>tr|A0A0K3C7E6|A0A0K3C7E6_PHOTO BY PROTMAP: gi|814541105|emb|CEQ41545.1|
SPOSA6832_03302, partial [Sporidiobolus salmonicolor] OS=Rhodosporidium toruloides
GN=FGENESH: predicted gene_1.231 PE=4 SV=1;
>tr|A0A091LTQ7|A0A091LTQ7_CATAU DNA polymerase lambda OS=Cathartes aura
GN=N323_09789 PE=4 SV=1;
>tr|U5G1H1|U5G1H1_POPTR Uncharacterized protein OS=Populus trichocarpa
GN=POPTR_0010s10490g PE=4 SV=1;
>tr|H9GI65|H9GI65_ANOCA Uncharacterized protein OS=Anolis carolinensis GN=POLL PE=4 SV=2;
>tr|E5FGI6|E5FGI6_9PRIM DNA polymerase lambda (Fragment) OS=Lophocebus albigena GN=POLL
PE=2 SV=1;
>tr|E5FGJ3|E5FGJ3_PAPAN DNA polymerase lambda (Fragment) OS=Papio anubis GN=POLL PE=2
SV=1;
>tr|H2R0B4|H2R0B4_PANTR Polymerase (DNA directed), lambda OS=Pan troglodytes GN=POLL PE=2
SV=1;
>tr|A0A084QFP3|A0A084QFP3_9HYPO Uncharacterized protein OS=Stachybotrys chlorohalonata IBT
40285 GN=S40285_07550 PE=4 SV=1;
>tr|A0A0D6EPK6|A0A0D6EPK6_SPOSA SPOSA6832_03302-mRNA-1:cds (Fragment)
OS=Sporidiobolus salmonicolor GN=SPOSA6832_03302 PE=4 SV=1;
>tr|G5CAF7|G5CAF7_HETGA DNA polymerase lambda OS=Heterocephalus glaber GN=GW7_15886
PE=4 SV=1;
>tr|A0A1S3AEZ4|A0A1S3AEZ4_ERIEU DNA polymerase lambda OS=Erinaceus europaeus GN=POLL
PE=4 SV=1;
>tr|A0A067NJT6|A0A067NJT6_PLEOS Uncharacterized protein OS=Pleurotus ostreatus PC15
GN=PLEOSDRAFT_1041924 PE=4 SV=1;
>tr|A0A091J613|A0A091J613_9AVES DNA polymerase lambda OS=Egretta garzetta GN=Z169_01746
PE=4 SV=1;
>sp|Q5RKI3|DPOLL_RAT DNA polymerase lambda OS=Rattus norvegicus GN=Poll PE=2 SV=1;
>sp|Q9QXE2|DPOLL_MOUSE DNA polymerase lambda OS=Mus musculus GN=Poll PE=2 SV=1;
>tr|A0A1S3FAT0|A0A1S3FAT0_DIPOR DNA polymerase lambda OS=Dipodomys ordii GN=Poll PE=4
SV=1;
>tr|L8Y5V2|L8Y5V2_TUPCH DNA polymerase lambda OS=Tupaia chinensis GN=TREES_T100016491
PE=4 SV=1;
>tr|H2MC49|H2MC49_ORYLA Uncharacterized protein OS=Oryzias latipes PE=4 SV=1;
>tr|G9KHQ2|G9KHQ2_MUSPF Polymerase, lambda (Fragment) OS=Mustela putorius furo PE=2 SV=1;
>tr|A0A0C7BXF2|A0A0C7BXF2_9FUNG Uncharacterized protein OS=Rhizopus microsporus
GN=RMATCC62417_10444 PE=4 SV=1;
>tr|A0A094KZ04|A0A094KZ04_ANTCR DNA polymerase lambda OS=Antrostomus carolinensis
GN=N321_07972 PE=4 SV=1;
>tr|A0A091P384|A0A091P384_9PASS DNA polymerase lambda OS=Acanthisitta chloris
GN=N310_03460 PE=4 SV=1;
>tr|A0A091MSF3|A0A091MSF3_CARIC DNA polymerase lambda OS=Cariama cristata
GN=N322_09127 PE=4 SV=1;
>tr|A0A093GZ12|A0A093GZ12_PICPB DNA polymerase lambda OS=Picoides pubescens
GN=N307_08772 PE=4 SV=1;
>tr|A0A091UJR4|A0A091UJR4_NIPNI DNA polymerase lambda OS=Nipponia nippon GN=Y956_01422
PE=4 SV=1;
>tr|A0A091TC67|A0A091TC67_PHALP DNA polymerase lambda OS=Phaethon lepturus
GN=N335_05056 PE=4 SV=1;
>tr|M3YZQ5|M3YZQ5_MUSPF Uncharacterized protein OS=Mustela putorius furo GN=POLL PE=4
SV=1;
>tr|A0A091DMJ9|A0A091DMJ9_FUKDA DNA polymerase lambda OS=Fukomys damarensis
GN=H920_06249 PE=4 SV=1;
>tr|B3S2V4|B3S2V4_TRIAD Putative uncharacterized protein (Fragment) OS=Trichoplax adhaerens
GN=TRIADDRAFT_28268 PE=4 SV=1;
>tr|Q5JQP8|Q5JQP8_HUMAN DNA polymerase lambda OS=Homo sapiens GN=POLL PE=1 SV=1;
>tr|A0A0V0VHR8|A0A0V0VHR8_9BILA DNA polymerase lambda OS=Trichinella sp. T9
GN=T09_7096 PE=4 SV=1;
>tr|A0A0C3PWZ5|A0A0C3PWZ5_PISTI Uncharacterized protein OS=Pisolithus tinctorius Marx 270
GN=M404DRAFT_943795 PE=4 SV=1;
>tr|F7B5D1|F7B5D1_CHICK Uncharacterized protein OS=Gallus gallus GN=POLL PE=4 SV=1;
>tr|R0LAZ3|R0LAZ3_ANAPL DNA polymerase lambda (Fragment) OS=Anas platyrhynchos
GN=Anapl_11657 PE=4 SV=1;
>tr|U3IRR6|U3IRR6_ANAPL Uncharacterized protein OS=Anas platyrhynchos GN=POLL PE=4 SV=1;
>tr|A0A093JIL1|A0A093JIL1_FULGA DNA polymerase lambda OS=Fulmarus glacialis
GN=N327_08286 PE=4 SV=1;
>tr|A0A091PFQ4|A0A091PFQ4_HALAL DNA polymerase lambda OS=Haliaeetus albicilla
GN=N329_11480 PE=4 SV=1;
>tr|A0A091NUB5|A0A091NUB5_APAVI DNA polymerase lambda OS=Apaloderma vittatum
GN=N311_06514 PE=4 SV=1;
>tr|A0A091QAM1|A0A091QAM1_LEPDC DNA polymerase lambda OS=Leptosomus discolor
GN=N330_07141 PE=4 SV=1;
>tr|C3YJ28|C3YJ28_BRAFL Putative uncharacterized protein (Fragment) OS=Branchiostoma floridae
GN=BRAFLDRAFT_235602 PE=4 SV=1;
>tr|F1S8U1|F1S8U1_PIG Uncharacterized protein OS=Sus scrofa GN=POLL PE=4 SV=1;
>sp|Q9UGP5|DPOLL_HUMAN DNA polymerase lambda OS=Homo sapiens GN=POLL PE=1 SV=1;
>tr|A0A0C9ZIQ9|A0A0C9ZIQ9_9HOMO Unplaced genomic scaffold scaffold_56, whole genome
shotgun sequence OS=Pisolithus microcarpus 441 GN=PISMIDRAFT_680433 PE=4 SV=1;
>tr|A0A093C072|A0A093C072_9AVES DNA polymerase lambda OS=Pterocles gutturalis
GN=N339_07278 PE=4 SV=1;
>tr|A0A091RPW8|A0A091RPW8_9GRUI DNA polymerase lambda OS=Mesitornis unicolor
GN=N332_09822 PE=4 SV=1;
>tr|A0A091U380|A0A091U380_PHORB DNA polymerase lambda OS=Phoenicopterus ruber ruber
GN=N337_08802 PE=4 SV=1;
>tr|A0A0V1I8S4|A0A0V1I8S4_9BILA DNA polymerase lambda OS=Trichinella zimbabwensis
GN=MRPL30 PE=4 SV=1;
>tr|U3K1T5|U3K1T5_FICAL Uncharacterized protein OS=Ficedula albicollis GN=POLL PE=4 SV=1;
>tr|F8PSZ2|F8PSZ2_SERL3 Putative uncharacterized protein OS=Serpula lacrymans var. lacrymans
(strain S7.3) GN=SERLA73DRAFT_50337 PE=4 SV=1;
>tr|A0A091KJF3|A0A091KJF3_9GRUI DNA polymerase lambda OS=Chlamydotis macqueenii
GN=N324_09478 PE=4 SV=1;
>tr|H9FT36|H9FT36_MACMU DNA polymerase lambda isoform a OS=Macaca mulatta GN=POLL PE=2
SV=1;
>tr|R7TPZ3|R7TPZ3_CAPTE Uncharacterized protein OS=Capitella teleta GN=CAPTEDRAFT_213338
PE=4 SV=1;
>tr|A0A093FDU6|A0A093FDU6_TYTAL DNA polymerase lambda OS=Tyto alba GN=N341_04059
PE=4 SV=1;
>tr|A0A0A0A008|A0A0A0A008_CHAVO DNA polymerase lambda OS=Charadrius vociferus
GN=N301_14659 PE=4 SV=1;
>tr|M7AL53|M7AL53_CHEMY DNA polymerase lambda OS=Chelonia mydas GN=UY3_17041 PE=4
SV=1;
>tr|A0A146S619|A0A146S619_FUNHE DNA nucleotidylexotransferase OS=Fundulus heteroclitus PE=4
SV=1;
>tr|B9HVB4|B9HVB4_POPTR DNA polymerase lambda family protein OS=Populus trichocarpa
GN=POPTR_0010s10490g PE=4 SV=1;
>tr|Q4S1W9|Q4S1W9_TETNG Chromosome undetermined SCAF14764, whole genome shotgun
sequence OS=Tetraodon nigroviridis GN=GSTENG00025356001 PE=4 SV=1;
>tr|A0A094KN48|A0A094KN48_9AVES DNA polymerase lambda OS=Podiceps cristatus
GN=N338_07866 PE=4 SV=1;
>tr|A0A060YCF7|A0A060YCF7_ONCMY Uncharacterized protein OS=Oncorhynchus mykiss
GN=GSONMT00007442001 PE=4 SV=1;
>tr|H0X619|H0X619_OTOGA Uncharacterized protein OS=Otolemur garnettii GN=POLL PE=4 SV=1;
>tr|A0A0S7LXJ4|A0A0S7LXJ4_9TELE DPOLL OS=Poeciliopsis prolifica GN=DPOLL PE=4 SV=1;
>tr|A0A091V6S1|A0A091V6S1_OPIHO DNA polymerase lambda OS=Opisthocomus hoazin
GN=N306_14567 PE=4 SV=1;
>tr|V3ZZB0|V3ZZB0_LOTGI Uncharacterized protein (Fragment) OS=Lottia gigantea
GN=LOTGIDRAFT_72491 PE=4 SV=1;
>tr|A0A0C3C9Q4|A0A0C3C9Q4_HEBCY Uncharacterized protein OS=Hebeloma cylindrosporum h7
GN=M413DRAFT_446505 PE=4 SV=1;
>tr|I3KRF4|I3KRF4_ORENI Uncharacterized protein OS=Oreochromis niloticus GN=wbp11 PE=4 SV=1;
>tr|A0A093CA12|A0A093CA12_TAUER DNA polymerase lambda OS=Tauraco erythrolophus
GN=N340_11190 PE=4 SV=1;
>tr|A0A0D2QDZ0|A0A0D2QDZ0_GOSRA Uncharacterized protein OS=Gossypium raimondii
GN=B456_002G164200 PE=4 SV=1;
>tr|K9J1X8|K9J1X8_DESRO Putative dna polymerase iv family x OS=Desmodus rotundus PE=2 SV=1;
>tr|A0A146YFR1|A0A146YFR1_FUNHE DNA polymerase lambda OS=Fundulus heteroclitus PE=4
SV=1;
>tr|A0A0V1KGE2|A0A0V1KGE2_TRIPS DNA polymerase lambda OS=Trichinella pseudospiralis
GN=Mrp130 PE=4 SV=1;
>tr|S9WVS1|S9WVS1_CAMFR DNA polymerase lambda isoform a OS=Camelus ferus
GN=CB1_000642026 PE=4 SV=1;
>tr|R7VD68|R7VD68_CAPTE Uncharacterized protein OS=Capitella teleta GN=CAPTEDRAFT_169992
PE=4 SV=1;
>tr|D8S132|D8S132_SELML Putative uncharacterized protein (Fragment) OS=Selaginella moellendorffii
GN=SELMODRAFT_106459 PE=4 SV=1;
>tr|A0A0D2U5A1|A0A0D2U5A1_CAPO3 Uncharacterized protein OS=Capsaspora owczarzaki (strain
ATCC 30864) GN=CAOG_001654 PE=4 SV=1;
>tr|A0A0N5DI78|A0A0N5DI78_TRIMR Uncharacterized protein OS=Trichuris muris PE=4 SV=1;
>tr|A0A0V1EYE1|A0A0VIEYE1_TRIPS DNA polymerase lambda OS=Trichinella pseudospiralis
GN=POLL PE=4 SV=1;
>tr|A0A146YDH9|A0A146YDH9_FUNHE DNA polymerase lambda OS=Fundulus heteroclitus PE=4
SV=1;
>tr|A0A1S3L5G2|A0A1S3L5G2_SALSA DNA polymerase lambda-like OS=Salmo salar
GN=LOC106564589 PE=4 SV=1;
>tr|Q7SXH7|Q7SXH7_DANRE Poll protein OS=Danio rerio GN=poll PE=2 SV=1;
>tr|M3ZCZ7|M3ZCZ7_XIPMA Uncharacterized protein OS=Xiphophorus maculatus PE=4 SV=1;
>tr|A0A146YDI0|A0A146YDI0_FUNHE DNA polymerase lambda OS=Fundulus heteroclitus PE=4
SV=1;
>tr|D8Q9V7|D8Q9V7_SCHCM Putative uncharacterized protein OS=Schizophyllum commune (strain
H4-8 / FGSC 9210) GN=SCHCODRAFT_57262 PE=4 SV=1;
>tr|Q6P0S1|Q6P0S1_DANRE Polymerase (DNA directed), lambda OS=Danio rerio GN=poll PE=2 SV=1;
>tr|B8JIE9|B8JIE9_DANRE Polymerase (DNA directed), lambda OS=Danio rerio GN=poll PE=4 SV=1;
>tr|A0A0V0YDC0|A0A0V0YDC0_TRIPS DNA polymerase lambda OS=Trichinella pseudospiralis
GN=Mrp130 PE=4 SV=1;
>tr|A0A146NC85|A0A146NC85_FUNHE DNA polymerase lambda OS=Fundulus heteroclitus PE=4
SV=1;
>tr|A0A147AV47|A0A147AV47_FUNHE DNA polymerase lambda OS=Fundulus heteroclitus PE=4
SV=1;
>tr|A0A0V1MQR6|A0A0V1MQR6_9BILA DNA polymerase lambda OS=Trichinella papuae
GN=MRPL30 PE=4 SV=1;
>tr|A0A061E5V2|A0A061E5V2_THECC DNA polymerase lambda (POLL) isoform 5 OS=Theobroma
cacao GN=TCM_010205 PE=4 SV=1;
>tr|M7WSS0|M7WSS0_RHOT1 Beta dna polymerase OS=Rhodosporidium toruloides (strain NP11)
GN=RHTO_01992 PE=4 SV=1;
>tr|A0A061APG6|A0A061APG6_RHOTO RHTO0S04e05468g1_1 OS=Rhodosporidium toruloides
GN=RHTO0S_04e05468g PE=4 SV=1;
>tr|A0A061E5Q9|A0A061E5Q9_THECC DNA polymerase lambda (POLL) isoform 4 OS=Theobroma
cacao GN=TCM_010205 PE=4 SV=1;
>tr|A0A0V0RKB0|A0A0V0RKB0_9BILA DNA polymerase lambda OS=Trichinella nelsoni GN=POLL
PE=4 SV=1;
>tr|A0A091G5J5|A0A091G5J5_9AVES DNA polymerase lambda OS=Cuculus canorus GN=N303_14197
PE=4 SV=1;
>tr|A0A1A7XC13|A0A1A7XC13_9TELE Polymerase (DNA directed), lambda OS=Aphyosemion
striatum GN=POLL PE=4 SV=1;
>tr|A0A1A8VFI7|A0A1A8VFI7_NOTFU Polymerase (DNA directed), lambda (Fragment)
OS=Nothobranchius furzeri GN=POLL PE=4 SV=1;
>tr|A0A1A7YLF9|A0A1A7YLF9_9TELE Polymerase (DNA directed), lambda (Fragment)
OS=Aphyosemion striatum GN=POLL PE=4 SV=1;
>tr|A0A1A8AAH4|A0A1A8AAH4_NOTFU Polymerase (DNA directed), lambda (Fragment)
OS=Nothobranchius furzeri GN=POLL PE=4 SV=1;
>tr|D8R0V4|D8R0V4_SELML Putative uncharacterized protein (Fragment) OS=Selaginella
moellendorffii GN=SELMODRAFT_83116 PE=4 SV=1;
>tr|A0A135TQY6|A0A135TQY6_9PEZI Uncharacterized protein OS=Colletotrichum nymphaeae SA-01
GN=CNYM01_13026 PE=4 SV=1;
>tr|A0A0C9TIH7|A0A0C9TIH7_PAXIN Unplaced genomic scaffold PAXINscaffold_14, whole genome
shotgun sequence OS=Paxillus involutus ATCC 200175 GN=PAXINDRAFT_77120 PE=4 SV=1;
>tr|A0A087VCA5|A0A087VCA5_BALRE DNA polymerase lambda OS=Balearica regulorum
gibbericeps GN=N312_03659 PE=4 SV=1;
>tr|W5LHN1|W5LHN1_ASTMX Uncharacterized protein OS=Astyanax mexicanus PE=4 SV=1;
>tr|A0A0V1MQQ1|A0A0V1MQQ1_9BILA DNA polymerase lambda OS=Trichinella papuae
GN=MRPL30 PE=4 SV=1;
>tr|A0A0D2NNZ0|A0A0D2NNZ0_GOSRA Uncharacterized protein OS=Gossypium raimondii
GN=B456_002G164200 PE=4 SV=1;
>tr|A0A0V0ZUW5|A0A0V0ZUW5_9BILA DNA polymerase lambda OS=Trichinella patagoniensis
GN=POLL PE=4 SV=1;
>tr|A0A1E1MHN8|A0A1E1MHN8_RHYSE Related to DNA polymerase Tdt-N OS=Rhynchosporium
secalis GN=RSE6_09304 PE=4 SV=1;
>tr|A0A1B9GPF0|A0A1B9GPF0_9TREE DNA polymerase mu subunit OS=Kwoniella heveanensis
BCC8398 GN=I316_05288 PE=4 SV=1;
>tr|A0A1B9H8T1|A0A1B9H8T1_9TREE DNA polymerase mu subunit OS=Kwoniella heveanensis CBS
569 GN=I317_06553 PE=4 SV=1;
>tr|A0A022QLW0|A0A022QLW0_ERYGU Uncharacterized protein OS=Erythranthe guttata
GN=MIMGU_mgv1a026593mg PE=4 SV=1;
>tr|A0A074YMR2|A0A074YMR2_9PEZI Uncharacterized protein OS=Aureobasidium subglaciale EXF-
2481 GN=AUEXF2481DRAFT_217585 PE=4 SV=1;
>tr|M7WGS7|M7WGS7_RHOT1 Beta dna polymerase OS=Rhodosporidium toruloides (strain NP11)
GN=RHTO_04182 PE=4 SV=1;
>tr|S9RKH6|S9RKH6_SCHOY DNA polymerase Xfamily OS=Schizosaccharomyces octosporus (strain
yFS286) GN=SOCG_01968 PE=4 SV=1;
>tr|A0A1S3KIF7|A0A1S3KIF7_LINUN DNA polymerase lambda-like isoform X1 OS=Lingula unguis
GN=LOC106181965 PE=4 SV=1;
>tr|A0A0V0ZUB3|A0A0V0ZUB3_9BILA DNA polymerase lambda OS=Trichinella patagoniensis
GN=POLL PE=4 SV=1;
>tr|A0A0P7UXQ3|A0A0P7UXQ3_9TELE Uncharacterized protein (Fragment) OS=Scleropages formosus
GN=Z043_105872 PE=4 SV=1;
>tr|A0A1A8MRC5|A0A1A8MRC5_9TELE Polymerase (DNA directed), lambda (Fragment)
OS=Nothobranchius pienaari GN=POLL PE=4 SV=1;
>tr|J3MCS5|J3MCS5_ORYBR Uncharacterized protein OS=Oryza brachyantha PE=4 SV=1;
>tr|A0A1A8DI13|A0A1A8DI13_9TELE Polymerase (DNA directed), lambda OS=Nothobranchius
kadleci GN=POLL PE=4 SV=1;
>tr|H2ULE5|H2ULE5_TAKRU Uncharacterized protein OS=Takifugu rubripes GN=poll PE=4 SV=1;
>tr|A0A087XHB2|A0A087XHB2_POEFO Uncharacterized protein OS=Poecilia formosa PE=4 SV=1;
>tr|A0A1A8P4I0|A0A1A8P4I0_9TELE Polymerase (DNA directed), lambda OS=Nothobranchius
rachovii GN=POLL PE=4 SV=1;
>tr|A0A1A8QT58|A0A1A8QT58_9TELE Polymerase (DNA directed), lambda OS=Nothobranchius
rachovii GN=POLL PE=4 SV=1;
>tr|A0A1A8M358|A0A1A8M358_9TELE Polymerase (DNA directed), lambda OS=Nothobranchius
pienaari GN=POLL PE=4 SV=1;
>tr|A0A1A8II81|A0A1A8II81_NOTKU Polymerase (DNA directed), lambda OS=Nothobranchius
kuhntae GN=POLL PE=4 SV=1;
>tr|L8G8G3|L8G8G3_PSED2 Uncharacterized protein OS=Pseudogymnoascus destructans (strain ATCC
MYA-4855 / 20631-21) GN=GMDG_03600 PE=4 SV=1;
>tr|A0A177A4J2|A0A177A4J2_9PEZI Uncharacterized protein OS=Pseudogymnoascus destructans
GN=VC83_05832 PE=4 SV=1;
>tr|A0A010RYY6|A0A010RYY6_9PEZI Uncharacterized protein OS=Colletotrichum fioriniae PJ7
GN=CFIO01_13046 PE=4 SV=1;
>tr|A0A1A8KDR9|A0A1A8KDR9_NOTKU Polymerase (DNA directed), lambda (Fragment)
OS=Nothobranchius kuhntae GN=POLL PE=4 SV=1;
>tr|A0A1A8R1Y2|A0A1A8R1Y2_9TELE Polymerase (DNA directed), lambda OS=Nothobranchius
pienaari GN=POLL PE=4 SV=1;
>tr|A0A0C3E7J0|A0A0C3E7J0_9HOMO Uncharacterized protein OS=Scleroderma citrinum Foug A
GN=SCLCIDRAFT_116236 PE=4 SV=1;
>tr|K7FIX4|K7FIX4_PELSI Uncharacterized protein OS=Pelodiscus sinensis GN=POLL PE=4 SV=1;
>tr|K7FIW4|K7FIW4_PELSI Uncharacterized protein OS=Pelodiscus sinensis GN=POLL PE=4 SV=1;
>tr|A0A0V0VI93|A0A0V0VI93_9BILA DNA polymerase lambda OS=Trichinella sp. T9 GN=T09_7096
PE=4 SV=1;
>tr|T1ERD2|T1ERD2_HELRO Uncharacterized protein OS=Helobdella robusta
GN=HELRODRAFT_161346 PE=4 SV=1;
>tr|A0A1A6A4X0|A0A1A6A4X0_9TREE DNA polymerase mu subunit OS=Kwoniella dejecticola CBS
10117 GN=1303_04440 PE=4 SV=1;
>tr|A0A1A8QJY3|A0A1A8QJY3_9TELE Polymerase (DNA directed), lambda OS=Nothobranchius
rachovii GN=POLL PE=4 SV=1;
>tr|A0A166QWC5|A0A166QWC5_9HOMO Nucleotidyltransferase OS=Fibulorhizoctonia sp. CBS
109695 GN=FIBSPDRAFT_853496 PE=4 SV=1;
>tr|A0A135TFZ2|A0A135TFZ2_9PEZI Uncharacterized protein OS=Colletotrichum simmondsii
GN=CSIM01_09367 PE=4 SV=1;
>sp|Q09693|DPO4_SCHPO DNA polymerase type-X family protein pol4 OS=Schizosaccharomyces
pombe (strain 972 / ATCC 24843) GN=pol4 PE=3 SV=1;
>tr|A0A0V1P3A7|A0A0V1P3A7_9BILA DNA polymerase lambda OS=Trichinella sp. T8 GN=POLL
PE=4 SV=1;
>tr|A0A0VILPY1|A0A0VILPY1_9BILA DNA polymerase lambda OS=Trichinella nativa GN=POLL
PE=4 SV=1;
>tr|A0A1E1L8R7|A0A1E1L8R7_9HELO Related to DNA polymerase Tdt-N OS=Rhynchosporium
commune GN=RCO7_07163 PE=4 SV=1;
>tr|A0A1B9G359|A0A1B9G359_9TREE DNA polymerase mu subunit OS=Kwoniella bestiolae CBS
10118 GN=I302_05273 PE=4 SV=1;
>tr|A0A1A8GWF2|A0A1A8GWF2_9TELE Polymerase (DNA directed), lambda (Fragment)
OS=Nothobranchius korthausae GN=POLL PE=4 SV=1;
>tr|A0A0V1P3B3|A0A0V1P3B3_9BILA DNA polymerase lambda OS=Trichinella sp. T8 GN=POLL
PE=4 SV=1;
>tr|A0A0V1LPY7|A0A0V1LPY7_9BILA DNA polymerase lambda OS=Trichinella nativa GN=POLL
PE=4 SV=1;
>tr|A0A1A8FGG8|A0A1A8FGG8_9TELE Polymerase (DNA directed), lambda (Fragment)
OS=Nothobranchius korthausae GN=POLL PE=4 SV=1;
>tr|A0A0V1IBA1|A0A0V1IBA1_9BILA DNA polymerase lambda OS=Trichinella zimbabwensis
GN=MRPL30 PE=4 SV=1;
>tr|X6MW96|X6MW96_RETFI DNA-directed DNA polymerase lambda (Fragment) OS=Reticulomyxa
filosa GN=RFI_18997 PE=4 SV=1;
>tr|F6VFI0|F6VFI0_HORSE Uncharacterized protein OS=Equus caballus GN=POLL PE=4 SV=1;
>tr|A0A1E1K5W1|A0A1E1K5W1_9HELO Related to DNA polymerase Tdt-N OS=Rhynchosporium
agropyri GN=RAG0_03773 PE=4 SV=1;
>tr|A0A0D2MBV9|A0A0D2MBV9_GOSRA Uncharacterized protein OS=Gossypium raimondii
GN=B456_002G164200 PE=4 SV=1;
>tr|A0A1D1UV65|A0A1D1UV65_RAMVA Uncharacterized protein OS=Ramazzottius varieornatus
GN=RvY_04493-1 PE=4 SV=1;
>tr|M2MR12|M2MR12_BAUCO Uncharacterized protein OS=Baudoinia compniacensis (strain UAMH
10762) GN=BAUCODRAFT_85556 PE=4 SV=1;
>tr|A0A0V1D247|A0A0V1D247_TRIBR DNA polymerase lambda OS=Trichinella britovi GN=POLL
PE=4 SV=1;
>tr|A0A0V0WMC1|A0A0V0WMC1_9BILA DNA polymerase lambda OS=Trichinella sp. T6 GN=POLL
PE=4 SV=1;
>tr|G0QLY3|GOQLY3_ICHMG DNA-directed polymerase lambda, putative (Fragment)
OS=Ichthyophthirius multifiliis (strain G5) GN=IMG5_038620 PE=4 SV=1;
>tr|A0A093ZS11|A0A093ZS11_9PEZI Uncharacterized protein OS=Pseudogymnoascus sp. VKM F-3775
GN=V491_04165 PE=4 SV=1;
>tr|A0A0P4VZD0|A0A0P4VZD0_9EUCA Uncharacterized protein OS=Scylla olivacea PE=4 SV=1;
>tr|G3Q2Q6|G3Q2Q6_GASAC Uncharacterized protein OS=Gasterosteus aculeatus PE=4 SV=1;
>tr|A0A1A8Q5T7|A0A1A8Q5T7_9TELE Polymerase (DNA directed), mu (Fragment)
OS=Nothobranchius rachovii GN=POLM PE=4 SV=1;
>tr|A0A0V1D263|A0A0V1D263_TRIBR DNA polymerase lambda OS=Trichinella britovi GN=POLL
PE=4 SV=1;
>tr|A0A0V1BLL4|A0A0V1BLL4_TRISP DNA polymerase lambda OS=Trichinella spiralis GN=POLL
PE=4 SV=1;
>tr|A0A1B8CG84|A0A1B8CG84_9PEZI Uncharacterized protein OS=Pseudogymnoascus sp. WSF 3629
GN=VE00_03848 PE=4 SV=1;
>tr|E2LZ52|E2LZ52_MONPE Uncharacterized protein (Fragment) OS=Moniliophthora perniciosa (strain
FA553 / isolate CP02) GN=MPER_12617 PE=4 SV=1;
>tr|A0A072TY09|A0A072TY09_MEDTR DNA polymerase lambda-like protein OS=Medicago truncatula
GN=MTR_7g039450 PE=4 SV=1;
>tr|A0A176WNC6|A0A176WNC6_MARPO Uncharacterized protein OS=Marchantia polymorpha subsp.
polymorpha GN=AXG93_1487s1150 PE=4 SV=1;
>tr|A0A194W3X9|A0A194W3X9_9PEZI DNA polymerase type-X family protein pol4 OS=Valsa mali
GN=VM1G_06647 PE=4 SV=1;
>tr|A0A0V1LPX0|A0A0V1LPX0_9BILA DNA polymerase lambda OS=Trichinella nativa GN=POLL
PE=4 SV=1;
>tr|A0A0D9WNQ5|A0A0D9WNQ5_9ORYZ Uncharacterized protein OS=Leersia perrieri PE=4 SV=1;
>tr|A0A0V1BMJ0|A0A0V1BMJ0_TRISP DNA polymerase lambda OS=Trichinella spiralis GN=POLL
PE=4 SV=1;
>tr|A0A139AY58|A0A139AY58_GONPR Nucleotidyltransferase OS=Gonapodya prolifera JEL478
GN=M427DRAFT_276455 PE=4 SV=1;
>tr|A0A1B8FTY4|A0A1B8FTY4_9PEZI Uncharacterized protein OS=Pseudogymnoascus sp. 03VT05
GN=VE02_03686 PE=4 SV=1;
>tr|A0A059D505|A0A059D505_EUCGR Uncharacterized protein OS=Eucalyptus grandis
GN=EUGRSUZ_B02561 PE=4 SV=1;
>tr|H3D3G8|H3D3G8_TETNG Uncharacterized protein OS=Tetraodon nigroviridis PE=4 SV=1;
>tr|A0A1S3DX16|A0A1S3DX16_CICAR DNA polymerase beta isoform X2 OS=Cicer arietinum
GN=LOC101499677 PE=4 SV=1;
>tr|A0A1S2Z862|A0A1S2Z862_CICAR DNA polymerase beta isoform X1 OS=Cicer arietinum
GN=LOC101499677 PE=4 SV=1;
>tr|A0A0J8BWQ9|A0A0J8BWQ9_BETVU Uncharacterized protein OS=Beta vulgaris subsp. vulgaris
GN=BVRB_8g181170 PE=4 SV=1;
>tr|A0A0B0MKA5|A0A0B0MKA5_GOSAR DNA polymerase lambda OS=Gossypium arboreum
GN=F383_21525 PE=4 SV=1;
>tr|A0A1B8EAE0|A0A1B8EAE0_9PEZI Uncharacterized protein OS=Pseudogymnoascus sp. 23342-1-I1
GN=VE03_02256 PE=4 SV=1;
>tr|A0A194V4W0|A0A194V4W0_9PEZI DNA polymerase type-X family protein pol4 OS=Valsa mali
var. pyri GN=VP1G_06122 PE=4 SV=1;
>sp|Q67VC8|DPOLL_ORYSJ DNA polymerase lambda OS=Oryza sativa subsp. japonica GN=POLL
PE=1 SV=1;
>tr|A0A0S7IWG7|A0A0S7IWG7_9TELE TDT OS=Poeciliopsis prolifica GN=TDT PE=4 SV=1;
>tr|A0A1B8F877|A0A1B8F877_9PEZI Uncharacterized protein OS=Pseudogymnoascus sp. 05NY08
GN=VF21_01476 PE=4 SV=1;
>tr|S2KCR4|S2KCR4_MUCC1 Uncharacterized protein OS=Mucor circinelloides f. circinelloides (strain
1006PhL) GN=HMPREF1544_03081 PE=4 SV=1;
>tr|A0A1E1XQK5|A0A1E1XQK5_9ACAR Putative dna polymerase lambda OS=Amblyomma sculptum
PE=2 SV=1;
>tr|A0A086T1M2|A0A086T1M2_ACRC1 DNA polymerase type-X family protein-like protein
OS=Acremonium chrysogenum (strain ATCC 11550 / CBS 779.69 / DSM 880 / JCM 23072 / IMI 49137)
GN=ACRE_059880 PE=4 SV=1;
>tr|A0A0V0WM43|A0A0V0WM43_9BILA DNA polymerase lambda OS=Trichinella sp. T6 GN=POLL
PE=4 SV=1;
>tr|B9FSE5|B9FSE5_ORYSJ Uncharacterized protein OS=Oryza sativa subsp. japonica GN=OsJ_20743
PE=4 SV=1;
>tr|A0A094D6X3|A0A094D6X3_9PEZI Uncharacterized protein OS=Pseudogymnoascus sp. VKM F-
4281 (FW-2241) GN=V493_01816 PE=4 SV=1;
>tr|A0A0B2NZB6|A0A0B2NZB6_GLYSO DNA polymerase lambda OS=Glycine soja
GN=glysoja_000287 PE=4 SV=1;
>tr|I1JCZ1 |I1JCZ1_SOYBN Uncharacterized protein OS=Glycine max GN=LOC100820492 PE=4 SV=2;
>tr|A0A135S367|A0A135S367_9PEZI Uncharacterized protein OS=Colletotrichum salicis
GN=CSAL01_00528 PE=4 SV=1;
>tr|A0A131XSZ4|A0A131XSZ4_IXORI Putative dna polymerase lambda OS=Ixodes ricinus PE=2 SV=1;
>tr|F7HD66|F7HD66_MACMU Uncharacterized protein OS=Macaca mulatta GN=POLL PE=4 SV=2;
>tr|A0A0F7SFU3|A0A0F7SFU3_PHARH DNA polymerase IV (Family X) OS=Phaffia rhodozyma PE=4
SV=1;
>tr|A0A0V1G2E9|A0A0V1G2E9_TRIPS DNA polymerase lambda OS=Trichinella pseudospiralis
GN=POLL PE=4 SV=1;
>tr|A0A0V1I9X7|A0A0V1I9X7_9BILA DNA polymerase lambda OS=Trichinella zimbabwensis
GN=MRPL30 PE=4 SV=1;
>tr|A0A0S3T9T5|A0A0S3T9T5_PHAAN Uncharacterized protein OS=Vigna angularis var. angularis
GN=Vigan.11G102700 PE=4 SV=1;
>tr|A0A1S3V956|A0A1S3V956_VIGRR DNA polymerase beta isoform X2 OS=Vigna radiata var. radiata
GN=LOC106772788 PE=4 SV=1;
>tr|V4VGR5|V4VGR5_9ROSI Uncharacterized protein OS=Citrus clementina
GN=CICLE_v10031184mg PE=4 SV=1;
>tr|A0A067H4W7|A0A067H4W7_CITSI Uncharacterized protein OS=Citrus sinensis
GN=CISIN_1g0093031mg PE=4 SV=1;
>tr|A0A0V1EY07|A0A0V1EY07_TRIPS DNA polymerase lambda OS=Trichinella pseudospiralis
GN=POLL PE=4 SV=1;
>tr|A0A1S3V9I3|A0A1S3V9I3_VIGRR DNA polymerase beta isoform X1 OS=Vigna radiata var. radiata
GN=LOC106772788 PE=4 SV=1;
>tr|A0A1G4ATK0|A0A1G4ATK0_9PEZI Uncharacterized protein OS=Colletotrichum orchidophilum
GN=CORC01_12287 PE=4 SV=1;
>tr|G7KDW6|G7KDW6_MEDTR DNA polymerase lambda-like protein OS=Medicago truncatula
GN=MTR_5g040170 PE=4 SV=1;
>tr|A0A0V0YEE1|A0A0V0YEE1_TRIPS DNA polymerase lambda OS=Trichinella pseudospiralis
GN=Mrp130 PE=4 SV=1;
>tr|D8SG82|D8SG82_SELML Putative uncharacterized protein OS=Selaginella moellendorffii
GN=SELMODRAFT_155063 PE=4 SV=1;
>tr|A0A0V1BLX4|A0A0V1BLX4_TRISP DNA polymerase lambda OS=Trichinella spiralis GN=POLL
PE=4 SV=1;
>tr|S8A5W2|S8A5W2_DACHA Uncharacterized protein OS=Dactylellina haptotyla (strain CBS 200.50)
GN=H072_8085 PE=4 SV=1;
>tr|A0A0V0IDD6|A0A0V0IDD6_SOLCH Putative DNA polymerase lambda-like OS=Solanum
chacoense PE=4 SV=1;
>tr|M1ER80|M1ER80_MUSPF Deoxynucleotidyltransferase, terminal (Fragment) OS=Mustela putorius
furo PE=2 SV=1;
>tr|A0A094H117|A0A094H117_9PEZI Uncharacterized protein OS=Pseudogymnoascus sp. VKM F-4520
(FW-2644) GN=V502_09208 PE=4 SV=1;
>tr|A0A094G3V5|A0A094G3V5_9PEZI Uncharacterized protein OS=Pseudogymnoascus sp. VKM F-
4518 (FW-2643) GN=V500_04993 PE=4 SV=1;
>tr|W4GGX8|W4GGX8_9STRA Uncharacterized protein OS=Aphanomyces astaci GN=H257_07734
PE=4 SV=1;
>tr|A0A179FSW3|A0A179FSW3_METCM DNA polymerase beta OS=Pochonia chlamydosporia 170
GN=VFPPC_04534 PE=4 SV=1;
>tr|A0A1J3FA13|A0A1J3FA13_NOCCA DNA polymerase lambda (Fragment) OS=Noccaea caerulescens
GN=LC_TR10189_c0_g1_i1_g.35893 PE=4 SV=1;
>tr|G2Q7Z8|G2Q7Z8_MYCTT Uncharacterized protein OS=Myceliophthora thermophila (strain ATCC
42464 / BCRC 31852 / DSM 1799) GN=MYCTH_2300738 PE=4 SV=1;
>tr|A0A067JBU8|A0A067JBU8_JATCU Uncharacterized protein OS=Jatropha curcas GN=JCGZ_21772
PE=4 SV=1;
>tr|K1VA66|K1VA66_TRIAC Beta DNA polymerase OS=Trichosporon asahii var. asahii (strain CBS
8904) GN=A1Q2_04783 PE=4 SV=1;
>tr|J5RIV1|J5RIV1_TRIAS Beta DNA polymerase OS=Trichosporon asahii var. asahii (strain ATCC
90039 / CBS 2479 / JCM 2466 / KCTC 7840 / NCYC 2677 / UAMH 7654) GN=A1Q1_00793 PE=4
SV=1;
>tr|B8B4F7|B8B4F7_ORYSI Putative uncharacterized protein OS=Oryza sativa subsp. indica
GN=OsI_22314 PE=4 SV=1;
>tr|M3AHI8|M3AHI8_PSEFD Uncharacterized protein OS=Pseudocercospora fijiensis (strain CIRAD86)
GN=MYCFIDRAFT_162909 PE=4 SV=1;
>tr|A0A132B5Z1|A0A132B5Z1_9HELO Nucleotidyltransferase OS=Phialocephala scopiformis
GN=LY89DRAFT_602043 PE=4 SV=1;
>tr|F0WG97|F0WG97_9STRA DNA polymerase lambdalike protein putative OS=Albugo laibachii Nc14
GN=AINc14C89G5627 PE=4 SV=1;
>tr|N1RVS6|N1RVS6_FUSC4 Putative DNA polymerase family X C2F7.06c OS=Fusarium oxysporum f.
sp. cubense (strain race 4) GN=FOC4_g10006769 PE=4 SV=1;
>tr|X0JME1|X0JME1_FUSOX DNA polymerase IV OS=Fusarium oxysporum f. sp. cubense tropical race
4 54006 GN=FOIG_06662 PE=4 SV=1;
>tr|A0A0V0VHS7|A0A0V0VHS7_9BILA DNA polymerase lambda OS=Trichinella sp. T9
GN=T09_7096 PE=4 SV=1;
>tr|A0A0L1HPA7|A0A0L1HPA7_9PLEO Dna polymerase beta-like protein OS=Stemphylium lycopersici
GN=TW65_05101 PE=4 SV=1;
>tr|A0A094CYS0|A0A094CYS0_9PEZI Uncharacterized protein OS=Pseudogymnoascus sp. VKM F-
4516 (FW-969) GN=V497_05751 PE=4 SV=1;
>tr|A0A147BMR3|A0A147BMR3_IXORI Putative dna polymerase lambda OS=Ixodes ricinus PE=4
SV=1;
>tr|A0A1I8H3S8|A0A1I8H3S8_9PLAT Uncharacterized protein OS=Macrostomum lignano PE=4 SV=1;
>tr|A0A1R3FX17|A0A1R3FX17_9ROSI Uncharacterized protein OS=Corchorus olitorius
GN=COLO4_38096 PE=4 SV=1;
>tr|A0A0L0DBR4|A0A0L0DBR4_THETB Poll protein OS=Thecamonas trahens ATCC 50062
GN=AMSG_06060 PE=4 SV=1;
>tr|N1PTU7|N1PTU7_DOTSN Uncharacterized protein OS=Dothistroma septosporum (strain NZE10 /
CBS 128990) GN=DOTSEDRAFT_71509 PE=4 SV=1;
>tr|A0A0V0TT93|A0A0V0TT93_9BILA DNA polymerase lambda OS=Trichinella murrelli GN=POLL
PE=4 SV=1;
>tr|F8NSL8|F8NSL8_SERL9 Putative uncharacterized protein OS=Serpula lacrymans var. lacrymans
(strain S7.9) GN=SERLADRAFT_447668 PE=4 SV=1;
>tr|A0A0V0TT82|A0A0V0TT82_9BILA DNA polymerase lambda OS=Trichinella murrelli GN=POLL
PE=4 SV=1;
>tr|A0A093ZF85|A0A093ZF85_9PEZI Uncharacterized protein OS=Pseudogymnoascus sp. VKM F-4246
GN=V492_01628 PE=4 SV=1;
>tr|V9KZ36|V9KZ36_CALMI DNA polymerase lambda-like protein OS=Callorhinchus milii PE=2
SV=1;
>tr|V4KDK3|V4KDK3_EUTSA Uncharacterized protein OS=Eutrema salsugineum
GN=EUTSA_v10009477mg PE=4 SV=1;
>tr|A0A1S3BFG3|A0A1S3BFG3_CUCME DNA polymerase beta isoform X3 OS=Cucumis melo
GN=LOC103489040 PE=4 SV=1;
>tr|A0A1J3E083|A0A1J3E083_NOCCA DNA polymerase lambda (Fragment) OS=Noccaea caerulescens
GN=GA_TR19700_c1_g1_i1_g.65036 PE=4 SV=1;
>tr|I1Q121|I1Q121_ORYGL Uncharacterized protein OS=Oryza glaberrima PE=4 SV=1;
>tr|A0A093Y3X2|A0A093Y3X2_9PEZI Uncharacterized protein OS=Pseudogymnoascus sp. VKM F-
3557 GN=V490_02785 PE=4 SV=1;
>tr|A0A1B9IZN9|A0A1B9IZN9_9TREE DNA polymerase mu subunit OS=Kwoniella mangroviensis
CBS 10435 GN=L486_00649 PE=4 SV=1;
>tr|A0A0K6FNH4|A0A0K6FNH4_9HOMO Uncharacterized protein OS=Rhizoctonia solani GN=dntt
PE=4 SV=1;
>tr|F7CJ14|F7CJ14_CALJA Uncharacterized protein OS=Callithrix jacchus GN=POLL PE=4 SV=1;
>tr|A0A1S3Q0V2|A0A1S3Q0V2_SALSA DNA polymerase lambda-like isoform X1 OS=Salmo salar
GN=LOC106588764 PE=4 SV=1;
>tr|K3XW78|K3XW78_SETIT Uncharacterized protein OS=Setaria italica GN=LOC101782419 PE=4
SV=1;
>tr|R0K168|R0K168_SETT2 Uncharacterized protein OS=Setosphaeria turcica (strain 28A)
GN=SETTUDRAFT_139964 PE=4 SV=1;
>tr|F9F942|F9F942_FUSOF Uncharacterized protein OS=Fusarium oxysporum (strain Fo5176)
GN=FOXB_02917 PE=4 SV=1;
>tr|X0IJN7|X0IJN7_FUSOX DNA polymerase IV OS=Fusarium oxysporum f. sp. conglutinans race 2
54008 GN=FOPG_03355 PE=4 SV=1;
>tr|S4R571|S4R571_PETMA Uncharacterized protein OS=Petromyzon marinus PE=4 SV=1;
>tr|A0A136J6M4|A0A136J6M4_9PEZI Uncharacterized protein OS=Microdochium bolleyi
GN=Micbo1qcDRAFT_232520 PE=4 SV=1;
>tr|I1GZF6|I1GZF6_BRADI Uncharacterized protein OS=Brachypodium distachyon GN=LOC100827137
PE=4 SV=1;
>tr|K5UUC0|K5UUC0_PHACS Uncharacterized protein OS=Phanerochaete carnosa (strain HHB-10118-
sp) GN=PHACADRAFT_176005 PE=4 SV=1;
>tr|A0A1D6NRF5|A0A1D6NRF5_MAIZE DNA polymerase lambda (POLL) OS=Zea mays
GN=ZEAMMB73_Zm00001d044780 PE=4 SV=1;
>tr|A0A1Q3DD56|A0A1Q3DD56_CEPFO NTP_transf_2 domain-containing protein/DNA_pol_lambd_f
domain-containing protein OS=Cephalotus follicularis GN=CFOL_v3_33823 PE=4 SV=1;
>tr|X0DAE4|X0DAE4_FUSOX DNA polymerase IV OS=Fusarium oxysporum f. sp. raphani 54005
GN=FOQG_06295 PE=4 SV=1;
>tr|X0L8Y4|X0L8Y4_FUSOX DNA polymerase IV OS=Fusarium oxysporum f. sp. vasinfectum 25433
GN=FOTG_10014 PE=4 SV=1;
>tr|W9PMW0|W9PMW0_FUSOX DNA polymerase IV OS=Fusarium oxysporum f. sp. pisi HDV247
GN=FOVG_08521 PE=4 SV=1;
>tr|N4TUZ3|N4TUZ3_FUSC1 Putative DNA polymerase family X C2F7.06c OS=Fusarium oxysporum f.
sp. cubense (strain race 1) GN=FOC1_g10006650 PE=4 SV=1;
>tr|W9J0J8|W9J0J8_FUSOX DNA polymerase IV OS=Fusarium oxysporum FOSC 3-a
GN=FOYG_00559 PE=4 SV=1;
>tr|A0A194YIA1|A0A194YIA1_SORBI Uncharacterized protein OS=Sorghum bicolor
GN=SORBI_010G097500 PE=4 SV=1;
>tr|C7Z1J3|C7Z1J3_NECH7 Putative uncharacterized protein OS=Nectria haematococca (strain 77-13-4 /
ATCC MYA-4622 / FGSC 9596 / MPVI) GN=NECHADRAFT_50658 PE=4 SV=1;
>tr|A0A177BXP7|A0A177BXP7_9PLEO DNA polymerase beta OS=Paraphaeosphaeria sporulosa
GN=CC84DRAFT_389629 PE=4 SV=1;
>tr|A0A109FBN5|A0A109FBN5_9BASI Nucleotidyltransferase OS=Rhodotorula sp. JG-1b
GN=RHOSPDRAFT_36897 PE=4 SV=1;
>tr|Q5JQP4|Q5JQP4_HUMAN DNA polymerase lambda OS=Homo sapiens GN=POLL PE=1 SV=1;
>tr|E5SCZ0|E5SCZ0_TRISP DNA polymerase lambda OS=Trichinella spiralis GN=Tsp_01603 PE=4
SV=1;
>sp/Q9UGP5-2|DPOLL_HUMAN Isoform 2 of DNA polymerase lambda OS=Homo sapiens GN=POLL;
>tr|F4Q5G1|F4Q5G1_DICFS Phosphatase tensin type domain-containing protein OS=Dictyostelium
fasciculatum (strain SH3) GN=DFA_08207 PE=4 SV=1;
>tr|W9KQZ0|W9KQZ0_FUSOX DNA polymerase IV OS=Fusarium oxysporum Fo47 GN=FOZG_02867
PE=4 SV=1;
>tr|A0A0D7BH09|A0A0D7BH09_9HOMO Nucleotidyltransferase OS=Cylindrobasidium torrendii
FP15055 ss-10 GN=CYLTODRAFT_226281 PE=4 SV=1;
>tr|A0A1E3QZQ5|A0A1E3QZQ5_9ASCO Uncharacterized protein OS=Babjeviella inositovora NRRL Y-
12698 GN=BABINDRAFT_159558 PE=4 SV=1;
>tr|A0A063CCJ7|A0A063CCJ7_9HYPO DNA polymerase beta OS=Ustilaginoidea virens
GN=UV8b_354 PE=4 SV=1;
>tr|A0A0E9NFZ0|A0A0E9NFZ0_9ASCO Uncharacterized protein OS=Saitoella complicata NRRL Y-
17804 GN=G7K_2923-t1 PE=3 SV=1;
>tr|A0A1S4CC54|A0A1S4CC54_TOBAC DNA polymerase beta-like isoform X3 OS=Nicotiana tabacum
GN=LOC107817483 PE=4 SV=1;
>tr|M0SDF3|M0SDF3_MUSAM Uncharacterized protein OS=Musa acuminata subsp. malaccensis PE=4
SV=1;
>tr|J9I2Q5|J9I2Q5_9SPIT Helix-hairpin-helix motif family protein OS=Oxytricha trifallax
GN=OXYTRI_11966 PE=4 SV=1;
>tr|A0A1C1WVK1|A0A1C1WVK1_9PEZI DNA polymerase IV OS=Diaporthe helianthi
GN=DHEL01_08189 PE=4 SV=1;
>tr|R7TPP6|R7TPP6_CAPTE Uncharacterized protein OS=Capitella teleta GN=CAPTEDRAFT_227708
PE=4 SV=1;
>tr|A0A094H196|A0A094H196_9PEZI Uncharacterized protein OS=Pseudogymnoascus sp. VKM F-4519
(FW-2642) GN=V501_06803 PE=4 SV=1;
>tr|A0A179HAH2|A0A179HAH2_9HYPO DNA polymerase beta OS=Purpureocillium lilacinum
GN=VFPBJ_00561 PE=4 SV=1;
>tr|A0A167HMH0|A0A167HMH0_9BASI Nucleotidyltransferase OS=Calocera viscosa TUFC12733
GN=CALVIDRAFT_488572 PE=4 SV=1;
>tr|A0A0D3GEN6|A0A0D3GEN6_9ORYZ Uncharacterized protein OS=Oryza barthii PE=4 SV=1;
>tr|A0A0F8A5P0|A0A0F8A5P0_9HYPO Uncharacterized protein OS=Hirsutella minnesotensis 3608
GN=HIM_04821 PE=4 SV=1;
>tr|E5A138|E5A138_LEPMJ Similar to terminal deoxynucleotidyl transferase OS=Leptosphaeria
maculans (strain JN3 / isolate v23.1.3 / race Av1-4-5-6-7-8) GN=LEMA_P104650.1 PE=4 SV=1;
>tr|E3S5Q0|E3S5Q0_PYRTT Putative uncharacterized protein OS=Pyrenophora teres f. teres (strain 0-1)
GN=PTT_17985 PE=4 SV=1;
>tr|T0Q5B0|T0Q5B0_9STRA Uncharacterized protein OS=Saprolegnia diclina VS20 GN=SDRG_09530
PE=4 SV=1;
>tr|B4DEF5|B4DEF5_HUMAN cDNA FLJ55191, highly similar to DNA polymerase lambda (EC 2.7.7.7)
OS=Homo sapiens PE=2 SV=1;
>tr|J9IGR6|J9IGR6_9SPIT Helix-hairpin-helix motif family protein OS=Oxytricha trifallax
GN=OXYTRI_08498 PE=4 SV=1;
>tr|A0A1S2Z857|A0A1S2Z857_CICAR DNA polymerase beta isoform X3 OS=Cicer arietinum
GN=LOC101499677 PE=4 SV=1;
>tr|A0A1S4CCE0|A0A1S4CCE0_TOBAC DNA polymerase beta-like isoform X1 OS=Nicotiana tabacum
GN=LOC107817483 PE=4 SV=1;
>tr|A0A0E0PVK3|A0A0E0PVK3_ORYRU Uncharacterized protein OS=Oryza rufipogon PE=4 SV=1;
>tr|A0A0E0HN86|A0A0E0HN86_ORYNI Uncharacterized protein OS=Oryza nivara PE=4 SV=1;
>tr|A0A0E0A7A5|A0A0E0A7A5_9ORYZ Uncharacterized protein OS=Oryza glumipatula PE=4 SV=1;
>tr|A0A061E7L2|A0A061E7L2_THECC DNA polymerase lambda isoform 1 OS=Theobroma cacao
GN=TCM_010205 PE=4 SV=1;
>tr|K7V573|K7V573_MAIZE DNA polymerase lambda (POLL) OS=Zea mays
GN=ZEAMMB73_Zm00001d044780 PE=4 SV=1;
>tr|Q0U373|Q0U373_PHANO Uncharacterized protein OS=Phaeosphaeria nodorum (strain SN15 / ATCC
MYA-4574 / FGSC 10173) GN=SNOG_13791 PE=4 SV=2;
>tr|A0A074XD59|A0A074XD59_9PEZI Nucleotidyltransferase OS=Aureobasidium namibiae CBS
147.97 GN=M436DRAFT_48818 PE=4 SV=1;
>tr|A0A178W586|A0A178W586_ARATH Pol(lambda) OS=Arabidopsis thaliana
GN=AXX17_At1g10600 PE=4 SV=1;
>sp|Q9FNY4|DPOLL_ARATH DNA polymerase lambda OS=Arabidopsis thaliana GN=POLL PE=1
SV=1;
>tr|G7DWL3|G7DWL3_MIXOS Uncharacterized protein OS=Mixia osmundae (strain CBS 9802 / IAM
14324 / JCM 22182 / KY 12970) GN=Mo01628 PE=4 SV=1;
>tr|A0A1S4A2R6|A0A1S4A2R6_TOBAC DNA polymerase beta-like isoform X2 OS=Nicotiana tabacum
GN=LOC107793193 PE=4 SV=1;
>tr|A0A094B2A4|A0A094B2A4_9PEZI Uncharacterized protein OS=Pseudogymnoascus sp. VKM F-
4513 (FW-928) GN=V494_05533 PE=4 SV=1;
>tr|D3AVG3|D3AVG3_POLPA Uncharacterized protein OS=Polysphondylium pallidum GN=PPL_00073
PE=4 SV=1;
>tr|K7DF77|K7DF77_PANTR Polymerase (DNA directed), lambda OS=Pan troglodytes GN=POLL PE=2
SV=1;
>tr|A0A0J9URH9|A0A0J9URH9_FUSO4 DNA polymerase IV OS=Fusarium oxysporum f. sp.
lycopersici (strain 4287 / CBS 123668 / FGSC 9935 / NRRL 34936) GN=FOXG_04238 PE=4 SV=1;
>tr|W7M835|W7M835_GIBM7 DNA polymerase IV OS=Gibberella moniliformis (strain M3125 / FGSC
7600) GN=FVEG_07358 PE=4 SV=1;
>tr|X0AK76|X0AK76_FUSOX DNA polymerase IV OS=Fusarium oxysporum f. sp. melonis 26406
GN=FOMG_02943 PE=4 SV=1;
>tr|A0A1S4CCF8|A0A1S4CCF8_TOBAC DNA polymerase beta-like isoform X2 OS=Nicotiana tabacum
GN=LOC107817483 PE=4 SV=1;
>tr|A0A1S4A326|A0A1S4A326_TOBAC DNA polymerase beta-like isoform X1 OS=Nicotiana tabacum
GN=LOC107793193 PE=4 SV=1;
>tr|A0A0D2XJX2|A0A0D2XJX2_FUSO4 Uncharacterized protein OS=Fusarium oxysporum f. sp.
lycopersici (strain 4287 / CBS 123668 / FGSC 9935 / NRRL 34936) PE=4 SV=1;
>tr|K4BWD9|K4BWD9_SOLLC Uncharacterized protein OS=Solanum lycopersicum PE=4 SV=1;
>tr|B7QK41|B7QK41_IXOSC DNA polymerase lambda, putative OS=Ixodes scapularis
GN=IscW_ISCW023259 PE=4 SV=1;
>tr|A0A0C3FSL2|A0A0C3FSL2_9HOMO Uncharacterized protein OS=Piloderma croceum F 1598
GN=PILCRDRAFT_820500 PE=4 SV=1;
>tr|A0A1D6NRF4|A0A1D6NRF4_MAIZE DNA polymerase lambda (POLL) OS=Zea mays
GN=ZEAMMB73_Zm00001d044780 PE=4 SV=1;
>tr|A0A061E6T3|A0A061E6T3_THECC DNA polymerase lambda isoform 2 OS=Theobroma cacao
GN=TCM_010205 PE=4 SV=1;
>tr|A0A1A8SE80|A0A1A8SE80_9TELE Polymerase (DNA directed), mu (Fragment)
OS=Nothobranchius rachovii GN=POLM PE=4 SV=1;
>tr|A0A103YLI5|A0A103YLI5_CYNCS BRCT domain-containing protein OS=Cynara cardunculus var.
scolymus GN=Ccrd_010263 PE=4 SV=1;
>tr|A0A1L7TEU6|A0A1L7TEU6_9HYPO Related to DNA polymerase Tdt-N OS=Fusarium mangiferae
GN=FMAN_11389 PE=4 SV=1;
>tr|A0A166E9I7|A0A166E9I7_DAUCA Uncharacterized protein OS=Daucus carota subsp. sativus
GN=DCAR_007103 PE=4 SV=1;
>tr|A0A1D6NRF0|A0A1D6NRF0_MAIZE DNA polymerase lambda (POLL) OS=Zea mays
GN=ZEAMMB73_Zm00001d044780 PE=4 SV=1;
>tr|A0A0C3HYP6|A0A0C3HYP6_9PEZI Uncharacterized protein OS=Oidiodendron maius Zn
GN=OIDMADRAFT_107688 PE=4 SV=1;
>tr|D7KKS2|D7KKS2_ARALL DNA polymerase lambda OS=Arabidopsis lyrata subsp. lyrata
GN=ARALYDRAFT_312091 PE=4 SV=1;
>tr|A0A1Q8S7P7|A0A1Q8S7P7_9PEZI DNA polymerase type-X family protein pol4 OS=Colletotrichum
chlorophyti GN=CCHL11_01226 PE=4 SV=1;
>tr|F7W0K8|F7W0K8_SORMK WGS project CABT00000000 data, contig 2.17 OS=Sordaria macrospora
(strain ATCC MYA-333 / DSM 997 / K(L3346) / K-hell) GN=SMAC_04011 PE=4 SV=1;
>tr|A0A0P5CMZ8|A0A0P5CMZ8_9CRUS Putative DNA-directed DNA/RNA polymerase mu (Fragment)
OS=Daphnia magna PE=4 SV=1;
>tr|A0A165G478|A0A165G478_9BASI Nucleotidyltransferase OS=Calocera cornea HHB12733
GN=CALCODRAFT_433999 PE=4 SV=1;
>tr|I1JCZ2|I1JCZ2_SOYBN Uncharacterized protein OS=Glycine max GN=LOC100820492 PE=4 SV=2;
>tr|D7TNM4|D7TNM4_VITVI Putative uncharacterized protein OS=Vitis vinifera
GN=VIT_01s0026g00650 PE=4 SV=1;
>tr|A0A1L7TZF3|A0A1L7TZF3_GIBIN Related to DNA polymerase Tdt-N OS=Gibberella intermedia
GN=FPRN_08252 PE=4 SV=1;
>tr|A0A0V1KG85|A0A0V1KG85_TRIPS DNA polymerase lambda OS=Trichinella pseudospiralis
GN=MrpL30 PE=4 SV=1;
>tr|A0A166QBQ4|A0A166QBQ4_9PEZI DNA polymerase beta OS=Colletotrichum tofieldiae
GN=CT0861_04203 PE=4 SV=1;
>tr|A0A1B8GUD2|A0A1B8GUD2_9PEZI Uncharacterized protein OS=Pseudogymnoascus verrucosus
GN=VE01_02963 PE=4 SV=1;
>tr|A8K860|A8K860_HUMAN cDNA FLJ77175, highly similar to Homo sapiens DNA polymerase
lamda2 mRNA OS=Homo sapiens PE=2 SV=1;
>tr|B4DE17|B4DE17_HUMAN cDNA FLJ53301, highly similar to DNA polymerase lambda (EC 2.7.7.7)
OS=Homo sapiens PE=2 SV=1;
>tr|B3KXT3|B3KXT3_HUMAN cDNA FLJ46002 fis, clone SMINT2011509, highly similar to DNA
polymerase lambda (EC 2.7.7.7) OS=Homo sapiens PE=2 SV=1;
>tr|A0A1L7W175|A0A1L7W175_GIBIN Related to DNA polymerase Tdt-N OS=Fusarium proliferatum
ET1 GN=FPRO_11862 PE=4 SV=1;
>tr|A0A0D7BNV9|A0A0D7BNV9_9HOMO Nucleotidyltransferase OS=Cylindrobasidium torrendii
FP15055 ss-10 GN=CYLTODRAFT_368502 PE=4 SV=1;
>tr|A0A0V1EYG3|A0A0V1EYG3_TRIPS DNA polymerase lambda OS=Trichinella pseudospiralis
GN=POLL PE=4 SV=1;
>tr|B2WHB0|B2WHB0_PYRTR DNA polymerase beta OS=Pyrenophora tritici-repentis (strain Pt-1C-
BFP) GN=PTRG_09369 PE=4 SV=1;
>tr|W6YYW4|W6YYW4_COCMI Uncharacterized protein OS=Bipolaris oryzae ATCC 44560
GN=COCMIDRAFT_41005 PE=4 SV=1;
>tr|F7CWH7|F7CWH7_MONDO Uncharacterized protein OS=Monodelphis domestica GN=POLL PE=4
SV=2;
>tr|W2ZH69|W2ZH69_PHYPR Uncharacterized protein OS=Phytophthora parasitica P10297
GN=F442_07139 PE=4 SV=1;
>tr|A0A0W8CA00|A0A0W8CA00_PHYNI DNA polymerase lambda OS=Phytophthora nicotianae
GN=AM587_10005054 PE=4 SV=1;
>tr|W2X7E5|W2X7E5_PHYPR Uncharacterized protein OS=Phytophthora parasitica CJ01A1
GN=F441_07085 PE=4 SV=1;
>tr|A0A081AFN9|A0A081AFN9_PHYPR Uncharacterized protein OS=Phytophthora parasitica P1976
GN=F444_07141 PE=4 SV=1;
>tr|W2QFB7|W2QFB7_PHYPN Uncharacterized protein OS=Phytophthora parasitica (strain INRA-310)
GN=PPTG_10157 PE=4 SV=1;
>tr|V9FC02|V9FC02_PHYPR Uncharacterized protein OS=Phytophthora parasitica P1569
GN=F443_07072 PE=4 SV=1;
>tr/W2LF93|W2LF93_PHYPR Uncharacterized protein OS=Phytophthora parasitica GN=L914_06888
PE=4 SV=1;
>tr|S0EEQ9|S0EEQ9_GIBF5 Related to DNA polymerase Tdt-N OS=Gibberella fujikuroi (strain CBS
195.34 / IMI 58289 / NRRL A-6831) GN=FFUJ_12212 PE=4 SV=1;
>tr|K3VLK2|K3VLK2_FUSPC Uncharacterized protein OS=Fusarium pseudograminearum (strain
CS3096) GN=FPSE_05383 PE=4 SV=1;
>tr|I1RE23|I1RE23_GIBZE Uncharacterized protein OS=Gibberella zeae (strain PH-1 / ATCC MYA-
4620 / FGSC 9075 / NRRL 31084) GN=FG01896.1 PE=4 SV=1;
>tr|A0A165FWW0|A0A165FWW0_9PEZI Terminal deoxynucleotidyl transferas-like protein OS=Xylona
heveae TC161 GN=L228DRAFT_248190 PE=4 SV=1;
>tr|A0A178AH86|A0A178AH86_9PLEO Nucleotidyltransferase OS=Stagonospora sp. SRC1lsM3a
GN=IQ06DRAFT_379775 PE=4 SV=1;
>tr|Q5QJV5|Q5QJV5_HUMAN DNA polymerase lamda2 OS=Homo sapiens PE=2 SV=1;
>tr|A0A0I9Y516|A0A0I9Y516_GIBFU DNA polymerase Tdt-N OS=Gibberella fujikuroi
GN=LW93_5943 PE=4 SV=1;
>tr|A0A0K9QPB2|A0A0K9QPB2_SPIOL Uncharacterized protein OS=Spinacia oleracea
GN=SOVF_161430 PE=4 SV=1;
>tr|A0A1B6DMG4|A0A1B6DMG4_9HEMI Uncharacterized protein OS=Clastoptera arizonana
GN=g.16118 PE=4 SV=1;
>tr|A0A0G2TCQ4|A0A0G2TCQ4_SINCH DNA-directed DNA/RNA polymerase mu-like protein
(Fragment) OS=Siniperca chuatsi PE=2 SV=1;
>tr|S3CES3|S3CES3_GLAL2 Nucleotidyltransferase OS=Glarea lozoyensis (strain ATCC 20868 /
MF5171) GN=GLAREA_11578 PE=4 SV=1;
>tr|G1X3Y6|G1X3Y6_ARTOA Uncharacterized protein OS=Arthrobotrys oligospora (strain ATCC 24927
/ CBS 115.81 / DSM 1491) GN=AOL_s00043g410 PE=4 SV=1;
>tr|A0A1B8B5I4|A0A1B8B5I4_FUSPO Uncharacterized protein OS=Fusarium poae GN=FPOA_01921
PE=4 SV=1;
>tr|K3WBD7|K3WBD7_PYTUL Uncharacterized protein OS=Pythium ultimum DAOM BR144 PE=4
SV=1;
>tr|A0A0P1AR42|A0A0P1AR42_9STRA Dna polymerase lambda-like protein OS=Plasmopara halstedii
PE=4 SV=1;
>tr|I1G678|I1G678_AMPQE Uncharacterized protein OS=Amphimedon queenslandica
GN=LOC100640740 PE=4 SV=1;
>tr|A0A0V1I9V0|A0A0V1I9V0_9BILA DNA polymerase lambda OS=Trichinella zimbabwensis
GN=MRPL30 PE=4 SV=1;
>tr|A0A166IY76|A0A166IY76_9HOMO Nucleotidyltransferase OS=Peniophora sp. CONT
GN=PENSPDRAFT_682328 PE=4 SV=1;
>tr|F9X556|F9X556_ZYMTI DNA polymerase beta-like protein OS=Zymoseptoria tritici (strain CBS
115943 / IPO323) GN=POLX2 PE=4 SV=1;
>tr|A0A1J7J8L3|A0A1J7J8L3_9PEZI Nucleotidyltransferase OS=Coniochaeta ligniaria NRRL 30616
GN=CONLIGDRAFT_643718 PE=4 SV=1;
>tr|M3B059|M3B059_SPHMS Nucleotidyltransferase OS=Sphaerulina musiva (strain SO2202)
GN=SEPMUDRAFT_148552 PE=4 SV=1;
>tr|T0RL69|T0RL69_9STRA Uncharacterized protein OS=Saprolegnia diclina VS20 GN=SDRG_09530
PE=4 SV=1;
>tr|R0GTX0|R0GTX0_9BRAS Uncharacterized protein (Fragment) OS=Capsella rubella
GN=CARUB_v10012462mg PE=4 SV=1;
>tr|A0A163C683|A0A163C683_DIDRA DNA binding OS=Didymella rabiei GN=ST47_g6594 PE=4
SV=1;
>tr|A0A1B6IPI3|A0A1B6IPI3_9HEMI Uncharacterized protein (Fragment) OS=Homalodisca liturata
GN=g.26729 PE=4 SV=1;
>tr|G2QRI9|G2QRI9_THITE Uncharacterized protein OS=Thielavia terrestris (strain ATCC 38088 /
NRRL 8126) GN=THITE_2106802 PE=4 SV=1;
>tr|A0A0L0HB69|A0A0L0HB69_SPIPN Uncharacterized protein OS=Spizellomyces punctatus DAOM
BR117 GN=SPPG_06523 PE=4 SV=1;
>tr|A0CMJ3|A0CMJ3_PARTE Uncharacterized protein OS=Paramecium tetraurelia
GN=GSPATT00008489001 PE=4 SV=1;
>tr|L7IL19|L7IL19_MAGOY DNA polymerase beta OS=Magnaporthe oryzae (strain Y34)
GN=OOU_Y34scaffold00140g29 PE=4 SV=1;
>tr|G4MN08|G4MN08_MAGO7 DNA polymerase beta OS=Magnaporthe oryzae (strain 70-15 / ATCC
MYA-4617 / FGSC 8958) GN=MGG_06908 PE=4 SV=1;
>tr|L7JI54|L7JI54_MAGOP DNA polymerase beta OS=Magnaporthe oryzae (strain P131)
GN=OOW_P131scaffold00328g30 PE=4 SV=1;
>tr|V4SV18|V4SV18_9ROSI Uncharacterized protein OS=Citrus clementina GN=CICLE_v10031184mg
PE=4 SV=1;
>tr|L7I3D8|L7I3D8_MAGOY DNA polymerase lambda OS=Magnaporthe oryzae (strain Y34)
GN=OOU_Y34scaffold00619g46 PE=4 SV=1;
>tr|G4MSU5|G4MSU5_MAGO7 Uncharacterized protein OS=Magnaporthe oryzae (strain 70-15 / ATCC
MYA-4617 / FGSC 8958) GN=MGG_04577 PE=4 SV=1;
>tr|L7IU60|L7IU60_MAGOP DNA polymerase lambda OS=Magnaporthe oryzae (strain P131)
GN=OOW_P131scaffold01358g84 PE=4 SV=1;
>tr|A0A178DYJ1|A0A178DYJ1_9PLEO Nucleotidyltransferase OS=Pyrenochaeta sp. DS3sAY3a
GN=IQ07DRAFT_622852 PE=4 SV=1;
>tr|A0A061EDJ3|A0A061EDJ3_THECC DNA polymerase lambda (POLL) isoform 3 OS=Theobroma
cacao GN=TCM_010205 PE=4 SV=1;
>tr|A0A0A1V1A1|A0A0A1V1A1_9HYPO DNA polymerase X family protein OS=Metarhizium robertsii
GN=X797_003415 PE=4 SV=1;
>tr|A0A161W8A7|A0A161W8A7_9PEZI Dna polymerase beta protein OS=Colletotrichum incanum
GN=CI238_02506 PE=4 SV=1;
>tr|A0A197K957|A0A197K957_9FUNG Nucleotidyltransferase OS=Mortierella elongata AG-77
GN=K457DRAFT_69808 PE=4 SV=1;
>tr|A0A1I8HZS6|A0A1I8HZS6_9PLAT Uncharacterized protein OS=Macrostomum lignano PE=4 SV=1;
>tr|A0A0D2R796|A0A0D2R796_GOSRA Uncharacterized protein OS=Gossypium raimondii
GN=B456_002G164200 PE=4 SV=1;
>tr|A0A1I8I2E2|A0A1I8I2E2_9PLAT Uncharacterized protein OS=Macrostomum lignano PE=4 SV=1;
>tr|A0A0G2FFQ8|A0A0G2FFQ8_9PEZI Putative dna polymerase beta OS=Diaporthe ampelina
GN=UCDDA912_g07030 PE=4 SV=1;
>tr|A0A074W8N4|A0A074W8N4_9PEZI DNA polymerase beta-like protein OS=Aureobasidium
melanogenum CBS 110374 GN=M437DRAFT_39375 PE=4 SV=1;
>tr|A0A1B6LW89|A0A1B6LW89_9HEMI Uncharacterized protein OS=Graphocephala atropunctata
GN=g.50861 PE=4 SV=1;
>tr|A0A1Q3ES10|A0A1Q3ES10_LENED Dna polymerase lambda OS=Lentinula edodes
GN=LENED_012117 PE=4 SV=1;
>tr|F8WDE4|F8WDE4_HUMAN DNA-directed DNA/RNA polymerase mu OS=Homo sapiens
GN=POLM PE=1 SV=1;
>tr|E2AH73|E2AH73_CAMFO DNA polymerase beta (Fragment) OS=Camponotus floridanus
GN=EAG_09061 PE=4 SV=1;
>tr|C9JF34|C9JF34_HUMAN DNA-directed DNA/RNA polymerase mu (Fragment) OS=Homo sapiens
GN=POLM PE=1 SV=1;
>tr|M2PG81|M2PG81_CERS8 Uncharacterized protein OS=Ceriporiopsis subvermispora (strain B)
GN=CERSUDRAFT_157745 PE=4 SV=1;
>tr|R7YS72|R7YS72_CONA1 Uncharacterized protein OS=Coniosporium apollinis (strain CBS 100218)
GN=W97_03739 PE=4 SV=1;
>tr|V7C0P8|V7C0P8_PHAVU Uncharacterized protein (Fragment) OS=Phaseolus vulgaris
GN=PHAVU_004G0710000g PE=4 SV=1;
>tr|A0A0W0FEC6|A0A0W0FEC6_9AGAR Uncharacterized protein OS=Moniliophthora roreri
GN=WG66_12791 PE=4 SV=1;
>tr|K1PLM1|K1PLM1_CRAGI DNA polymerase lambda OS=Crassostrea gigas GN=CGI_10001943
PE=4 SV=1;
>tr|A0A1D2VPH7|A0A1D2VPH7_9ASCO Nucleotidyltransferase OS=Ascoidea rubescens DSM 1968
GN=ASCRUDRAFT_67555 PE=4 SV=1;
>tr|A0A067C9L0|A0A067C9L0_SAPPC Uncharacterized protein OS=Saprolegnia parasitica (strain CBS
223.65) GN=SPRG_07053 PE=4 SV=1;
>tr|V2YCS0|V2YCS0_MONRO Dna polymerase lambda OS=Moniliophthora roreri (strain MCA 2997)
GN=Moror_16092 PE=4 SV=1;
>tr|A0A1J7GWS4|A0A1J7GWS4_LUPAN Uncharacterized protein OS=Lupinus angustifolius
GN=TanjilG_25590 PE=4 SV=1;
>tr|A0A0J7KSX3|A0A0J7KSX3_LASNI Metallophosphoesterase 1 OS=Lasius niger GN=RF55_6460
PE=4 SV=1;
>tr|A0A0W7VNN6|A0A0W7VNN6_9HYPO High-affinity nickel transporter OS=Trichoderma gamsii
GN=TGAM01_05509 PE=4 SV=1;
>tr|A0A0A1TBX7|A0A0A1TBX7_9HYPO Uncharacterized protein OS=Torrubiella hemipterigena
GN=VHEMI10101 PE=4 SV=1;
>tr|A0A0N8H665|A0A0N8H665_9HYPO Uncharacterized protein OS=Neonectria ditissima
GN=AK830_g8452 PE=4 SV=1;
>tr|A0A0V1MQQ6|A0A0V1MQQ6_9BILA DNA polymerase lambda OS=Trichinella papuae
GN=MRPL30 PE=4 SV=1;
>tr|G2X3I6|G2X3I6_VERDV DNA polymerase lambda OS=Verticillium dahliae (strain VdLs.17 / ATCC
MYA-4575 / FGSC 10137) GN=VDAG_04573 PE=4 SV=1;
>tr|A0A093Y1B2|A0A093Y1B2_9PEZI Uncharacterized protein (Fragment) OS=Pseudogymnoascus sp.
VKM F-3808 GN=0988_04227 PE=4 SV=1;
>tr|A0A1C1X2L6|A0A1C1X2L6_9PEZI High-affinity nickel transporter (Fragment) OS=Diaporthe
helianthi GN=DHEL01_08081 PE=4 SV=1;
>tr|A0A165D1N9|A0A165D1N9_9APHY Uncharacterized protein OS=Laetiporus sulphureus 93-53
GN=LAESUDRAFT_814364 PE=4 SV=1;
>tr|A0A139ABS3|A0A139ABS3_GONPR Nucleotidyltransferase OS=Gonapodya prolifera JEL478
GN=M427DRAFT_112983 PE=4 SV=1;
>tr|A0A139I956|A0A139I956_9PEZI Uncharacterized protein OS=Pseudocercospora musae
GN=AC579_8383 PE=4 SV=1;
>tr|A0A1S4A2W8|A0A1S4A2W8_TOBAC DNA polymerase beta-like isoform X3 OS=Nicotiana
tabacum GN=LOC107793193 PE=4 SV=1;
>tr|A0A0F4ZAE6|A0A0F4ZAE6_9PEZI Uncharacterized protein OS=Thielaviopsis punctulata
GN=TD95_003167 PE=4 SV=1;
>tr|G3HHH7|G3HHH7_CRIGR DNA polymerase beta OS=Cricetulus griseus GN=I79_010094 PE=4
SV=1;
>tr|A0A0B1PLB7|A0A0B1PLB7_9BILA Uncharacterized protein OS=Trichuris suis GN=D918_06892
PE=4 SV=1;
>tr|A0A026WYD7|A0A026WYD7_CERBI DNA polymerase beta OS=Cerapachys biroi
GN=X777_15051 PE=4 SV=1;
>tr|A0A0P5HWD7|A0A0P5HWD7_9CRUS Putative DNA-directed DNA/RNA polymerase mu
(Fragment) OS=Daphnia magna PE=4 SV=1;
>tr|A0A085M6E7|A0A085M6E7_9BILA Uncharacterized protein (Fragment) OS=Trichuris suis
GN=M513_06284 PE=4 SV=1;
>tr|A0A085NR57|A0A085NR57_9BILA Uncharacterized protein (Fragment) OS=Trichuris suis
GN=M514_06284 PE=4 SV=1;
>tr|A0A1D1UJX5|A0A1D1UJX5_RAMVA Uncharacterized protein OS=Ramazzottius varieornatus
GN=RvY_02488-1 PE=4 SV=1;
>tr|A0A015KMQ8|A0A015KMQ8_9GLOM Pol4p OS=Rhizophagus irregularis DAOM 197198w
GN=RirG_174710 PE=4 SV=1;
>tr|U9UPA5|U9UPA5_RHIID Uncharacterized protein OS=Rhizophagus irregularis (strain DAOM
181602 / DAOM 197198 / MUCL 43194) GN=GLOINDRAFT_321289 PE=4 SV=1;
>tr|H2XQC3|H2XQC3_CIOIN Uncharacterized protein OS=Ciona intestinalis PE=4 SV=1;
>tr|A0A077ZAV7|A0A077ZAV7_TRITR DNA polymerase lambda OS=Trichuris trichiura
GN=TTRE_0000524701 PE=4 SV=1;
>tr|W3XQL7|W3XQL7_9PEZI Uncharacterized protein OS=Pestalotiopsis fici W106-1 GN=PFICI_01384
PE=4 SV=1;
>tr|A0A0M9EXF4|A0A0M9EXF4_9HYPO Dna polymerase iv OS=Fusarium langsethiae
GN=FLAG1_05496 PE=4 SV=1;
>tr|A0A0C3L267|A0A0C3L267_9HOMO Uncharacterized protein (Fragment) OS=Tulasnella calospora
MUT 4182 GN=M407DRAFT_72655 PE=4 SV=1;
>tr|A0A067H4G2|A0A067H4G2_CITSI Uncharacterized protein (Fragment) OS=Citrus sinensis
GN=CISIN_1g0093031mg PE=4 SV=1;
>tr|G3SQY2|G3SQY2_LOXAF Uncharacterized protein OS=Loxodonta africana GN=POLB PE=4 SV=1;
>tr|A0A067GSH6|A0A067GSH6_CITSI Uncharacterized protein (Fragment) OS=Citrus sinensis
GN=CISIN_1g0093031mg PE=4 SV=1;
>tr|U4LGA6|U4LGA6_PYROM Similar to DNA polymerase lambda acc. no. Q4R380 OS=Pyronema
omphalodes (strain CBS 100304) GN=PCON_10034 PE=4 SV=1;
>tr|A0A0C3S642|A0A0C3S642_PHLGI Uncharacterized protein OS=Phlebiopsis gigantea 11061_1 CR5-
6 GN=PHLGIDRAFT_480151 PE=4 SV=1;
>tr|E9EUG6|E9EUG6_METRA Nucleotidyltransferase OS=Metarhizium robertsii (strain ARSEF 23 /
ATCC MYA-3075) GN=MAA_03665 PE=4 SV=2;
>tr|C9J222|C9J222_HUMAN DNA-directed DNA/RNA polymerase mu (Fragment) OS=Homo sapiens
GN=POLM PE=1 SV=1;
>tr|C7YGY5|C7YGY5_NECH7 Putative uncharacterized protein OS=Nectria haematococca (strain 77-13-
4 / ATCC MYA-4622 / FGSC 9596 / MPVI) GN=NECHADRAFT_74784 PE=4 SV=1;
>tr|H0ZI77|H0ZI77_TAEGU Uncharacterized protein OS=Taeniopygia guttata GN=POLL PE=4 SV=1;
>tr|L9L936|L9L936_TUPCH DNA polymerase beta OS=Tupaia chinensis GN=TREES_T100019179
PE=4 SV=1;
>tr|A0A1R2BTP8|A0A1R2BTP8_9CILI Uncharacterized protein OS=Stentor coeruleus
GN=SteCoe_19671 PE=4 SV=1;
>tr|S4RCE7|S4RCE7_PETMA Uncharacterized protein OS=Petromyzon marinus PE=4 SV=1;
>tr|I1RAT5|I1RAT5_GIBZE Uncharacterized protein OS=Gibberella zeae (strain PH-1 / ATCC MYA-
4620 / FGSC 9075 / NRRL 31084) GN=FG00621.1 PE=4 SV=1;
>tr|A0A136JFC5|A0A136JFC5_9PEZI Uncharacterized protein OS=Microdochium bolleyi
GN=Micbo1qcDRAFT_201184 PE=4 SV=1;
>tr|A0A0B4IIB3|A0A0B4IIB3_9HYPO DNA-directed DNA polymerase X (Fragment) OS=Metarhizium
majus ARSEF 297 GN=MAJ_03673 PE=4 SV=1;
>tr|A0A0B4H1V8|A0A0B4H1V8_9HYPO DNA-directed DNA polymerase X OS=Metarhizium
guizhouense ARSEF 977 GN=MGU_03773 PE=4 SV=1;
>tr|A0A139HWX0|A0A139HWX0_9PEZI Uncharacterized protein OS=Mycosphaerella eumusae
GN=AC578_7095 PE=4 SV=1;
>tr|A0A0D2A6C0|A0A0D2A6C0_9PEZI Uncharacterized protein OS=Verruconis gallopava
GN=PV09_06595 PE=4 SV=1;
>tr|F1PKP7|F1PKP7_CANLF Uncharacterized protein OS=Canis lupus familiaris GN=POLB PE=4
SV=1;
>sp|P06766|DPOLB_RAT DNA polymerase beta OS=Rattus norvegicus GN=Polb PE=1 SV=4;
>tr|B2B4U9|B2B4U9_PODAN Podospora anserina S mat+ genomic DNA chromosome 2, supercontig 2
OS=Podospora anserina (strain S / ATCC MYA-4624 / DSM 980 / FGSC 10383) GN=PODANS_2_2540
PE=4 SV=1;
>tr|A0A0G0A1N0|A0A0G0A1N0_TRIHA Uncharacterized protein OS=Trichoderma harzianum
GN=THAR02_01643 PE=4 SV=1;
>tr|E0VPG0|E0VPG0_PEDHC DNA polymerase beta, putative OS=Pediculus humanus subsp. corporis
GN=8232106 PE=4 SV=1;
>tr|T1J7P5|T1J7P5_STRMM Uncharacterized protein OS=Strigamia maritima PE=4 SV=1;
>tr|L8I911|L8I911_9CETA DNA polymerase beta OS=Bos mutus GN=M91_05776 PE=4 SV=1;
>tr|G1SF51|G1SF51_RABIT Uncharacterized protein OS=Oryctolagus cuniculus GN=POLB PE=4 SV=1;
>tr|H0XES4|H0XES4_OTOGA Uncharacterized protein OS=Otolemur garnettii GN=POLB PE=4 SV=1;
>sp|Q8K409|DPOLB_MOUSE DNA polymerase beta OS=Mus musculus GN=Polb PE=1 SV=3;
>sp|Q27958|DPOLB_BOVIN DNA polymerase beta OS=Bos taurus GN=POLB PE=2 SV=3;
>tr|A0PC13|A0PC13_COPCI DNA polymerase lambda OS=Coprinopsis cinerea GN=pollambda PE=4
SV=1;
>tr|A0A0D9NN19|A0A0D9NN19_METAN Uncharacterized protein OS=Metarhizium anisopliae BRIP
53293 GN=H634G_08840 PE=4 SV=1;
>tr|G9NH38|G9NH38_HYPAI Uncharacterized protein OS=Hypocrea atroviridis (strain ATCC 20476 /
IMI 206040) GN=TRIATDRAFT_51201 PE=4 SV=1;
>tr|G9NDQ2|G9NDQ2_HYPVG Uncharacterized protein OS=Hypocrea virens (strain Gv29-8 / FGSC
10586) GN=TRIVIDRAFT_51446 PE=4 SV=1;
>tr|G9KHM9|G9KHM9_MUSPF Polymerase, beta (Fragment) OS=Mustela putorius furo PE=2 SV=1;
>tr|U6CV23|U6CV23_NEOVI DNA polymerase beta OS=Neovison vison GN=DPOLB PE=2 SV=1;
>tr|M3YMQ7|M3YMQ7_MUSPF Uncharacterized protein OS=Mustela putorius furo GN=POLB PE=4
SV=1;
>tr|Q9HAJ3|Q9HAJ3_HUMAN cDNA FLJ11538 fis, clone HEMBA1002746, weakly similar to DNA
POLYMERASE BETA (EC 2.7.7.7) OS=Homo sapiens PE=2 SV=1;
>tr|A0A077ZZ96|A0A077ZZ96_STYLE Helix-hairpin-helix motif family protein OS=Stylonychia lemnae
GN=Contig18226.g19364 PE=4 SV=1;
>tr|K9IID0|K9IID0_DESRO Putative dna polymerase iv family x OS=Desmodus rotundus PE=2 SV=1;
>tr|A0A1A8U639|A0A1A8U639_NOTFU Deoxynucleotidyltransferase, terminal (Fragment)
OS=Nothobranchius furzeri GN=DNTT PE=4 SV=1;
>tr|A0A026WUA4|A0A026WUA4_CERBI DNA polymerase beta OS=Cerapachys biroi
GN=X777_16780 PE=4 SV=1;
>tr|A0A178AM56|A0A178AM56_9PLEO Uncharacterized protein OS=Stagonospora sp. SRC1lsM3a
GN=IQ06DRAFT_278382 PE=4 SV=1;
>tr|L5KAJ8|L5KAJ8_PTEAL DNA polymerase beta OS=Pteropus alecto GN=PAL_GLEAN10021536
PE=4 SV=1;
>tr|G1Q072|G1Q072_MYOLU Uncharacterized protein OS=Myotis lucifugus GN=POLB PE=4 SV=1;
>tr|G1LEW7|G1LEW7_AILME Uncharacterized protein OS=Ailuropoda melanoleuca GN=POLB PE=4
SV=1;
>tr|A0A1B6JS91|A0A1B6JS91_9HEMI Uncharacterized protein OS=Homalodisca liturata GN=g.26727
PE=4 SV=1;
>tr|R4XCI6|R4XCI6_TAPDE Putative DNA polymerase POL4 OS=Taphrina deformans (strain PYCC
5710 / ATCC 11124 / CBS 356.35 / IMI 108563 / JCM 9778 / NBRC 8474) GN=TAPDE_003785 PE=4
SV=1;
>tr|A0A0C2X4X0|A0A0C2X4X0_AMAMU Uncharacterized protein OS=Amanita muscaria Koide
BX008 GN=M378DRAFT_163339 PE=4 SV=1;
>tr|G2YW92|G2YW92_BOTF4 Similar to terminal deoxynucleotidyl transferase OS=Botryotinia
fuckeliana (strain T4) GN=BofuT4_P150220.1 PE=4 SV=1;
>tr|M7USU8|M7USU8_BOTF1 Putative dna polymerase beta protein OS=Botryotinia fuckeliana (strain
BcDW1) GN=BcDW1_1262 PE=4 SV=1;
>tr|G0SDS2|G0SDS2_CHATD DNA polymerase-like protein OS=Chaetomium thermophilum (strain
DSM 1495 / CBS 144.50 / IMI 039719) GN=CTHT_0052790 PE=4 SV=1;
>tr|G9NIX2|G9NIX2_HYPAI Uncharacterized protein (Fragment) OS=Hypocrea atroviridis (strain ATCC
20476 / IMI 206040) GN=TRIATDRAFT_174080 PE=4 SV=1;
>tr|A0A023GLT6|A0A023GLT6_9ACAR Putative dna polymerase iv family x (Fragment)
OS=Amblyomma triste PE=2 SV=1;
>tr|T1DLI8|T1DLI8_CROHD DNA polymerase beta-like protein OS=Crotalus horridus PE=2 SV=1;
>tr|Q5SBJ1|Q5SBJ1_CANLF DNA polymerase beta (Fragment) OS=Canis lupus familiaris PE=2 SV=1;
>tr|H2ZGS9|H2ZGS9_CIOSA Uncharacterized protein OS=Ciona savignyi PE=4 SV=1;
>tr|V7C0T6|V7C0T6_PHAVU Uncharacterized protein (Fragment) OS=Phaseolus vulgaris
GN=PHAVU_004G0710000g PE=4 SV=1;
>tr|A0A0N8AG30|A0A0N8AG30_9CRUS DNA-directed DNA/RNA polymerase mu (Fragment)
OS=Daphnia magna PE=4 SV=1;
>tr|A0A0P4W7N0|A0A0P4W7N0_9EUCA Uncharacterized protein OS=Scylla olivacea PE=4 SV=1;
>tr|M3XC91|M3XC91_FELCA Uncharacterized protein OS=Felis catus GN=POLB PE=4 SV=1;
>tr|F2UQK2|F2UQK2_SALR5 Putative uncharacterized protein OS=Salpingoeca rosetta (strain ATCC
50818 / BSB-021) GN=PTSG_10190 PE=4 SV=1;
>tr|A0A1Q5UEI8|A0A1Q5UEI8_9EURO DNA polymerase type-X family protein pol4 OS=Penicillium
subrubescens GN=PENSUB_3710 PE=4 SV=1;
>tr|E9IUZ2|E9IUZ2_SOLIN Putative uncharacterized protein (Fragment) OS=Solenopsis invicta
GN=SINV_02512 PE=4 SV=1;
>tr|N1Q5N8|N1Q5N8_PSEFD Uncharacterized protein (Fragment) OS=Pseudocercospora fijiensis (strain
CIRAD86) GN=MYCFIDRAFT_1485 PE=4 SV=1;
>tr|A0A0S6XKL8|A0A0S6XKL8_9FUNG Uncharacterized protein OS=fungal sp. No.11243
GN=ANO11243_043170 PE=4 SV=1;
>tr|A0A0L0S8H4|A0A0L0S8H4_ALLMA Uncharacterized protein OS=Allomyces macrogynus ATCC
38327 GN=AMAG_04435 PE=4 SV=1;
>tr|A0A0M9VPF2|A0A0M9VPF2_9BASI Dna polymerase mu OS=Malassezia pachydermatis
GN=Malapachy_3911 PE=4 SV=1;
>tr|H0EG73|H0EG73_GLAL7 Putative DNA polymerase lambda OS=Glarea lozoyensis (strain ATCC
74030 / MF5533) GN=M7I_1483 PE=4 SV=1;
>tr|A0A0K9QMR5|A0A0K9QMR5_SPIOL Uncharacterized protein OS=Spinacia oleracea
GN=SOVF_161430 PE=4 SV=1;
>tr|I3KRF3|I3KRF3_ORENI Uncharacterized protein OS=Oreochromis niloticus GN=wbp11 PE=4 SV=1;
>tr|A0A178W5U8|A0A178W5U8_ARATH Pol(lambda) OS=Arabidopsis thaliana
GN=AXX17_At1g10600 PE=4 SV=1;
>tr|A0A1P8APE6|A0A1P8APE6_ARATH DNA polymerase lambda (POLL) OS=Arabidopsis thaliana
GN=Pol{lambda} PE=4 SV=1;
>tr|S3DHN1|S3DHN1_GLAL2 Nucleotidyltransferase OS=Glarea lozoyensis (strain ATCC 20868 /
MF5171) GN=GLAREA_12302 PE=4 SV=1;
>tr|F6TYB0|F6TYB0_CIOIN Uncharacterized protein OS=Ciona intestinalis PE=4 SV=2;
>tr|I3M6T3|I3M6T3_ICTTR Uncharacterized protein OS=Ictidomys tridecemlineatus GN=POLB PE=4
SV=1;
>tr|G5BT41|G5BT41_HETGA DNA polymerase beta OS=Heterocephalus glaber GN=GW7_08532 PE=2
SV=1;
>tr|W5UI78|W5UI78_ICTPU DNA polymerase beta OS=Ictalurus punctatus GN=polb PE=2 SV=1;
>tr|W6MJI8|W6MJI8_9ASCO Uncharacterized protein OS=Kuraishia capsulata CBS 1993
GN=KUCA_T00002413001 PE=4 SV=1;
>tr|A0A0A2VE68|A0A0A2VE68_BEABA Putative DNA polymerase family X C2F7.06c OS=Beauveria
bassiana D1-5 GN=BBAD15_g8907 PE=4 SV=1;
>tr|A0A0B7JPX6|A0A0B7JPX6_BIOOC Uncharacterized protein (Fragment) OS=Bionectria ochroleuca
GN=BN869_000003098_1 PE=4 SV=1;
>tr|A0A069DYM9|A0A069DYM9_9HEMI Putative dna polymerase iv family x OS=Panstrongylus
megistus PE=2 SV=1;
>tr|A0A060T9I9|A0A060T9I9_BLAAD ARAD1D15576p OS=Blastobotrys adeninivorans
GN=GNLVRS02_ARAD1D15576g PE=4 SV=1;
>tr|G1RQX7|G1RQX7_NOMLE Uncharacterized protein OS=Nomascus leucogenys GN=POLB PE=4
SV=1;
>tr|H2QW35|H2QW35_PANTR Polymerase (DNA directed), beta OS=Pan troglodytes GN=POLB PE=2
SV=1;
>sp|P06746|DPOLB_HUMAN DNA polymerase beta OS=Homo sapiens GN=POLB PE=1 SV=3;
>tr|Q6C9C2|Q6C9C2_YARLI YALI0D12364p OS=Yarrowia lipolytica (strain CLIB 122 / E 150)
GN=YALI0_D12364g PE=4 SV=1;
>tr|A0A1H6PY30|A0A1H6PY30_YARLL YALIA101S12e01794g1_1 OS=Yarrowia lipolytica
GN=YALIA101_S12E01794G PE=4 SV=1;
>tr|A0A1D8NEA0|A0A1D8NEA0_YARLL Uncharacterized protein OS=Yarrowia lipolytica
GN=YALI1_D15367g PE=4 SV=1;
>tr|A0A1B8EDN0|A0A1B8EDN0_9PEZI Uncharacterized protein OS=Pseudogymnoascus sp. 23342-1-11
GN=VE03_01116 PE=3 SV=1;
>tr|A0A094BR33|A0A094BR33_9PEZI Uncharacterized protein OS=Pseudogymnoascus sp. VKM F-
4246 GN=V492_03652 PE=3 SV=1;
>tr|K0KG88|K0KG88_WICCF DNA nucleotidylexotransferase OS=Wickerhamomyces ciferrii (strain F-
60-10 / ATCC 14091 / CBS 111 / JCM 3599 / NBRC 0793 / NRRL Y-1031) GN=BN7_3734 PE=4 SV=1;
>tr|K3VGJ0|K3VGJ0_FUSPC Uncharacterized protein OS=Fusarium pseudograminearum (strain
CS3096) GN=FPSE_06415 PE=4 SV=1;
>tr|F7IKW6|F7IKW6_CALJA DNA polymerase beta OS=Callithrix jacchus GN=POLB PE=2 SV=1;
>tr|A0A094FIQ0|A0A094FIQ0_9PEZI Uncharacterized protein OS=Pseudogymnoascus sp. VKM F-4516
(FW-969) GN=V497_04262 PE=4 SV=1;
>tr|A0A093XQA1|A0A093XQA1_9PEZI Uncharacterized protein OS=Pseudogymnoascus sp. VKM F-
3557 GN=V490_05206 PE=4 SV=1;
>tr|A0A0P7BR28|A0A0P7BR28_9HYPO Uncharacterized protein OS=Neonectria ditissima
GN=AK830_g2708 PE=4 SV=1;
>tr|A0A1D6NRF3|A0A1D6NRF3_MAIZE DNA polymerase lambda (POLL) OS=Zea mays
GN=ZEAMMB73_Zm00001d044780 PE=4 SV=1;
>tr|A0A0G4II62|A0A0G4II62_PLABS Uncharacterized protein OS=Plasmodiophora brassicae
GN=PBRA_003577 PE=4 SV=1;
>tr|A0A0S6XJT2|A0A0S6XJT2_9FUNG Uncharacterized protein OS=fungal sp. No.11243
GN=ANO11243_041640 PE=4 SV=1;
>tr|F6WYN9|F6WYN9_ORNAN Uncharacterized protein OS=Ornithorhynchus anatinus GN=POLL
PE=4 SV=2;
>tr|F6WYP8|F6WYP8_ORNAN Uncharacterized protein OS=Ornithorhynchus anatinus GN=POLL PE=4
SV=2;
>tr|A0A094G0N2|A0A094G0N2_9PEZI Uncharacterized protein OS=Pseudogymnoascus sp. VKM F-
4518 (FW-2643) GN=V500_02402 PE=3 SV=1;
>tr|A0A1B8F8Y4|A0A1B8F8Y4_9PEZI Uncharacterized protein OS=Pseudogymnoascus sp. 05NY08
GN=VF21_01182 PE=4 SV=1;
>tr|T0L037|T0L037_COLGC Uncharacterized protein OS=Colletotrichum gloeosporioides (strain Cg-14)
GN=CGLO_01555 PE=4 SV=1;
>tr|A0A061H8U8|A0A061H8U8_9BASI Uncharacterized protein OS=Anthracocystis flocculosa PF-1
GN=PFL1_03308 PE=4 SV=1;
>tr|A0A0L7QQR3|A0A0L7QQR3_9HYME DNA polymerase beta OS=Habropoda laboriosa
GN=WH47_05677 PE=4 SV=1;
>tr|S7MBS5|S7MBS5_MYOBR DNA polymerase beta (Fragment) OS=Myotis brandtii
GN=D623_10030529 PE=4 SV=1;
>tr|A0A093ZRF7|A0A093ZRF7_9PEZI Uncharacterized protein OS=Pseudogymnoascus sp. VKM F-
3775 GN=V491_06339 PE=3 SV=1;
>tr|A0A094BPD5|A0A094BPD5_9PEZI Uncharacterized protein OS=Pseudogymnoascus sp. VKM F-
4513 (FW-928) GN=V494_04866 PE=3 SV=1;
>tr|M7T317|M7T317_EUTLA Putative dna polymerase protein OS=Eutypa lata (strain UCR-EL1)
GN=UCREL1_1708 PE=4 SV=1;
>tr|A0A101M8Z9|A0A101M8Z9_9EURO Uncharacterized protein OS=Penicillium freii
GN=ACN42_g11030 PE=4 SV=1;
>tr|A0A0U5FQX5|A0A0U5FQX5_9EURO Uncharacterized protein OS=Aspergillus calidoustus
GN=ASPCAL00639 PE=4 SV=1;
>tr|H2PQ72|H2PQ72_PONAB Uncharacterized protein OS=Pongo abelii GN=POLB PE=4 SV=1;
>tr|A0A0D0EBV6|A0A0D0EBV6_9HOMO Unplaced genomic scaffold scaffold_77, whole genome
shotgun sequence OS=Paxillus rubicundulus Ve08.2h10 GN=PAXRUDRAFT_824032 PE=4 SV=1;
>tr|A0A0N0BKH1|A0A0N0BKH1_9HYME DNA polymerase beta OS=Melipona quadrifasciata
GN=WN51_05442 PE=4 SV=1;
>tr|A0A094H7H0|A0A094H7H0_9PEZI Uncharacterized protein OS=Pseudogymnoascus sp. VKM F-
4520 (FW-2644) GN=V502_08840 PE=3 SV=1;
>tr|J9HWK2|J9HWK2_9SPIT Helix-hairpin-helix motif family protein OS=Oxytricha trifallax
GN=OXYTRI_10227 PE=4 SV=1;
>tr|A0A1J8QB20|A0A1J8QB20_9HOMO Uncharacterized protein OS=Rhizopogon vesiculosus
GN=AZE42_05230 PE=4 SV=1;
>tr|A0A1S3KHL6|A0A1S3KHL6_LINUN DNA polymerase lambda-like isoform X2 OS=Lingula unguis
GN=LOC106181965 PE=4 SV=1;
>tr|A0A1L9PIQ4|A0A1L9PIQ4_ASPVE Uncharacterized protein OS=Aspergillus versicolor CBS 583.65
GN=ASPVEDRAFT_130713 PE=4 SV=1;
>tr|Q53EV2|Q53EV2_HUMAN Polymerase (DNA directed), beta variant (Fragment) OS=Homo sapiens
PE=2 SV=1;
>tr|A0A1B8CGP1|A0A1B8CGP1_9PEZI Uncharacterized protein OS=Pseudogymnoascus sp. WSF 3629
GN=VE00_03720 PE=4 SV=1;
>tr|G2YKX9|G2YKX9_BOTF4 Uncharacterized protein OS=Botryotinia fuckeliana (strain T4)
GN=BofuT4_P080720.1 PE=4 SV=1;
>tr|G3R1G2|G3R1G2_GORGO Uncharacterized protein OS=Gorilla gorilla gorilla GN=POLB PE=4
SV=1;
>tr|G3SIQ7|G3SIQ7_GORGO Uncharacterized protein OS=Gorilla gorilla gorilla GN=POLB PE=4
SV=1;
>tr|A0A135M080|A0A135M080_PENPA DNA polymerase family X OS=Penicillium patulum
GN=PGRI_077250 PE=4 SV=1;
>tr|V5L328|V5L328_9VIRU Putative DNA polymerase family X OS=Hirudovirus strain Sangsue
GN=HIRU_S640 PE=4 SV=1;
>tr|A0A140E0M9|A0A140E0M9_MIMIV DNA polymerase family x protein OS=Samba virus PE=4
SV=1;
>tr|A0A165XF78|A0A165XF78_MIMIV Putative DNA polymerase family X OS=Mimivirus Bombay
PE=4 SV=1;
>tr|A0A0U2SWJ7|A0A0U2SWJ7_9VIRU DNA polymerase family X OS=Niemeyer virus PE=4 SV=1;
>tr|G8ED36|G8ED36_9VIRU DNA polymerase family X OS=Acanthamoeba castellanii mamavirus
GN=MAMA_L395 PE=4 SV=1;
>tr|J3IZ33|J3IZ33_9VIRU DNA polymerase family X OS=Acanthamoeba polyphaga lentillevirus
GN=L262 PE=4 SV=1;
>tr|E3VZU8|E3VZU8_MIMIV DNA polymerase family X OS=Acanthamoeba polyphaga mimivirus
GN=L318 PE=4 SV=1;
>tr|A0A1E1EVX4|A0A1E1EVX4_9VIRU Putative DNA polymerase family X OS=Acanthamoeba
castellanii mimivirus PE=4 SV=1;
>sp|Q7T6Y4|DPOLX_MIMIV Probable DNA polymerase family X OS=Acanthamoeba polyphaga
mimivirus GN=MIMI_L318 PE=1 SV=2;
>tr|A0A1B8G098|A0A1B8G098_9PEZI Uncharacterized protein OS=Pseudogymnoascus sp. 03VT05
GN=VE02_02586 PE=4 SV=1;
>tr|A0A067GS95|A0A067GS95_CITSI Uncharacterized protein (Fragment) OS=Citrus sinensis
GN=CISIN_1g0093031mg PE=4 SV=1;
>tr|H6U746|H6U746_9SAUR DNA polymerase beta (Fragment) OS=Pogona vitticeps GN=POLB PE=2
SV=1;
>tr|A0A096N1P6|A0A096N1P6_PAPAN Uncharacterized protein OS=Papio anubis GN=POLB PE=4
SV=1;
>tr|A0A0D9RRA2|A0A0D9RRA2_CHLSB Uncharacterized protein OS=Chlorocebus sabaeus GN=POLB
PE=4 SV=1;
>tr|G7PBR6|G7PBR6_MACFA DNA polymerase beta OS=Macaca fascicularis GN=EGM_17277 PE=4
SV=1;
>tr|I0FSR3|I0FSR3_MACMU DNA polymerase beta OS=Macaca mulatta GN=POLB PE=2 SV=1;
>tr|A0A094GKB1|A0A094GKB1_9PEZI Uncharacterized protein OS=Pseudogymnoascus sp. VKM F-
103 GN=V499_07542 PE=3 SV=1;
>tr|A0A094CAY7|A0A094CAY7_9PEZI Uncharacterized protein OS=Pseudogymnoascus sp. VKM F-
4515 (FW-2607) GN=V496_08955 PE=3 SV=1;
>tr|A0A094GUE6|A0A094GUE6_9PEZI Uncharacterized protein OS=Pseudogymnoascus sp. VKM F-
4517 (FW-2822) GN=V498_00536 PE=3 SV=1;
>tr|A0A1E4RU82|A0A1E4RU82_CYBJA Nucleotidyltransferase OS=Cyberlindnera jadinii NRRL Y-
1542 GN=CYBJADRAFT_169885 PE=4 SV=1;
>tr|A0A135U6J3|A0A135U6J3_9PEZI Uncharacterized protein OS=Colletotrichum salicis
GN=CSAL01_12504 PE=4 SV=1;
>tr|Q5JQP2|Q5JQP2_HUMAN DNA polymerase lambda (Fragment) OS=Homo sapiens GN=POLL PE=1
SV=1;
>rf 1 5prime-gi|460163 [Gallus gallus]-3prime;
>rf 1 5prime-gi|494987 [Xenopus laevis]-3prime;
>rf 1 5prime-gi|1354475 [Oncorhynchus mykiss]-3prime;
>rf 1 5prime-gi|12802441 [Mus musculus]-3prime;
>rf 1 5prime-gi|28852989 [Ambystoma mexicanum]-3prime;
>rf 1 5prime-gi|38603668 [Takifugu rubripes]-3prime;
>rf 1 5prime-gi|40218593 [Ginglymostoma cirratum]-3prime;
>rf 1 5prime-gi|73998101 [Canis lupus familiaris]-3prime;
>rf 1 5prime-gi|139001476 [Lemur catta]-3prime;
>rf 1 5prime-gi|139001511 [Otolemur garnettii]-3prime;
>rf 1 5prime-gi|149704611 [Equus caballus]-3prime;
>rf 1 5prime-gi|164451472 [Bos taurus]-3prime;
>rf 1 5prime-gi|169642654 [Xenopus (Silurana) tropicalis]-3prime;
>rf 1 5prime-gi|291394899 [Oryctolagus cuniculus]-3prime;
>rf 1 5prime-gi|327280070 [Anolis carolinensis]-3prime;
>rf 1 5prime-gi|344274915 [Loxodonta africana]-3prime;
>rf 1 5prime-gi|348588114 [Cavia porcellus]-3prime;
>rf 1 5prime-gi|351697151 [Heterocephalus glaber]-3prime;
>rf 1 5prime-gi|355562663 [Macaca mulatta]-3prime;
>rf 1 5prime-gi|395501816 [Sarcophilus harrisii]-3prime;
>rf 1 5prime-gi|395508711 [Sarcophilus harrisii]-3prime;
>rf 1 5prime-gi|395850042 [Otolemur garnettii]-3prime;
>rf 1 5prime-gi|397467153 [Pan paniscus]-3prime;
>rf 1 5prime-gi|403278452 [Saimiri boliviensis boliviensis]-3prime;
>rf 1 5prime-gi|410903980 [Takifugu rubripes]-3prime;
>rf 1 5prime-gi|410975770 [Felis catus]-3prime;
>rf 1 5prime-gi|432092624 [Myotis davidii]-3prime;
>rf 1 5prime-gi|432113117 [Myotis davidii]-3prime;
>rf 1 5prime-gi|444708211 [Tupaia chinensis]-3prime;
>rf 1 5prime-gi|460417122 [Pleurodeles waltl]-3prime;
>rf 1 5prime-gi|466001476 [Orcinus orca]-3prime;
>rf 1 5prime-gi|471358897 [Trichechus manatus latirostris]-3prime;
>rf 1 5prime-gi|478528402 [Ceratotherium simum simum]-3prime;
>rf 1 5prime-gi|488530524 [Dasypus novemcinctus]-3prime;
>rf 1 5prime-gi|499037612 [Maylandia zebra]-3prime;
>rf 1 5prime-gi|504135178 [Ochotona princeps]-3prime;
>rf 1 5prime-gi|505844004 [Sorex araneus]-3prime;
>rf 1 5prime-gi|505845913 [Sorex araneus]-3prime;
>rf 1 5prime-gi|507537868 [Jaculus jaculus]-3prime;
>rf 1 5prime-gi|507572662 [Jaculus jaculus]-3prime;
>rf 1 5prime-gi|507622751 [Octodon degus]-3prime;
>rf 1 5prime-gi|507640406 [Echinops telfairi]-3prime;
>rf 1 5prime-gi|507669049 [Echinops telfairi]-3prime;
>rf 1 5prime-gi|507930719 [Condylura cristata]-3prime;
>rf 1 5prime-gi|507940587 [Condylura cristata]-3prime;
>rf 1 5prime-gi|511850623 [Mustela putorius furo]-3prime;
>rf 1 5prime-gi|512856623 [Xenopus (Silurana) tropicalis]-3prime;
>rf 1 5prime-gi|512952456 [Heterocephalus glaber]-3prime;
>rf 1 5prime-gi|524918754 [Mesocricetus auratus]-3prime;
>rf 1 5prime-gi|527251632 [Melopsittacus undulatus]-3prime;
>rf 1 5prime-gi|528493137 [Danio rerio]-3prime;
>rf 1 5prime-gi|528493139 [Danio rerio]-3prime;
>rf 1 5prime-gi|529438486 [Falco peregrinus]-3prime;
>rf 1 5prime-gi|530565557 [Chrysemys picta bellii]-3prime;
>rf 1 5prime-gi|532017142 [Microtus ochrogaster]-3prime;
>rf 1 5prime-gi|532099471 [Ictidomys tridecemlineatus]-3prime;
>rf 1 5prime-gi|533166077 [Chinchilla lanigera]-3prime;
>rf 1 5prime-gi|533189443 [Chinchilla lanigera]-3prime;
>rf 1 5prime-gi|537205041 [Cricetulus griseus]-3prime;
>rf 1 5prime-gi|537263119 [Cricetulus griseus]-3prime;
>rf 1 5prime-gi|543247043 [Geospiza fortis]-3prime;
>rf 1 5prime-gi|543731985 [Columba livia]-3prime;
>rf 1 5prime-gi|291404551 [Oryctolagus cuniculus]-3prime;
>rf 1 5prime-gi|301763246 [Ailuropoda melanoleuca]-3prime;
>rf 1 5prime-gi|478507321 [Ceratotherium simum simum]-3prime;
>rf 1 5prime-gi|543351492 [Pseudopodoces humilis]-3prime;
>rf 1 5prime-gi|544420267 [Macaca fascicularis]-3prime;
>rf 1 5prime-gi|545193630 [Equus caballus]-3prime;
>rf 1 5prime-gi|548384565 [Pundamilia nyererei]-3prime;
>rf 1 5prime-gi|551487466 [Xiphophorus maculatus]-3prime;
>rf 1 5prime-gi|551523268 [Xiphophorus maculatus]-3prime;
>rf 1 5prime-gi|554582962 [Myotis brandtii]-3prime;
>rf 1 5prime-gi|554588252 [Myotis brandtii]-3prime;
>rf 1 5prime-gi|556778822 [Pantholops hodgsonii]-3prime;
>rf 1 5prime-gi|556990133 [Latimeria chalumnae]-3prime;
>rf 1 5prime-gi|557297894 [Alligator sinensis]-3prime;
>rf 1 5prime-gi|558116760 [Pelodiscus sinensis]-3prime;
>rf 1 5prime-gi|558207237 [Myotis lucifugus]-3prime;
>rf 1 5prime-gi|560895997 [Camelus ferus]-3prime;
>rf 1 5prime-gi|560897502 [Camelus ferus]-3prime;
>rf 1 5prime-gi|562857949 [Tupaia chinensis]-3prime;
>rf 1 5prime-gi|562876575 [Tupaia chinensis]-3prime;
>rf 1 5prime-gi|564229057 [Alligator mississippiensis]-3prime;
>rf 1 5prime-gi|564236372 [Alligator mississippiensis]-3prime;
>rf 1 5prime-gi|564384286 [Rattus norvegicus]-3prime;
>rf 1 5prime-gi|573884994 [Lepisosteus oculatus]-3prime;
>rf 1 5prime-gi|2149634 [Monodelphis domestica]-3prime (pD441-NH);
>rf 1 5prime-gi|40037389 [Raja eglanteria]-3prime (pD441-NH);
>rf 1 5prime-gi|46369889 [Danio rerio]-3prime (pD441-NH);
>rf 1 5prime-gi|139001490 [Microcebus murinus]-3prime (pD441-NH);
>rf 1 5prime-gi|148708614 [Mus musculus]-3prime (pD441-NH);
>rf 1 5prime-gi|149040157 [Rattus norvegicus]-3prime (pD441-NH);
>rf 1 5prime-gi|311271684 [Sus scrofa]-3prime (pD441-NH);
>rf 1 5prime-gi|334313404 [Monodelphis domestica]-3prime (pD441-NH);
>rf 1 5prime-gi|345330196 [Ornithorhynchus anatinus]-3prime (pD441-NH).

What is claimed is:

1-24. (canceled)

25. A method of nucleic acid synthesis, which comprises the steps of:
(a) providing an immobilised initiator sequence;
(b) adding a nucleotide blocked at the 3′ position by either a 3′-O-2-(cyanoethoxy)methyl, 3′-O-(2-cyanoethyl), 3′-O-azidomethyl, 3′-aminoxy, or 3′-O-allyl group to said initiator sequence using a modified terminal transferase enzyme in which the BRCA-1 C-terminal (BRCT) domain is absent and a nucleoside triphosphate having a 3′-O-2-(cyanoethoxy)methyl, 3′-O-(2-cyanoethyl), 3′-O-azidomethyl, 3′-aminoxy, or 3′-O-allyl group;
(c) removal of all reagents from the initiator sequence;
(d) cleaving the blocking group from the nucleotide added in step (b) to said initiator sequence; and
(e) removal of the cleaving agent;
wherein greater than 1 nucleotide is added by repeating steps (b) to (e).

26. The method as defined in claim 25, wherein the initiator sequence is between 5 and 100 nucleotides long.

27. The method as defined in claim 25, wherein the initiator sequence is between 5 and 20 nucleotides long.

28. The method as defined in claim 25, wherein the modified terminal transferase enzyme is derived from the wild type sequence of L. oculatus, S. harrisii, S. scrofa, O. garnetti, C. lanigera, D. novemcinctus, M. domestica, P. nyererei, M. brandtii or M. musculus.

29. The method as defined in claim 25, wherein the modified terminal transferase enzyme is derived from the wild type sequence of M. musculus.

30. The method as defined in claim 25, wherein the modified terminal transferase enzyme is derived from the wild type sequence of L. oculatus.

31. The method as defined in claim 25, wherein the modified terminal transferase enzyme comprises 21% of the wild type mass N-terminal truncation.

32. The method as defined in claim 25, wherein the nucleotide is blocked at the 3′ position by a 3′-aminoxy group.

33. The method as defined in claim 25, wherein the method is performed in a microfluidic device.

34. A method of nucleic acid synthesis which is performed in a microfluidic device comprising the steps of:
(a) providing an initial initiator sequence bound to a surface within a microfluidic device;
(b) adding a nucleotide blocked at the 3′ position by either a 3′-O-2-(cyanoethoxy)methyl, 3′-O-(2-cyanoethyl), 3′-O-azidomethyl, 3′-aminoxy, or 3′-O-allyl group to said initiator sequence using a modified terminal transferase enzyme in which the BRCA-1 C-terminal (BRCT) domain is absent and a nucleoside triphosphate having a 3′-O-2-(cyanoethoxy)methyl, 3′-O-(2-cyanoethyl), 3′-O-azidomethyl, 3′-aminoxy, or 3′-O-allyl group;
(c) removal of all reagents from the initiator sequence;
(d) cleaving the blocking group from the reversibly blocked nucleotide added in step (b) to said initiator sequence in the presence of a cleaving agent; and
(e) removal of the cleaving agent;
wherein greater than 1 nucleotide is added by repeating steps (b) to (e).

35. The method as defined in claim 34, wherein said microfluidic device is selected from a continuous-flow microfluidic device, droplet-based microfluidic device, digital microfluidic device, programmable digital microfluidic device, microarray device (such as a DNA chip), optofluidic device and acoustic droplet ejection (ADE) device.

36. A kit comprising:
a) an immobilised initiator sequence;
b) a nucleoside triphosphate having a 3′-O-2-(cyanoethoxy)methyl, 3′-O-(2-cyanoethyl), 3′-O-azidomethyl, 3′-aminoxy, or 3′-O-allyl group;
c) a modified terminal transferase enzyme in which the BRCA-1 C-terminal (BRCT) domain is absent and;
(d) a cleaving agent.

37. The kit according to claim 36, containing a microfluidic device.

38. The kit according to claim 37, wherein the device is selected from a continuous-flow microfluidic device, droplet-based microfluidic device, digital microfluidic device, programmable digital microfluidic device, microarray device (such as a DNA chip), optofluidic device and acoustic droplet ejection (ADE) device.

39. The kit according to claim 37, wherein the modified terminal transferase enzyme is derived from the wild type sequence of M. musculus.

40. The kit according to claim 37, wherein the modified terminal transferase enzyme is derived from the wild type sequence of L. oculatus.

41. The kit according to claim 37, wherein the nucleoside triphosphate has a 3′-aminoxy group.


SEQUENCE LISTING (88 sequences)

SEQ ID NO: 1 - protein, 494 aa, Lepisosteus oculatus
MLHIPIFPPIKKRQKLPESRNSCKYEVKFSEVAIFLVERKMGSSRRKFLTNLARSKGFRIEDVLSDAVTHVVAEDNSADELWQWLQNSSLGDLSKIEVLDISWFTECMGAGKPVQVEARHCLVKSCPVIDQYLEPSTVETVSQYACQRRTTMENHNQIFTDAFAILAENAEFNESEGPCLAFMRAASLLKSLPHAISSSKDLEGLPCLGDQTKAVIEDILEYGQCSKVQDVLCDDRYQTIKLFTSVFGVGLKTAEKWYRKGFHSLEEVQADNAIHFTKMQKAGFLYYDDISAAVCKAEAQAIGQIVEETVRLIAPDAIVTLTGGFRRGKECGHDVDFLITTPEMGKEVWLLNRLINRLQNQGILLYYDIVESTFDKTRLPCRKFEAMDHFQKCFAIIKLKKELAAGRVQKDWKAIRVDFVAPPVDNFAFALLGWTGSRQFERDLRRFARHERKMLLDNHALYDKTKKYLKKKTTNNYLALNDVCSDLSEWHYKG

SEQ ID NO: 2 - protein, 502 aa, Salmo salar
MIHASMLPRVKKRPRPVEAGARGQEEVKFNEVTVYLVERKMGSSRRSFLTSLARSKGFRVEYVLSDEVTHVVAEDNRAGALWAWLRGSGLRDVSRLQVLDISWFTDSMREGRPVTVETRHCIPDTSSTVPECSPPIAAANVSQYACLRRTTTENHNKIFTDVLEELAENSEFNESKGPCLAFRRAASVLKSLPSAVHCLGAIQGLPCLGEHTKAVMEEILIFGRSFKVEEVQSDERYQALKLFTSVFGVGPKTAEKWYRRGLRSLKEILAEPNIQLNRMQRAGFLYYRDISKAVSKAEAKALRSIIEETAHWIAPDSILALTGGFRRGKEYGHDVDFLLTMPVMGKEEGLLLRVIDRLRDQGILLYCEHQGSTFDMSKLPSRRFEAMDHFEKCFLILRLEEGQVEGDGGLQKDPGESRGWRAVRVDLVAPPVDRYAFALLGWTGSRQFERDLRRFASKERGMCLDNHALYDKTKKLFLPATSEEDIFAHLGLEYVEPWQRNA

SEQ ID NO: 3 - protein, 501 aa, Salmo salar
MNHAGMLARVKKRKRPVEAGAQGQVEVKFKEVTLYLVERKMGSSRRNFLTSLARSKGFRVEDVLSDDVTHVVAEDNQAEVLWAWLMGHGLRDVSRLAVLDISWFTDSMREGRPVRVETRHRIQNTPTVTDCSPPTAVANVSQYACQRRTTTENHNKIFTDVMEELAESSEFNESKGPCLAFRQAASVLKSLPSAVQCLEAIQGLPCLGEHTKAVMEEILTFGRSFKVEELRCDERYQALKLFTSVFGVGPKTAEKWYRRGLRSLKEILTEPSIQLNRMQRAGFLYYSDISKAVSKAEAEALGCIIEETVHWIAPDAVLALTGGFRRGKEYGHDVDFLLTMPEMGKEEGLLLHVIDRLRDQGILLYCEYQGSTFDVSKLPSCRFEDMDHFQKCFLILRLEQGQVEGERGQQRDPGDSRGWRAVRVDLVAPPVDRYAFALLGWTGSRQFGRDLRAFAQKERQMLLDNHALYDKTKQLFLPVTTEEDIFAHLGLEYVEPWQRNA

SEQ ID NO: 4 - protein, 500 aa, Salmo salar
MNHAGMLARVKKRKRPVEAGAQGQVEVKFKEVTLYLVERKMGSSRRNFLTSLARSKGFRVEDVLSDDVTHVVAEDNQAEVLWAWLMGHGLRDVSRLAVLDISWFTDSMREGRPVRVETRHRIQNTPTVTDCSPPTAVANVSQYACQRRTTTENHNKIFTDVMEELAESSEFNESKGPCLAFRQAASVLKSLPSAVQCLEAIQGLPCLGEHTKAVMEEILTFGRSFKVEELRCDERYQALKLFTSVFGVGPKTAEKWYRRGLRSLKEILTEPSIQLNRMQRAGFLYYSDISKAVSKAEAEALGCIIEETVHWIAPDAVLALTGGFRRGKEYGHDVDFLLTMPEMGKEEGLLLHVIDRLRDQGILLYCEYQGSTFDVSKLPSCRFEDMDHFQKCFLILRLEQGQVEGERGQQRDPGDSRGWRAVRVDLVAPPVDRYAFALLGWTGSRFGRDLRAFAQKERQMLLDNHALYDKTKQLFLPVTTEEDIFAHLGLEYVEPWQRNA

SEQ ID NO: 5 - protein, 501 aa, Ictalurus punctatus
MFQTTLSTFLRKRRQPELTCAQPQKEMKFREVTVYLVERRMGKSRRNFLTSLARSKGFSVDNTLSSKVTHIVAEDNPAHELWPWLQEQGIADLGKMNVLDIAWFTQSMKAGRPIPVEAQHRIQKPSVQPKPEAGPPNSPWLTVSQYACQRRTTLYNHNKILTDALEVLAENYEFIESIGPCLGFRRAASMLKSLPAPLRNINDTEGLPCLGPETKAVIQDIFECGSSSKVEEVLTDERYRTLKIFTSVFGVGPKTAEKWYRKGLRSLEQIASDSSIHLNKMQIAGFQYYEDISKPVSKAEAEAVGHIIKEIAGCFSPDVTMTLTGGFRRGKEFGHDVDFLLTVPRPGKEDGLLPAVINQLRTQGLLLYSDFQESTFDLSSLPNRRFEAMDHFQKCFLIVKLKKDQVVGQQAEQRCGRDWKAVRVDLVAPPAERYAFALLGWSGSTQFERDLRRFSRLERNMLLDNHALFDKTTNTFLQAKTEEDIFTHLGLDYIEPWQRNA

SEQ ID NO: 6 - protein, 501 aa, Oncorhynchus mykiss
MNHAGMLALVKKRKRPVEAGAQGQVEVKFKEVTLELVERKMGSSRRNFLTRLARSKGFRVEDVLSDDVTHVVAEDNQAEVLWAWLMGHGLRDVSRLALLDISWFTDSMREGRPVRVETRHSIQNTPTGTDCSPPTAVANVSQYACQRRTTTENHNNKIFTDVMEELAESSEFNESKGPCLAFRQAASVLKSLPSAVHCLKAIQGLPCLGEHTKAVMEEILTFGRSFKVEEIRCDERYQALKLFTSVFGVGPKTAEKWYRRGLRSLQEILTEPNIQLNRMQRAGFLYYSDISKAVSKAEAKAVGCIIEDTFHWIAPDAILALTGGFRRGKEYGHDVDFLLTMPEIGKDEGLLLHVIDRLKDQGILLYCDYQGSTFDVSKLPSCRFEDMDCFQKCFLILRLEQGQVEGERGLQRDPGDSRGWRAVRVDLVAPPVDRYAFALLGWTGSRFGRDLRTFAQKERQMLLDNHALYDKTKKLCLLATTEEDIFTHLGLEYVEPWQRNA

SEQ ID NO: 7 - protein, 522 aa, Ginglymostoma cirratum
MSLAGSLGGMGIIPKRKRQKVTEVCSSQSKHQVRFQDLTIFIVERKMGSSRRSFLMDLARKKGFRVEDVMSDSVTHIVTENNSWDEIWDWIQNLKLLNADKLKMLNISWFTDSMAAGKPVEIEERHKLQVQKMLQSNSPLPPPVVTISQYACQRRSTLNNRNKIFTDALEILAENFEFNENESAYVAFARATSLLKSLPYTISKMAALDGLPCFGDQTRAIIEEILEDGVSSKVDDLLCDEKYKARKLFTSVFGVGLKTADKWYGQGFRTLEAVKASKDLKFTKMQKAGFLYYEDINNAVTRPEAEAVAQIIETIVHNYAPDAIVTLTGGFRRGKETGHDVDFLISCPETMDENFLRKIVNKLDFRGLLLYYDMVEATFEKRKLSSQKYDAMDHFQKCFLILKLNKALVKNRVLSMSSVSAARPTDEGAEPEVKTQIKDWKAIRVDLVIVPTQQFAYALLGWTGSRQFERDLRRYTNHEKSMILDNHGLYDRKKKIFLNAKTEEEIFAHLDLEYIEPWERNA

SEQ ID NO: 8 - protein, 509 aa, Astyanax mexicanus
CEVMFQTALSTLLRKRRRPESTPALPQQGKKFGEVMVYLVERRMGKSRRNFLTSLARSKGFCVDNTLSDKVTHIVAEGISANELWPWLEEQNLPQLDKTNVLDITWFTESMRADRPVPVEEQHRIQCRCVPVPVCPPEGYDPVSDSPVPTVSQYACQRRTTLDNPNKVLTDALEVLVENCEFNDSMGPCSGFRRAASVLKSLTTALSCLQDTAGLPCLGEESKTIIEEIIDCGSSSRVEDILSDERYQTLKLFTSVFGVGPKTAEKWYRRGLRSLEQVYSDSSLHFNRMQTAGFIYYEDISKPVTVAEARAVGCIIEETASYYSSGVSISLTGGFRRGKQFGHDVDFILTVPEPGKEDGLLPAVIDTLRSQGILLYSDFQESTFDLNKLPSRRFEAMDHFQKCFLILKLRAGLVEGQQVDPGCRRDWRAVRVDLVAPPAERYAFCLLGWSGSTQFERDLRRFARLERGMLLDNHALYDKTTNTFLQAKTEEDIFAHLGLDYIEPWQRNA

SEQ ID NO: 9 - protein, 510 aa, Dipodomys ordii
MDPLPTAHSGPRKKRPRQMGALMSSTPQDVKFRDLVLFILEKKMGTSRRAFLMELARSKGFRVENELSDSVTHIVAENNSGSDVLEWLQVQNIKASSQLEFLDISWLVECMGAGKPVQTTGKHQLVVRRDHPPSSNPGLQNTLPQTAQKISQYACERRTTLNNWNHVFTDALDILAENSEFRENESCCLAFMRASSVLKSLPFTVSSMKDTEGIPCLGDKVKCIIEEIIEDGESSEVKAVLNDERYQSFKLFTSVFGVGLKTSEKWFQMGFRTLSKVRSDKSLKFTRMQKAGFLYYEDLVSSVTKAEAEAVSMLVKEAVRTFLPSALVTLTGGFRRGKKIGHDVDFLITSPEVTEEKEQQLLHQVTNLWEKKGLLLYCDHVESTFEKLKLPSRKVDALDHFQKCFLILKLDHQRVNSDQSSQQEGKTWKAIRVDLVMCPYERRAFALLGWTGSRQFERDLRRYATHERKMMLDNHALYDKAKRIFLKAESEEEIFAHLGLDYIEPWERNA

SEQ ID NO: 10 - protein, 510 aa, Rattus norvegicus
MDPLQAVHLGPRKKRPRQTGASGASTPHDIRFRDLVLFILEKKMGTTRRAFLMELARRKGFRVENELSDSVTHIVAENNSGSDVLEWLQLQNIKASSEFELLDISWLIECMGAGKPVEMAGRHQLVVRRNPSLSPVPGSQTVPPPLMQKISQYACQRRTTLNNHNQLFTDAFDILAENYEFRENEVSCLPFMRAASVLKSLSFPIVSMKDIEGIPCLGDKVKCVIEGIIEDGESSEVKAVLNDERYKSFKLFTSVFGVGLKTAEKWFRMGFRTLSKIKSDKSLRFTHMQKAGFLYYEDLVSCVNRAEAEAVSMLVKEAVVAFLPDALVTMTGGFRRGKMTGHDVDFLITSPEATEEEEQQLLHKVTNFWRQQGLLLYCDIIESTFEKFKLPSRKVDALDHFQKCFLILKLHRGLVRSEESGQQEGKDWKAIRVDLVMCPYERRAFALLGWTGSRQFERDLRRYATHERKMMLDNHALYDKTKRVFLEAESEEEIFAHLGLDYIEPWERNA

SEQ ID NO: 11 - protein, 510 aa, Rattus norvegicus
MDPLQAVHLGPRKKRPRQTGASGASTPHDIRFRDLVLFILEKKMGTTRRAFLMELARRKGFRVENELSDSVTHIVAENNSGSDVLEWLQLQNIKASSEFELLDISWLIECMGAGKPVEMAGRHQLVVRRNPSLSPVPGSQTVPPPPMQKISQYACQRRTTLNNHNQLFTDAFDILAENYEFRENEVSCLPFMRAASVLKSLSFPIVSMKDIEGIPCLGDKVKCVIEGIIEDGESSEVKAVLNDERYKSFKLFTSVFGVGLKTAEKWFRMGFRTLSKIKSDKSLRFTHMQKAGFLYYEDLVSCVNRAEAEAVSMLVKEAVVAFLPDALVTMTGGFRRGKMTGHDVDFLITSPEATEEEEQQLLHKVTNFWRQQGLLLYCDIIESTFEKFKLPSRKVDALDHFQKCFLILKLHRGLVRSEESGQQEGKDWKAIRVDLVMCPYERRAFALLGWTGSRQFERDLRRYATHERKMMLDNHALYDKTKRVFLEAESEEEIFAHLGLDYIEPWERNA

SEQ ID NO: 12 - protein, 501 aa, synthetic construct
MFHTPILPRTRKRSRAEQEVARPGREEVKFVNVRLYLVEMKMGHSRRSFLTQLARSKGFMVEDDLSDSVTHVVSENSQASVLWAWLKDSGPANLPSMHVVNITWFTDSMKERRPVAVETRHLIQDTLPMLPEGRKVVAVATVSQYACQRRTTTTNHNAVFTDAFEVLAECYEFNQMEGRCLAFRRAASVLKSLPRVLSSLEDTHHLPCLGDHAKAIIDELLQHGRAFDVQKVLSDERYQTLKLFTSVFGVGPKTAEKWYRTGLRAFEHILADRSIHLNHMQQNGFLHYEDISRAVSKAEARALTKVIDEVVHAITPDAIVALTGGFRRGKEFGHDVDIIFTTLEVGKEENLLLDVIKSLENQGVLLYCDYQAATFDTAKLPAHSFEAMDHFAKCFLILRLEASQVEGGLNSGEEDSRGWRAVRVDLVSPPMNRYAFALLGWTGSRQFERDMRRFARKERRMLLDNHGLFDKNKKEFLAATTEKDIFDHLGLEYMEPWQRNA

SEQ ID NO: 13 - protein, 509 aa, Dipodomys ordii
MDPLPTAHSGPRKKRPRQMGALMSSTPQDVKFRDLVLFILEKKMGTSRRAFLMELARSKGFRVENELSDSVTHIVAENNSGSDVLEWLQVQNIKASSQLEFLDISWLVECMGAGKPVQTTGKHQLVVRRDHPPSSNPGLQNTLPQTAQKISQYACERRTTLNNWNHVFTDALDILAENSEFRENESCCLAFMRASSVLKSLPFTVSSMKDTEGIPCLGDKVKCIIEEIIEDGESSEVKAVLNDERYQSFKLFTSVFGVGLKTSEKWFQMGFRTLSKVRSDKSLKFTRMQKAGFLYYEDLVSSVTKAEAEAVSMLVKEAVRTFLPSALVTLTGGFRRGKKIGHDVDFLITSPEVTEEKEQQLLHQVTNLWEKKGLLLYCDHVESTFEKLKLPSRKVDALDHFQKCFLILKLDHQRVNSDQSSQQEGKTWKAIRVDLVMCPYERRAFALLGWTGSRFERDLRRYATHERKMMLDNHALYDKAKRIFLKAESEEEIFAHLGLDYIEPWERNA

SEQ ID NO: 14 - protein, 502 aa, Myotis lucifugus
MDSLQMAHSGPRKKRPRQMGASMASPPQDIKFRDLVLFILEKKMGTTRRAFLMELARRKGFRVENELSDSVTHIVAENNSGSDVLEWLQVQKVRARSQPELLDVSWLIECMSAGKPVATTGQHQLVVSVLVVISTVRGLIKISPYACQRRTTLNNRNHIFTDAFEILAENCEFRENEGSCLAFMRAASVLKSLPFTIISMKDTEGIPCLGDKVKSIIEEIIEDGESSEVKAVLNDERYQSFKLFTSVFGVGLKTSEKWFRMGFRTLSKIRSDKTLKFTRMQKAGFFYYEDLVSCVTKAEAEAVGVLVKEAVWAFLPDAFVTVTGGFRRGKKIGHDVDFLITSPGSTEEEEQQLLPKVINLWERKGLLLYYDLVESTFEKFKLPSRKVDALDHFQKCFLILKLHHQRVDGGKSSQQEGKTWKAIRVDLVMCPYERRAFALLGWTGSRQFERDLRRYATHERKMMLDNHALYDKTKRIVLEAESEEDIFAHLGLDYIDPWERNA

SEQ ID NO: 15 - protein, 509 aa, synthetic construct
MDPPRASHLSPRKKRPRQTGASMASSPQDIKFQDLVVFILEKKMGTTRRAFLMELARRKGFRVENELSDSVTHIVAENNSGSDVLEWLQVQKIQVSSQPELLDVSWLIECIGAGKPVEMTGKHQLVVRRDYSDSTNPGPPKTPPIAVQKISQYACQRRTTLNNCNQIFTDAFDILAENCEFRENEDSCVTFMRAASVLKSLPFTIISMKDTEGIPCLGSKVKGIIEEIIEDGESSEVKAVLNDERYQSFKLFTSVFGVGLKTSEKWFRMGFRTLSKVRSDKSLKFTRMQKAGFLYYEDLVSCVTRAEAEAVSVLVKEAVWAFLPDAFVTMTGGFRRGKKMGHDVDFLITSPGSTEDEEQLLQKVMNLWENKGLLLYYDLVESTFEKLRLPSRKVDALDHFQKCFLIFKLPRQRVDSDQSSWQEGKTWKAIRVDLVLCPYERRAFALLGWTGSRQFERDLRRYATHERKMILDNHALYDKTKRIFLKAESEEEIFAHLGLDYIEPWERNA

SEQ ID NO: 16 - protein, 510 aa, Macaca fascicularis
MDPPQTSPLSPRKKRPRQMGALMASSPQDIKFQDLVVFILEKKMGTTRRAFLMELARRKGFRVENELSDSVTHIVAENNSGSDVLEWLQVQKIQVSSQPELLDVSWLIECIGAGKPVEMTGKHQLVVRRDYSDSTNPGPPKTLPTAVQKISQYACQRRTTLNNCNQIFTDAFDILAENCEFRENEDSCVTFMRAASVLKSLPFTIISMKDTEGIPCLGSKVKCIIEEIIEDGESSEVKAVLNDERYQSFKLFTSVFGVGLKTSEKWFRMGFRTLSKVRSDESLKFTRMQRAGFLYYEDLVSCVTRAEAEAVSVLVKEAVQAFLPDAFVTMTGGFRRGKKMGHDVDFLITSPGSTEDEEQQLLQKVMNLWEKKGLLLYYDLVESTFEKLRLPSRKVDALDHFQKCFLIFKLPLQRVDSDQSSWQEGKTWKAIRVDLVMCPYERRAFALLGWTGSRQFERDLRRYATHERKMILDNHALYDKTKRIFLKAESEEEIFAHLGLDYIEPWERNA

SEQ ID NO: 17 - protein, 510 aa, Macaca mulatta
MDPPQTSPLSPRKKRPRQTGALMASSPQDIKFQDLVVFILEKKMGTTRRAFLMELARRKGFRVENELSDSVTHIVAENNSGSDVLEWLQVQKIQVSSQPELLDVSWLIECIGAGKPVEMTGKHQLVVRRDYSDSTNPGPPKTLPTAVQKISQYACQRRTTLNNCNQIFTDAFDILAENCEFRENEDSCVTFMRAASVLKSLPFTIISMKDTEGIPCLGSKVKCIIEEIIEDGESSEVKAVLNDERYQSFKLFTSVFGVGLKTSEKWFRMGFRTLSKVRSDESLKFTRMQRAGFLYYEDLVSCVTRAEAEAVSVLVKEAVQAFLPDAFVTMTGGFRRGKKMGHDVDFLITSPGSTEDEEQQLLQKVMNLWEKKGLLLYYDLVESTFEKLRLPSRKVDALDHFQKCFLIFKLPLQRVDSDQSSWQEGKTWKAIRVDLVMCPYERRAFALLGWTGSRQFERDLRRYATHERKMILDNHALYDKTKRIFLKAESEEEIFAHLGLDYIEPWERNA

SEQ ID NO: 18 - protein, 500 aa, Oreochromis niloticus
MFHAPIVPRARKRSRPAEASAPRREGVKFEDVRLYLVERKMGRSRRSFLTELARSKGFIVEDVLSDVVTHVVSEDSQASSLWAWLKGGSVKNLPVMHVLDISWFTDSMREGKPVAVETRHLIQETLPASPEATTPTPVSTVSQYACQRRTTTQNNNKIFTDAFEVLAESHEFNDMEGPCLAFRRAASVLKSLPWTVQNLRVTEDLPCLGEHSMCVIEEILQHGRSFEVEKILSDERYQILKLFTSVFGVGPKTAEKWYRRGLRSFSDVLAEPSIHLNRMQQSGFLHYGDISRAVSKAEAQALGNIIDEAVHAITPDAILTLTGGFRRGKDFGHDVDFIVTTPQLGKEERLLTSVIDRLKHQGILLYCEYQASTFDESKLPSHRFEAMDHFAKCFLILRLEDSQVDGGLQTAEEDRRGWRAVRVDLVSPPVDRYAFTLLGWSGSRQFERDLRRFARMERRMLLDNHALYDKTKKSKVKRFVSHSLAFQNLCREWSPWRTSA

SEQ ID NO: 19 - protein, 510 aa, Papio anubis
MDPPQTSHLSPRKKRPRQTGALMASSPQDIKFQDLVVFILEKKMGTTRRTFLMELARRKGFRVENELSDSVTHIVAENNSGSDVLEWLQVQKIQVSSQPELLDVSWLIECIGAGKPVEMTGKHQLVVRRDYSDSTSPGPPKTLPTAVQKISQYACQRRTTLNNCNQIFTDAFDILAENCEFRENEDSCVTFMRAASVLKSLPFTIISMKDTEGIPCLGSKVKCIIEEIIEDGESSEVKAVLNDERYQSFKLFTSVFGVGLKTSEKWFRMGFRTLSKVRSDESLKFTRMQRAGFLYYEDLVSCVTRAEAEAVSVLVKEAVRAFLPDAFVTMTGGFRRGKKMGHDVDFLITSPGSTEDEEQQLLQKVMNLWEKKGLLLYYDLVESTFEKLRLPSRKVDALDHFQKCFLIFKLPLQRVDSHQSSWQEGKTWKAIRVDLVMCPYERRAFALLGWTGSRQFERDLRRYATHERKMILDNHALYDKTKRIFLKAESEEEIFAHLGLDYIEPWERNA

SEQ ID NO: 20 - protein, 509 aa, Nomascus leucogenys
MDPPQASHLSPRKKRPRQTGALMASSPEDIKFQDLVVFILEKKMGTTRRAFLMELARRKGFRVENELSDSVTHIVAENNSGSDVLEWLQVQKIQVSSQPELLDVSWLIECIGAGKPVEMTGKHQLVVRRDYSDSTNPGPPKTPPIAVQKISQYACQRRTTLNNCNQIFTDAFDILAENCEFRENEDSCVAFMRAASVLKSLPFTIISMKDTEGIPCLGSKEKGIIEEIIEDGESSEVKAVLNDERYQSFKLFTSVFGVGLKTSEKWFRMGFRTLSKVRSDKSLKFTQMQKAGFLYYEDLVSCVTRAEAEAVSVLVKEAVWAFLPDASITMTGGFRRGKKMGHDVDFLITSPGSTEDEEQLLQKVMNLWEKKGLLLYYDLVESTFEKLRLPSRKVDALDHFQKCFLIFKLPRQRVDGDQSSWQEGKTWKAIRVDLVLCPYERRAFALLGWTGSRQFERDLRRYATHERKMILDNHALYDKTKRIFLKAESEEEIFAHLGLDYIEPWERNA

SEQ ID NO: 21 - protein, 509 aa, Bos taurus
MDPLCTASSGPRKKRPRQVGASMASPPHDIKFQNLVLFILEKKMGTTRRNFLMELARRKGFRVENELSDSVTHIVAENNSGSEVLEWLQVQNIRASSQLELLDVSWLIESMGAGKPVEITGKHQLVVRTDYSATPNPGFQKTPPLAVKKISQYACQRKTTLNNYNHIFTDAFEILAENSEFKENEVSYVTFMRAASVLKSLPFTIISMKDTEGIPCLGDKVKCIIEEIIEDGESSEVKAVLNDERYQSFKLFTSVFGVGLKTSEKWFRMGFRSLSKIMSDKTLKFTKMQKAGFLYYEDLVSCVTRAEAEAVGVLVKEAVWAFLPDAFVTMTGGFRRGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 22 - protein, 520 aa, Bos taurus
MAQQRQHQRLPMDPLCTASSGPRKKRPRQVGASMASPPHDIKFQNLVLFILEKKMGTTRRNFLMELARRKGFRVENELSDSVTHIVAENNSGSEVLEWLQVQNIRASSQLELLDVSWLIESMGAGKPVEITGKHQLVVRTDYSATPNPGFQKTPPLAVKKISQYACQRKTTLNNYNHIFTDAFEILAENSEFKENEVSYVTFMRAASVLKSLPFTIISMKDTEGIPCLGDKVKCIIEEIIEDGESSEVKAVLNDERYQSFKLFTSVFGVGLKTSEKWFRMGFRSLSKIMSDKTLKFTKMQKAGFLYYEDLVSCVTRAEAEAVGVLVKEAVWAFLPDAFVTMTGGFRRGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 23 - protein, 510 aa, Mus musculus
MDPLQAVHLGPRKKRPRQLGTPVASTPYDIRFRDLVLFILEKKMGTTRRAFLMELARRKGFRVENELSDSVTHIVAENNSGSDVLEWLQLQNIKASSELELLDISWLIECMGAGKPVEMMGRHQLVVNRNSSPSPVPGSQNVPAPAVKKISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKRVFLEAESEEEIFAHLGLDYIEPWERNA

SEQ ID NO: 24 - protein, 510 aa, Sus scrofa
MDPPQTVPSSPRKKRPRQVGASMASPAHNIKFRELVLFILEKKMGTTRRTFLMELARRKGFRVENELSDSVTHIVAENNSGSEVLEWLQAQKIRASSQLTLLDVSWLIESMGAGKPVEMTGKHQLVVRTDCSASPSPGSQNTLPPAVKKISQYACQRRTTLNNCNHIFTDAFEVLAENYEFRENETFCLAFMRAASVLKSLPFTIISMKDTEGIPCLGDKVKCVIEEIIEDGESSEVKAVLNDERYQSFKLFTSVFGVGLKTSERWFRMGFRSLSKIRSDKTLKFTRMQKAGFLYYEDLVSCVTRAEAEAVGVLVKEAVQAFLPDAFVTMTGGFRRGKKMGHDVDFLITSPGSTDDEEQQLLPKVVNLWEREGLLLYCDLVESTLEKSKLPSRNVDALDHFQKCFLILKLHHQRVDSGMSSQQEGKTWKAIRVDLVMCPYELRAFALLGWTGSRQFERDLRRYATHERKMILDNHALYDKTKRIFLKAESEEEIFAHLGLDYLEPWERNA

SEQ ID NO: 25 - protein, 510 aa, Mus musculus; identical to SEQ ID NO: 23

SEQ ID NO: 26 - protein, 509 aa, Equus caballus
MDPFQMAHSSPRKKRPRQMSTSMASPPHDIKFRDLVLFILEKKMGTTRRAFLMELARRKGFRVENELSDSVTHIVAENNSGSDVLEWLQVQNIKAGSQPELLDVSWLIECMRAGKPVEMTGKHQLVVRRDDSAGPNPGPQETPPLVKKISPYACQRRTTLDNCNQVFTDAFDVLAENYEFRENESSCLTFMRAASVLKSLPFTIISMKDIEGIPCLEDKAKCVIEEIIEDGESSEVKTVLNDERYQSFKLFTSVFGVGLKTSEKWFRMGFRTLSKIRSDKTLKFTKMQKAGFLYYEDLVSCVTRPEAEAVSVLVKEAVWAFLPDAFVTMTGGFRRGKKIGHDVDFLITSPGSTEEEEQELLSKVINLWERKELLLYYDLVESTFEKSKLPSRKVDALDHFQKCFLILKLHHQRVDSGKSSQQEGKTWKAIRVDLVVCPYENHAFALLGWTGSRQFERDLRRYATHERKMILDNHALYDKTKRIFLKAESEEEIFAHLGLDYIEPWERNA

SEQ ID NO: 27 - protein, 509 aa, Pan troglodytes
MDPPRASHLSPRKKRPRQTGALMASSPQDIKFQDLVVFILEKKMGTTRRAFLMELARRKGFRVENELSDSVTHIVAENNSGSDVLEWLQVQKIQVSSQPELLDVSWLIECIGAGKPVEMTGKHQLVVRRDYSDSTNPGPPKTPPIAVQKISQYACQRRTTLNNCNQIFTDAFDILAENCEFRENEDSCVTFMRAASVLKSLPFTIISMKDTEGIPCLGSKVKGIIEEIIEDGESSEVKAVLNDERYQSFKLFTSVFGVGLKTSEKWFRMGFRTLSKVRSDKSLKFTRMQKAGFLYYEDLVSCVTRAEAEAVSVLVKEAVWAFLPDAFVTMTGGFRRGKKMGHDVDFLITSPGSTEDEEQLLQKVMNLWEKKGLLLYYDLVESTFEKLRLPSRKVDALDHFQKCFLIFKLPRQRVDSDQSSWQEGKTWKAIRVDLVLCPYERRAFALLGWTGSRQFERDLRRYATHERKMILDNHALYDKTKRIFLKAESEEEIFAHLGLDYIEPWERNA

SEQ ID NO: 28 - protein, 517 aa, synthetic construct
QRQHQRLPMDPLCTASSGPRKKRPRQVGASMASPPHDIKFQNLALFILEKKMGTTRRNFLMELARRKGFRVENELSDSVTHIVAENNSGSEVLEWLQVQNIRASSQLELLDVSWLIESMGAGKPVEITGKHQLVVRTDYSATPNPGFQKTPPLAVKKISQYACQRKTTLNNQNHIFTDAFEILAENSEFKENEVSYVTFMRAASVLKSLPFTIISMKDTEGIPCLGDKVKCIIEEIIEDGESSEVKAVLNDEQYQSFKLFTSVFGVGLKTSEKWFRMGFRSLSKIMSDKTLKFTKMQKAGFLYYEDLVSCVTRAEAEAVGVLVKEAVWAFLPDAFVTMTGGFRRGKKIGHDVDFLITSPGSAEDEEQLLPKVINLWEKKGLLLYYDLVESTFEKFKLPSRQVDTLDHFQKCFLILKLHHQRVDSSKSNQQEGKTWKAIRVDLVMCPYENRAFALLGWTGSRQFERDIRRYATHERKMMLDNHALYDKTKRVFLKAESEEEIFAHLGLDYIEPWERNA

SEQ ID NO: 29 - protein, 509 aa, Pongo abelii
MDPPRASHLSPRKKRPRQTGALMASSPQDIKFQDLVIFILEKKMGTTRRAFLMELARRKGFRVENELSDSVTHIVAENNSGSDVLEWLQVQKIQVSSQPELLDISWLIECIGAGKPVEMTGKHQLVVRRDYSDSTNPGPPKTPPIAVQKISQYACQRRTTLNNCNQIFTDAFDILAENCEFRENEDSCVTFMRAASVLKSLPFTIISMKDTEGIPCLGSKVKGIIEEIIEDGESSEVKAVLNDERYQSFKLFTSVFGVGLKTSEKWFRMGFRTLSKVRLDKSLKFTRMQKAGFLYYEDLVSCVTRAEAEAVSVLVKEAVWAFLPDAFVTMTGGFRRGKKMGHDVDFLITSPGSTEDEEQLLQKVMNLWEKKGLLLYYDLVESTFEKLRLPSRKVDALDHFQKCFLIFKLPRQRVDSDQSSWQEGKTWKAIRVDLVLCPYECRAFALLGWTGSRQFERDLRRYATHERKMILDNHALYDKTKRIFLKAESEEEIFAHLGLDYIEPWERNA

SEQ ID NO: 30 - protein, 509 aa, Ailuropoda melanoleuca
MDPLQMVHSGPRKKRPRQMGTSMVSPPHDIKFQDLVLYILEKKMGTTRRAFLMELARRKGFRVENELSDSITHIVAENNSGSEVLEWLQVQNIKASSQLELLDVSWLIESMGAGKPVEMTGKHQLVRRDYSASPNPELQKTPPLAVKKISQYACQRRTTLNNCNHIFTDAFEVLAENYEFRENEVFCLAFMRAASVLKSLPFTIISMKDTEGIPCLGDKVKCVIEEIIEDGESSEVKAVLNDERYQSFKLFTSVFGVGLKTSEKWFRMGFRTLSKIKSDKTLKFTPMQKAGFLYYEDLVSCVTRAEAEAVGVLVKEAVWAFLPDAFVTMTGGFRRGKKIGHDVDFLITSPGSTDEDEEQLLPKVINLWERKGLLLYCDFVESTFEKLKTPSRKVDALDHFQKCFLILKLHHQRVDSGKCSQQDGKTWKAIRVDLVMCPYERRAFALLGWTGSRQFERDLRRYATHERKMMLDNHALYDKTKKIFLKAESEEEIFAHLGLDYIEPWERNA

SEQ ID NO: 31 - protein, 509 aa, Homo sapiens
MDPPRASHLSPRKKRPRQTGALMASSPQDIKFQDLVVFILEKKMGTTRRAFLMELARRKGFRVENELSDSVTHIVAENNSGSDVLEWLQAQKVQVSSQPELLDVSWLIECIRAGKPVEMTGKHQLVVRRDYSDSTNPGPPKTPPIAVQKISQYACQRRTTLNNCNQIFTDAFDILAENCEFRENEDSCVTFMRAASVLKSLPFTIISMKDTEGIPCLGSKVKGIIEEIIEDGESSEVKAVLNDERYQSFKLFTSVFGVGLKTSEKWFRMGFRTLSKVRSDKSLKFTRMQKAGFLYYEDLVSCVTRAEAEAVSVLVKEAVWAFLPDAFVTMTGGFRRGKKMGHDVDFLITSPGSTEDEEQLLQKVMNLWEKKGLLLYYDLVESTFEKLRLPSRKVDALDHFQKCFLIFKLPRQRVDSDQSSWQEGKTWKAIRVDLVLCPYERRAFALLGWTGSRQFERDLRRYATHERKMILDNHALYDKTKRIFLKAESEEEIFAHLGLDYIEPWERNA

SEQ ID NO: 32 - protein, 510 aa, Callithrix jacchus
MDPPRTSHLSPRKKRPRQTGALMASSPQDIKFQDLVIFILEKKMGTTRRAFLTELARRKGFRVENELSNSVTHIVAENNSGSDVLEWLQVQKIQASSRPELLDVSWLIECIGAGKPVEMTGKHQLVVGRDYSDSTNPGLLKTPPIAVQKISQYACQRRTTLNNFNQIFTDAFDILAENCEFRENEDSCVTFMRAASVLKSLPFTIISMKDTEGIPCLGSKVKGIIEEIIEDGESSEVKAVLNDERYQSFKLFTSVFGVGLKTSEKWFRMGFRTLSKVRSEKSLTFTRMQRAGFLYYEDLVSCVTRAEAEAVSVLVKEAVWAFLPDAFITMTGGFRRGKKMGHDVDFLITSPGSTEDEEQQLLQKVMNLWEKKGLLLYYDLVESTFEKLRLPSRKVDALDHFQKCFLIFKLPRQRLDSDQPSWQEGKTWKAIRVDLVMCPYERRAFALLGWTGSRQFERDLRRYATHERKMILDNHALYDKTKRIFLKAESEEEIFTHLGLDYIEPWERNA

SEQ ID NO: 33 - protein, 516 aa, Erinaceus europaeus
MDALPVVHSSPRKKRSRLMGASVAYPPYDIKFHNLVLFILEKKMGSSRRAFLMELARRKGFRVEDELSDSITHIVAENNTGSEVLEWLQVQDIKISSQLELLDVSWLVECMRAGNPVVITGKHQLVSYTVKSDASFGSNPGSQNTPPLAIKKISQYACQRRTSLNNCNHIFTDALDILAENHEFRENEVSCVAFMRAASVLKSLPFTIISMKDTKGIPCLGDKAKCVIEEIIEDGESSEVKAILNDERYQSFKLFTSVFGVGLKTSEKWFRMGFRTLNKIMSDKTLKLTRMQKAGFLYYEDLVSCVAKAEADAVSVLVQEAVWAFLPDAMVTMTGGFRRGKKLGHDVDFLITSPGATEEEEQQLLPKVINFWERKGLLLYHDLVESTFEKLKLPSRKVDALDHFQKCFLILKLHLQHVNGVGNSKTGQQEGKNWKAIRVDLVMCPYERRAFALLGWTGSRQFERDLRRFATHERKMMLDNHALYDKTKRIFLKAESEEEIFAHLGLDYIDPWERNA

SEQ ID NO: 34 - protein, 510 aa, Loxodonta africana
MDPLQMAHTGPRKKRARPMGASMATTPQDIKFQDLVLFILEKKMGTSRRAFLMELARRKGFRVENELSDSVTHIVAENNSGSDVLEWLQVQKIKASSQLELLDVSWLIECMGAGKPVEMTGKHQLVVRGDYSASSNPSPQKTPPPAVKKISQYACQRRTTLNNHNNIFTDAFEILAENCEFRENEGSYVVFMRAASVLKSLPFTIISMKDTQGIPCLEDKAKCVIEDIIEDGESSEVKAVLNDERYKSFKLFTSVFGVGLKTSEKWFRMGFRSLSKIRTDKTLKFTEMQEAGFLYYEDLVSCVTKAEADAVSVLVKEAVWAFLPDAFVTMTGGFRRGKKVGHDVDFLITSQGSTEEEQQQLLHKVLNLWKKEGLLLYSDLIESTFEKLKLPSRKVDALDHFQKCFLILKLHHQRIDNSKSSQLQGKTWKAIRVDLVMCPYECHAFALLGWTGSRQFERDIRRYASHERKMILDNHALYDKTKRIFLKAGSEEEIFAHLGLDYIEPWERNA

SEQ ID NO: 35 - protein, 513 aa, synthetic construct
MPLPTNPLQMVHLGPRKKKPRQMGASMVCPSHDIKFQDLVLYILEKKMGTTRRAFLMELARRKGFRVEDELSDSITHIVAENNSGSEVLEWLQGQNHKVSSQLELLDVSWLIESMAAGTPVETTGKHQLVKRDYSANPNPELQKTPPLVIKKISQYACQRRTTLNNYNQIFTDAFEVLAENYEFRENEVFCLAFMRAASVLKSLPFTIISMKDTEGIPCLEDKVKCVIEEIIEDGESSEVKAVLNDERYQSFKLFTSVFGVGLKTSEKWFRMGYRTLNKIKLDKTLKFTPMQKAGFLYYEDLVSCVTRAEAEAVGMLVKEAVWAFLPEAFVTMTGGFRRGKKIGHDVDFLITSPGSTDEDEEQLLPKVINLWERKGLLLYCDLVESTFEKLKLPSRKVDALDHFQKCFLILKLHHQRVDSGKCSQQEGKTWKAIRVDLVMCPYERRAFALLGWTGSRQFERDLRRYATHERKMILDNHALYDKTKKIFLKAESEEEIFAHLGLDYIEPWERNA

SEQ ID NO: 36 - protein, 511 aa, Otolemur garnettii
MDPLHMAHSGPRKKRPRQTAASMVSTPQDIKFRDLVLFILEKKMGTTRRTFLMELARTKGFRVENEFSDSVTHIIAENNSGSDVLEWIQVQKIKAGSQMEVLDVSWLIECMRAGKPVEMTGKHQLVVRGDYSPSPNPAPQKTPPLAVQKISQYACQRRTTLNNCNHIFTDAFEIMAENYEFRENEGYSAAFMRAASVLKSLPFTIISMKDTEGVPCLGDNVKCIIEEIIEEGESSEVKAVLNDERYQSFKLFTSVFGVGLKTSEKWFRMGFRTLSKIRSDKSLRFTRMQQAGFLYYEDLVSCVTRAEAEAVGVLVKEAVRAFLPDAFVTMTGGFRRGKNIGHDVDFLITSPGSTEEEEQQLLHKIMDLWEKKGLLLYCDLVESTFEKLKLPSRKVDALDHFQKCFLIFKLHHQRVVDSEQSNQQEGKTWKAIRVDLVMCPYERRAYALLGWTGSRQFERDLRRYATHERKMILDNHGLWDKTKRIFLKAESEEEIFAHLGLDYIEPWERNA

SEQ ID NO: 37 - protein, 512 aa, Ailuropoda melanoleuca
PMDPLQMVHSGPRKKRPRQMGTSMVSPPHDIKFQDLVLYILEKKMGTTRRAFLMELARRKGFRVENELSDSITHIVAENNSGSEVLEWLQVQNIKASSQLELLDVSWLIESMGAGKPVEMTGKHQLVRRDYSASPNPELQKTPPLAVKKISQYACQRRTTLNNCNHIFTDAFEVLAENYEFRENEVFCLAFMRAASVLKSLPFTIISMKDTEGIPCLGDKVKCVIEEIIEDGESSEVKAVLNDERYQSFKLFTSVFGVGLKTSEKWFRMGFRTLSKIKSDKTLKFTPMQKAGFLYYEDLVSCVTRAEAEAVGVLVKEAVWAFLPDAFVTMTGGFRRIRGKKIGHDVDFLITSPGSTDEDEEQLLPKVINLWERKGLLLYCDFVESTFEKLKTPSRKVDALDHFQKCFLILKLHHQRVDSGKCSQQDGKTWKAIRVDLVMCPYERRAFALLGWTGSRQFERDLRRYATHERKMMLDNHALYDKTKKIFLKAESEEEIFAHLGLDYIEPWERNA

SEQ ID NO: 38 - protein, 514 aa, Ovis aries
MAQPEAASASPMDPLCTASSGPRKKRPRQVGASMASPPHDIKFQNLVLFILEKKMGTTRRNFLMELARRKGFRVENELSDSVTHIVAENNSGSEVLEWLQVQNIRASSQLELLDVSWLIESMGAGKPVEITGKHQLVVRTDYSATPNPGFQKISQYACQRKTTLNNYNHIFTDAFEILAENSEFKENEVSYVTFMRAASVLKSLPFTIISMRDTEGIPCLGDKVKCIIEEIIEDGESSEVKAVLNDERYQSFKLFTSVFGVGLKTSEKWFRMGFRSLNKIMSDKTLKFTKMQKAGFLYYEDLVSCVTRAEAEAVGVLVKEAVWAFLPDAFVTMTGGFRRIRGKKIGHDVDFLITSPGSAEDEEQLLPKVINFWEKKGLLLYYDLVESTFEKFKLPSRQVDTLDHFQKCFLILKLHHQRVDSGKSNQQEGKTWKAIRVDLVMCPYENHAFALLGWTGSRQFERDIRRYATHERKMMLDNHALYDKTKRMFLKAESEEEIFAHLGLDYIEPWERNA

SEQ ID NO: 39 - protein, 509 aa, synthetic construct
MDPLQMAHSGPRKKRPRQMGAPMVSPPHNIKFQDLVLYILEKKMGTTRRAFLMELARRKGFRVDNEFSDSITHIVAENNSGSDVLEWLQVQNIKASSQLELLDISWLIESMGAGKPVEMTGKHQLMRRDYTASPNPELQKTLPVAVKKISQYACQRRTTLNNYNNVFTDAFEVLAENYEFRENEVFSLTFMRAASVLKSLPFTIISMKDTEGIPCLGDQVKCIIEEIIEDGESSEVKAVLNDERYQSFKLFTSVFGVGLKTSEKWFRMGFRTLSKIKSDKSLKFTPMQKAGFLYYEDLVSCVTRAEAEAVGVLVKEAVGAFLPDAFVTMTGGFRRGKKMGHDVDFLITSPGSTDEDEEQLLPKVINLWERKGLLLYCDLVESTFEKLKLPSRKVDALDHFQKCFLILKLHHQRVDGGKCSQQEGKTWKAIRVDLVMCPYERRAFALLGWTGSRQFERDLRRYASHERKMILDNHALYDKTKKIFLKAESEEEIFAHLGLDYIEPWERNA

SEQ ID NO: 40 - protein, 517 aa, Sarcophilus harrisii
MHRIRTTDSDHGKKRQKKMDAISSKLYEIKFHEFVLFILEKKMGATRRTFLMDLARKKGFRVESELSNSVTHIVAENNSGSDVLAWLEAHKLETTAHFELLDVSWLIECMKVGKPVDTKGKYQLVESSIASANPDPNEGMLKIQSPAMNAISPYACQRRTTLNNHNQRFTDAFEILAKNYEFRENHGHCLTFLRATSVLKCLPFAIVSMKDAEGLPWIGDEVKGIMEEIIEDGQSLEVQAVLNDERYQAFKLFTSVFGVGLKTAEKWYRMGFRTLSKIQSDKSLKFTKMQKAGFLYYEDLISCVSKAEADAVSLIVKEAVWTFLPDALITITGGFRRGKEFGHDVDFLITSPGGEKEQVDQLLQKVTNLWEKQGLLLYYDLMESTFEDLKLPSRKVDALDHFQKCFLILKLYCQRGDRSKWEGPEGSNGLQTKNWKAIRVDLVVCPYDRYAYALLGWSGSRQFERDLRRYATHEKKMMLDNHALYDKTKRTFLKAESEEEIFSHLGLEYIEPWERNA

SEQ ID NO: 41 - protein, 512 aa, Cavia porcellus
MDLLQAPHSGPRKKRPRKAGALMVSGAHEVRFGDLVLFILEKKMGSTRRAFLMDLARSKGFRVEDELRDLMLLPHLVAENNSGNDVLEWLQVQNIQASSRLELLDVSWLIECMGAGKPVEMTRKHQLVVRRGSPASPNPDAQKTPSRSVQRISEYACQRRTTLNNCNYAFTNAFEILAENCEFKENEDSYVTYIRAASVLKSLPFTIISMKDTEGIPCLGDRVKCIIEEIIEDGESSEVNAVLNDERYKSFKLFTSVFGVGLKTSEKWFRMGFRSLNKIKSDKSLKFTRMQKAGFLYYEDLVSCVTRAEAEAVSTLVKEAVWAFLPGAFISMTGGFRRGKEIGHDVDFLITSPEITEDEEQQVLHKVINLWENKGLLLYSDLVESTFEKLKLPSRKVDALDHFQKCFLILKLHHQREDNEKSSQQEEKTWKAIRVDLVMCPYERRAFALLGWTGSRQFERDLRRYATHERKMMLDNHALYDKTKRIFLKAESEEEIFAHLGLDYIEPWERNA

SEQ ID NO: 42 - protein, 534 aa, Ficedula albicollis
MDRFKAPTVTSMRKRQKGLHSPKLSCSYEIKFSSFVIFIMQRKMGMTRRSFLMELGRRKGFRVESELSDSVTHIVAENNSYLEVLDWLKGQAVGDSSRFELLDISWFTACMEAGRPVDSEMKYRLVEQCQSPPLSTPELEMPAFIATKVSQYSCQRKTTLDNYNKKFTDAFEVMAENYEFKENEIFCLEFLRAASLLKSLPFSVTRMKDIQGLPCMGDQVRDIIEEIIEEGESSRVKEVLNDERYKAFKQFTSVFGVGVKTSEKWYRMGLRTVEEVKADKTLKLSKMQKAGFLYYEDLVSCVSKAEADAVSLIVKNTVCSFLPDALVTITGGFRRGKNIGHDIDFLITNPGPREDDELLHKVIDLWKKQGLLLYCDIIESTFVKEQLPSRKVDAMDHFQKCFAILKLYQPRVDNSTCNTSKKLGMAEVKDWKAIRVDLVITPFEQYAYALLGWTGSRQFGRDLRRYAAHERKMILDNHALYDRRKGMTLKMKSLCINIGKLKKGLERIFLKSGSEEEIFAHLGLDYVQPWERNA

SEQ ID NO: 43 - protein, 509 aa, Felis catus
MDPLQMAHSGPRKKRPRQMGASMVSPPHDIKFRDLVLYILEKKMGTTRRAFLMELARRKGFRVENELSDSVTHIVAENNSGSDVLEWLQVQNVKASSQLELLDISWLIESMGAGEPVEVTRKHQLVRRDYSASPNPELQETPPLVVKKIPLYACQRRTTLNNFNHIFTDAFEVLAENYEFKENEISSATFMRAASVLKSLPFTIISMKDTEGIPCLGDKVKCVIEEIIEDGESSEVKAVLNDERYQSFKLFTSVFGVGLKTSEKWFRMGFRTLSKIKSDKTLKFTQMQKAGFLYYEDLVSCVTRAEAEAVGVLVKEAVWAFLPDAFVTMTGGFRRGKKIGHDVDFLITIPGSTDEEEEQLLPKVINLWQRKELLLYYDLVESTFEKLKLPSRKVDALDHFQKCFLILKLHHQRVDSGKCSQQEGKTWKAIRVDLVMCPYERRAFALLGWTGSRQFERDLRRYATHERKMILDNHALYDKTKKIFLKAESEEEIFAHLGLDYIEPWERNA

SEQ ID NO: 44 - protein, 508 aa, Homo sapiens
MDPPRASHLSPRKKRPRQTGALMASSPQDIKFQDLVVFILEKKMGTTRRAFLMELARRKGFRVENELSDSVTHIVAENNSGSDVLEWLQAQKVQVSSQPELLDVSWLIECIRAGKPVEMTGKHQLVVRRDYSDSTNPGPPKTPPIAVQKISQYACQRRTTLNNCNQIFTDAFDILAENCEFRENEDSCVTFMRAASVLKSLPFTIISMKDTEGIPCLGSKVKGIIEEIIEDGESSEVKAVLNDERYQSFKLFTSVFGVGLKTSEKWFRMGFRTLSKVRSDKSLKFTRMQKAGFLYYEDLVSCVTRAEAEAVSVLVKEAVWAFLPDAFVTMTGGFRRGKKMGHDVDFLITSPGSTEDEEQLLQKVMNLWEKKGLLLYYDLVESTFEKLRLPSRKVDALDHFQKCFLIFKLPRQRVDSDQSSWQEGKTWKAIRVDLVLCPYERRAFALLGWTGSRFERDLRRYATHERKMILDNHALYDKTKRIFLKAESEEEIFAHLGLDYIEPWERNA

SEQ ID NO: 45 - protein, 506 aa, Pelodiscus sinensis
MDKTRTPSLFPQRKKQKGMHPAVSLSSCKVKFNELVIFIMERKMGVTRRTFLTQLARRKGFRVENELSDSVTHIVAENNSYAEVLEWLRGHKMRDSSRFKLLDISWFTACMEAGKPVDSEKKYQLIVQQTYPATSYTPELETSSFVAGRVSQYACQRRTTLNNCNKKFTDAFEILAGNYEFKENAVLCLAFMRAASVLKSLPFTILRMHDLEGLPCMGDEIRAVIEEIIDEGESSRVKDVLTDGRYQSFKEFSSVFGVGLKTSEKWYRMGFRTLEDVKADKTLKLSRMQKAGFLHYEDLISYVSKVEADAVSVIVEDAVRTFLPDALVTITGGFRRGKKIGHDVDFLITSPELREEEEVLHRGVLLYYDLVESTFEQAKLPSRKVDAFDHYQKCFAILKCPQQRVDTSNCDTSKESEKAKAKAWKAIRVDLVVSPFEQYAYALLGWTGSRQFERDLRRYATHERKMMLDNHALYDKTKRIFLKAKSEDEIFAYLGLDYLEPWERNA

SEQ ID NO: 46 - protein, 515 aa, Erinaceus europaeus
MDALPVVHSSPRKKRSRLMGASVAYPPYDIKFHNLVLFILEKKMGSSRRAFLMELARRKGFRVEDELSDSITHIVAENNTGSEVLEWLQVQDIKISSQLELLDVSWLVECMRAGNPVVITGKHQLVSYTVKSDASFGSNPGSQNTPPLAIKKISQYACQRRTSLNNCNHIFTDALDILAENHEFRENEVSCVAFMRAASVLKSLPFTIISMKDTKGIPCLGDKAKCVIEEIIEDGESSEVKAILNDERYQSFKLFTSVFGVGLKTSEKWFRMGFRTLNKIMSDKTLKLTRMQKAGFLYYEDLVSCVAKAEADAVSVLVQEAVWAFLPDAMVTMTGGFRRGKKLGHDVDFLITSPGATEEEEQQLLPKVINFWERKGLLLYHDLVESTFEKLKLPSRKVDALDHFQKCFLILKLHLQHVNGVGNSKTGQQEGKNWKAIRVDLVMCPYERRAFALLGWTGSRFERDLRRFATHERKMMLDNHALYDKTKRIFLKAESEEEIFAHLGLDYIDPWERNA

SEQ ID NO: 47 - protein, 505 aa, Corvus brachyrhynchos
MDRFKAPAVISQRKRQKELHSPKLSCSYEIKFSNFVIFIMQRKMGKTRRMFLMELGRRKGFRVESELSDSVTHIVAENNSYLEVLDWLKGQAVGDSSRFELLDISWFTACMEAGRPVDSELKYHLMEQRQSPPLNVPELEMPAFTATKVSQYSCQRKTTLNNYNKKFTDAFEVMAENYEFKESEIFCLEFLRAASLLKSLPFPVTRMKDIQGLPCMGDQVRDIIEEIIEEGESSRVREVLNDERYKAFKQFTSVFGVGVKTSEKWYRMGLRTVEEVKADKTLKLSKMQKAGFLYYEDLVSCVSKAEADAVSLIVKNAVCTFLPDALVTITGGFRRGKKIGHDIDFLITSPGPGEDDELLHKGLLLYCDIIESTFVKEQLPSRKVDAMDHFQKCFAILKLCQPRVDNSTCNTSKKLEMAEVKDWKAIRVDLVITPFEQYAYALLGWTGSRQFGRDLRRYASHERKMVLDNHALYDRRKRIFLKAGSEEEIFAHLGLDYVEPWERNA

SEQ ID NO: 48 - protein, 519 aa, Sarcophilus harrisii
MHRIRTTDSDHGKKRQKKMDAISSKLYEIKFHEFVLFILEKKMGATRRTFLMDLARKKGFRVESELSNSVTHIVAENNSGSDVLAWLEAHKLETTAHFELLDVSWLIECMKVGKPVDTKGKYQLVVILVQRQEANPDPNEGMLKIQSPAMNAISPYACQRRTTLNNHNQRFTDAFEILAKNYEFRENHGHCLTFLRATSVLKCLPFAIVSMKDAEGLPWIGDEVKGIMEEIIEDGQSLEVQAVLNDERYQAFKLFTSVFGVGLKTAEKWYRMGFRTLSKIQSDKSLKFTKMQKAGFLYYEDLISCVSKAEADAVSLIVKEAVWTFLPDALITITGGFRRGKEFGHDVDFLITSPGGEKEQVDQLLQKVTNLWEKQGLLLYYDLMESTFEDLKLPSRKVDALDHFQKCFLILKLYCQRGDRSKWEGPEGSNGLQTKNWKAIRVDLVVCPYDRYAYALLGWSGSRQFERDLRRYATHEKKMMLDNHALYDKTKRTFLKAESEEEIFSHLGLEYIEPWERNA

SEQ ID NO: 49 - protein, 525 aa, Raja eglanteria
MSSVEKLASLGITPKRRKQKEEGLCGSHSQYEVRFRDLLIYVVERKMGSSRRMFLMDLARKKGFRVADIMSDSVTHIVTENNSWNEIWDWIQTQKMSDADKLNLLDISWFTDSMGAGKPVDIKEHHRLQMQKIQPLQSDVSSTSSVTHVSQYACQRKSTLDNKNKIFTDTLELLAENCEFDENAGSFVAYSRATSVLKSLPYPITGMNDLEGLPCIGDQTRVIIEELLEEGVCSKVESLLCDEKYKARKLFTSVFGVGVKTADKWYGLGFRTLEEIKARKDITFTKMQKAGFLYYEDILQAVKKSEAEAVIQIIGDIVGQCAPDAKVTLTGGFRRGKEVGHDVDLLITCLEEGNEEGVLHKAISKLDRHGLLLFCDVVEATMEKRQLPSRKYDAMDHFQKCFLILKLDKRLVNKRDYGLASGSAVRLTDKRTEDENKTMIKGWKAIRVDLVIVPSQQFAYALLGWSGSRQFERDLRRYCSQEKRMLLDNHGLYDKNTQEFLKAETEEEIFAHLGLEYIEPQERNA

SEQ ID NO: 50 - protein, 530 aa, Mus musculus
MDPLQAVHLGPRKKRPRQLGTPVASTPYDIRFRDLVLFILEKKMGTTRRAFLMELARRKGFRVENELSDSVTHIVAENNSGSDVLEWLQLQNIKASSELELLDISWLIECMGAGKPVEMMGRHQLVVNRNSSPSPVPGSQNVPAPAVKKISQYACQRRTTLNNYNQLFTDALDILAENDELRENEGSCLAFMRASSVLKSLPFPITSMKDTEGIPCLGDKVKSIIEGIIEDGESSEAKAVLNDERYKSFKLFTSVFGVGLKTAEKWFRMGFRTLSKIQSDKSLRFTQMQKAGFLYYEDLVSCVNRPEAEAVSMLVKEAVVTFLPDALVTMTGGFRRGKMTGHDVDFLITSPEATEDEEQQLLHKVTDFWKQQGLLLYCDILESTFEKFKQPSRKVDALDHFQKCFLILKLDHGRVHSEKSGQQEGKGWKAIRVDLVMCPYDRRAFALLGWTGSRQFERDLRRYATHERKMMLDNHALYDRTKGKTVTISPLDGKVSKLQKALRVFLEAESEEEIFAHLGLDYIEPWERNA

SEQ ID NO: 51 - protein, 487 aa, Latimeria chalumnae
MEKFIFPSLSPHKKKQKVTEPLKSFGNYEIKFKDIVIFIMERKMGSSRRMFLTELARKKGFQVESVLSDSVNHIVAENNSCAEVLEWIHKQNLRNNPKMEVLDITWFTESMGAGKPVEIEKRHRLMVRLNCNIPRSNSSNTPPESSSVVAISPYACQRRTSLNNYNKLFTDAFEVLAENYEMNENKGPYLGFMRAASMIKSLPYAISSMKDLEGLPCLGDQTKAVIEQCCQDTEKSTIKSCINFARSQSIILFTSVFGVGQKTAEKWFRKGLRTFEEVQVHKETKLTKMQIAGFLYYEDLSSFVTKPEADAIGQIIEDTVRLFMPDALVTLTGGFRREKKIGHDVDFLITTPVPGNENGLLEKVIDVLHQQGILLYCDVVESTFDKSRLPSRKVDALDHFQKCFAILKLLKQKVITSNCEEAEEPSNTSTKEWKAIRVDLVITPFDQYAFALLGWTGSRQFERDLRRFATHERKMMLDNHALFDKNK

SEQ ID NO: 52 - protein, 492 aa, Oryctolagus cuniculus
MGASRTSLPQDVKFRDLVLFILEKKMGTTRRAFLMELARRKGFRVENELSDSVTHIVAENNSGSDVLEWLQVQKVKDSSQLELLDVSWLIECMGAGRPVEMTGKHQLVVRQDYPASPNPGSQEAPALAVHRISEYACKRRTTLNNCNRIFTDAFEILAENSEFRENEDSYVTFIRAASVLKSLPFPIVSMKDTEGIPCLGDKVKGIIEEIIEEGESSEVKAVLNDERYQCFKLFTAVFGVGLKTSEKWFRMGFRTLSKIREDKSLKFTRMQQAGFRYYEDLVSCVTRAEAEAVDVLVKEAVRAYLPGAFITMTGGFRRGKKIGHDVDFLITSPESTEEDEQQLLHKVVNLWEKKGLLLYHDFMESTFEKLKQPSRKVDALDHFQKCFLILKLPHERVDSDRPSQQEGKNWKAIRVDLVMCPYECHAFALLGWTGSRQFERDLRRYATHERRMILDNHALYDKTKRMFLQAESEEEIFAHLGLDYIEPWERNA

SEQ ID NO: 53 - protein, 504 aa, synthetic construct
MDRIRAPAVFSQRKRQKGTHSPNLSCSYEIKFNKFVIFIMQRKMGMTRRTFLMELARRKGFRVENELSDSVTHIVAENNSYLEVQDWLRGQAVGDSSRFELLDISWFTACMEAGRPVDSELKYRLMEQDQSPPLNAPESEVPSFIASKVSQYSCQRKTTLNNYNQKFTDAFEIMAENYEFKENEIFCLEFLRAASVLKFLPFPVTTMKDIQGLPCMGDRVRDVIEEIIEEGESSRAKEVLSDERYQSFKQFTSVFGVGVKTSEKWYRMGLRTLEEVKADRTLKLSKMQKAGFLYYEDLVSCVSKAEADAVSLIVKNTVCRFLPDALVTITGGFRRGKEIGHDIDFLITNPGPREDDELLHKGLLLYCDMIESTFVKEQLPSRKVDAMDNFQKCFAILKLYQPRVDNSSYNMSKKFHIEVKDWKAIRVDLVITPFEQYAYALLGWTGSRQFGRDLRRFANHERKMILDNHALYDRRKRIFLKAGSEEEIFAHLGLDYVEPWERNA

SEQ ID NO: 54 - protein, 507 aa, Xenopus laevis
MNPLSQSALVPLRKKAKMAPISQSFCQHNVKFKEIVLFLVERKMGSSRRTFLMELARKRGFQTEIELSDSVTHIVAENNSGAEVLEWLQSKKLGFTVKTHILDISWFTECMEAGRPVEIQNRHLLPVQQDCSANFNPPLSSSCVQVSQYACQRCTTLQDTNRIFTDAFDILAEHFEFCENKGRTVAFLRASSLIKSLPFPITAMKELEGLPWLGDQMKGIIEEILEEGKSYKVLEVMNEERYKSFKQFTSVFGVGLKTSDKWFRMGFRTLEEIKNEKELKLTKMQKCGLLYYEDITSYVSRAEAETTEQLIKSIVWKFVPDAIVTLTGGFRRGKKKGHDVDILITCARKGKEKNILHNTMSVLKNRGLLLFYNIIESTFDETKLPSRHVDALDHFQKCFTILKLPKRQMDIGNIIDPHECERKNWKAVRLDLVITPYEQYPYALLGWTGSRQFERDLRRYATHEKRMMLDNHGLYDKTKNNFLKANNEEDIFKQLGLDYLEPWERNA

SEQ ID NO: 55 - protein, 507 aa, Xenopus laevis; identical to SEQ ID NO: 54

SEQ ID NO: 56 - protein, 498 aa, Takifugu rubripes
MFHATALPRMRKRPRPEEVACPGREDVKFRDVRLYLVEMKMGRSRRSFLTQLARSKGFMVEEVLSNRVTHVVSESSQAPVLWAWLKERAPQDLPNMHVVNITWFTDSMRESRPVAVETRHLIQDTLPAIPEGGAPAAEVSQYACQRRTTTDNYNVVFTDAFEVLAECYEFNQMDGRCLAFRRAASVLKSLPRGLSSLEETHSLPCLGGHAKAIIGEILQHGRAFDVEKVLSDERYQTLKLFTSVYGVGPKTAEKWYRSGLRSLDHILADQSIQLNHMQQNGFLHYGDISRAVSKAEARALTKAIGETVQAITPDALLALTGGFRRGKEFGHDVDIIFTTLELGMEENLLLAVIKSLEKQGILLYCDYQASTFDLTKLPTHSFEAMDHFAKCFLILRLEASQVEEGLNSPVEDIRGWRAVRVDLVSPPVDRYAFALLGWTGSRQFERDLRRFARKERRMLLDNHGLYDKTKEEFLAAGTEKDIFDHLGLEYMEPWQRNA

SEQ ID NO: 57 - protein, 504 aa, Gavia stellata
MDRIRAPAVFSQRKRQKAMHSPNLSCSYEIKFNKFVIFIMERKMGVTRRSFLMDLARRKGFRVESELSDSVTHIVAENNSYSEVLDWLKGQAVGDSSRFELLDISWFTACMEAGRPVDSEMKYRLMEQDQSPPLNTPESEVPSFIASKVSQYSCQRKTTLNNYNKKFTDAFEIMAENYEFKENEIFCLEFLRAASVLKFLPFPVTRMKDIQGLPCMGDRVRDVIEEIIEEGESSRAEEVLNDERYKSFKQFTSVFGVGVKTSEKWYRIGLRTLEEVKADKTLKLSKMQRAGFLYYEDLVSCVSKAEADAVSLIVKNTVCTFLPDALVTITGGFRRGKKIGHDIDFLITNPGPREDDELLHKGLLLYCDIIESTFVKEQLPSRKVDAMDNFQKCFAILKLYQPRVNSSYNTSKNFDMAEVKDWKAIRVDLVITPFEQYAYALLGWTGSRQFGRDLRRYATHERKMILDNHALYDRRKRIFLKARSEEEIFAHLGLDYVEPWERNA

SEQ ID NO: 58 - protein, 509 aa, Heterocephalus glaber
MDPLQTAHLGPRKKRPRQTGTLMASGPHNIRFGDLVLFILEKKMGTTRRAFLMELARKKGFRVENELSDSVTHIVAENNSGNDVLEWLQVQNIQASSQLELLDVSWLIECMGAGKPVEMTGRHQLVVRDSPASPNPGPQKTPSLAVQKIPEYACQRRTTLDNCNYIFTNAFEILAEDCEFRENEGFYVTYMRAASVLKSLPFTIISMKDTEGIPCLGGRVKCIIEEIIEDGESSEVKAVLNNERYKSFKLFTSVFGVGLKTSEKWFRMGFRSLSKIRSDKSLTFTRMQKAGFLYYEDLVSCVTRAEAEAVNMLVKEAVWTFLPGAFISMTGGFRRGKEIGHDVDFLITSPEVTEDEEQQLLHKVINLWEKKGLLLYSDLVESTFEKLKLPSRKVDALDHFQKCFLILKLHHQRVDNDKSPQQGGKTWKAIRVDLVMCPYERRAFALLGWTGSRQFERDLRRYATHERKMILDNHALYDKTKKTFLKAESEEEIFTHLGLDYIEPWERNA

SEQ ID NO: 59 - protein, 500 aa, Xenopus laevis
ALVPLRKKAKMAPISQSFCQHNVKFKEIVLFLLERKMGSSRRTFLMELARKRGFQTEIELSDSVTHIVAENNSGAEVLEWLQSKKLGFTVKTHILDISWFTECMEAGRPVEIQNRHLLPVQQDCSANFNPPLSSSCVQVSQYACQRCTTLQDTNRIFTDAFDILAEHFEFCENKGRTVAFLRASSLIKSLPFPITAMKELEGLPWLGDQMKGIIEEILEEGKSYKVLEVMNEERYKSFKQFTSVFGVGLKTSDKWFRMGFRTLEEIKNEKELKLTKMQKCGLLYYEDITSYVSRAEAETTEQLIKSIVWKFVPDAIVTLTGGFRRGKKKGHDVDILITCARKGKEKNILHNTMSVLKNRGLLLFYNIIESTFDETKLPSRHVDALDHFQKCFTILKLPKRQMDIGNIIDPHECERKNWKAVRLDLVITPYEQYPYALLGWTGSRQFERDLRRYATHEKRMMLDNHGLYDKTKNNFLKANNEEDIFKQLGLDYLEPWERNA

SEQ ID NO: 60 - protein, 473 aa, Poecilia formosa
MFHAPAAPRPRKRSKPGEDSVSRREEATFQDVRIFLVERKMGRSRRSFLTQLARSKGFVVEDILSDAVTHVVSEDSQSSSLWPWLKSRSLSDLSTVNVLDISWFTDSMKEGRPVPVETKHLIQDILPEAPKAAPVNKVSQYACQRRTTIENNNRIFTDAFEVLAENYEFNEIEGRCLAFRRAASVLKSLPWAVRSVGATQDLPCLGEHTKAVMKEILQYGRSFEVEKILSDERCQTLKLFTSVFGVGPKTAEKWYRRGLRSFSDVLAQPDIHLNRMQQSGFLHYGDISRAVSKAEARAVGNIIDEAVHVITPNAILALTGGFRRGKDFGHDVDYVVTTTELGKEENLLISIIESLKKQGLLLFSDYQASTFDLSKLPSHRFEAMDHFAKCFLILRLEGSRVEGGLQRAQGDGRGWRAVRVDLVSPPADRFAFTMLGWTGSRMFERDLRRFARLERQMLLDNHALFDKTKVLFI

SEQ ID NO: 61 - protein, 518 aa, Monodelphis domestica
MHRIRTIDSDFGKKRQKKMDNHISSMIYEIKFHEFVLFILEKKMGATRRTFLTDLARKKGFRVENELSNSVTHIVAENNSGSDVLAWLKTHKMEKTTQFELLDISWLIECMKVGKPVDTKGKYQLMESRVDSANPDPTAGTLNILPPTTKTISQYACQRRTTINNHNQRFTDAFEILAKNYEFKENDDTCLTFMRAISVLKCLPFEVVSLKDTEGLPWIGDEVKGIMEEIIEDGESLEVQAVLNDERYQSFKLFTSVFGVGLKTADKWYRMGFRTLNKIRSDKTLKLTKMQKAGLCYYEDLIDCVSKAEADAVSLLVQDAVWTFLPDALVTITGGFRRGKEFGHDVDFLITSPGAEKEQEDQLLQKVTNLWKKQGLLLYCDLIESTFEDLKLPSRKIDALDHFQKCFLILKLYHHKEDKRKWEMPTGSNESEAKSWKAIRVDLVVCPYDRYAFALLGWSGSRQFERDLRRYATHEKKMMLDNHALYDKTKKIFLKAKSEEEIFAHLGLEYIQPSERNA

SEQ ID NO: 62 - protein, 531 aa, Loxodonta africana
MDPLQMAHTGPRKKRARPMGASMATTPQDIKFQDLVLFILEKKMGTSRRAFLMELARRKGFRVENELSDSVTHIVAENNSGSDVLEWLQVQKIKASSQLELLDVSWLIECMGAGKPVEMTGKHQLVVRGDYSASSNPSPQKTPPPAVKKISQYACQRRTTLNNHNNIFTDAFEILAENCEFRENEGSYVVFMRAASVLKSLPFTIISMKDTQGIPCLEDKAKCVIEDIIEDGESSEVKAVLNDERYKSFKLFTSVFGVGLKTSEKWFRMGFRSLSKIRTDKTLKFTEMQEAGFLYYEDLVSCVTKAEADAVSVLVKEAVWAFLPDAFVTMTGGFRRGKKVGHDVDFLITSQGSTEEEQQQLLHKVLNLWKKEGLLLYSDLIESTFEKLKLPSRKVDALDHFQKCFLILKLHHQRIDNSKSSQLQGKTWKAIRVDLVMCPYECHAFALLGWTGSRQFERDIRRYASHERKMILDNHALYDKTKRWATLTLNPKAGSLSTLQEFIRIFLKAGSEEEIFAHLGLDYIEPWERNA

SEQ ID NO: 63 - protein, 505 aa, Taeniopygia guttata
MDRFKAPAVISQRKRQKGLHSPKLSCSYEIKFSNFVIFIMQRKMGMTRRMFLMELGRRKGFRIESELSESVTHIVAENNSYLEVLDWLKQQAVGDSSRFELLDISWFTACMEAGRPVDSEMKYRLMEQSPSPPLNTPELEMPALIATKVSQYSCQRKTTLNNYNKKFTDAFEVMAENYEFKENEIFSLEFLRAASLLKSLPFSVTSMKDIQGLPCVGDQVRDIIEEIIEEGESSRVKEVLNDERYKAFKQFTSVFGVGVKTSEKWYRMGLRTVEEVKAEKTLKLSKMQKAGILYYEDLVSCVSKAEADAVSLIVKNTVCTFLPDALVTITGGFRRGKNIGHDIDFLITNPGPREDDELLHKGLLLYCDIIESTFVKEKLPSRKVDAMDHFQKCFAILKLYQPRVDNSTCNTSKKLEMAEVKDWKAIRVDLVITPFEQYSYALLGWTGSRQFGRDLRRYAAHERKMILDNHALYDRRKRIFLKAGSEEEIFAHLGLDYVEPWERNA

SEQ ID NO: 64 - protein, 497 aa, Xiphophorus maculatus
LISKEMFHAPATPRPKKRSKPAQDSFCRREEATFQDVRIFLVERKMGRSRRSFLTQLARSKGFVVEDILSDAVTHVVSEDSQSSSLWAWLKGRSLSDLSTVNVLDISWFTDSMREGRPVPVETKHLIQVNMNEKMNPTAGKREAPKAAPVNTVSQYACQRRTTTENNNRIFTDAFEVLAENYEFNDIEGRCLAFRRAASVLKSLPWAVRCVGATQDLPCLGEHTKAVMKEILQYGRSFEVEKILSDERCQTLKLFTSVFGVGPKTAEKWYRRGLRSFSEVLAQPGIHLNRMQQSGFLHYGDISRAVSKAEARAVGNIIDEAVHVITPNAILALTGGFRRGKDFGHDVDFILTTTELGKEENLLISIIESLKKQGLLLFSDYQASTFDISKLPSHRFEAMDHFAKCFLILRLEGSLVEGGLQRAQGDGREWRAVRVDLVSPPVDRYAYTMLGWTGSRMFERDLRRFARLERQMLLDNHALYDKTKFSFEEKNADEHSF

SEQ ID NO: 65 - protein, 518 aa, Monodelphis domestica
MHRIRTIDSDFGKKRQKKMDNHISSMIYEIKFHEFVLFILEKKMGATRRTFLTDLARKKGFRVENELSNSVTHIVAENNSGSDVLAWLKTHKMEKTTQFELLDISWLIECMKVGKPVDTKGKYQLMESRVDSANPDPTAGTLNILPPTTKTISQYACQRRTTINNHNQRFTDAFEILAKNYEFKENDDTCLTFMRAISVLKCLPFEVVSLKDTEGLPWIGDEVKGIMEEIIEDGESLEVQAVLNDERYQSFKLFTSVFGVGLKTADKWYRMGFRTLNKIRSDKTLKLTKMQKAGLCYYEDLIDCVSKAEADAVSLLVQDAVWTFLPDALVTITGGFRRGKEFGHDVDFLITSPGAEKEQEDQLLQKVTNLWKKQGLLLYCDLIESTFEDLKLPSRKIDALDHFQKCFLILKLYHHKEDKRKWEMPTGSNESEAKSWKAIRVDLVVCPYDRYAFALLGWSGSRQFERDLRRYAIHEKKMMLDNHALYDKTKKIFLKAKSEEEIFAHLGLEYIQPSERNA

SEQ ID NO: 66 - protein, 505 aa, synthetic construct
MDRIRAPAISSQRKRQKTMHSPNLSCSYEIKFSKFVIFIMQRKMGMTRRTFLMELGRRKGFRVESELSDCVTHIVAENNSYPEVLDWLRGQAVGDSSRFELLDISWFTACMEAGRPVDSETKYRLVEQDQPLPLNTSESEVPSFIASKVSQYSCQRKTTLNNYNMKFTDAFEIMAENYEYKESEILCLEFLRAASVLKFLPFPVTRMKDIQGLPCMGDRVRDVIEEIVEEGESSRAKEVLNDERYKSFKQFTSVFGVGVKTSEKWYRKGLRTLEDVKADKTLKLSKMQKAGFLYYEDLVSCVSKAEADAVSLIVKNTVCTFLPDALVTITGGFRRGKKIGHDIDFLITNPGPREDDELLHKGLLLYCDIIESTFAKEQIPSRNIDAMDNYQKCFAILKLHQPRADNSSYNTSKKFDTAEVKDWKAIRVDLVITPFEQYAYALLGWTGSRQFGRDLRRYASHERKMMLDNHGLYDRRKRIFLKAGSEEEIFAHLGLDYVEPWERNA

SEQ ID NO: 67 - protein, 496 aa, Chinchilla lanigera
MLPKRRRARARSPGGAAASSALSSVLFPGVAIYLAEPRMGRSRRAFLTRLALSKGFRVLDAYSSEVTHVVMERTSAEEAVCWQEQKAAPPPGRPRPALLDISWFTESMAAGQPVPVEGRHRLEVAKPRKGPPNPAAAAAMPAYACQRSTPLMHHNSSLSEALETLAEAAGFEGSEGRLLSFHRAASVLKALPWPVTALSQLQGLPHFGEHSSSVIQELLERGVCEEVERVRCSERYRTMKLFTQIFGVGVKTANQWYQEGLRTLDELREQPQRLTRRQQAGLQHHLDLCTPVGRPDAEALQQLVEATVGQALPGATVTLTGGFRRGKLQGHDVDFLITHPEEGREVGLLPSVMSQLQSQGLVLYHQHQPSHLGDPGHLARQSHTMDAFERSLCILRLPKPSGAAKGADSEPRSTWKAVRVDLVVAPWSQFPFALLGWTGSKLFERELRRFSRKEKGLCLNSHGLFDPEQACFPVASEEDIFRHLGLEYLPPEQRNA

SEQ ID NO: 68 - protein, 510 aa, Dasypus novemcinctus
MDWLRIARSGPRKKRPRQMDASAVSSPHNIKFQDLVLFILEKKMGSTRRTFLTELARRKGFRVENELSESVTHIVAENNSGSDVLEWLQLQKIEASSQFELLDVSWLIECMGAGKPVETTGKHQLVVKQDCSASPDPGHQKTLPLAIKKISPYACQRRTTLNNQNQIFTDAFDILAENCELRENEESYLVFARAASVLKSLPFTIISMKDTEGIPCLGDKAKCVIEEIIEDGESSEVKAVLNDERYSSFKLFTSVFGVGLKTSEKWFRMGFRTLSQIRSNESLKLTRMQKAGFLYYEDLVSCVSRAEAEAVSVLVQEAVWAFLPDAFVTMTGGFRRGKKSGHDVDFLITSPGATEEEEEELLQKVINLWGKEGLLLYYDLVESTFEKLKLPSRKVDALDHFQKCFLILKLPYQRVDSGKSSWQEGKAWKAIRVDLVMCPYERRAFALLGWTGSRQFERDLRRYATHEQKMILDNHALYDKTKRIFLPAESEEEIFAHLGLDYIEPCERNA

SEQ ID NO: 69 - protein, 478 aa, synthetic construct
MFHTPIVPRARKRSRPAEASAPRRERVKFEDVRLYLVERKMGRSRRSFLTELARSKGFIVEDVLSDVVTHVVSEDSQASSLWAWLKGGPVKNLPVMHVLDIDTLAASPEATTPTPMSTVSQYACQRRTTTKNNNKIFTDAFEVLAESHEFNDMEGPCLAFRRAASVLKSLPWTVQNLRVTEDLPCLGEHSMCVIEEILQHGHSFEVEKILSDERYQILKLFTSVFGVGPKTAEKWYRRGLRSFSDVLAEPDIHLNRMQQSGFLHYGDISRAVSKAEAQALGNIIDEAVRAITPDAILTLTGGFRRGKDFGHDVDFIVTTPQLGKEERLLTSVIDRLKQQGILLYCEYQASTFDESKLPSHRFEAMDHFAKCFLILRLEDSQVEGGLQTAEEDRRGWRAVRVDLVSPPVDRYAFTLLGWSGSRQFERDLRRFARMERRMLLDNHALYDKTKKEFLAATTEKDIFAHLGLEYIEPWQRNA

SEQ ID NO: 70 - protein, 573 aa, Myotis brandtii
MDPRGILKAFPKRKKILANPSSKVLAKIPKREDEEEAREWLSSLRAHVVPAGIGRARAELFEKQIVQHGGQIYSAQAPGVTHIVVDDGMDCERALRLLRLPRLPPGAQLVKSAWLSLCLQERRLVDTAGFSIFIPNRYLDQPQLSKADQDSSPGACEALLRTVPSSPPTPPRPVSPPQRIEEAPNTQAQPVSDDDTSDGEETPVSAADLEALISGRYPIPPEGDGEPSPAPEGLNKWVCAQPSSQKATNHNTHITEKLEVLAKAYSVQGDKWRALGYDKAINALKSFHKPVTSYQEACSIPGIGKRMAEKIVEILESGHLRKLDHISDSVPVLELFSNIWGAGTKTAQMWYQQGFRSLEDIRNQASLTTQQAIGLKHYDDFLERMPREEAAEIEQTVRESAQAFNPGLLCVACGSYRRGKATCGDVDVLLTHPDGRSHQGIFSRLLDSLRQRGFLTDDLVSQEENGQQQKYLGVCQLPGPGRRHRRLDIIIVPYSEFACALLYFTGSAHFNRSMRALAKTKGMSLSEHALSAAVVRDTRGLKVGSGRVLPTPTEKDVFRLLGLPYREPAERDW

SEQ ID NO: 71 - protein, 314 aa, Myotis brandtii
LANFEKNVNQAIHKYNAYRKAASVIAKYPHKIKSGAEAKKLPGVGTKIAEKIDEFLATGKLRKLEKIRQDDTSSSINFLTRVTGIGPSAARKFVDEGIKTLEDLRKNEDKLNHHQRIGLKYFEDFEKRIPREEMLQMQDIVLNEVKKVDSEYIATVCGSFRRGAESSGDMDVLLTHPSFTSESNKQPKLLHRVVEQLQKVCFITDTLSKGETKFMGVCQLPSKNDGKEYPHRRIDIRLIPKDQYYCGVLYFTGSDIFNKNMRTHALEKGFTINEYTIRPLGVTGVAGEPLPVDSEKDIFDYIQWKYREPKDRSE

SEQ ID NO: 72 - protein, 764 aa, Lepisosteus oculatus
MVPLKRRKTVRNDVNCERNEVIKFPDVVIFLLERRMGASRRAFLTRLGRNKGFRVEDCYSDAVTHVVSENNTGEEVVDWLDRQIPGGWTPRPVHLLDISWFTESMGAARPLDVQDAHRLKVKAVSQAGGGTRTVSPYACGRRTPLQHHNRALTDALEVLAENERFRKPPGSSVATGRPDVLYRPLGFKLGCTPPPRCGKHSQQNRVLWIALQSSQRRLSLYTDYTEAFQLLRLPPFHFSLIKCTTDNLTQRGLQVLALRTRSQREFSSGQGTGVRHYEDLSTPITKEEAHAIGQIVEEAVHTVLPGAELTITGGFRRGKKTGHDVDFLITHPEEGKEVGLLPKVISWLDSQDLLLYHRVKDNTYSESKVQLARSQSSMDHFERCFSIFRLNRPLARPPEPGASSGGTAQESAASGGHCSGEPRSWKAVRVDLVVTPVSQFAFGLLGWTGSQHFERELRRWAGQEKHMTLNSHALYDRTQGSHRAKDHHQTIATSMASSPPLSLSRWLSPLHHRAHTRERYCEPSAPPTESMAIASHEKGEIQAHMPVEYSRGGKRISLHTLEPEIASGTEEEETSGYESEGGQSASPADPPGGSSSSPPTPPGRRPRTAFTSEQISRLERTFKKHAYLGTREKEELCRKLNLSEKQIKNWFQNRRMKLKRTLQDALAQACHVKVASQLLHYPELQAFGPSAYSGYYPNQDSTAAYLPLPGLPYAPSQALGHLSALPLEAQVHPYSVPPFVMPPHSAGTGSPPVMARYHPYAPRY

SEQ ID NO: 73 - protein, 560 aa, Lepisosteus oculatus
MEARGIVKAFRKVKRIGHQLEKEEEPQNKKQQKELVTGTWLNGICAYIIQTGIGNARATIFQTQIVQNGGQVVDTFSPCVTHVIVDDSMNYDRALRLLKVDKLPPAVQLVKCSWLSLCITEKKLLNTAGYSVFIPDRNLDSNHEQTNNKQKTEKEATVSKTVQIEESASLNTIISSHRAQTSDDDGSDTEEAGVSQKDLEALLTGCYPTTEEPSPAQPDPVTGKWVCAQSSKAKNDNHNQHITDKLEVLAKAYTHQGDKWRALGYSKAINALKSYHKPVSSYEEACKIRGIGKRMAEKIKEILESGNLRKLDHIGESVPVLELFTNIWGVGSKTAQMWYQQGFRTLEDIRTKATLTSQQVIGLKHYDDFLDRMPREEAAEIEKTVKEAALSLNPGLLAVACGSYRRGKPTCGDVDILITHPDGKSHKGIFSKILHILHQSGFLTDDLVSHEENGEQKKYLGVCRLPGPESCHRRLDIIVVPYSEFACAILYFTGSAHFNRSMRALAKTKNMSLSEHSLNKDVLRQGSLKVSTGAPLPTPTEKDVFRHLGLPYREPQDRDW

SEQ ID NO: 74 - protein, 579 aa, Lepisosteus oculatus
MEARGIVKAFRKVKRIGHQLEKEEEPQNKKQQKELVTGTWLNGICAYIIQTGIGNARATIFQTQIVQNGGQVVDTFSPCVTHVIVDDSMNYDRALRLLKVDKLPPAVQLVKCSWLSLCITEKKLLNTAGYSVFIPDRNLDSNHEQVKVNSLNGNGSALGIHVQQTNNKQKTEKEATVSKTVQIEESASLNTIISSHRAQTSDDDGSDTEEAGVSQKDLEALLTGCYPTTEEPSPAQPDPVTGKWVCAQSSKAKNDNHNQHITDKLEVLAKAYTHQGDKWRALGYSKAINALKSYHKPVSSYEEACKIRGIGKRMAEKIKEILESGNLRKLDHIGESVPVLELFTNIWGVGSKTAQMWYQQGFRTLEDIRTKATLTSQQVIGLKHYDDFLDRMPREEAAEIEKTVKEAALSLNPGLLAVACGSYRRGKPTCGDVDILITHPDGKSHKGIFSKILHILHQSGFLTDDLVSHEENGEQKKYLGVCRLPGPESCHRRLDIIVVPYSEFACAILYFTGSAHFNRSMRALAKTKNMSLSEHSLNKDVLRQGSLKVSTGAPLPTPTEKDVFRHLGLPYREPQDRDW

SEQ ID NO: 75 - protein, 499 aa, Sarcophilus harrisii
MALVPPKRRRGAGRGEEAGGAAAPSPAPLRFPGLTIYLAERHMGRSRRAFLTGLARAKGFRVDQAYSPEVTHVVMEGSSATEASGWLDRVLGASGSLPRPLLLDISWFTESMGQGKPVPVEGRHCLGVPLPTRSQADPGCLPAYACQRHSPLNHHNLCFTEALDTLAEAAGFEGSEGRFLSFRRAASVLKALPGPITSISQLRGLPHFGDHSSRIVQELLECGVSSEVERIKQSERYQTMKLFTHIFGVGVKTADKWYRDGLRSLADLQGQTRKLSRQQEAGICHFEDLNTLVWRHEAEAIQRVVEKAVRQVLPGATVTLTGGFRRGKLQGHDVDFLITHPVEGQEEGLLPKVMDLLESQGFVLYRHTQSNHYQDLKDPAQSTSLFDAYERCFSILRLPDPTAAFRPEAGEEPCRDGKAVRVDLVVAPASQFAFALLGWTGSQHFERELRRFSRAEKQLLLNSHGLYVPGKKESFPAASEEDIFRHLGLEYIAPEYRNA

SEQ ID NO: 76 - protein, 590 aa, Sarcophilus harrisii
MPRPRPCHPLLAAAPEREGPGWALAMEPRGVLKAFPRRKKRSSGSDRSALLKIPKKEGLEGGEWLSPLQVHVLPAGIGRARAEIFEKQIIQHGGRICSPQAPGITHIVVDEAVDCERALRLLKLSQLPLGVQIVKSAWLSQCLQEQKLVDTTGFSIFIPDRYLDETDNQVTSFQPGCSGTSAQAGLPSAAAFAPPQEPHSELNTQTQPNSYESSDEEEVQVTPADLEALITGQYPSTPEGDAMPCLAPTVSDKWVCAQPSSQKRTNHNSHITEKLEVLAKAYAVQGDRWRSLSYSKAINALKSFHKPVSSYQEACGIPGIGKRMAEKIMEIVESGHLRKLDHISDSVPVLELFSNIWGVGSKTAQMWYQQGFRTLEDIESRATLTSQQAIGLKHYEDFLKRIPREEASEIEQTVREAAHALNPGLLSVACGSYRRGKATCGDVDVLVTHPDGRSHQGIFGQLLDTLRQQGFLTDDLVSQDDNGQQQKYLGVCRLPGPGRLHRRLDIIVVPYSEFACALLYFTGSAYFNRSMRALAKTKGMSLSERSLYTAVVRDGRGLKVGPGRALPTPTEKDVFRLLGLPYREPSERDW

SEQ ID NO: 77 - protein, 319 aa, Sus scrofa
MRAASVLKSLPFTIISMKDTEGIPCLGDKVKCVIEEIIEDGESSEVKAVLNDERYQSFKLFTSVFGVGLKTSERWFRMGFRSLSKIRSDKTLKFTRMQKAGFLYYEDLVSCVTRAEAEAVGVLVKEAVQAFLPDAFVTMTGGFRRGKKMGHDVDFLITSPGSTDDEEQQLLPKVVNLWEREGLLLYCDLVESTLEKSKLPSRNVDALDHFQKCFLILKLHHQRVDSGMSSQQEGKTWKAIRVDLVMCPYELRAFALLGWTGSRQFERDLRRYATHERKMILDNHALYDKTKRIFLKAESEEEIFAHLGLDYLEPWERNA

SEQ ID NO: 78 - protein, 454 aa, Sus scrofa
MLPRRRRARVGPPEAAPSSAARFPGVAIYLAEPRMGRSRRAFLTRLALSKGFRVLDAYSPEVTHVVMEGTSAEEAISWQEHRTPSLPPGCSHPALLDVSWFTESMAAGQPVPVERRHRLEVAVPREELPSPVWMLPYACQRPTPLTHHNASLSEALETLAEAADFDGSKGRHVSFCRAASVLKALPSPVTALSQLQGLPHFGEHSRRVIQELLEHGVCEEVERVRLSERYQTMKLFTQIFGVGVRTADQWYQEGLRTLDDLREQPQRLTKQQKAALPGPERPDPAVRSGGPAAGGGGSCGAGPAGGHRNAGRRLPEGLVLYHQHQRGQQGDPTHLAQKPHAMDAFEMSLCIFRLPRPPEAAVGGPREPCPPWKAVRVDLVVTPISQFPFALLGWTGSKHFERELRRFSRKERGLWLNSHGLFDSEQKMPFHVASEEDIFGLLGLEYLPPEQRNA

SEQ ID NO: 79 - protein, 575 aa, Sus scrofa
MDPRGILKAFPKRRKIHTSPSSKALAKIPKREDGEEAGEWLSSVRAHVVPTGIGRARAELFEKQIVQHGGQLCPAQAPGVTHIVVDEGMDCERALRLLRLPRLPPGAQLVKSAWLSLCLQERRLVDTAGFSIFIPERYLDQAQLSKAGNDSSTSPGARETPLRTALSPPSPPTRPVSPSERTEEFASIQAQPGSDGDTSDGEETQVSAADLEALISGRYPTPLEEDGEPSPAPKGLDKWVCAQPSSQKATNHNPHITEKLEVLAKAYSVQGDKWRALGYAKAINALKSFHKPVTSYQEACAIPGIGKRMAEKILEILESGHLRKLDHISESVPVLQLFSNIWGAGTKTAQLWYHQGFRSLEDIRNQASLTTQQAIGLKHYHDFLDRMPREEASEIEQTVREAAQAFNPGLLCVACGSYRRGRATCGDVDVLLTHPDGRSHQGIFSRLLDSLRQRGFLTDDLVSQEQHGQQQKYLGVCQLPGPGRRHRRLDIIVVPYSEFACALLYFTGSAHFNRSMRALAKTKGMSLSEHALSTAVVRDAHGLKVGLGRVLPTPTEKDVFRLLGLPYREPAERDW

SEQ ID NO: 80 - protein, 491 aa, Otolemur garnettii
MLPKRRRARIGSPGGNAASSERPSTRFPGITIYLVERRMGRSRRAFLTRLARSKGFRVLDACSSEVTHIVMEQTSAEEAVCWQEHRATAAPSECTPATLLDISWLTESLAAGQPVPMESRHRLEVAEPRKAPPSSIWMPAYACQRPTPLTHHNISLSEALETLAEAAGFEGREGHSLTFLRAASVLRALPRPVVALTQLRGLPHFGEHSFRVVQELLEHGVCEEVERVRHSERFQTMKLFTQIFGVGVRTADRWYQEGLRTLDDLREQPQRLTQQQKAGVQYYQDLSTPVLQPDAEALQQLVEAAVEQVLSGATVTLTGGFRRGKLQGHDVDFLITHPEEGQEAGLLPRVIRCLQDQGLVLYQQYQHSLYGAPGHHSHTMDAFERSFCIFRLPQPPGASVREDPSCPAWKAVRVDLVVAPISQFPFALLGWTGSKLFQRELRRFSRKEKGLCLNSHGLFNPEQNTVFHVASEEDIFRHLGLEYLPPEQRNA

SEQ ID NO: 81 - protein, 571 aa, Otolemur garnettii
MDPRGILKAFPKRKKIHANPSSKALAKIPKREEGEEAGEWLSSLRAHIVPTGIGRARAELFKKQIVQHGGRICPAQDPGVTHIVVDEGMDYERALRLLRLPQLPLGAHLVKSAWLSLCLQERRLVDVAGFSIFIPNRYLDQPQPNKTEQDSTPSVHEAVLMTALSPPSTRPVSPPQKAEEAPSAQPQPISDDETSDSEEMRVSTADLEALISGHYPTPLEGDREPTLAPDHLDKWVCAQSSSQKATNHNLHITEKLEVLAKAYRVQGDKWRALGYAKAINALKSFHKPVTSYQEACSIPGIGKRMAEKIIEILESGHLRKLDHISESVPVLELFSNIWGAGTKTALMWYHQGFRSLEDIRSQASLTIQQAIGLKHYDDFLERMPREEAAEIEQTVREAAHAFNPGLLCVACGSFRRGKVTCGDVDVLITHPDGQSHQGIFTLLLDTLRHQGFLTDDLVSQEENGQQKKYLGVCQLPGPGRRHRRLDIIIVPYSEFACALLYFTGSAHFNRSMRALAKTKGMSLSEHALSTDVVRNTQGFKVGPGRVLPTPTEKDVFRLLGLPYREPAERDW

SEQ ID NO: 82 - protein, 335 aa, Otolemur garnettii
MSKRKAPQETLNGGITDMLTELANFEKNVNQAIHKYNAYRKAASVIAKYPHKIKSGAEAKKLPGVGTKIAEKIDEFLATGKLRKLEKIRQDDTSSSINFLTRVTGIGPSAARKFVDEGIKTLEDLRKNEDKLNHHQRIGLKYFEDFEKRIPREEMLQMQDIVLNEVKKVDSEYIATVCGSFRRGAESSGDMDVLLTHPSFTSESPKQPKLLHRVVEQLQKVLFITDTLSKGETKFMGVCQLPRNSDEREYPHRRIDIRLIPKDQYYCGVLYFTGSDIFNKNMRAHALEKGFTINEYTIRPLGVTGVAGEPLPVDSERDIFEYIQWKYREPKDRSE

SEQ ID NO: 83 - protein, 460 aa, Monodelphis domestica
MASVPLKRRRGRSFGEEAQGAAAPSPPLSRFPEFTLYLAERRMGRMRRAFLTELARGKGFRVDEVYSPQVTHVLMEDASGAEASDYLDRVLGASQSLQKPLLLDISWLTESIGRGKPVPVEAKYCLGIPELLKNQVPPVSMPAYACQRHTPLNHHNFHLTEALETLAEAADFEGSQGRFISFHRAASVLKALPDPITNMSQLHGLPHIGDHSSRIIQELLEHGVSNEVETIKLSKRYQTMKLFTQIFGVGVKTADRWYQEGLRTLDDLQKHSRKLTRQQEAGIHHFEDLNTPVYRHEADAIQQIVEEVVQQMLPGARVILAGGFRRGKPHGHDVDFLITHPVEGLEAGLLSKVMGRLESQGLVLYRHTQSPKNPDNTAFQSTAMDDYEKCFSILWFPKSPTTSSHLEAGESSRDGKAVRVDFVVTPISQFAFALLGWTGSQYFERELRRFSLNREEAAAE

SEQ ID NO: 84 - protein, 545 aa, Monodelphis domestica
MEPRGILKAFPKRKKMRTASDGSGLLKIPKKEKAEAGEWLSPLQVHVLPVGIGRARAEIFEKQIIQHGGQICSPQAPGITHIVVDETVDGERALRLLKLPQLPLGAQLVKSAWLSQCLKEQKLVDTDGFHVVFIPNRYLDKADISFQPGPSGTSAQAGLSSVAPLAPPQEPHSRLGIQAQANSDDEDSDEEEVRVTPDDLEALITGRYPSTPEGDTEPSLAPNLSEKWVCAQPSSQKMTNHNLHITEKLEVLAKAYAVQGDRWRTLGYSKAINALKSFPKPVSSYQEACGIPGIGKRMAEKIMEIVESGHLRKLDHISDSVPILELFSNIWGVGAKTAQMWYQQGFRTLEDIQSQATLSTQQAIGLKHYEDFLKRIPREEAAEIEKTVRETAHTLNPGLLSVACGSYRRGKATCGDVDVLVTHPDGRSHQGIFSQLLDALRKRGFLTDDLVSQDDNGQQQKYLGVCQLPGPGRHHRRLDIIVVPYRSLVKIQSVVTILNWTGNSDCESLLSPICLAIGAGHIHVSLLHYAGFWFLLWFAPPLVPV

SEQ ID NO: 85 - protein, 467 aa, Myotis brandtii
MDSLQMAHSGPRKKRPRQMGASMASPPQDIKFRDLVLFILEKKMGTTRRAFLMELARRKGFRVENELSDSVTHIVAENNSGSDVLEWLQVQKVRASSQPELLDVSWLIECMSAGKPVATTGQHQLVDAFEVLAENCEFRENEGSCLAFMRAASVLKSLPFTIISMKDTEGIPCLGDKVKSIIEEIIEDGESSEVKAVLNDERYQSFKLFTSVFGVGLKTSEKWFRMGFRTLSKIRSDKTLKFTRMQKAGFLYYEDLVSCVTKAEAEAVGVLVKEAVWAFLPDAFVTVTGGFRRGKKIGHDVDFLITSPGSTEEEEQQLLPKVINLWERKGLLLYYDLVESTFEKFKLPSRKVDALDHFQKCFLILKLHHQRVDGGKSSQQEGKTWKAIRVDLVMCPYERRAFALLGWTGSRQFERDLRRYATHERKMMLDNHALYDKTKVEEMRFFYKETEFRNSGEVSVMYPRDLP

SEQ ID NO: 86 - protein, 467 aa, Myotis brandtii
MNSAPLAEPRLAGAEWSRRVAGLGTGSFPLPSSEVTHVVMEQTSAEEAVRWQESRPAPPPGGTHPALLDISWFTESMAAGQPVPVEGRHCLQVAVSREVLPNPVWMPPYACQRPTPLTHHNTSLSEALEMLAEAAGFAGSEGRLLSFSRAASVLKALPCPVTALSQLQGLPHFGGHSCRVIQELLEHGVCEEVERVQRSERYQSMKLFTRIFGVGVRTADQWYREGLRTLDDVWKQVQRLTQQQKAGLQHYQDLSSPVQRPDAEALRQVVEAAVGWALPRATVTLAGGFRRPTLRGKLQGHDVDFLITHPEEGQEVGLLPRVMHYLEQQGLVLYQQHQRSPSGDPARLAPKGHSMDTFEQSFCIFRLPRPPRTAEGGTWSPHPSWKAVRVDLVVAPISQFPFALLGWTGSKHFERELRRFSRKERGLWLNSHGLFDPEQKTFFQAATEEDIFRHLGLAYLPPEQRNA

SEQ ID NO: 87 - protein, 307 aa, synthetic construct
  REGION 1..307: note = Synthetic Sequence
QEIADILEELADLLELLGGNPFRVRAYRKAARALESLPEPIESLEEAKKLPGIGKKIAEKIEEILETGKLRKLEELREDVPPGLLLLLRVPGVGPKTARKLYELGIRTLEDLRKAAGAKLEQNILIGLEYYEDFQQRIPREEALAIAEIIKEALREVDPVLQVEIAGSYRRGKETVGDIDILVTHPDATSRGLLEKVVDALVELGFVTEVLSKGDTKASGILKLPGGWKGRRVDLRVVPPEEFGAALLYFTGSKQFNRALRRLAKEKGLKLNEYGLFDGVDGERLPGETEEEIFEALGLPYIEPELR

SEQ ID NO: 88 - protein, 72 aa, synthetic construct
  REGION 1..72: note = Synthetic Sequence
GLTFVITGDLPSEERDELKELIEKLGGKVTSSVSKKTTHVIVGSDAGPKKLLKAIKLGIPIVTPEWLLDCLK